(19) United States
(12) Patent Application Publication
(10) Pub. No.: US 2005/0143372 A1
(43) Pub. Date: Jun. 30, 2005

(54) Compounds, pharmaceutical compositions and methods of use therefor

(76) Inventors: Shomir Ghosh, Brookline, MA (US); Michael A. Patane, Reading, MA (US); Kenneth G. Carson, Needham, MA (US); I-Cheng Shannon Chi, Redwood City, CA (US); Qing Ye, Marlborough, MA (US); Amy M. Elder, Arlington, MA (US); Tracy J. Jenkins, Belmont, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/490,223

(22) PCT Filed: Oct. 30, 2002
(86) PCT No.: PCT/US02/34845

Related U.S. Application Data
(60) Provisional application No. 60/340,663, filed on Oct. 30, 2001.

(51) Int. Cl. A61K 31/55; A61K 31/445; A61K 31/495

(52) U.S. Cl. 514/218; 514/252.12; 514/317; 514/408; 540/575; 544/399; 546/232; 546/229; 546/230

(57) ABSTRACT

The invention relates to compounds having the formula (I). Preferred compounds are antagonists of C—C chemokine receptor 8. The invention also relates to a method for treating a subjected having an inflammatory disorder or viral disorder comprising administering to a subject in need thereof an effective amount of a compound of the invention.

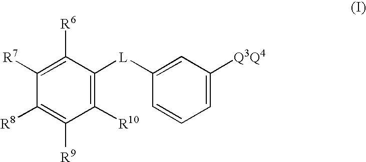

### RELATED APPLICATION

This application claims the benefit of U.S. Provisional Application No. 60/340,663, filed on Oct. 30, 2001, the entire teachings of which are incorporated herein by reference.

### BACKGROUND OF THE INVENTION

Migration of leukocytes from blood vessels into diseased tissues is important to the initiation of normal disease-fighting inflammatory responses. This process is known as leukocyte recruitment. This process, however, also is involved in the onset and progression of inflammatory and autoimmune disease states. The pathology of these diseases results from the attack of the body's immune defenses on normal, healthy tissues. Thus, blocking leukocyte recruitment to target tissues in inflammatory and autoimmune diseases is a desirable therapeutic intervention.

Leukocyte recruitment is mediated at a molecular level by chemoattractant receptors. These receptors are on the surface of leukocytes and bind chemoattractant cytokines that are secreted by cells at the site of the damage or infection. Receptor binding activates leukocytes, increases adhesiveness of the adhesion molecules that mediate transendothelial migration, and promotes directed migration of the cells towards the source of the chemoattractant cytokine.

It has been determined that there is a large family (>20 members) of structurally related chemoattractant cytokines. These molecules share the ability to stimulate cell migration and have been termed chemokines. Each chemokine contains four cysteine residues and two internal disulfide bonds. Chemokines can be grouped into two subfamilies, based on whether the two amino terminal cysteine residues (C) are adjacent to each other (C—C) or are separated by an amino acid (C—X—C).

All of the identified chemokine receptors belong to the seven transmembrane G protein-coupled receptor family (Murdoch and Finn, Blood, 2000, 95:3032). These receptors mediate the binding and signaling of more than one chemokine. To date 18 human chemokine receptors have been identified. Of these receptors, 5 bind C—X—C chemokines (CXC1-CXC5) and 9 purportedly bind C—C chemokines (CCR1-CCR9) (Murdoch and Finn, Blood, 2000, 95:3032). Chemokine receptors also serve as coreceptors for Human Immunodeficiency Virus (HIV) entry into cells. This came from the observation that RANTES, MIP-1α, and MIP-1β suppressed infection of susceptible cells in vitro by macrophage-tropic primary HIV-1 isolates. The chemokine receptor CXCR-4 was found to support infection and cell fusion to CD4+ cells by laboratory-adapted, T-tropic HIV-1 strains.

The human CCR8 receptor has been shown to interact with the human chemokine I-309. This chemokine is a potent monocyte chemoattractant and inhibits apoptosis in thymic cells. The CCR8 receptor is constitutively expressed in monocytes in the spleen and thymus, but not in other peripheral blood leukocytes (Tiffany et al., J. Exp. Med., 1997, 186:165). This data appears to be in agreement with the role of 1-309 in monocyte activation and thymic cell survival. Additionally, CCR8 is preferentially expressed in Th2-polarized cells and is transiently increased after T-cell receptor and C28 engagement, suggesting that CCR8 plays a role in the control of Th2 responses and that up-regulation of CCR8 after antigen encounter may contribute to the proper positioning of activated T-cells within sites of antigenic challenge or specialized areas of lymphoid tissues (Zingoni et al., J. Immunol., 1998, 161:547; D'Ambrosio et al., J. Immunol., 1998, 161:5111). CCR8 also has been shown to serve as a co-receptor for HIV-1.

With the significant role that CCR8 plays in Th2 mediated response, there is a continuing need to develop compounds and pharmaceutical compositions that may be used in the treatment of Th2 inflammatory conditions.

### SUMMARY OF THE INVENTION

The invention relates to compounds that have C—C chemokine receptor antagonizing activity and to a method for treating a subject having an inflammatory or viral disorder (e.g., a chemokine associated disorder, immunological disorder, neurological disorder, viral disorder, asthma) using such compounds. The compounds have the general formula:

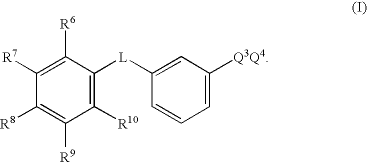

In one aspect, the compound has the formula:

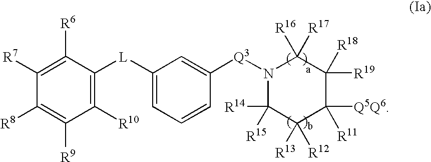

In another aspect, the compound has the formula:

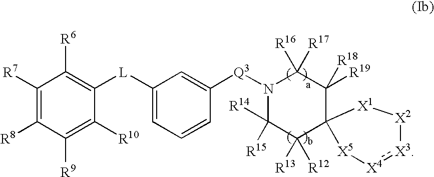

In another aspect, the compound is of Formula I wherein Q⁴ is selected from the group consisting of: 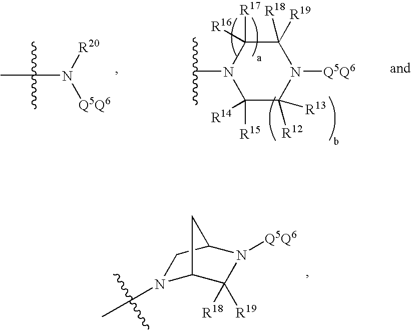
and R⁶ is selected from the group consisting of halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂C(═O)R¹, SO₂, SO₂NR¹R², trifluoromethyl, aryl, aralkyl, heteroaryl and heteroaralkyl.

The invention further relates to a method for treating an inflammatory disorder or viral disorder. The method comprises administering to a subject in need thereof an effective amound of a compound described herein.

The invention further relates to pharamaceutical or physiological compositions comprising a compound as described herein.

The invention further relates to the use of the compounds described herein in therapy (including palliative, curative and prophylactic therapy) or diagnosis, and to the use of such compounds for the manufacture of a medicament for the treatment of a particular disease or condition as described herein (e.g., a chemokine associated disorder, immunological disorder, neurological disorder, viral disorder, asthma).

### DETAILED DESCRIPTION OF THE INVENTION

The invention is directed to novel compounds described herein as compounds of Groups 1 to 11, and to therapeutic methods that employ the compounds described herein.

Group 1

Compounds of Group 1 are defined in Formula (I): 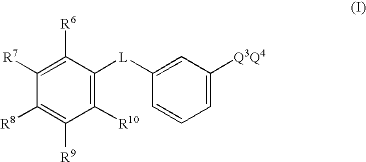
wherein
L is selected from the group consisting of a O, S, NR^{a}, bond, SO₂, —C(═O), and (CR′R″)ₘ;
m is from 1 to 8;
R′ and R″ independently are selected from the group consisting of hydrogen, optionally substituted alkyl, cyano, and optionally substituted alkenyl;
R⁶, R⁷, R⁸, R⁹ and R¹⁰ are independently selected from the group consisting of hydrogen, hydroxyl, halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂C(═O)R¹, SO₂, SO₂NR², trifluoromethyl, aryl, aralkyl, heteroaryl and heteroaralkyl;
R¹ and R² are independently hydrogen or optionally substituted alkyl;
Q³ is selected from the group consisting of a bond, optionally substituted alkyl, optionally substituted alkenyl, alkynyl, —C(═O)—(CH₂)_{c}, (CH₂)_{c}—C(═O), NR^{a}—C(═O)—(CH₂)_{c}, —C(═O)—NR^{a}—(CH₂)_{c}, NR^{a}, O, S, and SO₂;
c is 0, 1 or 2;
R^{a} is selected from hydrogen, optionally substituted alkyl, optionally substituted alkylaryl, or optionally substituted cycloalkyl; and

Q⁴ is selected from the group consisting of hydrogen, optionally substituted aromatic, optionally substituted heteroaromatic, optionally substituted non-aromatic heterocyclic, and optionally substituted amino.

Group 2

The invention is also directed to compounds of Group 1 wherein L is selected from the group consisting of O, NR^{a}, CR′R″ and S. Preferably, L is O.

Group 3

The invention is also directed to compounds of any of Groups 1 or 2, wherein R₆ is selected from the group consisting of halogen and C₁-C₁₀ alkoxy; and R₇-R₁₀ are hydrogen. Preferably, the C₁-C₁₀ alkoxy is a methoxy and the halogen is a chloro. Thus, in some compounds of Group 3, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

Group 4

Compounds of Group 4 are compounds of Groups 1 to 3, wherein Q³ is selected from the group consisting of a bond or optionally substituted alkyl. Thus, in some compounds of Group 4, Q³ is selected from the group consisting of a bond or optionally substituted alkyl and L is selected from the group consisting of O, NR^{a}, CR′R″ and S (preferably L is O). In other compounds of Group 4, Q³ is selected from the group consisting of a bond or optionally substituted alkyl, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

Group 5

The invention is also directed to compounds of Group 5, which are compounds of Groups 1 to 4, wherein Q³ is —CH₂— or CR′R″. Thus, in preferred compounds of Group 4, Q³ is —CH₂— or CR′R″ and L is selected from the group consisting of O, NR^{a}, CR′R″ and S (preferably L is O). In more preferred compounds of the invention, Q³ is —CH₂— or CR′R″, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred compounds of Group 5, R′ is cyano or methyl and R″ is hydrogen. Thus, in preferred compounds of Group 4, Q³ is —CH₂— or CR′R″ (wherein R′ is cyano or methyl and R″ is hydrogen), and L is selected from the group consisting of O, NR^{a}, CR′R″ or S (preferably L is O). In more preferred compounds of Group 5, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

Group 6

The invention is also directed to compounds of any of Groups 1 to 5, wherein Q⁴ is selected from 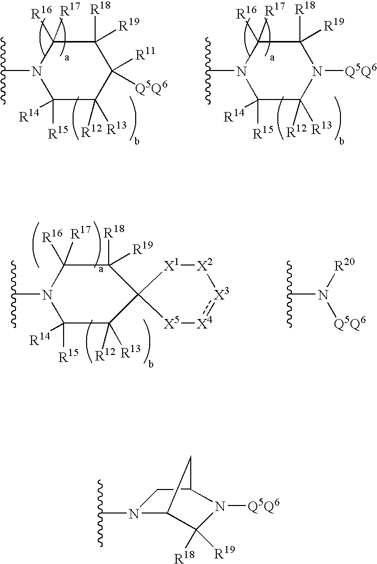
wherein
a is 0 to 3;
b is 0 to 3;
R¹¹, R¹², R¹³, R¹⁴, R¹⁵, R¹⁶, R¹⁷, R¹⁸, and R¹⁹ are each independently chosen from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂C(═O)R⁴¹, SO₂, SO₂NR⁴¹R⁴², trifluoromethyl, aryl, aralkyl, heteroaryl and heteroaralkyl;
any two of R¹², R¹³, R¹⁴ and R¹⁵ may be taken together to form an optionally substituted carbocyclic group optionally interrupted by one or more heteroatoms;
any two of R¹⁶, R¹⁷, R¹⁸, R¹⁹ may be taken together to form an optionally substituted carbocycle optionally interrupted by one or more heteroatoms; or
R¹⁸ or R¹⁹ together with Q⁵Q⁶ and the atoms to which they are bonded form an optionally substituted non-aromatic carbocyclic group, optionally substituted non-aromatic heterocyclic group, optionally substituted aryl ring or optionally substituted heteroaryl ring;
R²⁰ is selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂C(═O)R⁴¹, SO₂, SO₂NR⁴¹R⁴², trifluoromethyl, aryl, aralkyl, heteroaryl and heteroaralkyl;
X¹ is independently selected from the group consisting of CR²⁶R²⁷, NR²⁸, —(C═O), O and a bond;
X² is independently selected from the group consisting of CR²⁹R³⁰, NR³¹, —(C═O) and 0;
X³ is independently selected from the group consisting of CR³²R³³, —C(R³²)═, NR³⁴, ——N═, —(C═O) and 0;
X⁴ is independently selected from the group consisting of CR³⁵R³⁶, NR³⁷, ═N—, —(C═O) and O;
X⁵ is independently selected from the group consisting of CR³⁸R³⁹, NR⁴⁰, —(C═O) and O;
with the proviso that R³⁵ and R³⁸ or R³² and R³⁵ may be joined together via an optionally substituted C₁₋₆ alkyl bridge that may be optionally interrupted by one or more heteroatoms to form a non-aromatic carbocyclic or heterocyclic group, or R³⁵ and R³⁸ or R³² and R³⁵ may be joined together via an optionally substituted C₁₋₆ alkenyl bridge that may be optionally interrupted by one or more heteroatoms to form an aromatic ring;
R²⁶, R²⁷, R²⁹, R³⁰, R³², R³³, R³⁵, R³⁶, R³⁸ and R³⁹ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂C(═O)R⁴¹, SO₂, SO₂NR⁴¹R⁴², trifluoromethyl, aryl, aralkyl, heteroaryl and heteroaralkyl;
with the proviso that when X⁴ is CR³⁵R³⁶ and X³ is CR³²R³³ or X⁵ is CR³⁸R³⁹, R³⁵ and R³⁸ or R³² and R³⁵ optionally form a non-aromatic carbocyclic group, a non-aromatic heterocyclic group, aryl ring or heteroaryl ring;
R²⁸, R³¹, R³⁴, R³⁷ and R⁴⁰ are each independently selected from the group consisting of hydrogen, alkyl, SO₂R⁴³, aryl, and benzyl; hydrogen, hydroxyl, halogen, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido-C(═O)O(R⁴¹), —C(═O)(R⁴¹), —SO₂NR⁴¹R⁴², trifluoromethyl, aryl, aralkyl, heteroaryl or heteroaralkyl
Q⁵ is selected from the group consisting of a bond, —C(R⁴¹R⁴²)_{d}—C(═O)—NR⁴³—, (NR⁴³)_{z}—C(═O)—C(R⁴¹R⁴²)_{d}—C(═O)—C(R⁴¹R⁴²)_{d}—, —(CR⁴¹R⁴²)_{d}—C(═O)—, —(NR⁴³)_{z}—C(═O)—NR⁴⁴, —(NR⁴³)_{z}—C(═S)—NR⁴⁴—, —(NR⁴³)_{z}—C(═O)—CH═CH—, —(NR⁴³)_{z}—C(R⁴¹R⁴²)_{d}—, —C(═O)—CH═CH—CH₂—, —C(═O)—CH═CH—, —(NR⁴³)_{z}—C(R⁴¹R⁴²)_{d}—, —C(R⁴¹R⁴²)_{d}—NR⁴³—, —SO₂(CR⁴¹R⁴²)_{d}—, —(CR⁴¹R⁴²)^{d}—, —(R⁴³)_{z}—C(═O)—(CR⁴¹R⁴²)_{d}—, —C(═O)—(CR⁴¹R⁴²)_{d}—, —C(═O)—(CR⁴¹R⁴²)_{d}—O—, or is absent;
d is 0, 1, 2, 3, 4, 5, 6, 7 or 8;
z is 0 or 2;
R⁴¹, R⁴², R⁴³ and R⁴⁴ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴⁵), —C(═O)(R⁴⁵), —SO₂C(═O)R⁴⁵, SO₂, SO₂R⁴⁵, SO₂NR⁵⁴R⁵⁵, trifluoromethyl, aryl, aralkyl, heteroaryl and heteroaralkyl;
or R⁴¹ and R⁴² may be linked via a C₂-C₈ optionally substituted alkyl or alkenyl bridge where one or more carbons may be replaced by O, S, NR⁵⁴ or NR⁴⁶;
R⁵⁴ and R⁵⁵ are independently selected from the group consisting of hydrogen, optionally substituted alkyl, optionally substituted alkylaryl, optionally substituted cycloalkyl, —SO₂—(C₁₋₁₀ optionally substituted alkyl), —SO₂—(C₂₋₁₀ optionally substituted alkenyl, —SO₂—(C₂₋₁₀ optionally substituted alkynyl), —SO₂-aryl, optionally substituted aryl, and —SO₂-heteroaryl;
R⁴⁵ is selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted aryloxy, optionally substituted amino, optionally substituted amido, —O(CF₃), —C(═O)O(R⁴¹), —C(═O)(R⁴¹), —SO₂C(═O)R⁴¹, —SO₂, —SO₂NR⁴¹R⁴², trifluoromethyl, aryl, benzyl, aralkyl, heteroaryl and heteroaralkyl;
Q⁶ is an optionally substituted aromatic ring, optionally substituted non-aromatic heterocycle, optionally substituted alkyl, or an optionally substituted heteraromatic ring.

In preferred compounds of Group 6, Q 4 is as defined above, and Q³ is —CH₂— or CR′R″. In more preferred compounds of Group 6, Q⁴ is as defined above, Q³ is —CH₂— or CR′R″, and L is selected from the group consisting of O, NR^{a}, CR′R″ and S (preferably L is O). In more preferred compounds of the invention, Q⁴ is as defined above, Q³ is —CH₂— or CR′R″, L is selected from the group consisting of O, NR^{a}, CR′R″ and S (preferably L is O), and (a) R⁶ is alkoxy and R⁷-R¹⁰ are hydrogen; or (b) R⁶ is alkoxy and R⁷-R¹⁰ are hydrogen.

In more preferred compounds of Group 6, Q⁴ is as defined above and Q³ is —CH₂- or CR′R″ (wherein R′ is cyano or methyl and R″ is hydrogen). In additional preferred compounds, Q⁴ is as defined above, Q³ is —CH₂— or CR′R″ (wherein R′ is cyano or methyl and R″ is hydrogen), and L is selected from the group consisting of O, NR^{a}, CR′R″ and S (preferably L is O). In more preferred compounds of Group 6, Q⁴ is as defined above, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy and R⁷-R¹⁰ are hydrogen; or (b) R⁶ is alkoxy and R⁷-R¹⁰ are hydrogen.

Group 7

Compounds of Group 7 are compounds of any of Groups 1 to 6, wherein Q⁴¹ is

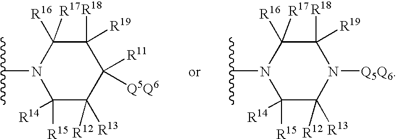

In preferred compounds of Group 7, Q⁴ is as defined above, and Q³ is —CH₂— or CR′R″. In more preferred compounds of Group 7, Q⁴ is as defined above, Q³ is —CH₂— or CR′R″, and L is selected from the group consisting of O, NR^{a}, CR′R″ and S (preferably L is O). In more preferred compounds of Group 7, Q⁴ is as defined above, Q³ is —CH₂— or CR′R″, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred compounds of Group 7, Q⁴ is as defined above and Q³ is —CH₂— or CR′R″ (wherein R′ is cyano or methyl and R″ is hydrogen). In additional preferred compounds, Q⁴ is as defined above, Q³ is —CH₂— or CR′R″ (wherein R′ is cyano or methyl and R″ is hydrogen), and L is selected from the group consisting of O, NR^{a}, CR′R″ and S (preferably L is O). In more preferred compounds of Group 7, Q⁴ is as defined above, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred embodiments of Group 7 as described above, Q⁴ is 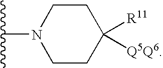
Group 8

The compounds of Group 8 are compounds of any of Groups 1 to 6, in which Q⁴ is selected from the group consisting of:

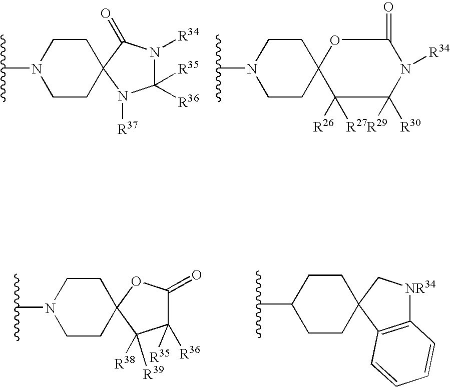

In preferred compounds of Group 8, Q⁴ is as defined above, and Q³ is —CH₂— or CR′R″. In more preferred compounds of Group 8, Q⁴ is as defined above, Q³ is —CH₂— or CR′R″, and L is selected from the group consisting of O, NR^{a}, CR′R″ and S (preferably L is O). In more preferred compounds of Group 8, Q⁴ is as defined above, Q³ is —CH₂— or CR′R″, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred compounds of Group 8, Q⁴ is as defined above and Q³ is —CH₂— or CR′R″ (wherein R′ is cyano or methyl and R″ is hydrogen). In additional preferred compounds, Q⁴ is as defined above, Q³ is —CH₂— or CR′R″ (wherein R′ is cyano or methyl and R″ is hydrogen), and L is selected from the group consisting of O, NR^{a}, CR′R″ and S (preferably L is O). In more preferred compounds of Group 8, Q⁴ is as defined above, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In preferred embodiments of Group 8, R³⁴ is SO₂Re. More preferably, R³⁴ is SO₂R⁴⁴ and R⁴⁴ is alkyl.

Group 9

The compounds of Group 9 are compounds of any of Groups 1 to 8, wherein Q⁵ is selected from the group consisting of: 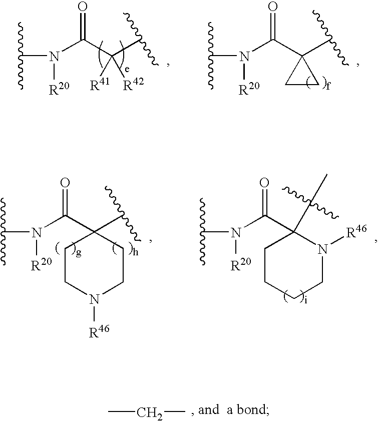
wherein
e is 1 to 3;
f is 1 to 7;
g is 0 to 3;
h is 0 to 3;
i is 0 or 1;
R²⁰ and R⁴⁶ are independently hydrogen, hydroxyl, halogen, optionally substituted alkyl, optionally substituted alkenyl, optionally substituted alkynyl, optionally substituted cycloalkyl, optionally cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂C(═O)R⁴¹, SO₂, SO₂NR⁴¹R⁴², trifluoromethyl, aryl, aralkyl, heteroaryl or heteroaralkyl.

In preferred embodiments of Group 9, Q⁵ is as defined above, Q⁴ is as defined in Group 7, and Q³ is —CH₂— and CR′R″. In more preferred compounds of Group 9, Q⁵ is as defined above, Q⁴ is as defined in Group 7, Q³ is —CH₂— or CR′R″, and L is selected from the group consisting of O, NR^{a}, CR′R″ and S (preferably L is O). In more preferred compounds of Group 9, Q⁵ is as defined above, Q⁴ is as defined in Group 7, Q³ is —CH₂— or CR′R″, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred compounds of Group 9, Q⁵ is as defined above, Q⁴ is as defined in Group 7 and Q³ is —CH₂— or CR′R″ (wherein R′ is cyano or methyl and R″ is hydrogen). In additional preferred compounds, Q⁵ is as defined above, Q⁴ is as defined in Group 7, Q³ is —CH₂— or CR′R″ (wherein R′ is cyano or methyl and R″ is hydrogen), and L is selected from the group consisting of O, Na, CR′R″ and S (preferably L is O). In more preferred compounds of Group 9, Q⁵ is as defined above, Q⁴ is as defined in Group 7, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In additional preferred embodiments of Group 9, Q⁵ is as defined above, Q⁴ is as defined in Group 8, and Q³ is —CH₂— or CR′R″. In more preferred compounds of Group 9, Q⁵ is as defined above, Q⁴ is as defined in Group 8, Q³ is —CH₂— or CR′R″, and L is selected from the group consisting of O, NR^{a}, CR′R″ and S (preferably L is O). In more preferred compounds of Group 9, Q⁵ is as defined above, Q⁴ is as defined in Group 8, Q³ is —CH₂— or CR′R″, L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In more preferred compounds of Group 9, Q⁵ is as defined above, Q⁴ is as defined in Group 8 and Q³ is —CH₂— or CR′R″ (wherein R′ is cyano or methyl and R″ is hydrogen). In additional preferred compounds, Q⁵ is as defined above, Q⁴ is as defined in Group 8, Q³ is —CH₂— or CR′R″ (wherein R′ is cyano or methyl and R″ is hydrogen), and L is selected from the group consisting of O, NR^{a}, CR′R″ and S (preferably L is O). In more preferred compounds of Group 9, Q⁵ is as defined above, Q⁴ is as defined in Group 8, Q³ is —CH₂— or CR′R″ (wherein R′ is cyano or methyl and R″ is hydrogen), L is selected from the group consisting of O, NR^{a}, CR′R″ and S (preferably L is O), and (a) R⁶ is alkoxy (preferably methoxy) and R⁷-R¹⁰ are hydrogen; or (b) R⁶ is halogen (preferably chloro) and R⁷-R¹⁰ are hydrogen.

In one embodiment of Group 9, Q⁵ is

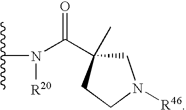

In an alternative embodiment of Group 9, Q⁵ is 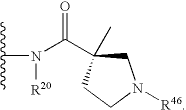
Group 10

The Group 10 compounds of the invention are compounds of Groups 6 to 9, wherein Q⁶ is selected from the group consisting of: 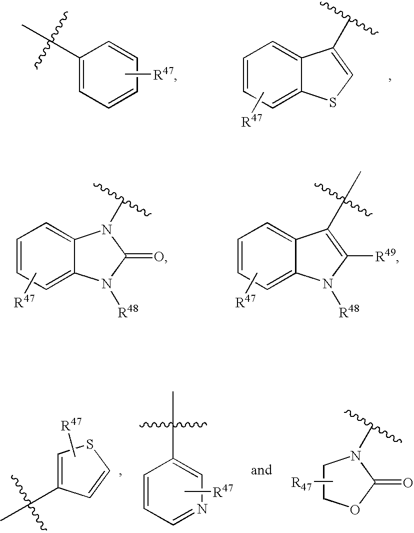
wherein
the selected group can be substituted with one or more substitutents, R⁴⁷, which are chosen independently for each position capable of substitution from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)⁴¹), —SO₂C(═O)R⁴¹, SO₂, SO₂NR⁴¹R⁴², trifluoromethyl, aryl, aralkyl, heteroaryl and heteroaralkyl;
R⁴⁸ is selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, —C(═O)O(R⁴¹), —C(═O)(R⁴¹), trifluoromethyl, aryl, aralkyl, heteroaryl and heteroaralkyl; and
R⁴⁹ is selected from the group consisting of hydrogen, hydroxyl, halogen, optionally substituted alkyl, optionally substituted alkenyl, optionally substituted alkynyl, optionally substituted cycloalkyl, optionally cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂C(═O)R⁴¹, SO₂, SO₂NR⁴¹R⁴², trifluoromethyl, aryl, aralkyl, heteroaryl and heteroaralkyl.

In one embodiment of Group 10 compounds, Q⁶ is 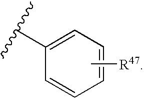
Group 11

The compounds of Group 11 are compounds of Group 10, wherein R⁴⁷ is chosen independently for each position capable of substitution from the group consisting of hydrogen, chloro, bromo, fluoro, iodo, CF₃, phenyl, —S(O)₂—N-alkyl, alkyl, and

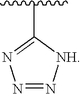

Certain compounds of the invention contain stereocenters and may be obtained as different stereoisomers (e.g., diastereomers and enantiomers). For example, as described above, in certain embodiments Q5 is 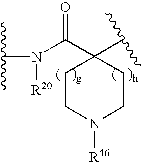
and when g is 1 and h is zero, Q5 can have a formula selected from 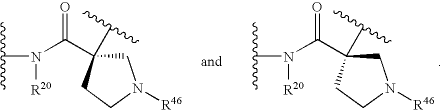
It is pointed out that the invention includes all isomeric forms and racemic mixtures of the disclosed compounds, and a method of treating a subject with both pure isomers and mixtures thereof, including racemic mixtures. Stereoisomers can be separated and isolated using any suitable method, such as chromatography. One stereoisomer may be more active than another. The desired isomer can be determined, for example, by screening.

In a particular aspect, the invention is a compound of Formula I wherein Q⁴ is 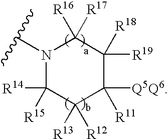
The compounds of this aspect have the formula 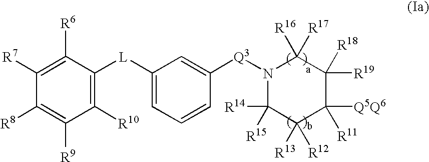
wherein Q³ is optionally substituted alkyl; and Q⁵ is selected from the group consisting of 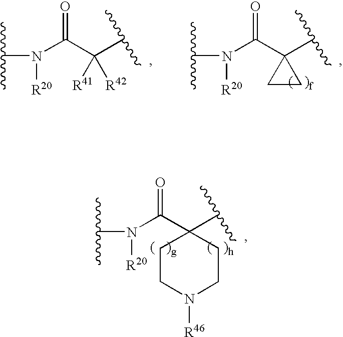
—CH₂—, and a bond.
R⁶-R¹⁰, R¹¹-R¹⁹, R⁴¹, R⁴², R⁴⁶, L, a, b, f, g, h and Q⁶ are as defined above for Formula I. I certain embodiments, R⁴⁶ is a substituted alkyl, such as —CH(CH₂)(CH₂)—COOH. In other embodiments, a is 1, b is 1, R¹² and R¹³ are methyl, and R¹⁴, R¹⁵, R¹⁶, R¹⁷, R¹⁸ and R¹⁹ are hydrogen. In another embodiment, the compound is defined by Formula Ia with the proviso that the compound is not

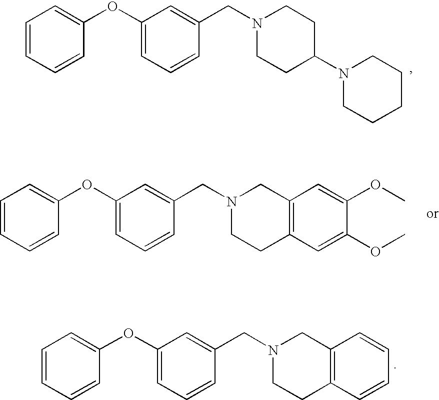

In another aspect, the invention is a compound of formula I wherein Q⁴ is 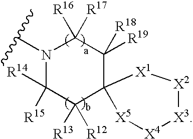
The compounds of this aspect have the formula 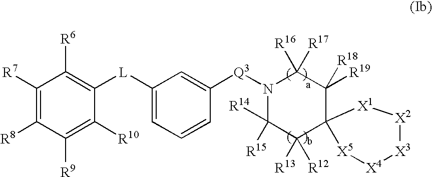
wherein Q³ is optionally substituted alkyl; and R⁶-R¹⁰, R¹²-R¹⁹, a, b, X¹, X², X³, X⁴ and X⁵ are as defined above for Formula I.

In another aspect, the invention is a compound of Formula I wherein Q⁴ is selected from the group consisting of 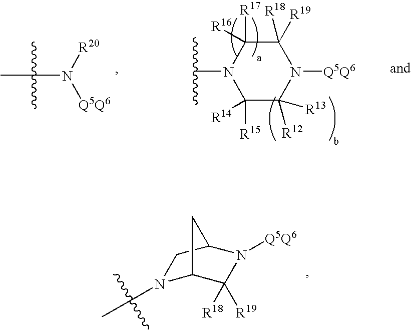
and R⁶ is selected from the group consisting of halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂C(═O)R¹, SO₂, SO₂NR¹R², trifluoromethyl, aryl, aralkyl, heteroaryl and heteroaralkyl.

Particularly preferred compounds are selected from the group consisting of

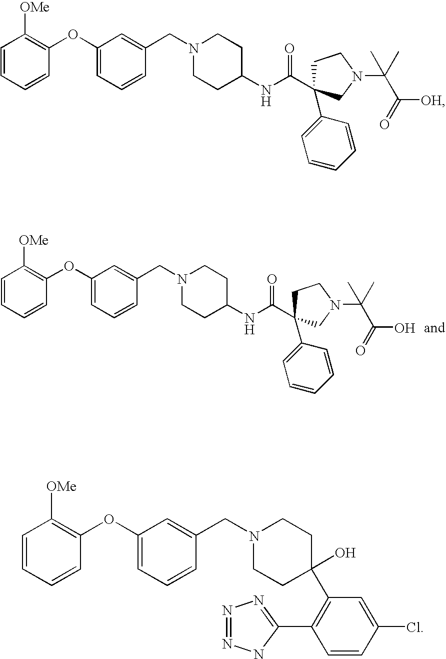

The present invention further contemplates a method for treating a chemokine associated disorder in a subject comprising administration to said subject an effective amount of any of the compounds of the invention as defined above. Preferably the chemokine associated disorder is treated through modulation of a β-chemokine receptor.

The present invention also contemplates methods where the chemokine associated disorder is a neurological disorder, immunological disorder, chemokine associated disorder is characterized by unwanted cellular proliferation, unwanted cellular migration, abnormal cellular signal transduction, abnormal amounts of chemokine stimulated chemotaxis, or a viral disorder.

In one embodiment the disorders are selected from the group consisting of Alzheimer's disease, dementias related to Alzheimer's disease, Parkison's disease, Lewy diffuse body disease, multiple sclerosis, amytrophic lateral sclerosis, progressive supranuclear palsy, epilepsy, Jakob-Creuztfeldt disease, stroke, traumatic injury to the brain, traumatic injury to the spinal cord, spinal crush, central nervous system trauma, peripheral nervous system trauma, immune thyroiditis, hyperthyriodism, type I diabetes mellitus, insulin related diabetes, Addison's disease, autoimmune oophoritis, autoimmune orchiitis, autoimmune hemolytic anemia, paroxysmal cold hemoglobinuria, autoimmune thrombocytopenia, autimmune neutropenia, pernicious anemia, autoimmune coagulopathies, myasthenia gravis, allerigic encephalomyelitis, pemphigus, bullous diseases, rheumatic carditis, Goodpasture's syndrome, T-cell leukemia, postcardiotomy syndrome, arthritis, rheimatoid arthritis, osteoarthritis, keratitis, parotitis, polymositis, dermatomyositis, scleroderma, acquired immune deficiency syndrome, lupus, multiple sclerosis, restinosis, idiopathic pulmonary fibrosis, allergic hypersensitivity disorders, allergic rhinitis, psoriasis, chronic contact dermatitis, sarcoidosis, dermatomyositis, skin pemphigoid, pemphigus vulgaris, p. foliacius, p. erthematosus, glomerulonephritides, vasculitides, cutaneous vasculitis, hypersensitivity vasculitis, hepatitis, systemic lupus erthematosus, myasthenia gravis, dermatitis, eczema, atopic dermatitis, allergic contact dermatitis, uricaria, reperfusion injury, transplant rejection, graft rejection, allograft rejection, artherosclerosis, asthma, inflammatory bowel disease, Crohn's disease, ulcerative colitis, arthritis, osteoarthritis, and rheumatoid arthritis.

The present invention also contemplates a pharmaceutical composition comprising a therapeutically effective amount of a compound of Formula (I) and a pharmaceutically or physiologically acceptable carrier, where the effective amount is effective to treat a chemokine associated disorder. The “effective amount” of a compound is an amount, sufficient to achieve a desired therapeutic and/or prophylactic effect, such as an amount which results in the prevention of or a decrease in the symptoms associated with an inflammatory or viral disorder. For example, the effective amount can result in the inhibition of one or more processes mediated by the binding of a chemokine to a receptor in a subject with an inflammatory or viral disorder. Examples of such processes include leukocyte migration, integrin activation, transient increases in the concentration of intracellular free calcium [Ca²⁺]ᵢ and granule release of proinflammatory mediators.

The invention contemplates a method for treating an inflammatory disease in a subject comprising administration to said subject an effective amount of a compound as defined above. Preferably, the inflammatory disease is a neurological disorder, an immunological disorder, or a viral disorder.

The disorders may be selected from the group consisting of Alzheimer's disease, dementias related to Alzheimer's disease, Parkison's disease, Lewy diffuse body disease, multiple sclerosis, amytrophic lateral sclerosis, progressive supranuclear palsy, epilepsy, Jakob-Creuztfeldt disease, stroke, traumatic injury to the brain, traumatic injury to the spinal cord, spinal crush, central nervous system trauma, peripheral nervous system trauma, immune thyroiditis, hyperthyriodism, type I diabetes mellitus, insulin related diabetes, Addison's disease, autoimmune oophoritis, autoimmune orchiitis, autoimmune hemolytic anemia, paroxysmal cold hemoglobinuria, autoimmune thrombocytopenia, autimmune neutropenia, pernicious anemia, autoimmune coagulopathies, myasthenia gravis, allerigic encephalomyelitis, pemphigus, bullous diseases, rheumatic carditis, Goodpasture's syndrome, postcardiotomy syndrome, arthritis, rheimatoid arthritis, osteoarthritis, keratitis, parotitis, polymositis, dermatomyositis, scleroderma, acquired immune deficiency syndrome, lupus, multiple sclerosis, restinosis, idiopathic pulmonary fibrosis, allergic hypersensitivity disorders, allergic rhinitis, psoriasis, chronic contact dermatitis, sarcoidosis, dermatomyositis, skin pemphigoid, pemphigus vulgaris, p. foliacius, p. erthematosus, glomerulonephritides, vasculitides, cutaneous vasculitis, hypersensitivity vasculitis, hepatitis, systemic lupus erthematosus, myasthenia gravis, dermatitis, eczema, atopic dermatitis, allergic contact dermatitis, uricaria, reperfusion injury, transplant rejection, graft rejection, allograft rejection, artherosclerosis, asthma, inflammatory bowel disease, Crohn's disease, ulcerative colitis, arthritis, osteoarthritis, and rheumatoid arthritis.

The invention also contemplates a pharmaceutical composition comprising a therapeutically effective amount of a compound of the invention as defined above and a pharmaceutically or physiologically acceptable carrier, where the effective amount is effective to treat an inflammatory disease or a viral disorder.

The present invention further contemplates pharmaceutical compositions comprising at least one compound encompassed by the formula of the present invention. Alternatively, the pharmaceutical composition may comprise a salt or prodrug of at least one compound encompassed by the formula of the present invention. The pharmaceutical composition comprises an inflammatory treating effective amount of at least one compound of the present invention. In a specific embodiment, the present invention contemplates a pharmaceutical composition comprising a CCR8 antagonist or anti-viral effective amount of at least one compound of the present invention. Dosage unit forms containing the pharmaceutical composition of the present invention also are provided.

Another embodiment of the present invention is a method of inhibiting CCR8 in a patient in need thereof by administering a CCR8 antagonist effective amount of the pharmaceutical composition of the present invention. In a specific embodiment, the compounds of the present invention are contemplated for the use of treating an inflammatory or anti-viral condition, which encompasses those conditions that are described below.

Definitions

As used herein, “optionally substituted” means that the chemical group that immediately follows the phrase is unsubstituted or “substited” as described herein. For example, an optionally substituted C₁-C₁₀ alkyl is a C₁-C₁₀ alkyl or a substituted C₁-C₁₀ alkyl.

As used herein, the term “substituted” means that the radical is substituted with one or more substituents selected from the group consisting of carboxy, hydroxy, alkyl, alkenyl, alkynyl, cycloalkyl, cycloalkenyl, cycloalkynyl, alkoxy, alkenyloxy, alkynyloxy, cycloalkoxy, cycloalkenyloxy, cycloalkynyloxy, nitro, halogen, cyano, amino, (di)alkyl amino, (di)alkenyl amino, (di)aryl amino, aryl, substituted aryl, non-aromatic heterocyclic, substituted non-aromatic heterocyclic, heteroaryl, substituted heteroaryl, aralkyl, heteroaralkyl, acyl, acyloxy, sulfonamide, sulfonyl, oxo, —SO₃H, —CHO—, —(CH₂)ₙ—NH₂, —(CH₂)ₙ—NH-alkyl, —(CH₂), —N(alkyl)₂, wherein n is an integer from 1 to 8.

As used herein, the term “alkyl” refers to a straight or branched hydrocarbon group having from one to twelve carbon atoms and a single radical. Suitable alkyl groups include, but are not limited to, methyl, ethyl, n-propyl, isopropyl, n-butyl, sec-butyl, tert-butyl, pentyl, isopentyl, neopentyl, hexyl, heptyl, octyl and the like. In some embodiments, preferred alkyl groups are those having from one to eight carbon atoms, and more preferred alkyl groups are those having from one to four carbon atoms. Any alkyl group (or alkyl moiety) may be substituted with one or more substituents independently selected for each position.

As used herein, the term “alkenyl” refers to a straight or branched hydrocarbon group that contains from one to twelve carbon atoms and a single radical, and has one or more double bonds between carbon atoms. Suitable alkenyl groups include, e.g., n-butenyl, cyclooctenyl and the like. In some embodiments, preferred alkenyl groups are those having from one to eight carbon atoms, and more preferred alkenyl groups are those having from one to four carbon atoms. Any alkenyl group (or alkenyl moiety) may be substituted.

As used herein, the term “alkynyl” refers to a straight or branched hydrocarbon group that contains from one to twelve carbon atoms and a single radical, and has one or more triple bonds between carbon atoms. Suitable alkynyl groups include, e.g., n-butynyl. In some embodiments, preferred alkynyl groups are those having from one to eight carbon atoms, and more preferred alkynyl groups are those having from one to four carbon atoms. Any alkynyl group (or alkynyl moiety) may be substituted.

As used herein, the term “cycloalkyl” means a non-aromatic mono or multicyclic hydrocarbon ring system of from 3 to 12 carbon atoms having a single radical. Preferred monocyclic cycloalkyl groups are those having from 3 to 6 carbon atoms, for example, cyclopropyl, cyclobutyl, cyclopentyl and cyclohexyl. The cycloalkyl group (or cycloalkyl moiety) as defined herein may optionally be substituted.

As used herein, the term “cycloalkenyl” means a non-aromatic mono or multicyclic hydrocarbon ring system of from 3 to 12 carbon atoms having a single radical and at least one C═C. Preferred cycloalkynyl groups are those having from 3 to 6 carbon atoms. The cycloalkenyl group (or cycloalkenyl moiety) as defined herein may optionally be substituted.

As used herein, the term “cycloalkynyl” means a non-aromatic mono or multicyclic hydrocarbon ring system of from 3 to 12 carbon atoms having a single radical and at least one C═C. Preferred cycloalkynyl groups are those having from 3 to 6 carbon atoms. The cycloalkynyl group (or cycloalkynyl moiety) as defined herein may optionally be substituted.

As used herein, the term “alkoxy” refers to the group —O-alkyl, wherein the alkyl moiety is as defined above. In some embodiments, preferred alkoxy groups are those having 1 to 8 carbon atoms. Suitable alkoxy groups include methoxy, ethoxy, propoxy, butoxy, and the like.

As used herein, the term “alkenyloxy” is a group —O-alkenyl, wherein the alkenyl moiety is as defined above. In some embodiments, preferred alkenyloxy groups are those having 1 to 8 carbon atoms in the alkenyl moiety.

As used herein, the term “alkynyloxy” is a group —O-alkynyl, wherein the alkynyl moiety is as defined above. In some embodiments, preferred alkynyloxy groups are those having 1 to 8 carbon atoms in the alkynyl moiety.

As used herein, the term “acyl” is a group —RC(═O)—, wherein R may be an alkyl, alkenyl, alkynyl, aryl, amino, amino alkyl, amino alkenyl, amino alkynyl or amino aryl moiety, as those terms are defined herein. In some embodiments, preferred alkyl, alkenyl and alkynyl R groups are those having from one to eight carbon atoms, more preferably from one to five carbon atoms. Exemplary aryl R groups are phenyl and naphthyl.

As used herein, the term “acyloxy” is a group O-acyl, wherein the acyl moiety is as described above.

As used herein, the term “sulfonamide” refers to the group SO₂NH—.

As used herein, the term “sulfonyl” refers to the group SO₂—.

As used herein, the term “halo” or “halogen” encompasses fluorine, chlorine, bromine and iodine.

As used herein, the term “ring system” refers to an aromatic or non-aromatic carbocyclic compound, in which one or more of the ring carbon atoms may be replaced by a heteroatom, such as nitrogen, oxygen or sulfur.

As used herein, the term “fused ring system” refers to ring systems wherein at least two adjacent carbon centers join one or more cyclic structures. A fused ring system as used herein may be aromatic or non-aromatic, or may be composed of separate aromatic and non-aromatic moieties.

As used herein, the term “spirocyclic” refers to a ring system in which a ring has one carbon atom in common with a second cyclic group.

As used herein, the term “polycyclic ring system” refers to ring systems having two or more cyclic compounds bonded in tandem. A polycyclic ring system as used herein may be aromatic or non-aromatic, or may be composed of separate aromatic and non-aromatic moieties.

As used herein, the term “non-aromatic heterocyclic” means a closed ring structure having from about five to about fifteen atoms in the ring, in which one or more of the atoms in the ring is an atom other than carbon, such as oxygen, nitrogen or sulfur. A heterocyclic group may be a fused or polycyclic ring system. Examples of suitable non-aromatic heterocyclic groups and substituted heterocyclic groups include, but are not limited to, piperidine, piperazine, pyrrolidine, imidazoline, tetrahydrofuranyl, tetrahydrothiophenyl, morpholino, tetrahydroquinoline and tetrahydroisoquinoline.

As used herein, the term “aryl” means an aromatic carbocyclic ring structure having from about five to about fifteen carbon atoms. An aryl group may be a fused or polycyclic ring system. Examples of suitable aryl groups include, phenyl, naphthyl and anthracyl.

As used herein, the term “heteroaryl” means a closed aromatic ring structure having from about five to about fifteen atoms in the ring, in which one or more of the atoms in the ring is an atom other than carbon, such as oxygen, nitrogen or sulfur. Examples of suitable heteroaryl groups and substituted heteroaryl groups include, but are not limited to, indole, quinoline, thiophene, pyridine, imidazole, quinoline, isoquinoline, benzothiophene, oxazole, benzimidazole, imidazole, tetrazole and azepine.

As used herein, the terms “arylalkyl” and “aralkyl” are used interchangeably, and refer to -alkyl-aryl, wherein the “alkyl” and “aryl” moieties are as defined herein.

As used herein, the term “heteroarylalkyl” and “heteroaralkyl” are used interchangeably, and refer to -alkyl-heteroaryl, wherein the “alkyl” and “heteroaryl” moieties are as defined herein.

The term “protected hydroxy” or “protected carboxy” refers to the use of a “hydroxy protecting group,” a substituent of a hydroxy group that is commonly employed to block or protect the hydroxy functionality (including the hydroxy functionality of a carboxyl group) while reactions are carried out on other functional groups on the compound. Examples of such hydroxy protecting groups include tetrahydropyranyl, 2-methoxyprop-2-yl, 1-ethoxyeth-1-yl, methoxymethyl, O-methoxyethoxymethyl, methylthiomethyl, t-butyl, t-amyl, trityl, benzyl, trimethylsilyl, and the like.

The term “protected amino” refers to the use of an “amino protecting group,” a substituent of an amino group that is commonly employed to block or protect the amino functionality or reactions that are carried out on the compounds.

As used herein, the term “amino” refers to the group —NH₂.

As used herein, the term “N-substituted amino” refers to an amino group in which the N atom of the amino group is once substituted. A non-limiting example of a suitable N-substituted amino groups includes alkylamino.

As used herein, the term “N,N-substituted amino” refers to an amino group in which the N atom of the amino group is twice substituted. Suitable N,N-substituted amino groups include dialkylamino.

As used herein, the symbol “—” represents a chemical bond. The symbol “ 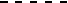
” represents an optional chemical bond, such that the symbol “ 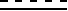
” indicates that the linked atoms can be joined by either a single or a double bond.

As used herein, the term “patient” refers to any animal (e.g., mammals, birds, fish) in need of therapy, such as humans, cows, dogs, cats, sheep, horses, chickens, pigs and the like. In an embodiment of this invention, the patient is in need of treatment of an inflammatory condition.

The compounds described herein can be prepared and administered as neutral compounds, salts, esters, amides and/or prodrugs. As used herein, the phrase “pharmaceutically or physiologically acceptable salts, esters, amides, and prodrugs” refers to those salts (e.g., carboxylate salts, amino acid addition salts), esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The compounds described herein can form pharmaceutically or physiologically acceptable acid addition and/or base salts. All of these forms are within the scope of the present invention.

Pharmaceutically or physiologically acceptable salts of the compounds described herein include salts derived from nontoxic inorganic acids such as hydrochloric, nitric, phosphoric, sulfuric, hydrobromic, hydroiodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, and the like. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate, gluconate, galacturonate and the like (see, for example, Berge S. M. et al., “Pharmaceutical Salts,” J. of Pharma. Sci., 1977;66:1).

Acid addition salts of compounds which contain a basic group (e.g., amine) can be prepared using suitable methods. For example, acid addition salts can be prepared by contacting the free base form of a compound with a sufficient amount of a desired acid to produce the salt in the conventional manner. The free base form can be regenerated by contacting the salt form with a base and isolating the free base in the conventional manner. The free base form of a compound can differ from a salt forms somewhat in certain physical properties such as solubility in polar solvents.

Pharmaceutically or physiologically acceptable base addition salts can be formed with suitable metals or amines, such as alkali and alkaline earth metals or organic amines. Examples of metals which are suitable for use as cations in base addition salts include sodium, potassium, magnesium, calcium and the like. Amines suitable for use as cations in base addition salts include N,N-dibenzylethylenediamine, chloroprocaine, choline, diethanolamine, dicyclohexylamine, ethylenediamine, N-methylglucamine, and procaine (see, for example, Berge, supra, 1977).

Base addition salts of compounds which contain an acidic group (e.g., carboxylic acid) can be prepared using suitable methods. For example, the free acid form of a compound can be contacted with a sufficient amount of the desired base to produce a salt in the conventional manner. The free acid form can be regenerated by contacting the salt form with a suitable acid and isolating the free acid in the conventional manner. The free acid form of a compound can differ from the base addition salt form somewhat in certain physical properties such as solubility in polar solvents.

Examples of pharmaceutically or physiologically acceptable, nontoxic esters of the compounds of this invention include C₁-C₆ alkyl esters. In certain embodiments, the alkyl group of the alkyl ester is a straight or branched chain C₁-C₆ alkyl group. Acceptable alkyl esters also include C₅-C₇ cycloalkyl esters as well as arylalkyl esters such as, but not limited to benzyl. C₁-C₄ esters are preferred. Esters of the compounds of the present invention can be prepared using any suitable method.

Examples of pharmaceutically acceptable, nontoxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines, and C₁-C₂ dialkyl secondary amines are preferred. Amides of the compounds of the invention may be prepared using any suitable method.

The term “prodrug” refers to compounds that can be transformed in vivo (e.g., following administration to an animal), by metabolic processes or other processes, to yield a compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

The terms “viral condition”, “viral disease”, or “viral disorder” refer to either an acute or chronic viral condition, which results from infectious causes. In particular embodiments, the viral condition, viral disease or viral disorder is associated with infection by simian immunodeficiency virus (SIV) or human immunodeficiency virus (HIV-1, HIV-2, including M-trophic and/or T-trophic strains), papilloma virus (e.g., human papilloma virus 16); flaviviruses such as Hepatitis B and Hepatitis C; Herpes virus (e.g., Herpes simplex virus (HSV-1, HSV-2), cytomegalovirus, Epstein-Barr virus, varicella-zoster virus, human herpes virus (e.g., HHV6, HHV7, HHV8,) herpes viruses which infect livestock, such as horses, cattle, pigs, chickens, turkeys and fish (e.g., pseudorabies virus, porcine cytomegalovirus)); parvovirus (e.g., parvo virus B19), human influenza virus A, human influenza virus B, rhinovirus, coronaviruses, enterovirus, human parainfluenza virus, respiratory syncytial virus (RSV), adenovirus (e.g., adenovirus-8), togavirus (e.g., rubella virus), paramyxovirus (e.g., Measles virus, Mumps virus), rhabdoviruses (e.g., rabies virus, molola virus, vesicular stomatitis virus), rotavirus, enteric calicivirus (e.g., Norwalk virus), enterovirus (e.g., coxsackievirus, echovirus, poliovirus), reovirus, lymphocyte choriomeningitis virus, bunyamwera virus, group C virus, tahyna virus, toscana virus, punta toro virus, dengue virus, orbivirus (e.g., Orungo virus, Tribec virus, Kemerova virus, Lipovnik virus), encephalitis viruses (e.g., California encephalitis virus, La Crosse encephalitis virus, St. Loius encephalitis virus, West Nile virus, eastern equine encephalitis virus, Japanese encephalitis virus), for example.

The terms “inflammatory condition”, “inflammatory disease”, or “inflammatory disorder” refer to either an acute or chronic inflammatory condition, which can result from infections or non-infectious causes. Various infectious conditions include meningitis, encephalitis, uveitis, colitis, dermatitis, and adult respiratory distress syndrome. Non-infectious causes include trauma (burns, cuts, contusions, crush injuries), autoimmune diseases, and organ rejection episodes. Thus, in specific embodiments, an inflammatory condition results from a condition selected from the group that includes: atherosclerosis (arteriosclerosis); autoimmune conditions, such as multiple sclerosis, systemic lupus erythematosus, polymyalgia rheumatica (PMR), rheumatoid athritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis, inflammatory bowel disease including Croin's Disease (regional enteritis) and ulcerative colitis, pernicious anemia, inflammatory dermatoses; usual interstitial pneumonitis (UI), asbestosis, silicosis, berylliosis, talcosis, the various forms all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis, with inflammatory edema; immediate hypersensitivity reactions including, but not limited to, asthma, hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute cholecystitis; and conditions producing transient ischemia anywhere along the gastrointestinal tract, bladder, heart, or other organ, especially those prone to rupture; sequelae of organ transplantation or tissue allograft, including allograft rejection in the acute time period following allogeneic organ or tissue transplantation and chronic host-versus-graft rejection.

As used herein, the term “treat” refers to reducing or completely removing an undesired condition. Therefore, as used in the context of the present invention the term means to reduce an inflammatory condition or to completely remove the condition. Assessment of the efficacy of the treatment may be determined by anyone of ordinary skill in the art using methods that are well known and identified.

Therapeutic Methods

The invention also provides methods for treating or preventing inflammatory conditions, by administration of at least one therapeutic of the invention. Such therapeutics include the aforementioned molecules, oligopeptides, proteins, and combinations thereof.

While not wishing to be bound by any particular theory or mechanism, it is believed that compounds of the invention are antagonists of a chemokine receptor. Preferably, the compounds antagonize the CCR8, and that therapeutic benefits derived from the method of the invention are the result of antagonism of CCR8 function. Thus, the compounds of the invention can be used to treat a patient having a condition involving cells which express CCR8 on their surface and which respond to signals transduced through CCR8, as well as the specific conditions recited herein.

To enhance the efficacy of the therapeutics contained in the invention, these treatments may be administered in conjunction with other therapies which block the function of other molecules involved in the inflammatory or viral pathway.

The subjects to which the present invention is applicable may be any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

Pharmaceutical Compositions

The invention also relates to pharmaceutical and/or physiological compositions which contain the compounds described herein. Such compositions can contain adjuvants such as preserving, wetting, emulsifying, and dispensing agents. Prevention of the action of microorganisms can be controlled by various antibacterial and antifungal agents, for example, parabens, chlorobutanol, phenyl, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound can be admixed with at least one inert customary excipient (or carrier) such as sodium citrate or dicalcium phosphate; or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol, and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Such solid compositions or solid compositions that are similar to those described can be employed as fillers in soft- and hard-filled gelatin capsules using excipients such as lactose or milk sugar as well as high molecular weight polyethyleneglycols, and the like.

Solid dosage forms such as tablets, dragees, capsules, pills, and granules can be prepared with coatings and shells, such as enteric coatings or other suitable coatings or shells. Several such coating and/or shells are well known in the art, and can contain opacifying agents, and can also be of such composition that they release the active compound or compounds in a certain part of the intestinal tract in a delayed manner. Examples of embedding compositions which can be used are polymeric substances and waxes. The active compounds can also be used in microencapsulated form, if appropriate, with one or more of the above-mentioned excipients.

Liquid dosage forms for oral administration include pharmaceutically or physiologically acceptable emulsions, solutions, suspensions, syrups, and elixirs. In addition to the active compounds, the liquid dosage forms can contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. If desired, the composition can also include adjuvants, such as wetting agents, emulsifying and suspending agents, sweetening, flavoring and/or perfuming agents.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

Suspensions, in addition to the active compounds, can contain suspending agents, such as, ethoxylated isostearyl alcohols, polyoxyethylene sorbitol and sorbitan esters, microcrystalline cellulose, aluminum metahydroxide, bentonite, agar-agar, tragacanth, and the like. Mixtures of suspending agents can be employed if desired. Compositions for rectal administrations are preferably suppositories which can be prepared by mixing the compounds of the present invention with suitable nonirritating excipients or carriers such as cocoa butter, polyethyleneglycol, or a suppository wax which are solid at ordinary temperatures but liquid at body temperature and therefore, melt in the rectum or vaginal cavity and release the active component.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Dosage forms for topical administration of a compound of this invention include ointments, powders, sprays and inhalants. The active component can be admixed under suitable conditions (e.g., sterile conditions) with a physiologically acceptable carrier and any preservatives, buffers, or propellants as may be required. Ophthalmic formulations, eye ointments, powders, and solutions are also contemplated as being within the scope of this invention.

The present invention further provides aerosol formulations and dosage forms. In general such dosage forms contain the compounds of the present invention in a pharmaceutically or physiologically acceptable diluent. Pharmaceutically or physiologically acceptable diluents include but are not limited to sterile water, saline, buffered saline, dextrose solution, and the like. In a specific embodiment, a diluent that may be used in the present invention or the pharmaceutical formulation of the present invention is phosphate buffered saline, or a buffered saline solution generally between the pH 7.0-8.0 range or water. The present invention further contemplates liquid aerosol formulations comprising the compound of the present invention and another therapeutically effective drug. It is also contemplated that the present aerosol formulation can be prepared as a dry powder formulation comprising a finely divided powder form of the compound and a dispersant.

The liquid aerosol formulation of the present invention may include, as optional ingredients, pharmaceutically or physiologically acceptable carriers, diluents, solubilizing or emulsifying agents, surfactants and excipients.

The formulations of the present embodiment may also include other agents useful for pH maintenance, solution stabilization, or for the regulation of osmotic pressure. Examples of the agents include but are not limited to salts, such as sodium chloride, or potassium chloride; and carbohydrates, such as glucose, galactose or mannose, and the like.

The present invention further contemplates dry powder formulations comprising the compound of the present invention and another therapeutically effective drug. Formulations for dispensing from a powder inhaler device will comprise a finely divided dry powder containing the compound of the present invention (or derivative) and may also include a bulking agent, such as lactose, sorbitol, sucrose, or mannitol in amounts which facilitate dispersal of the powder from the device, e.g., 50 to 90% by weight of the formulation.

The quantity of active component in a unit dose preparation may be varied or adjusted from 1 mg to about 1000 mg, according to the particular application and the potency of the active component. The composition can, if desired, also contain other compatible therapeutic agents. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

Administration

The pharmaceutical compositions of the present invention may be administered by a variety of routes such as intravenous, intratracheal, subcutaneous, oral, parenteral, buccal, sublingual, opthalmic, pulmonary, transmucosal, transdermal, and intramuscular. Unit dosage forms also can be administered in intranasal form via topical use of suitable intranasal vehicles, or via transdermal routes, using those forms of transdermal skin patches known to those of ordinary skill in the art. The pharmaceutical composition or unit dosage forms of the present invention may be administered to an animal, preferably a human being, in need of treatment of an inflammatory condition.

The pharmaceutical composition or unit dosage form of the present invention may be administered according to a dosage and administration regimen defined by routine testing in light of the guidelines given above in order to obtain optimal anti-inflammatory or anti-viral activity while minimizing toxicity or side-effects for a particular patient. However, such fine turning of the therapeutic regimen is routine in light of the guidelines given herein. The dosage of the active agents of the present invention may vary according to a variety of factors such as underlying disease state, the individual's condition, weight, sex and age and the mode of administration.

For combination therapy according to the invention, the active agents may initially be provided as separate dosage forms until an optimum dosage combination and administration regimen is achieved. The exact dosage and administration regimen utilizing the combination therapy of the present invention is selected in accordance with a variety of factors including type, species, age, weight, sex and medical condition of the patient; the severity and etiology of the inflammatory condition to be treated; the route of administration; the renal and hepatic function of the patient; the treatment history of the patient; and the responsiveness of the patient. Optimal precision in achieving concentrations of active agents within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein. The pharmaceutical composition or unit dosage form may be administered in a single daily dose, or the total daily dosage may be administered in divided doses. In addition, co-administration or sequential administration of other active agents may be desirable.

In a specific embodiment, pulmonary delivery of the present compounds (or derivatives thereof) is contemplated. The compounds (or derivative) are delivered to the lungs of a mammal while inhaling and traverses across the lung epithelial lining to the blood stream. Other reports of this include Adjei et al. Pharmaceutical Research, 7:565-569 (1990); Adjei et al., International Journal of Pharmaceutics, 63:135-144 (1990) (leuprolide acetate); Braquet et al., Journal of Cardiovascular Pharmacology, 13(suppl. 5): 143-146 (1989) (endothelin-1); Hubbard et al., Annals of Internal Medicine, Vol. 1, pp. 206-212 (1989) (α1-antitrypsin); Smith et al., J. Clin. Invest. 84:1145-1146 (1989) (α-1-proteinase); Oswein et al., “Aerosolization of Proteins”, Proceedings of Symposium on Respiratory Drug Delivery II, Keystone, Colo., March, (1990) (recombinant human growth hormone); Debs et al., J. Immunol. 140:3482-3488 (1988) (interferon-γ and tumor necrosis factor alpha); Platz et al., U.S. Pat. No. 5,284,656 (granulocyte colony stimulating factor)). A method and composition for pulmonary delivery of drugs for systemic effect is described in U.S. Pat. No. 5,451,569.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

All such devices require the use of formulations suitable for the dispensing of compounds of the present invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compounds may also be prepared in different formulations depending on the type of chemical modification or the type of device employed.

Formulations suitable for use with a nebulizer, either jet or ultrasonic, will typically comprise the compound of the present invention (or derivative) dissolved in water. The formulation may also include a buffer and a simple sugar (e.g., for stabilization and regulation of osmotic pressure). The nebulizer formulation may also contain a surfactant, to reduce or prevent surface induced aggregation of the compounds caused by atomization of the solution in forming the aerosol.

Formulations for use with a metered-dose inhaler device will generally comprise a finely divided powder containing the compounds (or derivative) suspended in a propellant with the aid of a surfactant. The propellant may be any conventional material employed for this purpose, such as a chloro fluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

The liquid aerosol formulations contain the compounds of the present invention and a dispersing agent in a physiologically acceptable diluent. The dry powder aerosol formulations of the present invention consist of a finely divided solid form of the compounds of the present invention and a dispersing agent. With either the liquid or dry powder aerosol formulation, the formulation must be aerosolized. That is, it must be broken down into liquid or solid particles in order to ensure that the aerosolized dose actually reaches the mucous membranes of the nasal passages or the lung. The term “aerosol particle” is used herein to describe the liquid or solid particle suitable for nasal or pulmonary administration, i.e., that will reach the mucous membranes. Other considerations, such as construction of the delivery device, additional components in the formulation, and particle characteristics are important. These aspects of nasal or pulmonary administration of a drug are well known in the art, and manipulation of formulations, aerosolization means and construction of a delivery device require at most routine experimentation by one of ordinary skill in the art.

Often, the aerosolization of a liquid or a dry powder formulation for inhalation into the lung will require a propellant. The propellant may be any propellant generally used in the art. Specific nonlimiting examples of such useful propellants are a chlorofluorocarbon, a hydro fluorocarbon, a hydochloro fluorocarbon, or a hydrocarbon, including trifluoromethane, dichlorodifluoromethane, dichlorotetrafluoroethanol, and 1,1,1,2-tetrafluoroethane, or combinations thereof.

In a specific embodiment, the dosage is administered as needed. One of ordinary skill in the art can readily determine a volume or weight of aerosol corresponding to this dosage based on the concentration of compound in an aerosol formulation of the invention.

The compounds and compositions of the present invention may be combined with other compounds and compositions, known to one of ordinary skill in the art, to treat any of the above described disease states. For example, compounds of the present invention may be administered prior, concurrently, or after administration of another compound. For example, the compounds of the present invention may be used in combination with compounds used in the treatment of AIDS and HIV. A non-comprehensive list of ADS and HIV drugs are shown in WO 00/42045.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents, based upon 100% total weight of the dosage.

Synthetic Method

Compounds of the present invention may be prepared according to the methods described herein by various synthetic schemes (as shown in the Experimental Section of the application). Alternatively, the compounds may be prepared by any method known to one of ordinary skill in the art.

### EXAMPLES

General. All reactions involving air-sensitive reagents were performed under a nitrogen atmosphere. Reagents were used as received from commercial suppliers unless otherwise noted. ¹H NMR data were recorded using the Bruker UltraShield 300 MHz/54 mm instrument equipped with Bruker B-ACS60 Auto Sampler or the Varian 300 MHz instrument. Intermediates and final compounds were purified by flash chromatography using one of the following instruments: 1. Biotage 4-channel Quad UV Flash Collector equipped with a Quad 1 Pump Module and the Quad 12/25 Cartridge module. 2. Biotage 12-channel Quad UV Flash Collector equipped with a Quad 3 Pump Module and a Quad 3 Cartridge module. 3. ISCO combi-flash chromatography instrument. LC/MS spectra were obtained using a MicroMass Platform LC (Phenomenx C18 column, 5 micron, 50×4.6 mm) equipped with a Gilson 215 Liquid Handler. Standard LC/MS conditions is as follows:

Formic acid-Standard conditions:

| | |
|---|---|
| % C (Water) | 95.0 |
| % D (Acetonitrile) | 5.0 |
| % Formic Acid | 0.1 |
| Flow (ml/min) | 3.500 |
| Stop Time (mins) | 4.4 |
| Min Pressure (bar) | 0 |
| Max Pressure (bar) | 400 |
| Oven Temperature Left(° C.) | 25.0 |
| Oven Temperature Right(° C.) | 25.0 |

HP1100 LC Pump Gradient Timetable
The gradient Timetable contains 5 entries which are:

| Time | A % | B % | C % | D % | Flow | Pressure |
|---|---|---|---|---|---|---|
| 0.00 | 0.0 | 0.0 | 95.0 | 5.0 | 3.500 | 400 |
| 3.50 | 0.0 | 0.0 | 0.0 | 100.0 | 3.500 | 400 |
| 4.30 | 0.0 | 0.0 | 0.0 | 100.0 | 3.500 | 400 |
| 4.40 | 0.0 | 0.0 | 95.0 | 5.0 | 4.000 | 400 |
| 5.00 | 0.0 | 0.0 | 95.0 | 5.0 | 4.000 | 400 |

LC-MS data were acquired using the “Formic acid-Standard” method unless otherwise noted.

### Preparation of 3-aryloxybenzaldehyde

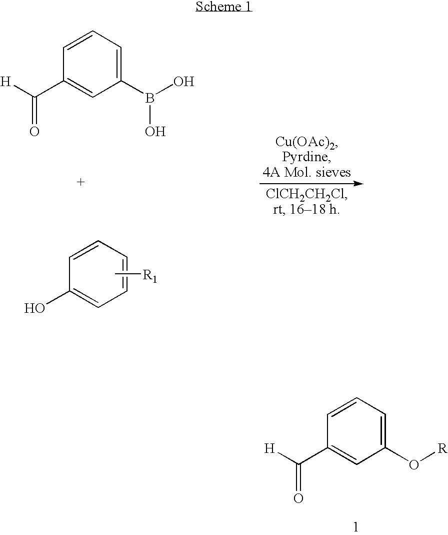

3-Formyl phenyl boronic acid (1.5 equiv) and the appropriate phenyl (1.0 equiv) was mixed with copper acetate (1.0 equiv), 4 Å molecular sieves and pyridine (5.0 equiv) in dichloroethane (0.1 M solution) and the resulting mixture was stirred vigorously for 18 h at ambient atmosphere and room temperature. The reaction mixture was filtered and concentrated. Column chromatography of the residue using hexane/ethyl acetate provided the corresponding 3-aryloxybenzaldehyde 1.

TABLE 1

| | |
|---|---|
| | 1 |
| 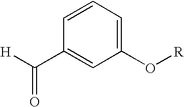 | |

| No | | R |
|---|---|---|
| 1-1 | 3-(2-Methoxy-phenoxy)-benzaldehyde | 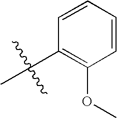 |
| 1-2 | 3-(2-Isopropyl-phenoxy)-benzaldehyde | 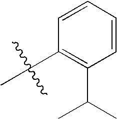 |
| 1-3 | 3-(2-Isopropoxy-phenoxy)-benzaldehyde | 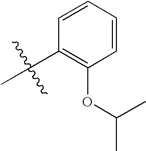 |
| 1-4 | 2-(3-Formyl-phenoxy)-benzoic acid methyl ester | 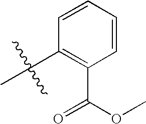 |
| 1-5 | 3-(2-Methoxy-4-propenyl-phenoxy)-benzaldehyde | 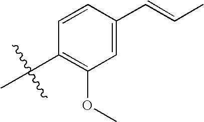 |
| 1-6 | 3-(2-Chloro-phenoxy)-benzaldehyde | 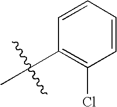 |
| 1-7 | 3-(2-Methylsulfanyl-phenoxy)-benzaldehyde | 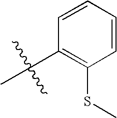 |
| 1-8 | 3-(2-Trifluoromethyl-phenoxy)-benzaldehyde | 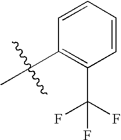 |
| 1-9 | 3-(2,6-Dimethyl-phenoxy)-benzaldehyde | 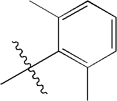 |
| 1-10 | 3-(2,6-Dimethoxy-phenoxy)-benzaldehyde | 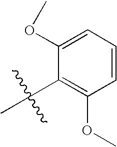 |
| 1-11 | 3-(2-tert-Butyl-phenoxy)-benzaldehyde | 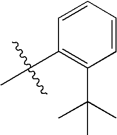 |
| 1-12 | 3-(2-Trifluoromethoxy-phenoxy)-benzaldehyde | 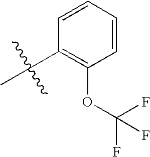 |
| 1-13 | 3-(2-Benzyloxy-phenoxy)-benzaldehyde | 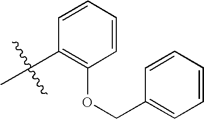 |
| 1-14 | 3-(2-Cyclopentyloxy-phenoxy)-benzaldehyde | 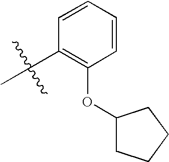 |
| 1-15 | 3-(2-Bromo-phenoxy)-benzaldehyde | 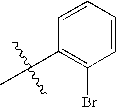 |
| 1-16 | 3-(2-Ethyl-phenoxy)-benzaldehyde | 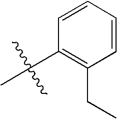 |
| 1-17 | 3-(4-Fluoro-2-methoxy-phenoxy)-benzaldehyde | 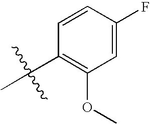 |
| 1-18 | 2-(3-Formyl-phenoxy)-benzonitrile | 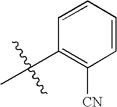 |
| 1-19 | 3-(2-Isoxazol-5-yl-phenoxy)-benzaldehyde | 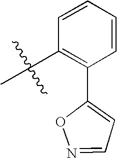 |
| 1-20 | 3-(2-Allyloxy-phenoxy)-benzaldehyde | 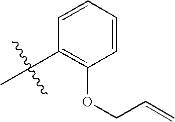 |
| 1-21 | 3-(2-Methoxy-5-methyl-phenoxy)- benzaldehyde | 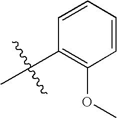 |
| 1-22 | 4-(3-Formyl-phenoxy)-3-methoxy-benzonitrile | 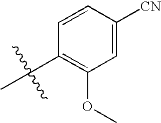 |

### Preparation of 3-aryloxy-benzyl Amines

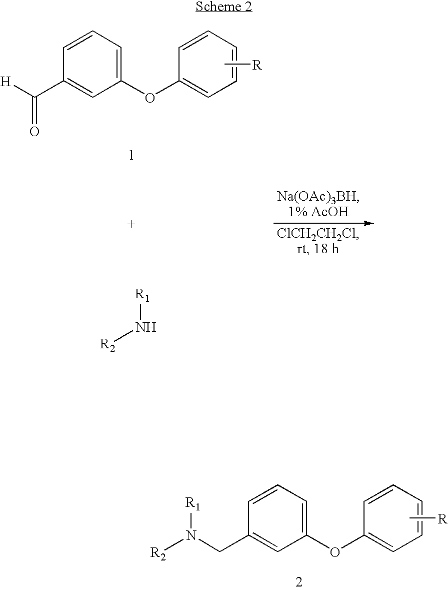

3-aryloxy benzaldehyde 1 was mixed with an approriate amine (1.2 eq.) and sodium triacetoxy borohydride (1.2 eq.) in dichloroethane containing acetic acid (1%) and the resulting mixture was stirred at room temperature overnight. The reaction mixture was diluted with CH₂Cl₂ and washed with saturated aqueous sodium bicarbonate solution and brine and dried over sodium sulfate. Column chromatography provided the corresponding 3-aryloxy-benzyl amine 2. 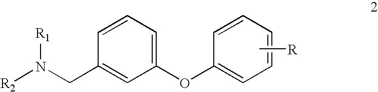
2-1: 8-[3-(2-Methoxy-phenoxy)-benzyl]-1-phenyl-1,3,8-triaza-spiro[4.5]decan-4-one
2-2: 8-[3-(2-Isopropyl-phenoxy)-benzyl]-1-phenyl-1,3,8-triaza-spiro[4.5]decan-4-one
2-3: 1-{1-[3-(2-Isopropyl-phenoxy)-benzyl]-piperidin-4-yl}-1,3-dihydro-benzoimidazol-2-one
2-4: 1-{1-[3-(2-Isopropoxy-phenoxy)-benzyl]-piperidin-4-yl}-1,3-dihydro-benzoimidazol-2-one
2-5: 8-[3-(2-Isopropoxy-phenoxy)-benzyl]-1-phenyl-1,3,8-triaza-spiro[4.5]decan-4-one
2-6: 2-{3-[4-(2-Oxo-2,3-dihydro-benzoimidazol-1-yl)-piperidin-1-ylmethyl]-phenoxy}-benzoic acid methyl ester
2-7: 2-[3-(4-Oxo-1-phenyl-1,3,8-triaza-spiro[4.5]dec-8-ylmethyl)-phenoxy]-benzoic acid methyl ester
2-8: 1-{1-[3-(2-Methoxy-4-propenyl-phenoxy)-benzyl]-piperidin-4-yl}-1,3-dihydro-benzoimidazol-2-one
2-9: 8-[3-(2-Methoxy-4-propenyl-phenoxy)-benzyl]-1-phenyl-1,3,8-triaza-spiro[4.5]decan-4-one
2-10: 1-{1-[3-(2-Chloro-phenoxy)-benzyl]-piperidin-4-yl}-1,3-dihydro-benzoimidazol-2-one
2-11: 8-[3-(2-Chloro-phenoxy)-benzyl]-1-phenyl-1,3,8-triaza-spiro[4.5]decan-4-one
2-12: 8-[3-(2-Methylsulfanyl-phenoxy)-benzyl]-1-phenyl-1,3,8-triaza-spiro[4.5]decan-4-one
2-13: 1-{1-[3-(2-Methylsulfanyl-phenoxy)-benzyl]-piperidin-4-yl}-1,3-dihydro-benzoimidazol-2-one
2-14: 1-Phenyl-8-[3-(2-trifluoromethyl-phenoxy)-benzyl]-1,3,8-triaza-spiro[4.5]decan-4-one
2-15:1-{1-[3-(2-Trifluoromethyl-phenoxy)-benzyl]-piperidin-4-yl}-1,3-dihydro-benzoimidazol-2-one
2-16: 1-{1{-[3-(2,6-Dimethyl-phenoxy)-benzyl]-piperidin-4-yl}-1,3-dihydro-benzoimidazol-2-one
2-17: 1-{1-[3-(2,6-Dimethoxy-phenoxy)-benzyl]-piperidin-4-yl}-1,3-dihydro-benzoimidazol-2-one
2-18: 8-[3-(2,6-Dimethoxy-phenoxy)-benzyl]-1-phenyl-1,3,8-triaza-spiro[4.5]decan-4-one
2-19: 1-{1-[3-(2-tert-Butyl-phenoxy)-benzyl]-piperidin-4-yl}-1,3-dihydro-benzoimidazol-2-one
2-20: 1-Phenyl-8-[3-(2-trifluoromethoxy-phenoxy)-benzyl]-1,3,8-triaza-spiro[4.5]decan-4-one
2-21: 1-{1-[3-(2-Trifluoromethoxy-phenoxy)-benzyl]-piperidin-4-yl}-1,3-dihydro-benzoimidazol-2-one
2-22: 8-[3-(2-Benzyloxy-phenoxy)-benzyl]-1-phenyl-1,3,8-triaza-spiro[4.5]decan-4-one
2-23: 1-{1-[3-(2-Benzyloxy-phenoxy)-benzyl]-piperidin-4-yl}-1,3-dihydro-benzoimidazol-2-one
2-24: 8-[3-(2-Cyclopentyloxy-phenoxy)-benzyl]-1-phenyl-1,3,8-triaza-spiro[4.5]decan-4-one
2-25: 1-{1-[3-(2-Cyclopentyloxy-phenoxy)-benzyl]-piperidin-4-yl}-1,3-dihydro-benzoimidazol-2-one
2-26: 8-[3-(2-Bromo-phenoxy)-benzyl]-1-phenyl-1,3,8-triaza-spiro[4.5]decan-4-one
2-27: 1-{[1-[3-(2-Bromo-phenoxy)-benzyl]-piperidin-4-yl}-1,3-dihydro-benzoimidazol-2-one
2-28: 8-[3-(2-Ethyl-phenoxy)-benzyl]-1-phenyl-1,3,8-triaza-spiro[4.5]decan-4-one
2-29: 1-{1-[3-(2-Ethyl-phenoxy)-benzyl]-piperidin-4-yl}-1,3-dihydro-benzoimidazol-2-one
2-30: 8-[3-(4-Fluoro-2-methoxy-phenoxy)-benzyl]-1-phenyl-1,3,8-triaza-spiro[4.5]decan-4-one
2-31: 1-{1-[3-(4-Fluoro-2-methoxy-phenoxy)-benzyl]-piperidin-4-yl}-1,3-dihydro-benzoimidazol-2-one
2-32: 2-[3-(4-Oxo-1-phenyl-1,3,8-triaza-spiro[4.5]dec-8-ylmethyl)-phenoxy]-benzonitrile
2-33: 2-{3-[4-(2-Oxo-2,3-dihydro-benzoimidazol-1-yl)-piperidin-1-ylmethyl]-phenoxy}-benzonitrile
2-34: 8-[3-(2-Isoxazol-5-yl-phenoxy)-benzyl]-1-phenyl-1,3,8-triaza-spiro[4.5]decan-4-one
2-35: 1-{1-[3-(2-Isoxazol-5-yl-phenoxy)-benzyl]-piperidin-4-yl}-1,3-dihydro-benzoimidazol-2-one
2-36: 8-[3-(2-Allyloxy-phenoxy)-benzyl]-1-phenyl-1,3,8-triaza-spiro[4.5]decan-4-one
2-37:1-{1-[3-(2-Allyloxy-phenoxy)-benzyl]-piperidin-4-yl}-1,3-dihydro-benzoimidazol-2-one
2-38: 3-Methoxy-4-[3-(4-oxo-1-phenyl-1,3,8-triaza-spiro[4.5]dec-8-ylmethyl)-phenoxy]-benzonitrile
2-39: 3-Methoxy-4-{3-[4-(2-oxo-2,3-dihydro-benzoimidazol-1-yl)-piperidin-1-ylmethyl]-phenoxy}-benzonitrile
2-40: 8-(3-Phenoxy-benzyl)-1-phenyl-1,3,8-triaza-spiro[4.5]decan-4-one
2-41: 1-[1-(3-Phenoxy-benzyl)-piperidin-4-yl]-1,3-dihydro-benzoimidazol-2-one

2-42:1-{1-[3-(2-Methoxy-phenoxy)-benzyl]-piperidin-4-yl}-1,3-dihydro-benzoimidazol-2-one

TABLE 2

| No. | R | 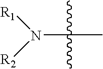 | Retention time (min) | M + 1 |
|---|---|---|---|---|
| 2-1 | 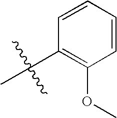 | 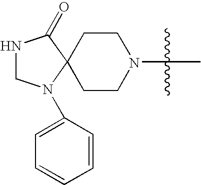 | 1.74 | 444 |
| 2-2 | 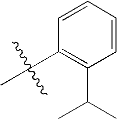 | 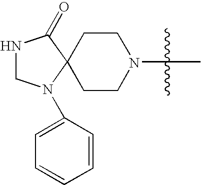 | 2.01 | 456.17 |
| 2-3 | 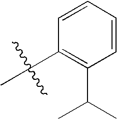 | 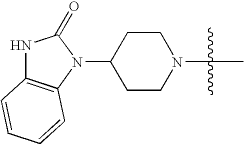 | 1.97 | 442.18 |
| 2-4 | 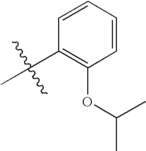 | 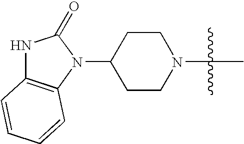 | 1.81 | 458.12 |
| 2-5 | 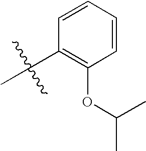 | 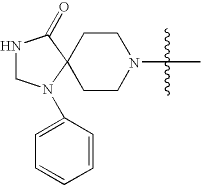 | 1.85 | 472.17 |
| 2-6 | 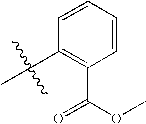 | 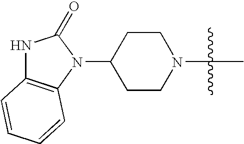 | 1.65 | 458.08 |
| 2-7 | 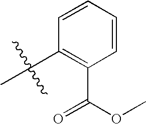 | 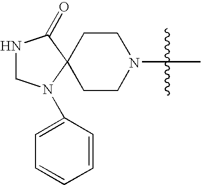 | 1.76 | 472.1 |
| 2-8 | 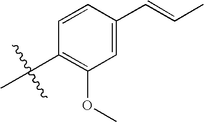 | 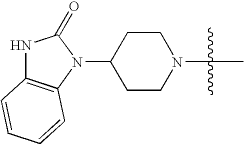 | 1.95 | 470.12 |
| 2-9 | 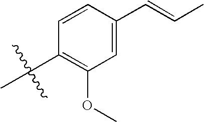 | 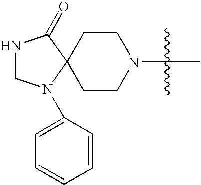 | 2.03 | 484.1 |
| 2-10 | 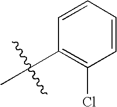 | 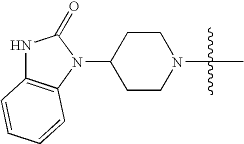 | 1.78 | 434.06 |
| 2-11 | 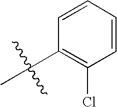 | 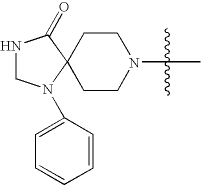 | 1.87 | 448.03 |
| 2-12 | 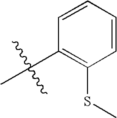 | 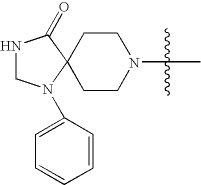 | 1.9 | 460.02 |
| 2-13 | 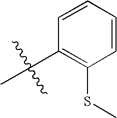 | 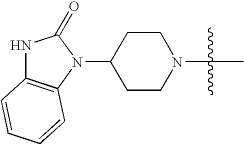 | 1.83 | 446.09 |
| 2-14 | 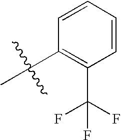 | 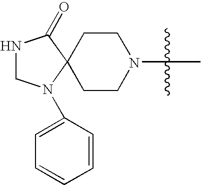 | 1.96 | 482.07 |
| 2-15 | 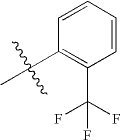 | 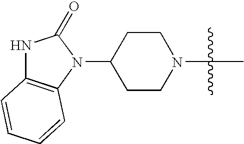 | 1.9 | 468.11 |
| 2-16 | 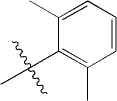 | 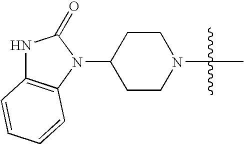 | 1.9 | 428.14 |
| 2-17 | 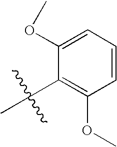 | 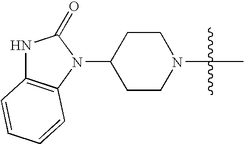 | 1.65 | 460.04 |
| 2-18 | 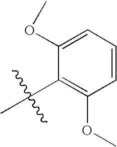 | 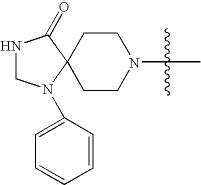 | 1.79 | 474.05 |
| 2-19 | 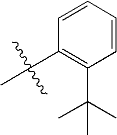 | 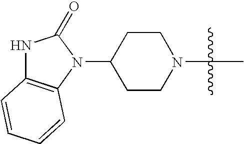 | 2.08 | 456.42 |
| 2-20 | 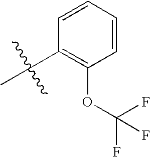 | 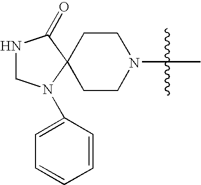 | 2.08 | 498.02 |
| 2-21 | 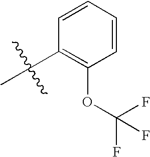 | 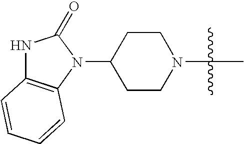 | 1.97 | 484.48 |
| 2-22 | 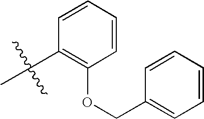 | 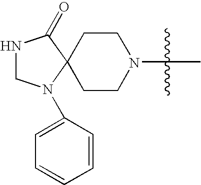 | 2.16 | 520.1 |
| 2-23 | 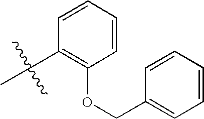 | 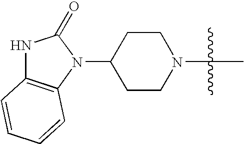 | 2.08 | 506.1 |
| 2-24 | 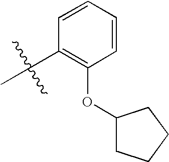 | 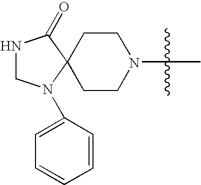 | 2 | 498.08 |
| 2-25 | 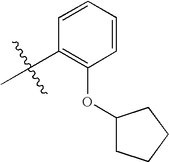 | 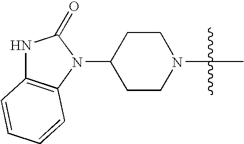 | 1.89 | 484.1 |
| 2-26 | 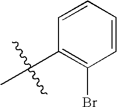 | 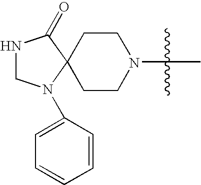 | 1.91 | 491.97 |
| 2-27 | 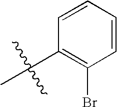 | 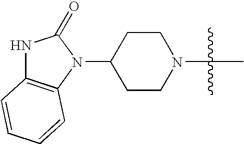 | 1.83 | 479.94 |
| 2-28 | 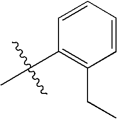 | 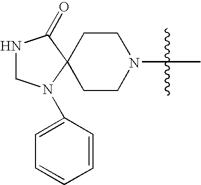 | 2.03 | 442.11 |
| 2-29 | 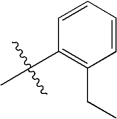 | 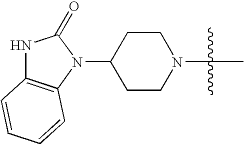 | 1.99 | 428.13 |
| 2-30 | 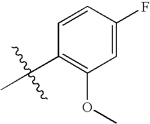 | 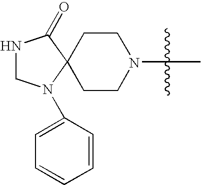 | 1.83 | 462.05 |
| 2-31 | 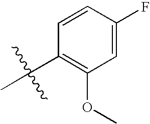 | 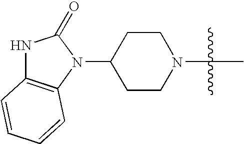 | 1.76 | 448.04 |
| 2-32 | 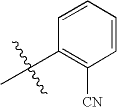 | 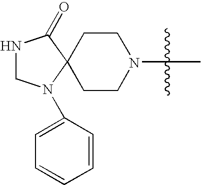 | 1.76 | 439.08 |
| 2-33 | 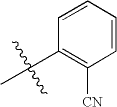 | 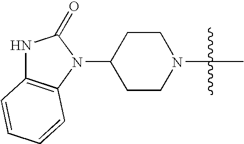 | 1.67 | 425.09 |
| 2-34 | 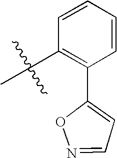 | 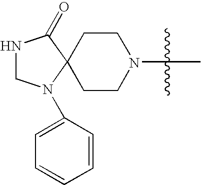 | 1.85 | 481.05 |
| 2-35 | 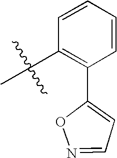 | 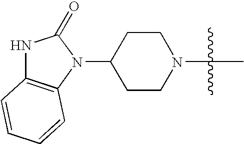 | 1.79 | 467.27 |
| 2-36 | 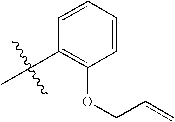 | 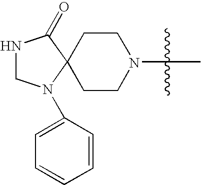 | 1.93 | 470.05 |
| 2-37 | 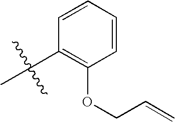 | 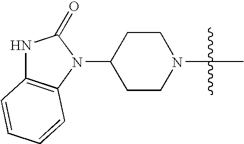 | 1.88 | 456.06 |
| 2-38 | 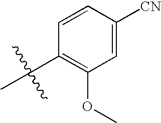 | 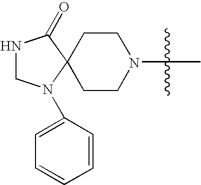 | 1.78 | 469.05 |
| 2-39 | 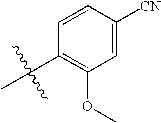 | 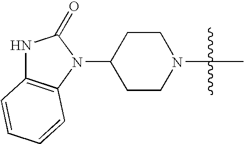 | 1.7 | 455.06 |
| 2-40 | 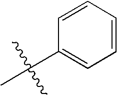 | 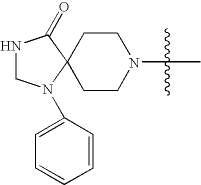 | 2.95 | 414.22 |
| 2-41 | 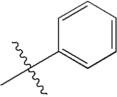 | 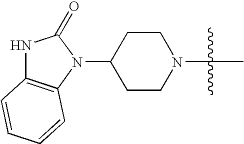 | 2.84 | 400.1 |
| 2-42 | 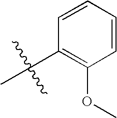 | 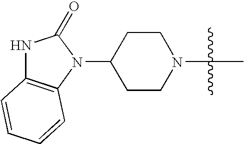 | 1.68 | 430 |

### Preparation of 3-aryloxy-(4′acylamino)-benzyl Piperidine

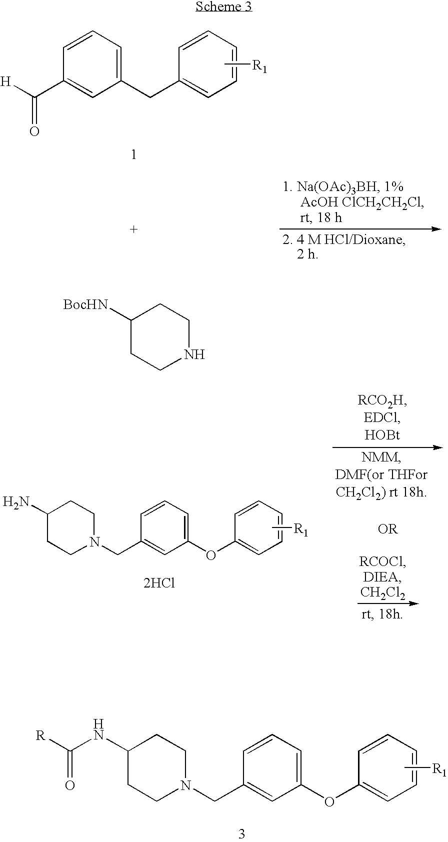

3-Aryloxy benzaldehyde 1 (1.2 eq) is mixed with 4N-Boc-amino-piperidine (1 eq.) and sodium triacetoxy borohydride (1.5 eq.) in dichloroethane containing acetic acid (1%) and the resulting mixture is stirred at room temperature overnight. The reaction mixture is diluted with CH₂Cl₂ and washed with saturated aqueous sodium bicarbonate solution and brine and dried over sodium sulfate. Column chromatography provides the corresponding N-Boc-benzyl amine. Removal of the Boc protecting group with 4M HCl/dioxane solution provides as the dihydrochloride salt.

The dihydrochloride salt (1 eq) was treated with the appropriate carboxylic acid (1.2 eq) in the presence of EDCI (1.2 eq), HOBt and N-methyl morpholine (4 eq) in DMF (or THF or CH₂Cl₂) for 16-18 h at room temperature. The solvent was evaporated and the residue was taken up in CH₂Cl₂ (or ethyl acetate) and washed with saturated aqueous sodium bicarbonate solution and brine and dried over sodium sulfate. Column chromatography provides the corresponding amide 3. Alternatively, the dihydrochloride salt was treated with the appropriate acid chloride (1.2 eq) in the presence of DIEA (4 eq) in CH₂Cl₂ for 18 h at room temperature. Workup as above and chromatography provides the desired amide 3. 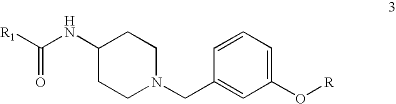
3-1: N-[1-(3-Phenoxy-benzyl)-piperidin-4-yl]-propionamide
3-2: N-[1-(3-Phenoxy-benzyl)-piperidin-4-yl]-2-phenyl-acetamide
3-3: N-[1-(3-Phenoxy-benzyl)-piperidin-4-yl]-benzamide
3-4: N-[1-(3-Phenoxy-benzyl)-piperidin-4-yl]-3-phenyl-propionamide
3-5: 2-(2-Bromo-phenyl)-N-[1-(3-phenoxy-benzyl)-piperidin-4-yl]-acetamide
3-6: 2-(3,4-Dimethoxy-phenyl)-N-[1-(3-phenoxy-benzyl)-piperidin-4-yl]-acetamide
3-7: 2-(3-Dimethoxy-phenyl)-N-[1-(3-phenoxy-benzyl)-piperidin-4-yl]-acetamide
3-8: N-[1-(3-Phenoxy-benzyl)-piperidin-4-yl]-2-thiophen-2-yl-acetamide
3-9: 2-Phenoxy-N-[1-(3-phenoxy-benzyl)-piperidin-4-yl]-acetamide
3-10: 3-Cyclopentyl-N-[1-(3-phenoxy-benzyl)-piperidin-4-yl]-propionamide
3-11: 3,5,5-Trimethyl-hexanoic acid [1-(3-phenoxy-benzyl)-piperidin-4-yl]-amide
3-12: 2-Cyclopentyl-N-[1-(3-phenoxy-benzyl)-piperidin-4-yl]-acetamide
3-13: N-[1-(3-Phenoxy-benzyl)-piperidin-4-yl]-2-(4-phenoxymethyl-phenyl)-acetamide
3-14: N-[1-(3-Phenoxy-benzyl)-piperidin-4-yl]-2,2-diphenyl-acetamide
3-15: 2-(1H-Indol-3-yl)-2-oxo-N-[1-(3-phenoxy-benzyl)-piperidin-4-yl]-acetamide
3-16: 2-(4-Chloro-phenyl)-N-[1-(3-phenoxy-benzyl)-piperidin-4-yl]-acetamide
3-17: N-[1-(3-Phenoxy-benzyl)-piperidin-4-yl]-2-(3-trifluoromethyl-phenyl)-acetamide
3-18: 2-(3,4-Dichloro-phenyl)-N-[1-(3-phenoxy-benzyl)-piperidin-4-yl]-acetamide
3-19: 2-(2-Methyl-1H-indol-3-yl)-N-[1-(3-phenoxy-benzyl)-piperidin-4-yl]-acetamide
3-20: N-[1-(3-Phenoxy-benzyl)-piperidin-4-yl]-2-o-tolyl-acetamide
3-21: 2-(2,6-Dichloro-phenyl)-N-{1-[3-(2-methoxy-phenoxy)-benzyl]-piperidin-4-yl}-acetamide
3-22: 2-(4-Fluoro-phenyl)-N-{1-[3-(2-methoxy-phenoxy)-benzyl]-piperidin-4-yl}-acetamide
3-23: 4-{1-[3-(2-Methoxy-phenoxy)-benzyl]-piperidin-4-ylcarbamoyl}-3,4-dihydro-1H-isoquinoline-2-carboxylic acid tert-butyl ester
3-24: 1-(4-Chloro-phenyl)-cyclopentanecarboxylic acid {1-[3-(2-methoxy-phenoxy)-benzyl]-piperidin-4-yl}-amide
3-25: N-{1-[3-(2-Methoxy-phenoxy)-benzyl]-piperidin-4-yl}-2-(3-methyl-isoxazol-5-yl)-acetamide
3-26: N-{1-[3-(2-Methoxy-phenoxy)-benzyl]-piperidin-4-yl}-2,2-diphenyl-acetamide
3-27: N-{1-[3-(2-Methoxy-phenoxy)-benzyl]-piperidin-4-yl}-2-(2-methyl-1H-indol-3-yl)-acetamide
3-28: N-{1-[3-(2-Methoxy-phenoxy)-benzyl]-piperidin-4-yl}-2-o-tolyl-acetamide
3-29: 2-Phenyl-N-{1-[3-(2-trifluoromethoxy-phenoxy)-benzyl]-piperidin-4-yl}-acetamide
3-30: N-{1-[3-(2-Chloro-phenoxy)-benzyl]-piperidin-4-yl}-2-phenyl-acetamide
3-31: N-{1-[3-(2-Benzyloxy-phenoxy)-benzyl]-piperidin-4-yl}-2-phenyl-acetamide
3-32: N-{1-[3-(4-Fluoro-phenoxy)-benzyl]-piperidin-4-yl}-2-phenyl-acetamide
3-33: 2-(4-Chloro-phenyl)-N-{1-[3-(2-methoxy-phenoxy)-benzyl]-piperidin-4-yl}-isobutyramide
3-34: 4-{1-[3-(2-Methoxy-phenoxy)-benzyl]-piperidin-4-ylcarbamoyl}-4-phenyl-piperidine-1-carboxylic acid tert-butyl ester
3-35: 1-Phenyl-cyclopentanecarboxylic acid {1-[3-(2-methoxy-phenoxy)-benzyl]-piperidin-4-yl}-amide
3-36: 2-(2-Bromo-phenyl)-N-{1-[3-(2-methoxy-phenoxy)-benzyl]-piperidin-4-yl}-acetamide
3-37: N-{1-[3-(4-Cyano-2-methoxy-phenoxy)-benzyl]-piperidin-4-yl}-2-phenyl-acetamide
3-38: N-{1-[3-(2-Cyclopentyloxy-phenoxy)-benzyl]-piperidin-4-yl}-2-phenyl-acetamide
3-39: N-{1-[3-(2-Bromo-phenoxy)-benzyl]-piperidin-4-yl}-2-phenyl-acetamide
3-40: N-{1-[3-(2-Ethyl-phenoxy)-benzyl]-piperidin-4-yl}-2-phenyl-acetamide
3-41: N-{1-[3-(4-Fluoro-2-methoxy-phenoxy)-benzyl]-piperidin-4-yl}-2-phenyl-acetamide
3-42: N-{1-[3-(2-Cyano-phenoxy)-benzyl]-piperidin-4-yl}-2-phenyl-acetamide
3-43: N-{1-[3-(2-Methoxy-phenoxy)-benzyl]-piperidin-4-yl}-2R-phenyl-propionamide
3-44: N-{1-[3-(2-Methoxy-phenoxy)-benzyl]-piperidin-4-yl}-2-phenyl-butyramide
3-45: 2-(4-Isobutyl-phenyl)-N-{1-[3-(2-methoxy-phenoxy)-benzyl]-piperidin-4-yl}-propionamide
3-46: N-{1-[3-(2-Methoxy-phenoxy)-benzyl]-piperidin-4-yl}-2-phenyl-propionamide
3-47: N-{1-[3-(2-Isoxazol-5-yl-phenoxy)-benzyl]-piperidin-4-yl}-2-phenyl-acetamide
3-48: N-{1-[3-(2-Allyloxy-phenoxy)-benzyl]-piperidin-4-yl}-2-phenyl-acetamide
3-49: N-{1-[3-(2-Methoxy-phenoxy)-benzyl]-piperidin-4-yl}-2-phenyl-propionamide
3-50: 1-(4-Chloro-phenyl)-cyclohexanecarboxylic acid {1-[3-(2-methoxy-phenoxy)-benzyl]-piperidin-4-yl}-amide
3-51: 1-p-Tolyl-cyclopentanecarboxylic acid {1-[3-(2-methoxy-phenoxy)-benzyl]-piperidin-4-yl}-amide
3-52: 1-(2-Fluoro-phenyl)-cyclopentanecarboxylic acid {1-[3-(2-methoxy-phenoxy)-benzyl]-piperidin-4-yl}-amide
3-53: 2-(4-Chloro-phenyl)-N-{1-[3-(2-methoxy-phenoxy)-benzyl]-piperidinyl}-propionamide
3-54: 1-(2,4-Dichloro-phenyl)-cyclopropanecarboxylic acid {1-[3-(2-methoxy-phenoxy)-benzyl]-piperidin-4-yl}-amide
3-55: 3-Oxo-indan-1-carboxylic acid {1-[3-(2-methoxy-phenoxy)-benzyl]-piperidin-4-yl}-amide
3-56: N-{1-[3-(2-Methoxy-phenoxy)-benzyl]-piperidin-4-yl}-2-(3-methyl-benzo[b]thiophen-2-yl)-acetamide
3-57: 2-Benzo[b]thiophen-3-yl-N-{1-[3-(2-methoxy-phenoxy)-benzyl]-piperidin-4-yl}-acetamide
3-58: N-{1-[3-(2-Methoxy-phenoxy)-benzyl]-piperidin-4-yl}-2-(5-methyl-2-phenyl-oxazol-4-yl)-acetamide
3-59: 1-(4-Methoxy-phenyl)-cyclopentanecarboxylic acid {1-[3-(2-methoxy-phenoxy)-benzyl]-piperidin-4-yl}-amide
3-60: 1-(2-Chloro-6-fluoro-phenyl)-cyclopentanecarboxylic acid {1-[3-(2-methoxy-phenoxy)-benzyl]-piperidin-4-yl}-amide
3-61: 1-(4-Fluoro-phenyl)-cyclopentanecarboxylic acid {1-[3-(2-methoxy-phenoxy)-benzyl]-piperidin-4-yl}-amide
3-62: 1-Phenyl-cyclopropanecarboxylic acid {1-[3-(2-methoxy-phenoxy)-benzyl]-piperidin-4-yl}-amide
3-63: N-{1-[3-(2-Methoxy-phenoxy)-benzyl]-piperidin-4-yl}-2-thiophen-3-yl-acetamide
3-64: 1-Phenyl-cyclopentanecarboxylic acid {1-[3-(2-chloro-phenoxy)-benzyl]-piperidin-4-yl}-amide
3-65: 4-{1-[3-(2-Chloro-phenoxy)-benzyl]-piperidin-4-ylcarbamoyl}-4-phenyl-piperidine-1-carboxylic acid tert-butyl ester
3-66: 1-(5-Methyl-2-phenyl-oxazol-4-yl)-cyclopentanecarboxylic acid {1-[3-(2-methoxy-phenoxy)-benzyl]-piperidin-4-yl}-amide
3-67: 1-Thiophen-2-yl-cyclopentanecarboxylic acid {1-[3-(2-methoxy-phenoxy)-benzyl]-piperidin-4-yl}-amide
3-68: N-{1-[3-(2-Methoxy-phenoxy)-benzyl]-piperidin-4-yl}-2-(5-methyl-1-phenyl-1H-pyrazol-3-yl)-acetamide
3-69: 4-Phenyl-tetrahydro-pyran-4-carboxylic acid {1-[3-(2-methoxy-phenoxy)-benzyl]-piperidin-4-yl}-amide
3-70: N-{1-[3-(2-Methoxy-5-methyl-phenoxy)-benzyl]-piperidin-4-yl}-2-phenyl-acetamide
3-71: 3-{1-[3-(2-Methoxy-phenoxy)-benzyl]-piperidin-4-ylcarbamoyl}-3-phenyl-pyrrolidine-1-carboxylic acid tert-butyl ester
3-72: 1-(5-Methyl-1-phenyl-1H-pyrazol-4-yl)-cyclopentanecarboxylic acid {1-[3-(2-methoxy-phenoxy)-benzyl]-piperidin-4-yl}-amide
3-73: 1-Benzo[b]thiophen-3-yl-cyclopentanecarboxylic acid {1-[3-(2-methoxy-phenoxy)-benzyl]-piperidin-4-yl}-amide
3-74: 2-[5-(4-Chloro-benzoyl)-thiophen-3-yl]-N-{1-[3-(2-methoxy-phenoxy)-benzyl]-piperidin-4-yl}-acetamide
3-75: N-{1-[3-(2-Methoxy-phenoxy)-benzyl]-piperidin-4-yl}-2-pyridin-3-yl-acetamide
3-76: 2,2,2-Trifluoro-N-({1-[3-(2-methoxy-phenoxy)-benzyl]-piperidin-4-ylcarbamoyl}-phenyl-methyl)-acetamide
3-77: 2-Oxo-1,2,3,4-tetrahydro-quinoline-4-carboxylic acid {1-[3-(2-methoxy-phenoxy)-benzyl]-piperidin-4-yl}-amide
3-78: 1-(4-Chloro-phenyl)-cyclopropanecarboxylic acid {1-[3-(2-methoxy-phenoxy)-benzyl]-piperidin-4-yl}-amide
3-79: 1-(4-Methoxy-phenyl)-cyclopropanecarboxylic acid {1-[3-(2-methoxy-phenoxy)-benzyl]-piperidin-4-yl}-amide
3-80: N-{1-[3-(2-Methoxy-phenoxy)-benzyl]-piperidin-4-yl}-2-thiophen-3-yl-isobutyramide
3-81: N-{1-[3-(2-Methoxy-phenoxy)-benzyl]-piperidin-4-yl}-2-(2-methyl-1H-indol-3-yl)-isobutyramide
3-82: 2-(1H-Indol-3-yl)-N-{1-[3-(2-methoxy-phenoxy)-benzyl]-piperidin-4-yl}-isobutyramide

3:83: N-{1-[3-(2-Methoxy-phenoxy)-benzyl]-piperidin-4-yl}-2-phenyl-acetamide

TABLE 3

| | | | Retention | |
|---|---|---|---|---|
| No. | R | R₁ | time (min) | M + 1 |
| 3-1 | 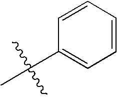 | 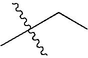 | 2.49 | 339 |
| 3-2 | 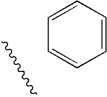 | 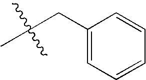 | 2.68 | 401 |
| 3-3 | 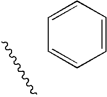 | 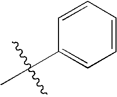 | 2.64 | 387 |
| 3-4 | 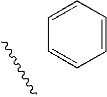 | 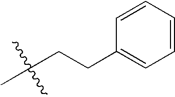 | 1.6 | 415 |
| 3-5 | 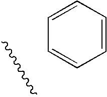 | 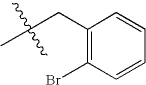 | 1.64 | 479 |
| 3-6 | 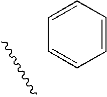 | 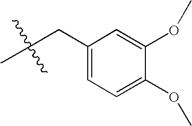 | 1.66 | 461 |
| 3-7 | 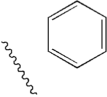 | 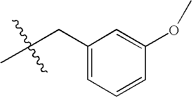 | 1.74 | 431 |
| 3-8 | 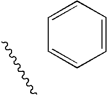 | 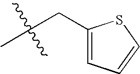 | 1.6 | 407 |
| 3-9 | 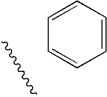 | 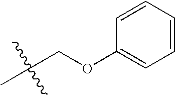 | 1.74 | 417 |
| 3-10 | 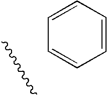 | 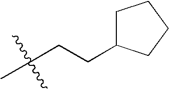 | 1.83 | 407 |
| 3-11 | 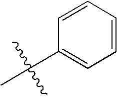 | 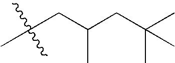 | 1.97 | 423 |
| 3-12 | 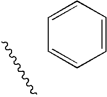 | 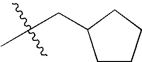 | 1.71 | 393 |
| 3-13 | 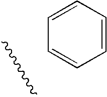 | 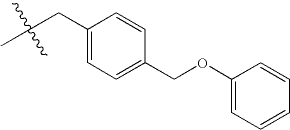 | 2.05 | 551 |
| 3-14 | 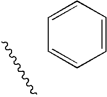 | 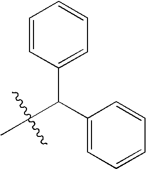 | 2.03 | 477 |
| 3-15 | 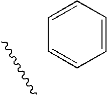 | 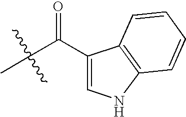 | 1.83 | 454 |
| 3-16 | 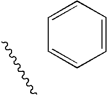 | 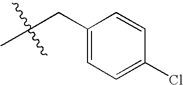 | 1.87 | 435 |
| 3-17 | 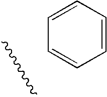 | 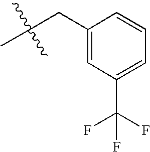 | 1.85 | 469 |
| 3-18 | 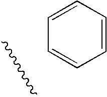 | 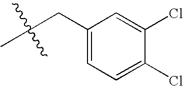 | 1.9 | 469 |
| 3-19 | 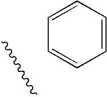 | 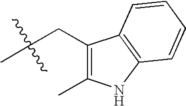 | 1.76 | 454 |
| 3-20 | 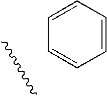 | 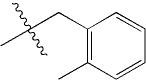 | 1.73 | 415 |
| 3-21 | 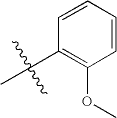 | 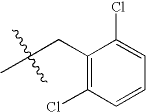 | 1.88 | 499 |
| 3-22 | 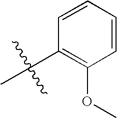 | 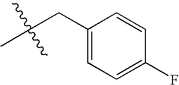 | 1.78 | 449 |
| 3-23 | 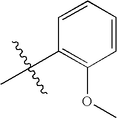 | 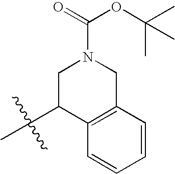 | 1.99 | 572 |
| 3-24 | 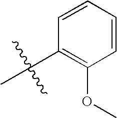 | 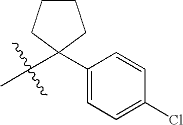 | 2.19 | 519 |
| 3-25 | 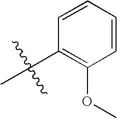 | 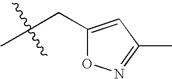 | 1.61 | 436 |
| 3-26 | 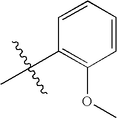 | 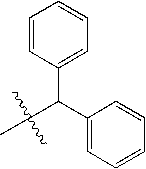 | 2.1 | 507 |
| 3-27 | 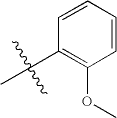 | 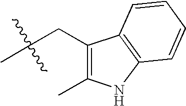 | 1.7 | 484 |
| 3-28 | 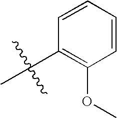 | 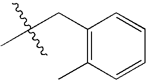 | 1.69 | 445 |
| 3-29 | 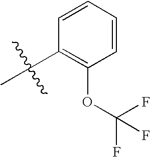 | 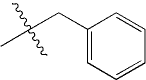 | 2.05 | 485 |
| 3-30 | 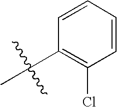 | 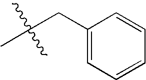 | 1.92 | 435 |
| 3-31 | 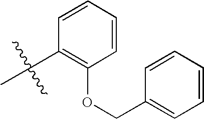 | 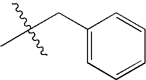 | 2.1 | 507 |
| 3-32 | 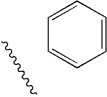 | 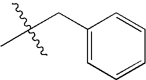 | 1.76 | 419 |
| 3-33 | 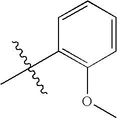 | 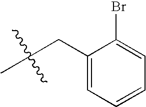 | 1.9 | 493 |
| 3-34 | 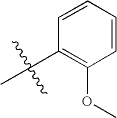 | 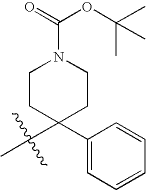 | 2.02 | 600 |
| 3-35 | 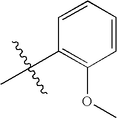 | 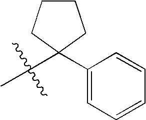 | 2.02 | 485 |
| 3-36 | 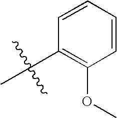 | 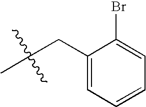 | 1.75 | 510 |
| 3-37 | 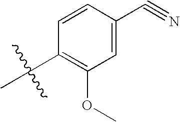 | 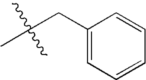 | 1.66 | 456 |
| 3-38 | 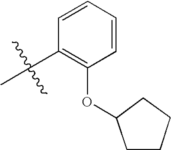 | 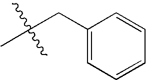 | 2.01 | 485 |
| 3-39 | 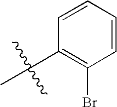 | 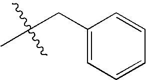 | 1.86 | 479 |
| 3-40 | 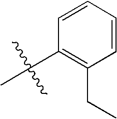 | 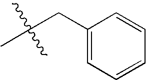 | 1.93 | 429 |
| 3-41 | 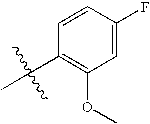 | 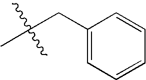 | 1.76 | 449 |
| 3-42 | 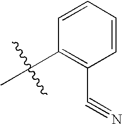 | 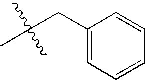 | 1.67 | 426 |
| 3-43 | 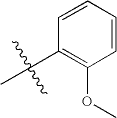 | 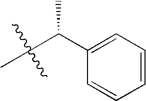 | 1.73 | 445 |
| 3-44 | 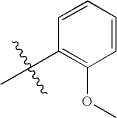 | 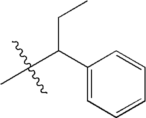 | 1.89 | 459 |
| 3-45 | 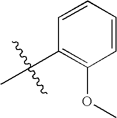 | 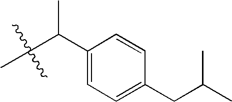 | 2.09 | 501 |
| 3-46 | 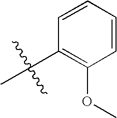 | 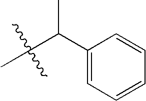 | 1.72 | 445 |
| 3-47 | 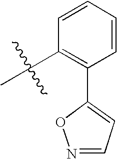 | 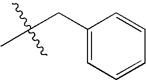 | 1.83 | 468 |
| 3-48 | 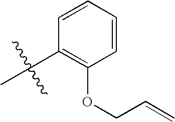 | 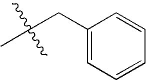 | 1.88 | 457 |
| 3-49 | 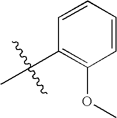 | 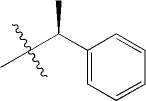 | 1.83 | 445 |
| 3-50 | 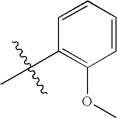 | 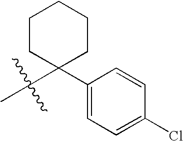 | 2.24 | 534 |
| 3-51 | 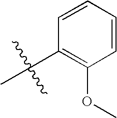 | 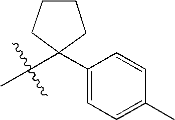 | 2.11 | 499 |
| 3-52 | 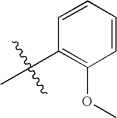 | 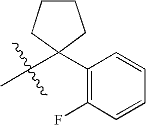 | 1.99 | 503 |
| 3-53 | 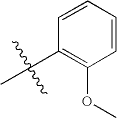 | 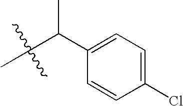 | 1.96 | 480 |
| 3-54 | 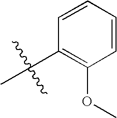 | 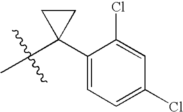 | 2.09 | 526 |
| 3-55 | 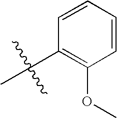 | 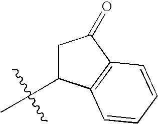 | 1.55 | 471 |
| 3-56 | 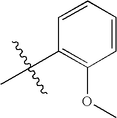 | 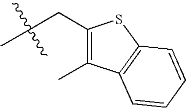 | 1.84 | 501 |
| 3-57 | 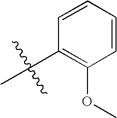 | 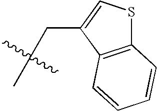 | 1.77 | 487 |
| 3-58 | 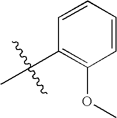 | 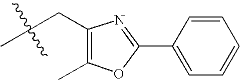 | 1.72 | 512 |
| 3-59 | 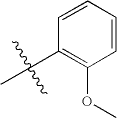 | 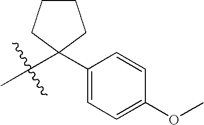 | 1.92 | 515 |
| 3-60 | 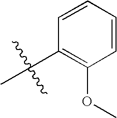 | 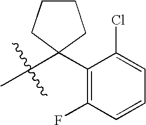 | 1.99 | 538 |
| 3-61 | 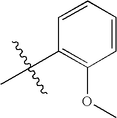 | 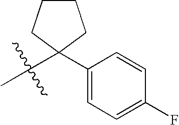 | 1.97 | 503 |
| 3-62 | 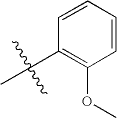 | 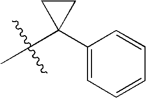 | 1.78 | 457 |
| 3-63 | 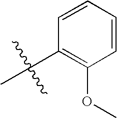 | 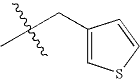 | 1.65 | 437 |
| 3-64 | 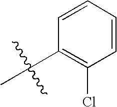 | 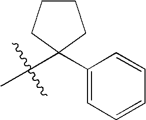 | 1.91 | 490 |
| 3-65 | 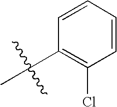 | 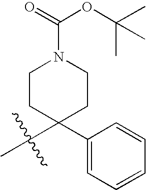 | 2.27 | 605 |
| 3-66 | 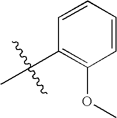 | 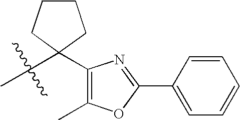 | 2.16 | 566 |
| 3-67 | 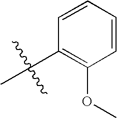 | 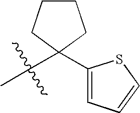 | 1.98 | 491 |
| 3-68 | 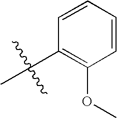 | 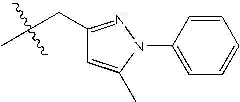 | 1.83 | 512 |
| 3-69 | 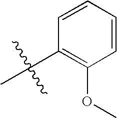 | 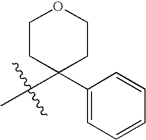 | 1.79 | 501 |
| 3-70 | 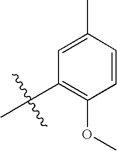 | 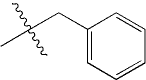 | 1.64 | 445 |
| 3-71 | 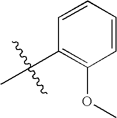 | 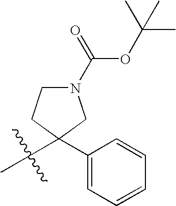 | 2.01 | 586 |
| 3-72 | 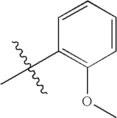 | 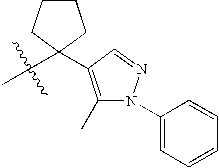 | 1.8 | 565 |
| 3-73 | 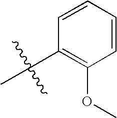 | 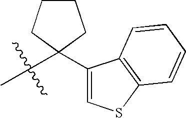 | 1.91 | 541 |
| 3-74 | 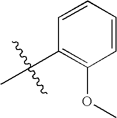 | 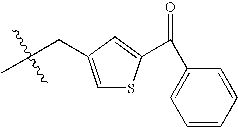 | 1.99 | 561 |
| 3-75 | 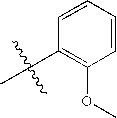 | 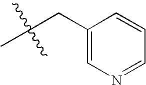 | 1.1 | 432 |
| 3-76 | 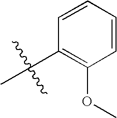 | 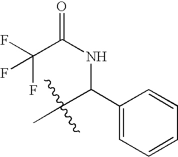 | 1.82 | 542 |
| 3-77 | 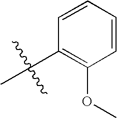 | 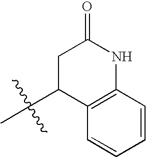 | 1.35 | 486 |
| 3-78 | 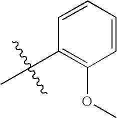 | 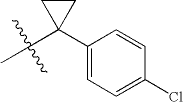 | 1.9 | 492 |
| 3-79 | 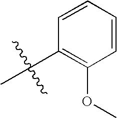 | 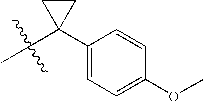 | 1.84 | 487 |
| 3-80 | 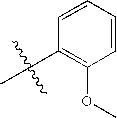 | 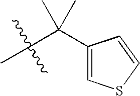 | 1.75 | 465 |
| 3-81 | 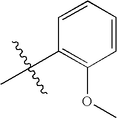 | 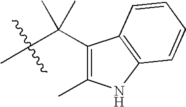 | 1.83 | 512 |
| 3-82 | 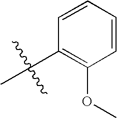 | 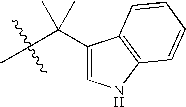 | 1.81 | 498 |
| 3-83 | 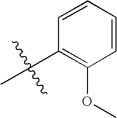 | 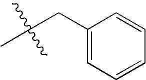 | 1.65 | 431 |

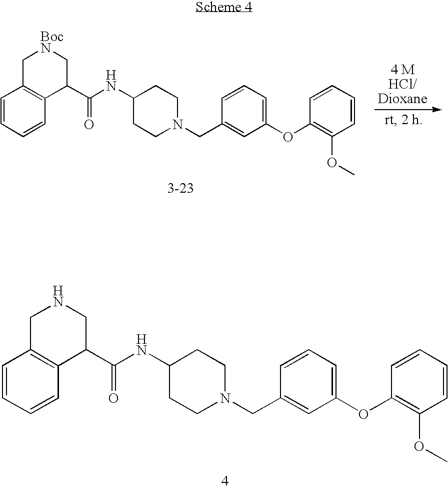

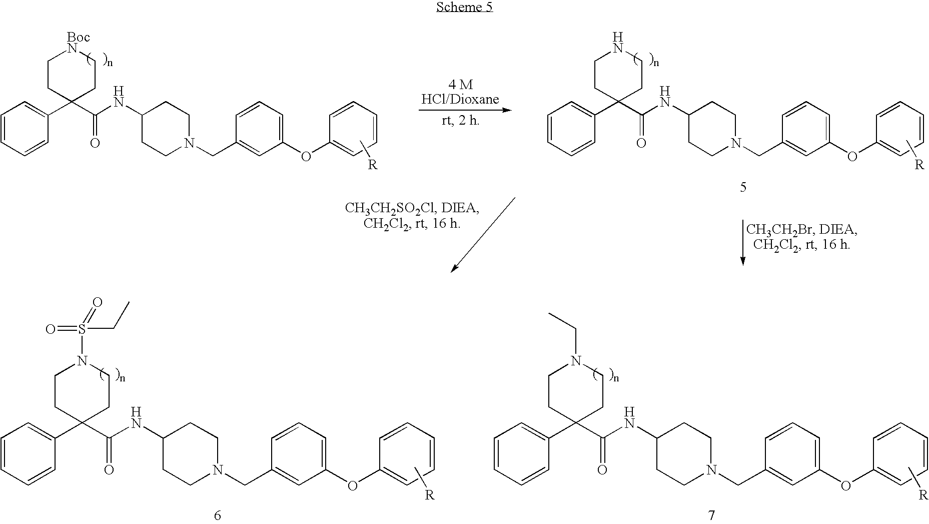

### 4:1,2,3,4-Tetrahydro-isoquinoline-4-carboxylic Acid {1-[3-(2-methoxy-phenoxy)-benzyl]-piperidin-4-yl}-amide

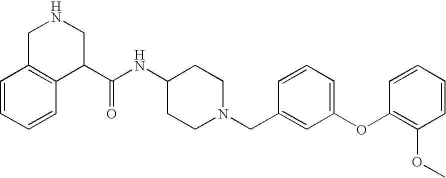

3-23 (0.12 g, 0.17 mmol) was treated with 4M HCl/dioxane for 2 h at room temperature. The solvent was evaporated and the residue was triturated with ether and filtered to give the dihydrochloride salt 4 (95 mg, 94%). ¹H NMR is consistent with assigned structure. LC/MS: UV Retention time: 1.33 min, M+1=472.

### 5-1: 4-Phenyl-piperidine-4-carboxylic Acid {1-[3-(2-methoxy-phenoxy)-benzyl]-piperidin-4-yl}-amide

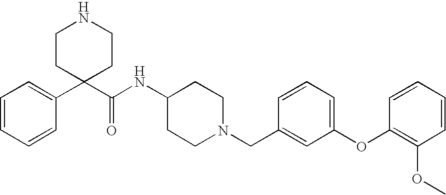

3-34 (0.4 g, 0.67 mmol) was treated with 4M HCl/dioxane for 2 h at room temperature. The solvent was evaporated and the residue was triturated with ether and filtered to give the dihydrochloride salt 5-1 (300 mg, 90%). ¹H NMR is consistent with assigned structure. LC/MS: TV Retention time: 1.36 min, M+1=500.

### 5-2: 3-Phenyl-pyrrolidine-3-carboxylic Acid {1-[3-(2-methoxy-phenoxy)-benzyl]-piperidin-4-yl}-amide

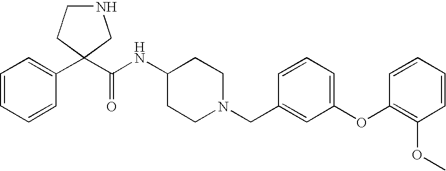

3-72 was treated with 4M HCl/dioxane for 2 h at room temperature. The solvent was evaporated and the residue was triturated with ether and filtered to give the dihydrochloride salt 5-2. ¹H NMR is consistent with assigned structure. LC/MS: UV Retention time: 1.19 min, M+1=486.

### 5-3: 4-Phenyl-piperidine-4-carboxylic Acid {1-[3-(2-chloro-phenoxy)-benzyl]-piperidin-4-yl}-amide

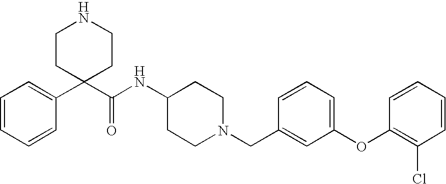

3-65 was treated with 4M HCl/dioxane for 2 h at room temperature. The solvent was evaporated and the residue was triturated with ether and filtered to give the dihydrochloride salt 5-3. ¹H NMR is consistent with assigned structure. LC/MS: UV Retention time: 1.42 min, M+1=505.

### 5-4: 4-Phenyl-piperidine-4-carboxylic Acid {1-[3-(2-methoxy-5-methyl-phenoxy)-benzyl]-piperidin-4-yl}-amide

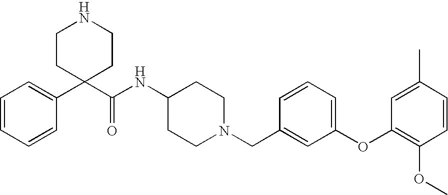

The amine was treated with 4M HCl/dioxane for 2 h at room temperature. The solvent was evaporated and the residue was triturated with ether and filtered to give the dihydrochloride salt 5-4. ¹H NMR is consistent with assigned structure. LC/MS: UV Retention time: 1.18 min, M+1=514.

### 6-1: 1-Ethanesulfonyl-4-phenyl-piperidine-4-carboxylic Acid {1-[3-(2-methoxy-phenoxy)-benzyl]-piperidin-4-yl}-amide

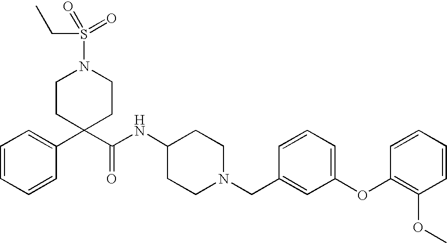

Compound 5-1 (0.225, 0.4 mmol) was mixed with DIEA (0.31 g, 2.4 mmol) and ethyl sulfonyl chloride (0.062 g, 0.48 mmol) in CH₂Cl₂ and the resulting solution was stirred at room temperature for 18 h. The reaction mixture is diluted with CH₂Cl₂ and washed with saturated aqueous sodium bicarbonate solution and brine and dried over sodium sulfate. Column chromatography (5-10% MeOH/CH₂Cl₂) provided the N-sulfonyl ethyl analog 6-1 (0.16 g, 68%). ¹H NMR is consistent with assigned structure. LC/MS: UV Retention time: 1.75 min, M+1=592.

### 6-2: 1-Ethanesulfonyl-3-phenyl-pyrrolidine-3-carboxylic acid {1-[3-(2-methoxy-phenoxy)-benzyl]-piperidin-4-yl}-amide

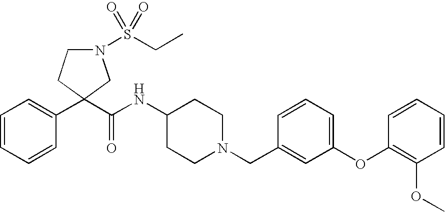

Compound 5-2 was mixed with DEEA and ethyl sulfonyl chloride in CH₂Cl₂ and the resulting solution was stirred at room temperature for 18 h. The reaction mixture was diluted with CH₂Cl₂ and washed with saturated aqueous sodium bicarbonate solution and brine and dried over sodium sulfate. Column chromatography provided the N-sulfonyl ethyl analog 6-2. ¹H NMR is consistent with assigned structure. LC/MS: UV Retention time: 1.80 min, M+1=578.

### 7-1: 1-Ethyl-4-phenyl-piperidine-4-carboxylic acid {1-[3-(2-methoxy-phenoxy)-benzyl]-piperidin-4-yl}-amide

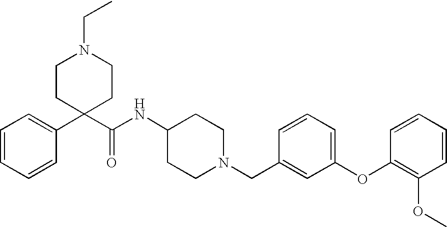

Compound 5-1 was mixed with DIEA and ethyl bromide in CH₂Cl₂ and the resulting solution was stirred at room temperature for 18 h. Standard work-up (as above) and column chromatography provided the corresponding N-ethyl analog 7-1. ¹H NMR is consistent with assigned structure. LC/MS: UV Retention time: 1.39 min, M+1=528.

### 7-2: 1-Ethyl-3-phenyl-pyrrolidine-3-carboxylic acid {1-[3-(2-methoxy-phenoxy)-benzyl]-piperidin-4-yl}-amide

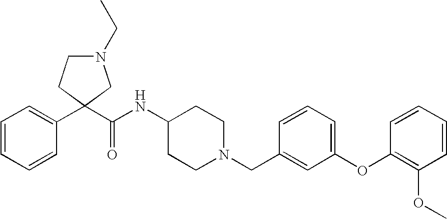

Compound 5-2 was mixed with DIEA and ethyl bromide in CH₂Cl₂ and the resulting solution was stirred at room temperature for 18 h. Standard work-up (as above) and column chromatography provided the corresponding N-ethyl analog 7-2. ¹H NMR is consistent with assigned structure. LC/MS: UV Retention time: 1.29 min, M+1=514.

### Preparation of phenyl-piperidine-4-carboxylic acid {1-[3-phenoxy-benzyl]-piperidin-4-yl}-amide

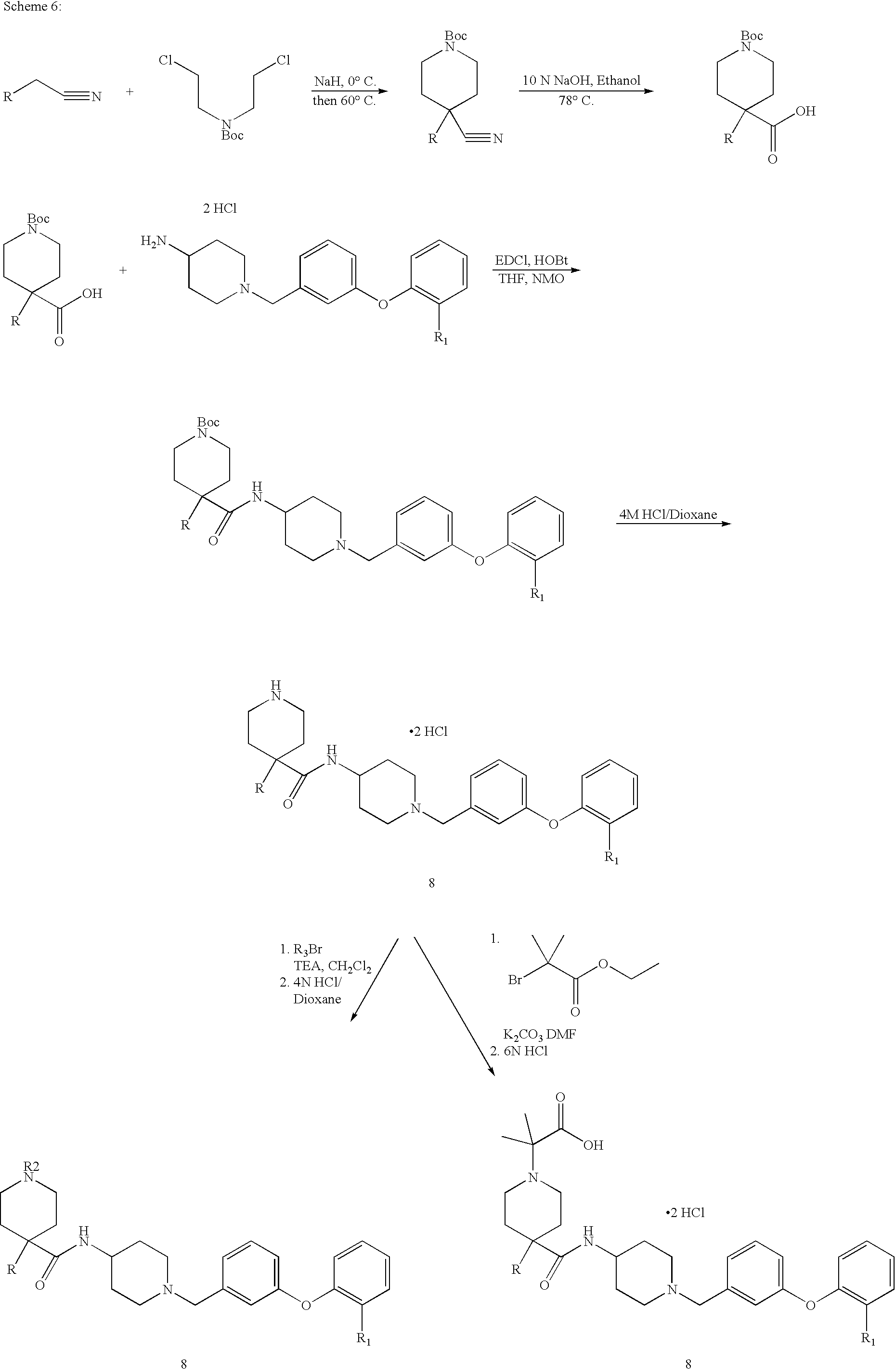

### Substituted 4-cyano-piperidine-1-carboxylic Acid Tert-Butyl Ester

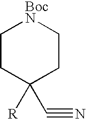

Substituted acetonitrile (1 mmol) was added along with bis-(2-chloro-ethyl)-carbamic acid tert-butyl ester (1.0 mmol) in DMF and cooled to 0° C. Sodium hydride (3.0 mmol) was added to the mixture portionwise over ˜20 min. The reaction was allowed to warm to room temperature and then heated to 60° C. for 16 h. The reaction was quenched by addition to ice water and the aqueous phase was extracted 3×EtOAc. The organics were collected together and washed 2× water, 1×brine and dried over MgSO₄, filtered and concentrated down. The product was purified by flash chromatography with 100% EtOAc to give the substituted 4-cyano-piperidine-1-carboxylic acid tert-butyl ester. ¹H NMR data is consistent with the assigned structure.

### Substituted 4-piperidine-1,4-dicarboxylic Acid Mono-Tert-Butyl Ester

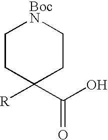

Substituted 4-cyanopiperidine-1-carboxylic acid tert-butyl ester was dissolved in ethanol and a 10N solution of NaOH was added. The reaction mixture was heated for 24 h at 60° C. The ethanol was removed in vacuo and the basic solution was washed with EtOAc and the aqueous layer was acidified with conc. HCl and extracted 3×EtOAc. The organics were collected together and dried over MgSO₄, filtered and concentrated down. The product was purified by flash chromatography to give the substituted 4-piperidine-1,4-dicarboxylic acid mono-tert-butyl ester. ¹H NMR data is consistent with the assigned structure.

### Substituted 4-piperidine-4-carboxylic Acid {1-[3-(2-chloro-phenoxy)-benzyl]-piperidin-4-yl}-amide

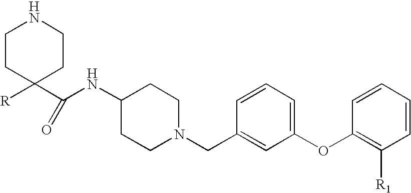

1-[3-(2-Chloro-phenoxy)-benzyl]-piperdin-4-ylamine (1.0 equ) and the corresponding substituted 4-phenyl-piperidine-1,4-dicarboxylic acid mono-tert-butyl ester acid (1.05 equ) were mixed with HOBt (1.5 equ.), EDCI (1.3 equ.) in THF with N-methyl morpholine (5.0 equ.). The reaction was allowed to stir at room temperature for 10 h. The reaction was diluted with ethyl acetate and washed with 1N HCl, 1N NaOH and brine. The organics were dried over Mg₂SO₄, filtered and concentrated down. The product was purified by flash chromatography with 100% EtOAc to give the corresponding N-{1-[3-(2-chloro-phenoxy)-benzyl]-piperdin-4-yl} acetamide.

The amine was treated with 4N HCl/dioxane for 2 h at room temperature. The solvent was evaporated and the residue was triturated with ether and filtered to give the dihydrochloride salt. ¹H NMR is consistent with assigned structure. 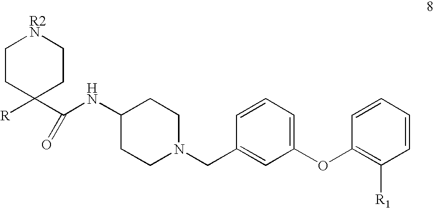
8-1: 4-p-Tolyl-piperidine-4-carboxylic acid {1-[3-(2-chloro-phenoxy)-benzyl]-piperidin-4-yl}-amide
8-2: 1-Ethyl-4-p-tolyl-piperidine-4-carboxylic acid {1-[3-(2-chloro-phenoxy)-benzyl]-piperidin-4-yl}-amide
8-3: 2-(4-{1-[3-(2-Chloro-phenoxy)-benzyl]-piperidin-4-ylcarbamoyl}-4-p-tolyl-piperidin-1-yl)-2-methyl-propionic acid
8-4: 4-(4-Fluoro-phenyl)-piperidine-4-carboxylic acid {1-[3-(2-chloro-phenoxy)-benzyl]-piperidin-4-yl}-amide
8-5: 1-Ethyl-4-(4-fluoro-phenyl)-piperidine-4-carboxylic acid {1-[3-(2-chloro-phenoxy)-benzyl]-piperidin-4-yl}-amide
8-6: 2-[4-{1-[3-(2-Chloro-phenoxy)-benzyl]-piperidin-4-ylcarbamoyl}-4-(4-fluoro-phenyl)-piperidin-1-yl]-2-methyl-propionic acid
8-7: 4-(3-Fluoro-phenyl)-piperidine-4-carboxylic acid {1-[3-(2-chloro-phenoxy)-benzyl]-piperidin-4-yl}-amide
8-8: 1-Ethyl-4-(3-fluoro-phenyl)-piperidine-4-carboxylic acid {1-[3-(2-chloro-phenoxy)-benzyl]-piperidin-4-yl}-amide
8-9: 4-(2-Fluoro-phenyl)-piperidine-4-carboxylic acid {1-[3-(2-chloro-phenoxy)-benzyl]-piperidin-4-yl}-amide
8-10:1-Ethyl-4-(2-fluoro-phenyl)-piperidine-4-carboxylic acid {1-[3-(2-chloro-phenoxy)-benzyl]-piperidin-4-yl}-amide
8-11: 4-Phenyl-piperidine-4-carboxylic acid {1-[3-(2-chloro-phenoxy)-benzyl]-piperidin-4-yl}-amide
8-12: 1-Ethyl-4-phenyl-piperidine-4-carboxylic acid {1-[3-(2-chloro-phenoxy)-benzyl]-piperidin-4-yl}-amide
8-13:1-Methanesulfonyl-4-phenyl-piperidine-4-carboxylic acid {1-[3-(2-chloro-phenoxy)-benzyl]-piperidin-4-yl}-amide
8-14: 2-(4-{1-[3-(2-Chloro-phenoxy)-benzyl]-piperidin-4-ylcarbamoyl}-4-phenyl-piperidin-1-yl)-2-methyl-propionic acid
8-15: 2-(4-{1-[3-(2-Chloro-phenoxy)-benzyl]-piperidin-4-ylcarbamoyl}-4-phenyl-piperidin-1-yl)-2-methyl-propionic acid ethyl ester
8-16: 4-(4-Bromo-phenyl)-piperidine-4-carboxylic acid {1-[3-(2-chloro-phenoxy)-benzyl]-piperidin-4-yl}-amide
8-17: 4-(4-Bromo-phenyl)-1-ethyl-piperidine-4-carboxylic acid {1-[3-(2-chloro-phenoxy)-benzyl]-piperidin-4-yl}-amide
8-18:1-Ethyl-4-thiophen-3-yl-piperidine-4-carboxylic acid {1-[3-(2-chloro-phenoxy)-benzyl]-piperidin-4-yl}-amide
8-19: 4-{1-[3-(2-Chloro-phenoxy)-benzyl]-piperidin-4-ylcarbamoyl}-4-thiophen-3-yl-piperidine-1-carboxylic acid tert-butyl ester
8-20: 4′-{1-[3-(2-Chloro-phenoxy)-benzyl]-piperidin-4-ylcarbamoyl}-3′,4′,5′,6′-tetrahydro-2′H-[3,4′]bipyridinyl-1′-carboxylic acid tert-butyl ester
8-21: 1-Isobutyl-4-phenyl-piperidine-4-carboxylic acid {1-[3-(2-chloro-phenoxy)-benzyl]-piperidin-4-yl}-amide
8-22: 1-Isopropyl-4-phenyl-piperidine-4-carboxylic acid {1-[3-(2-chloro-phenoxy)-benzyl]-piperidin-4-yl}-amide
8-23: 4-Phenyl-piperidine-1,4-dicarboxylic acid 4-({1-[3-(2-chloro-phenoxy)-benzyl]-piperidin-4-yl}-amide) 1-ethylamide
8-24: 4-{1-[3-(2-Chloro-phenoxy)-benzyl]-piperidin-4-ylcarbamoyl}-4-phenyl-piperidine-1-carboxylic acid ethyl ester
8-25: 1-(2-Cyclopentyl-acetyl)-4-phenyl-piperidine-4-carboxylic acid {1-[3-(2-methoxy-phenoxy)-benzyl]-piperidin-4-yl}-amide
8-26:1-(2-Carbamoyl-ethyl)-4-phenyl-piperidine-4-carboxylic acid {1-[3-(2-methoxy-phenoxy)-benzyl]-piperidin-4-yl}-amide
8-27: 1-Isobutyl-4-phenyl-piperidine-4-carboxylic acid {1-[3-(2-methoxy-phenoxy)-benzyl]-piperidin-4-yl}-amide

8-28: 1-Ethyl-4-phenyl-piperidine-4-carboxylic acid {1-[3-(2-methoxy-phenoxy)-benzyl]-piperidin-4-yl}-amide

TABLE 4

| | | | | Retention time | |
|---|---|---|---|---|---|
| No. | R | R₁ | R₂ | (min) | M + 1 |
| 8-1 | 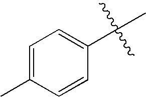 | —Cl | —H | 1.28 | 519 |
| 8-2 | 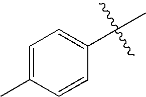 | —Cl | 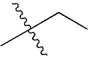 | 1.38 | 547 |
| 8-3 | 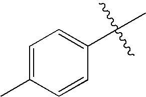 | —Cl | 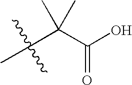 | 1.55 | 605 |
| 8-4 | 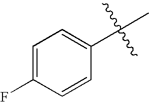 | —Cl | —H | 1.36 | 523 |
| 8-5 | 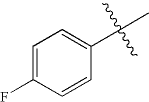 | —Cl | 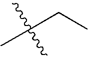 | 1.41 | 551 |
| 8-6 | 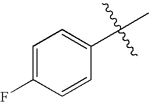 | —Cl | 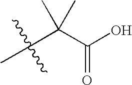 | 1.60 | 609 |
| 8-7 | 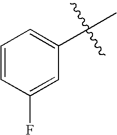 | —Cl | —H | 1.32 | 523 |
| 8-8 | 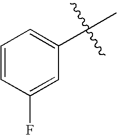 | —Cl | 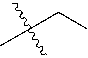 | 1.38 | 551 |
| 8-9 | 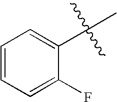 | —Cl | —H | 1.34 | 523 |
| 8-10 | 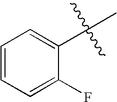 | —Cl | 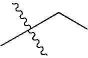 | 1.35 | 551 |
| 8-11 | 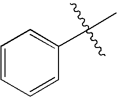 | —Cl | —H | 1.31 | 505 |
| 8-12 | 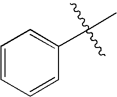 | —Cl | 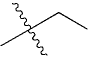 | 1.33 | 533 |
| 8-13 | 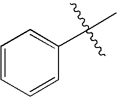 | —Cl | 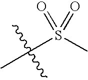 | 1.84 | 597 |
| 8-14 | 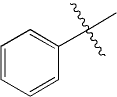 | —Cl | 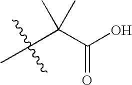 | 1.43 | 590 |
| 8-15 | 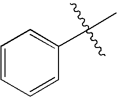 | —Cl | 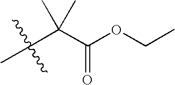 | 1.44 | 618 |
| 8-16 | 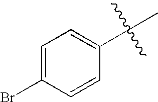 | —Cl | —H | 1.48 | 583 |
| 8-17 | 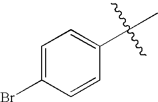 | —Cl | 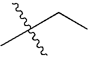 | 1.38 | 612 |
| 8-18 | 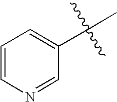 | —Cl | 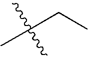 | 1.15 | 539 |
| 8-19 | 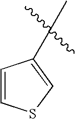 | —Cl | —H | 1.23 | 510 |
| 8-20 | 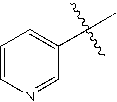 | —Cl | —H | 0.91 | 505 |
| 8-21 | 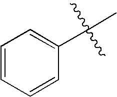 | —Cl | 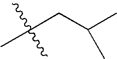 | 1.44 | 561 |
| 8-22 | 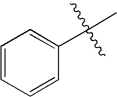 | —Cl | 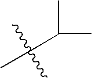 | 1.39 | 547 |
| 8-23 | 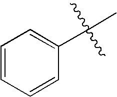 | —Cl | 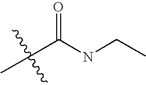 | 1.68 | 576 |
| 8-24 | 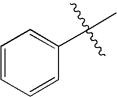 | —Cl | 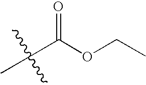 | 1.94 | 577 |
| 8-25 | 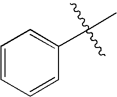 | —OMe | 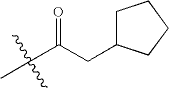 | 1.98 | 610 |
| 8-26 | 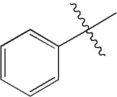 | —OMe | 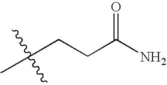 | 1.28 | 571 |
| 8-27 | 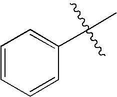 | —OMe | 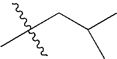 | 1.41 | 556 |
| 8-28 | 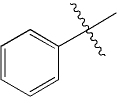 | —OMe | 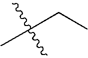 | 1.29 | 528 |

### Preparation of 1-(3-phenoxy-benzyl)piperdin-4-yl-1,3-dihydrobenzimdazol-2-one

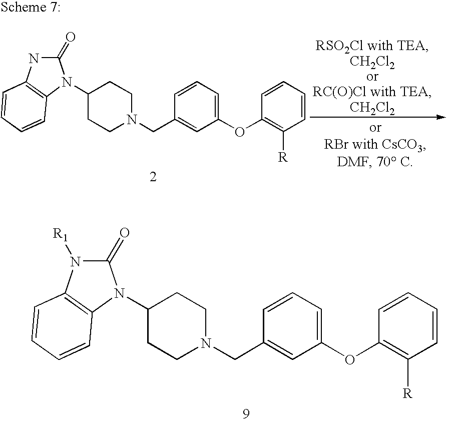

2 (1.0 equ.) was added to DMF followed by Cs₂CO₃ (1.5 equ.) and the corresponding alkyl bromide (1.5 equ.) in the case of the alkylation. The reaction was heated to 70° C. for 20 h. The reaction mixture was filtered and concentrated down. The residue was partitioned between ether and H₂O. The aqueous layer was extracted 3× with ether. The organics were collected together and washed 3× with water and dried over MgSO₄. The organics were filtered and concentrated down. The products were purified to give the desired products 9 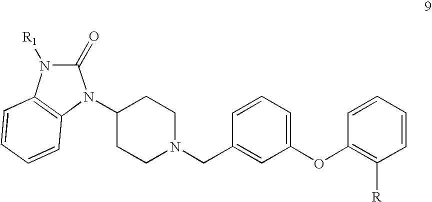
9-1:1-Ethanesulfonyl-3-[1-(3-phenoxy-benzyl)-piperidin-4-yl]-1,3-dihydro-benzoimidazol-2-one
9-2: 1-Ethyl-3-[1-(3-phenoxy-benzyl)-piperidin-4-yl]-1,3-dihydro-benzoimidazol-2-one
9-3:1-Ethyl-3-{1-[3-(2-methoxy-phenoxy)-benzyl]-piperidin-4-yl}-1,3-dihydro-benzoimidazol-2-one
9-4: 1-Isobutyl-3-[1-(3-phenoxy-benzyl)-piperidin-4-yl]-1,3-dihydro-benzoimidazol-2-one
9-5: 3-{2-Oxo-3-[1-(3-phenoxy-benzyl)-piperidin-4-yl]-2,3-dihydro-benzoimidazol-1-yl}-propionamide
9-6: 1-(2-Oxo-oxazolidin-5-ylmethyl)-3-[1-(3-phenoxy-benzyl)-piperidin-4-yl]-1,3-dihydro-benzoimidazol-2-one
9-7: 1-[1-(3-Phenoxy-benzyl)-piperidin-4-yl]-3-(2,2,2-trifluoro-ethyl)-1,3-dihydro-benzoimidazol-2-one
9-8: 1-[2-(1-Methyl-pyrrolidin-2-yl)-ethyl]-3-[1-(3-phenoxy-benzyl)-piperidin-4-yl]-1,3-dihydro-benzoimidazol-2-one
9-9: 1-Phenethyl-3-[1-(3-phenoxy-benzyl)-piperidin-4-yl]-1,3-dihydro-benzoimidazol-2-one
9-10: 1-Benzyl-3-{[1-[3-(2-methoxy-phenoxy)-benzyl]-piperidin-4-yl}-1,3-dihydro-benzoimidazol-2-one
9-11: 1-Pentanoyl-3-[1-(3-phenoxy-benzyl)-piperidin-4-yl]-1,3-dihydro-benzoimidazol-2-one
9-12: 1-[2-(4-Chloro-phenyl)-acetyl]-3-[1-(3-phenoxy-benzyl)-piperidin-4-yl]-1,3-dihydro-benzoimidazol-2-one
9-13: 1-(4-Chloro-phenylmethanesulfonyl)-3-[1-(3-phenoxy-benzyl)-piperidin-4-yl]-1,3-dihydro-benzoimidazol-2-one
9-14: 1-(2-Cyclopentyl-acetyl)-3-[1-(3-phenoxy-benzyl)-piperidin-4-yl]-1,3-dihydro-benzoimidazol-2-one
9-15: 1-(2-Morpholin-4-yl-ethyl)-3-[1-(3-phenoxy-benzyl)-piperidin-4-yl]-1,3-dihydro-benzoimidazol-2-one

9-16:1-(2-Diethylamino-ethyl)-3-[1-(3-phenoxy-benzyl)-piperidin-4-yl]-1,3-dihydro-benzoimidazol-2-one

TABLE 5

| | | | Retention | |
|---|---|---|---|---|
| | | | time | |
| No. | R | R₁ | (min) | M + 1 |
| 9-1 | —H | 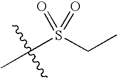 | 1.79 | 522.01 |
| 9-2 | —H | 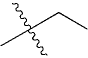 | 1.82 | 428 |
| 9-3 | —OMe | 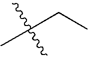 | 1.75 | 458 |
| 9-4 | —H | 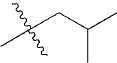 | 1.97 | 486.09 |
| 9-5 | —H | 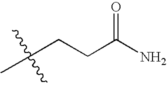 | 1.72 | 501.09 |
| 9-6 | —H | 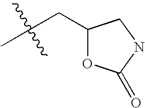 | 1.75 | 529.07 |
| 9-7 | —H | 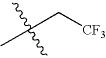 | 1.98 | 512.09 |
| 9-8 | —H | 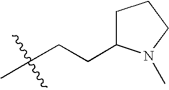 | 1.42 | 541.13 |
| 9-9 | —H | 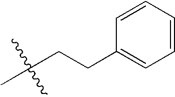 | 2.09 | 534.31 |
| 9-110 | —OMe | 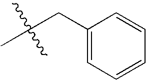 | 2.11 | 430 |
| 9-11 | —H | 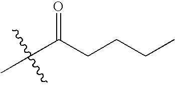 | 2.21 | 514.12 |
| 9-12 | —H | 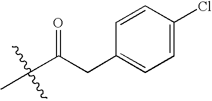 | 2.26 | 582.0 |
| 9-13 | —H | 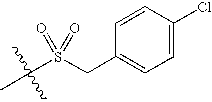 | 2.06 | 617.95 |
| 9-14 | —H | 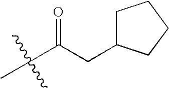 | 2.38 | 540.59 |
| 9-15 | —H | 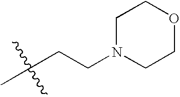 | 1.30 | 543.09 |
| 9-16 | —H | 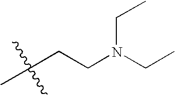 | 1.37 | 529.13 |

### Preparation of the 1-(3-phenoxy-benzyl)piperdin-4-yl-1,3-dihydrobenzimidazol-2-one

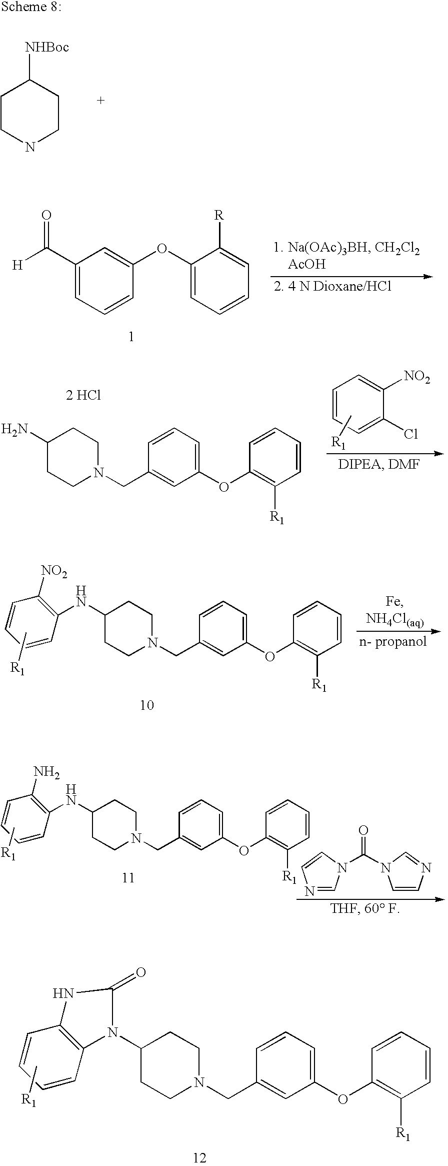

### 10: (2-nitrophenyl)-[phenoxy-benzyl)-piperdine-4-yl]-amine

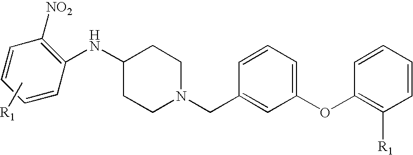

1-(3-phenoxy-benzyl)-piperdine-4-ylamine (1.0 equ) was added to DIPEA (5.5 equ.) in DMF, to the above was added the appropriate chloronitrobenzene (1.1 equ). The reaction mixture was heated to 100° C. for 48 h and then concentrated down. The residue was partitioned between ether and water. The organics were collected together and washed with 1N HCl, 3× water and 1× brine. The organics were dried over MgSO₄, filtered and concentrated down to give 10. The solid residues 10 were taken directly on to the following step.

### 11: (2-nitrophenyl)-[phenoxy-benzyl)-piperdine-4-yl]-amine

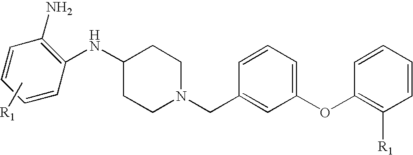

The corresponding aryl nitro 10 (1.0 equ) were reduced using iron and ammonium chloride. The aryl nitro 10 (1.0 equ) was added to 2-propanol, followed by a 0.34M solution of NH₄Cl (1.5 equ.) and 3 equ. of iron. The reaction was heated to 60° C. for 5 h, the color darkened considerable during this time. The reaction was filtered through Celite and concentrated down. The aqueous solution was extracted with methylene chloride 3×. The organics were washed with brine and dried over MgSO₄. The corresponding amines 11 were purified with MeOH/CH₂Cl₂.

### 12: 1-(3-phenoxy-benzyl)piperdin-4-yl-1,3-dihydrobenzimidazol-2-one

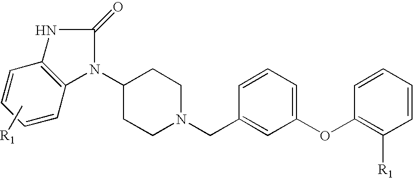

N-[1-(3-Phenoxy-benzyl)-piperidin-4-yl]-benzene-1,2-diamine 11 were cyclized with carbonyl diimidazole (CDI). The corresponding N-[1-(3-phenoxy-benzyl)-piperidin-4-yl]-benzene-1,2-diamine 11 compounds (1.0 equ.) were dissolved in THF and carbonyl diimidazole (1.5 equ) was added. The reaction was heated to reflux for 4 h. The reaction was diluted with EtOAc and washed with water and brine. The organic layer was dried over MgSO₄, filtered and concentrated down to give each of the corresponding substituted benzimidazole 12. Each compound 12 was purified using acetonitrile/H₂O with formic acid, to give the formate salts of 12.

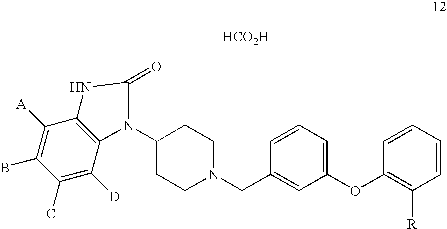

### 12-1: 4-Chloro-1-{1-[3-(2-methoxy-phenoxy)-benzyl]-piperidin-4-yl}-1,3-dihydro-benzoimidazol-2-one

12-2: 4-Chloro-1-[1-(3-phenoxy-benzyl)-piperidin-4-yl]-1,3-dihydro-benzoimidazol-2-one
12-3: 6-Chloro-1-[1-(3-phenoxy-benzyl)-piperidin-4-yl]-1,3-dihydro-benzoimidazol-2-one
12-4: 5-Acetyl-1-[1-(3-phenoxy-benzyl)-piperidin-4-yl]-1,3-dihydro-benzoimidazol-2-one
12-5: 5-Chloro-1-[1-(3-phenoxy-benzyl)-piperidin-4-yl]-1,3-dihydro-benzoimidazol-2-one
12-6: 1-[1-(3-Phenoxy-benzyl)-piperidin-4-yl]-5-trifluoromethyl-1,3-dihydro-benzoimidazol-2-one

12-7: 7-Chloro-1-[1-(3-phenoxy-benzyl)-piperidin-4-yl]-1,3-dihydro-benzoimidazol-2-one

TABLE 6

| | | | | | | Retention | |
|---|---|---|---|---|---|---|---|
| No. | A | B | C | D | R | time (min) | M + 1 |
| 12-1 | Cl | H | H | H | OMe | 1.73 | 464 |
| 12-2 | Cl | H | H | H | H | 1.79 | 434.13 |
| 12-3 | H | H | Cl | H | H | 2.01 | 434.03 |
| 12-4 | H | C(O)CH₃ | H | H | H | 1.58 | 442.07 |
| 12-5 | H | Cl | H | H | H | 1.75 | 434.00 |
| 12-6 | H | CF₃ | H | H | H | 1.99 | 468.03 |
| 12-7 | H | H | H | Cl | H | 1.99 | 434.02 |

### Preparation of 1-(3-phenoxy-benzyl)piperdin-4-yl]-1H-benzoimidazole

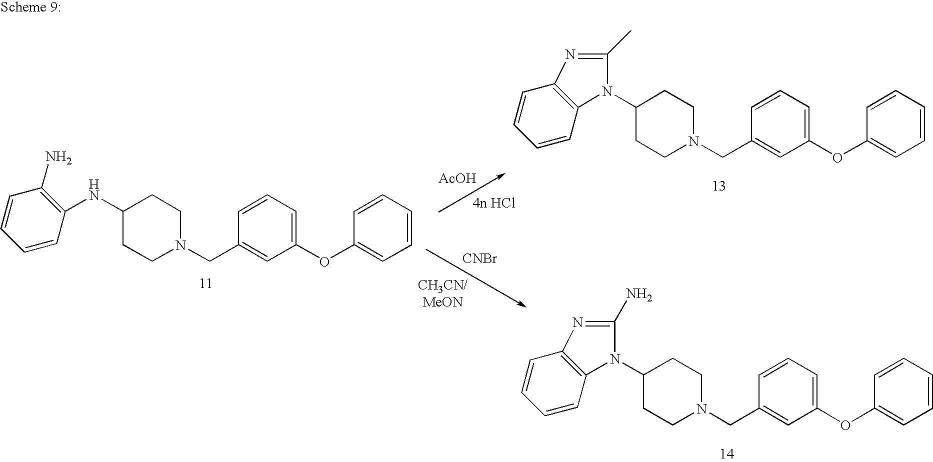

### 13: 2-Methyl-1-[1-(3-phenoxy-benzyl)-piperdin-4-yl]-1H-benzoimidazole

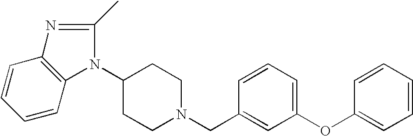

N-[1-(3-Phenoxy-benzyl)-piperidin-4-yl]-benzene-1,2-diamine, 11 (1.0 equ.) was mixed with 4N HCl (aq) and 1.0 equ of AcOH. The reaction mixture was allowed to heat at reflux for 10 h. The reaction mixture was neutralized with NaHCO₃ and extracted with methylene chloride (3×). The organics were collected together and washed with brine and dried over MgSO₄, filtered and concentrated down. The product was purified by flash chromatography with a gradient 100% CH₂Cl₂ to 96% CH₂Cl₂/4% MeOH to give a 39% yield of 13. Retention time 1.23, LCMS 398.05, ¹H NMR data is consistent with the assigned structure.

### 14: 1-[1-(3-Phenoxy-benzyl)-piperdin-4-yl]-1H-benzoimidazol-2-ylamine

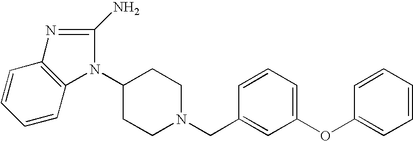

N-[1-(3-Phenoxy-benzyl)-piperidin-4-yl]-benzene-1,2-diamine, 11 was mixed with water and methanol and a solution of cyanogen bromide (1.0 equ, 5.0M solution in acetonitrile). The reaction was allowed to stir overnight at room temperature. Activated carbon was added to decolorize the reaction and filtered. The filtrate was brought to pH>9 with ammonium hydroxide and extracted with CH₂Cl₂. The aqueous layer was extracted 3× and the organics were collected together and washed with brine, dried over MgSO₄, filtered and concentrated down. The product was purified by flash chromatography with a gradient 100% CH₂Cl₂ to 90% CH₂Cl₂/10% MeOH to give a 29% yield of 14. Retention time 1.51, LCMS 399.07, ¹H NMR data is consistent with the assigned structure.

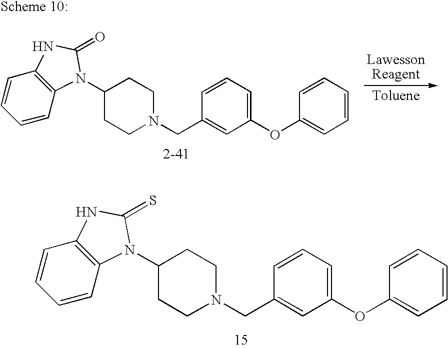

### 15: 1-[1-(3-Phenoxy-benzyl)-piperdin-4-yl]-1,3-dihydro-benzoimidazole-2-thione

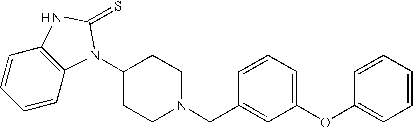

1-[1-(3-Phenoxy-benzyl)-piperdin-4-yl]-1,3-dihydro-benzoimidazol-2-one (1.0 equ.) was mixed with 1.1 equ of Lawesson reagent in toluene and heated to reflux overnight. The reaction mixture was concentrated down and purified by flash chromatography with a gradient 100% CH₂Cl₂ to 92% CH₂Cl₂/8% MeOH to give a 12% yield of 15. ¹H NMR data is consistent with the assigned structure.

### Preparation of 3-phenoxybenzylamines

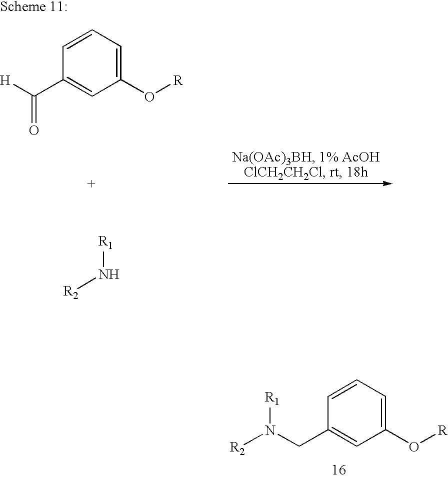

3-phenoxybenzaldehyde was mixed with an approriate amine (1.2 eq.) and sodium triacetoxy borohydride (1.2 eq.) in dichloroethane containing acetic acid (1%) and the resulting mixture was stirred at room temperature overnight. The reaction mixture was diluted with CH₂Cl₂ and washed with saturated aqueous sodium bicarbonate solution and brine and dried over sodium sulfate. Column chromatography provided the corresponding 3-aryloxy-benzyl amine 16. 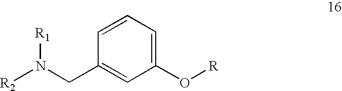
16-1: 4,5-Phenyl-9-(3-phenoxy-benzyl)-1-oxa-3,9-diaza-spiro[5.5]undec-4-en-2-one
16-2: 9-[3-(2-Methoxy-phenoxy)-benzyl]-4,5-phenyl-1-oxa-3,9-diaza-spiro[5.5]undec-4-en-2-one
16-3: 2-Benzyl-8-(−3-phenoxy-benzyl)-2,8-diaza-spiro[4.5]decan-3-one
16-4: 2-Benzyl-8-[3-(methoxy-phenoxy)-benzyl]-2,8-diaza-spiro[4.5]decan-3-one
16-5: 3, 4-Thiophen-9-(3-phenoxy-benzyl)-1-oxa-9-aza-spiro[5.5]undecen-2-one
16-6: 3, 4-Thiophen-9-[3-(2-methoxy-phenoxy)-benzyl]-1-oxa-9-aza-spiro[5.5]undecen-2-one
16-7: 4,5-Pyridine-9-(3-phenoxy-benzyl)-1-oxa-3,9-diaza-spiro[5.5]undec-4-en-2-one
16-8:1-(3-Phenoxy-benzyl)-4-phenyl-piperdin-4-ol
16-9: (4-Fluorophenyl)-[1-(3-phenoxy-benzyl)-piperdin-4-yl]-methanone
16-10: 4-Fluoro-phenyl)-{1-[3-(2-methoxy-phenoxy)-benzyl]piperdin-4-yl}methanone
16-11: 2-(4-Bromo-benzyl)-8-(3-phenoxy-benzyl)-2,8-diaza-spiro[4.5]decan-1-one
16-12: 2-Benzyl-9-(3-phenoxy-benzyl)-2,9-diaza-spiro[5.5]undecane
16-13: 4-(5-Furan-2-yl-1H-pyrazol-3-yl)-1-(3-phenoxy-benzyl)-piperdine
16-14: 4-(5-Furan-2-yl-1H-pyrazol-3-yl)-1-(3-(−2-methoxy-phenoxy)-benzyl]-piperdine
16-15: 2-[1-(3-Phenoxy-benzyl)-piperdin-4-yl]-ethanol
16-16: 2-[1-(3-(2-Methoxy-phenoxy)-benzyl]-piperdin-4-yl}-ethanol
16-17: 2-Benzyl-8-[3-phenoxy-benzyl]-1,2,8-triaza-spiro[4.5]decan-3-one
16-18: 2-Benzyl-8-[3-(2-methoxy-phenoxy)-benzyl]-1,2,8-triaza-spiro[4.5]decan-3-one
16-19: [1-(3-Phenoxy-benzyl)-piperdin-4-yl]-diphenyl-methanol
16-20: 1-(3-Phenoxy-benzyl)-4-phenyl-piperdine
16-21: [1-(3-Phenoxy-benzyl)-piperdin-4-yl]-phenyl-acetonitrile
16-22: 1-(3-Phenoxy-benzyl)-4-phenyl-piperdine-4-carbonitrile
16-23:1-(3-Phenoxy-benzyl)-4-phenyl-piperdine-4-carboxylic acid
16-24: 2-(3-Phenoxy-benzyl)-decahydro-isoquinoline
16-25: (4-Chloro-phenyl)-[1-(3-phenoxy-benzyl)-piperdin-4-yl]-methanone
16-26:1-[1-(3-Phenoxy-benzyl)-piperdin-4-yl]-1H-benzotriazole
16-27: 2-(3-Phenoxy-benzyl)-1,2,3,4-tetrahydroisoquinoline
16-28: 1-[1-(3-Phenoxy-benzyl)-4-phenyl-piperdin-4-yl-ethanone
16-29: 4-Benzyl-1-(3-phenoxy-benzyl)-piperdine
16-30: 3-(3-Phenoxy-benzyl)-1,2,3,4,5,6-hexahydro-1,5-methano-pyrido[1,2-a][1,5]diazocin-8-one
16-31: 2-(4-Chloro-phenyl)-5-(3-phenoxy-benzyl)-2,5-diaza-bicylco[2.2.1]heptane
16-32: [1-(3-Phenoxy-benzyl)-piperdin-4-yl]-phenyl-methanone
16-33: 1-(3-Phenoxy-benzyl)-azepane
16-34: 1,3,3-Trimethyl-6-(3-phenoxy-benzyl)-6-aza-bicyclo[3.2.1]octane
16-35: 4-[5-(4-Methoxy-phenyl)-1H-pyrazol-3-yl]-1-(3-phenoxy-benzyl)-piperdine
16-36: 2-[1-(3-Phenoxy-benzyl)-piperdin-4-yl]-benzothiazole
16-37: 1-(3-Phenoxy-benzyl)-pyrrolidine
16-38: 6-Fluoro-2-(3-phenoxy-benzyl)-2,3,4,9-tetrahydro-1H-O-carboline
16-39: 3-(3-Phenoxy-benzyl)-2,3,4,5-tetrahydro-1H-benzo[d]azepine
16-40: 4-Methyl-1-(3-phenoxy-benzyl)-4-phenyl-piperdine
16-41: 9-[3-(2-Methoxy-phenoxy)-benzyl]-2,3-phenyl-1-oxa-5,9-diaza-spiro[5.5]undec-2-en-4-one

16-42: 1-(3-Phenoxy-benzyl)-piperdine-3-carboxylic acid ethyl ester

TABLE 7

| | | | | |
|---|---|---|---|---|
| No. | R | 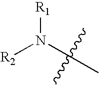 | Retention time (min) | M + 1 |

| | | | | |
|---|---|---|---|---|
| 16-1 | 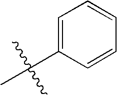 | 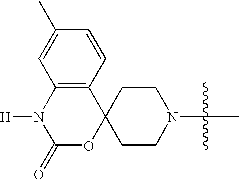 | 1.76 | 415.15 |
| 16-2 | 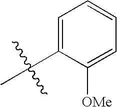 | 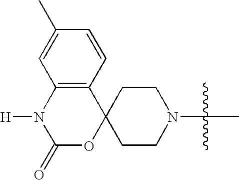 | 1.72 | 445 |
| 16-3 | 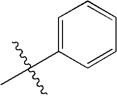 | 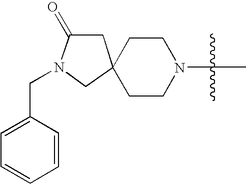 | 1.74 | 427.22 |
| 16-4 | 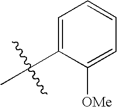 | 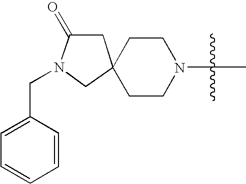 | 1.73 | 457 |
| 16-5 | 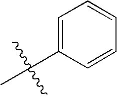 | 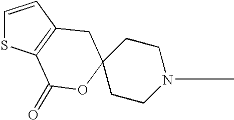 | 3.13 | 406.13 |
| 16-6 | 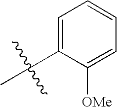 | 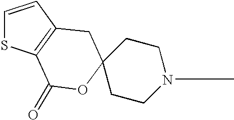 | 1.77 | 435 |
| 16-7 | 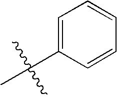 | 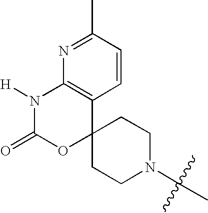 | 1.64 | 416.15 |
| 16-8 | 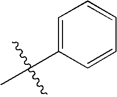 | 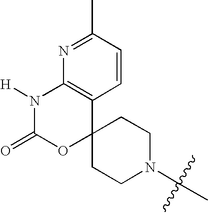 | 2.71 | 360.17 |
| 16-9 | 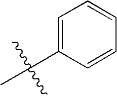 | 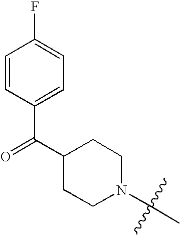 | 1.90 | 390.16 |
| 16-10 | 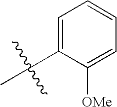 | 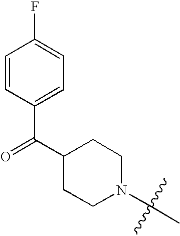 | 1.72 | 420 |
| 16-11 | 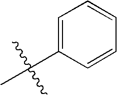 | 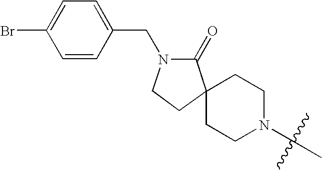 | 3.29 | 505.14 |
| 16-12 | 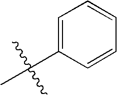 | 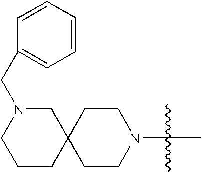 | 2.97 | 413.27 |
| 16-13 | 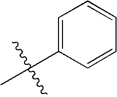 | 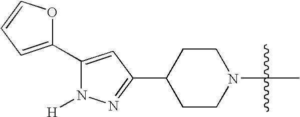 | 1.78 | 400.64 |
| 16-14 | 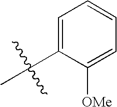 | 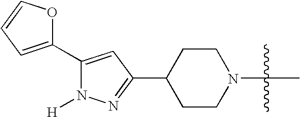 | 1.77 | 432 |
| 16-15 | 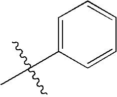 | 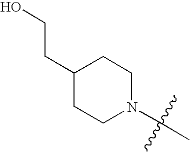 | 2.05 | 312.20 |
| 16-16 | 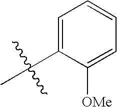 | 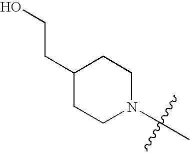 | 1.52 | 342.2 |
| 16-17 | 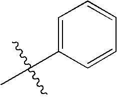 | 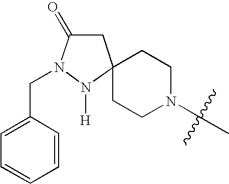 | 2.73 | 428.23 |
| 16-18 | 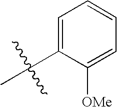 | 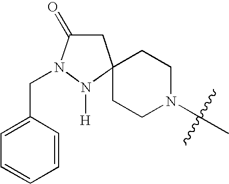 | 1.81 | 458 |
| 16-19 | 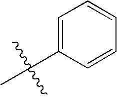 | 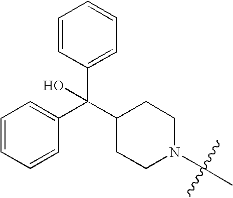 | 3.27 | 450.26 |
| 16-20 | 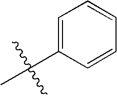 | 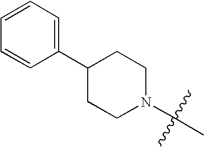 | 3.64 | 344.21 |
| 16-21 | 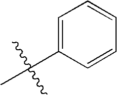 | 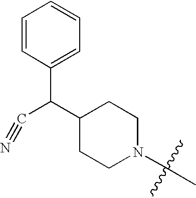 | 3.45 | 383.20 |
| 16-22 | 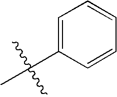 | 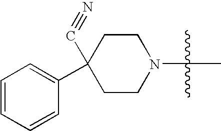 | 1.89 | 369.13 |
| 16-23 | 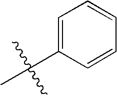 | 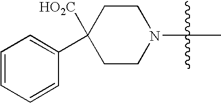 | 1.98 | 388.26 |
| 16-24 | 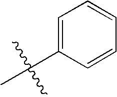 | 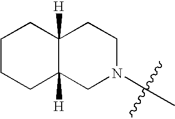 | 3.71 | 322.21 |
| 16-25 | 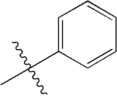 | 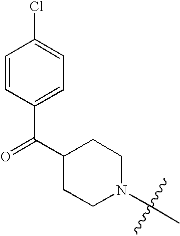 | 3.67 | 406.28 |
| 16-26 | 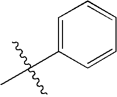 | 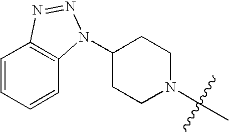 | 1.56 | 385.11 |
| 16-27 | 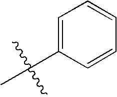 | 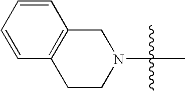 | 3.67 | 316.17 |
| 16-28 | 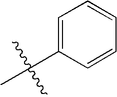 | 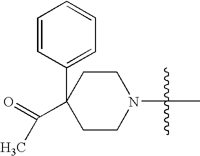 | 3.38 | 386.21 |
| 16-29 | 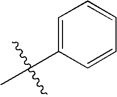 | 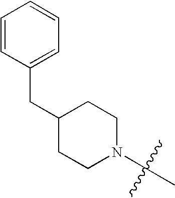 | 3.51 | 358.24 |
| 16-30 | 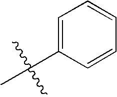 | 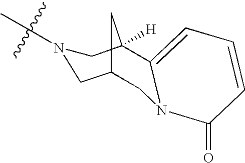 | 2.83 | 373.19 |
| 16-31 | 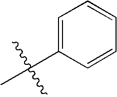 | 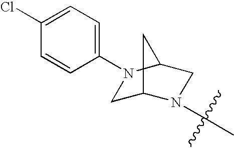 | 2.06 | 391.09 |
| 16-32 | 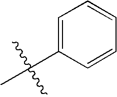 | 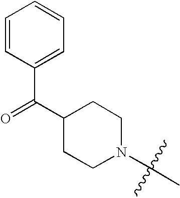 | 1.92 | 372.15 |
| 16-33 | 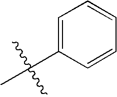 | 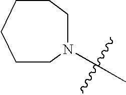 | 1.61 | 282.15 |
| 16-34 | 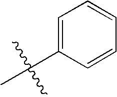 | 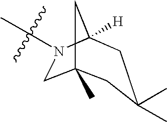 | 1.89 | 336.53 |
| 16-35 | 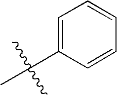 | 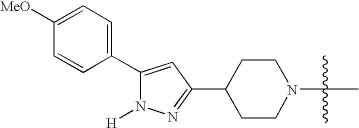 | 1.68 | 440.16 |
| 16-36 | 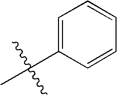 | 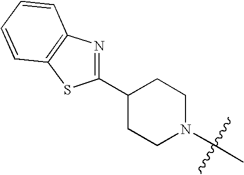 | 1.95 | 401.11 |
| 16-37 | 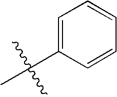 | 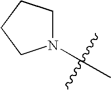 | 1.49 | 254.10 |
| 16-38 | 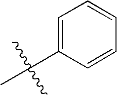 | 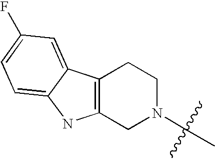 | 2.34 | 373.06 |
| 16-39 | 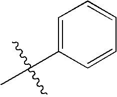 | 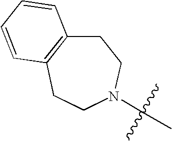 | 1.81 | 330.14 |
| 16-40 | 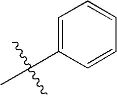 | 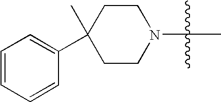 | 2.20 | 358.14 |
| 16-41 | 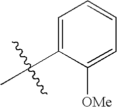 | 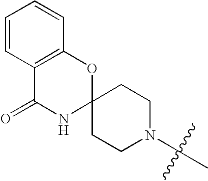 | 1.45 | 431.06 |
| 16-42 | 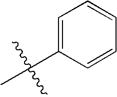 | 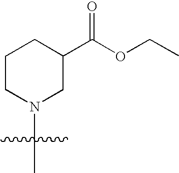 | 1.74 | 444 |

### Preparation of 4-Chloro-N-{1-[3-(2-methoxy-phenoxy)-benzyl]-piperidin-4-yl}-2-phenyl-butyramide

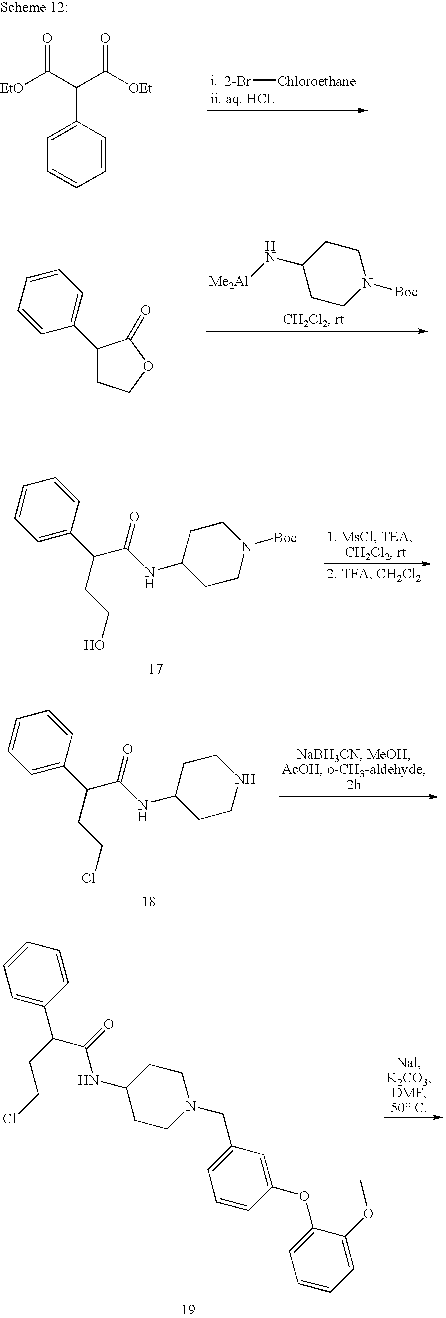

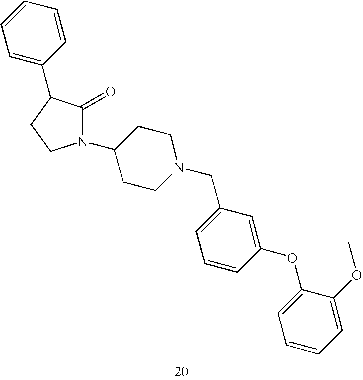

### 17: 4-(4-Hydroxy-2-phenyl-butyrylamino)-piperidine-1-carboxylic Acid Tert-Butyl Ester

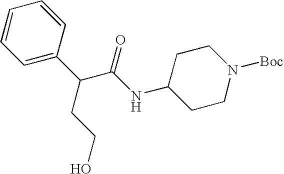

To an ice cooled solution of 4-amino-N-Boc-piperidone (1.04 g, 5.2 mmol) in dichloromethane (15 mL), was added Me₃Al (2M in toluene, 2.6 mL, 5.2 mmol). The resultant solution was stirred at rt for 0.5 h followed by the addition of α-phenyl-γ-butyrolactone (0.767 g, 4.7 mmol) as a solution in dichloromethane (5 mL). This solution was stirred overnight. The reaction mixture was quenched with saturated NH₄Cl solution and extracted with methylene chloride. The organic extracts were dried over MgSO₄, filtered and concentrated to provide the crude product as a white solid. Silica chromatography using (9:1 DCM: MeOH) provided the desired product 17 (0.82 g, 48%) as a white solid. ¹H NMR is consistent with assigned structure. Anal. Calcd for C₂₀H₃₀N₂O₄: C, 66.27, H, 8.34, N, 7.73. Found: C, 66.06; H, 8.41; N, 7.68.

### 18: 4-(4-Chloro-2-phenyl-butyrylamino)-piperidine-1-carboxylic Acid Tert-Butyl Ester

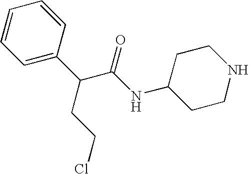

To a solution of 17 (149 mg, 0.45 mmol) in dichloromethane (3 mL) was added methanesulfonyl chloride (35 uL, 0.45 mmol) and TEA (62 uL, 0.45 mmol). The resultant mixture was stirred at rt for 6 h. The reaction solution was quenched with 1 N HCl solution and extracted with methylene chloride. The organic layer was dried over MgSO₄, filtered and concentrated to provide the crude product. Purification using silica gel chromatography (30% ethyl acetate in hexanes) provided the chloro product 18 (83 mg, 49%) as an oil. To a solution of the product in dichloromethane (5 mL), was added trifluoromethyl acetic acid (1 mL) and the reaction was stirred at rt for 1 h. The reaction solution was concentrated to provide the crude product that was used without further purification. ¹H NMR is consistent with assigned structure. LC-MS (ES+): 280, ret.time=1.10.

### 19: 4-Chloro-N-{1-[3-(2-methoxy-phenoxy)-benzyl]-piperidin-4-yl}-2-phenyl-butyramide

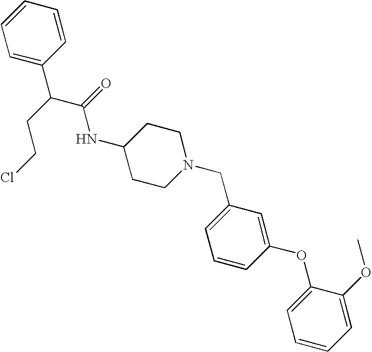

To a solution of 18 in dichloromethane (5 mL) was added trifluoromethyl acetic acid (1 mL). The resultant solution was stirred at rt for 1.5 h then concentrated in vacuo to provide the free amine as its TFA salt. This material was used directly in the next reaction without further purification. To a solution of the crude amine in methanol (5 mL) was added 3-(o-methoxyphenoxy)benzaldehyde 1-1 (350 mg, 2.2 mmol) and sodium cyanoborohydride (136 mg, 2.2 mmol). The reaction solution was adjusted to pH 6 by adding acetic acid then stirred at rt for 2 h. The reaction solution was quenched with saturated NaHCO₃ solution and extracted with diethyl ether. The organic extract was dried over MgSO₄, filtered and concentrated to provide the crude product. Purification using silica gel chromatography (20% ethyl acetate in hexanes) provided the desired product 19 (53 mg, 50%). ¹H NMR is consistent with assigned structure. LC-MS (ES+): 493, ret. time=1.70.

### 20:1-{1-[3-(2-Methoxy-phenoxy)-benzyl]-piperidin-4-yl}-3-phenyl-pyrrolidin-2-one

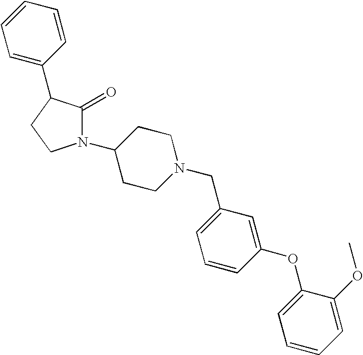

To a solution of 19 (53 mg, 0.10 mmol) in DMF (5 mL) was added a catalytic amount of potassium iodide and potassium carbonate (14 mg, 0.10 mmol). The resultant solution was stirred for 15 h at rt then heated for an additional 24 h. The reaction solution was cooled to rt and excess DMF was removed in vacuo. Flash chromatography using (9:1:0.5%, ethyl acetate: methanol, triethylamine) provided 20 (20 mg, 43%). ¹H NMR is consistent with assigned structure. LC-MS (ES+): 457, ret. time=1.07.

### 21-1, 21-2: 4-Hydroxy-N-{1-[3-(2-methoxy-phenoxy)-benzyl]-piperidin-4-yl}-2-phenyl-butyramide/4-Hydroxy-N-[1-(3-phenoxy-benzyl)-piperidin-4-yl]-2-phenyl-butyramide

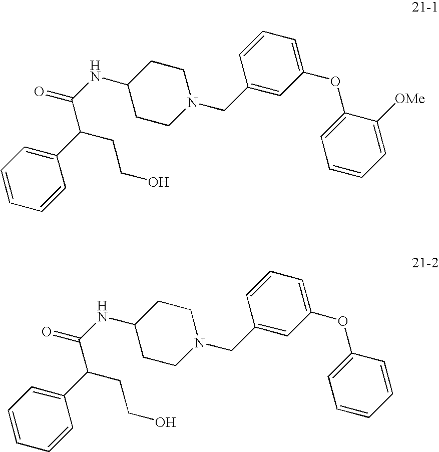

To a solution of 17 (544 mg, 1.43 mmol) in methylene chloride (10 mL) was added TFA (2 mL) and the resultant solution was stirred for 2 h at rt. Methylene chloride and TFA were removed iii vacuo and crude amine was used without further purification. 21-1: To a solution of the crude amine in methanol was added 3-(o-methoxyphenoxy)benzaldehyde (1-1) (814 mg, 3.6 mmol) and sodium cyanoborohydride (449 mg, 7.1 mmol). The reaction solution was adjusted to pH 6 by adding acetic acid then stirred at rt for 4 h. Additional portions of aldehyde 1-1 (1.6 g, 7.2 mmol) and NaBH₃CN (449 mg, 7.2 mmol) were added to the reaction solution and the solution was stirred overnight at rt. The reaction mixture was quenched with saturated NaHCO₃ solution and extracted with diethyl ether. The organic extract was dried over MgSO₄, filtered and concentrated to provide the crude product. HPLC chromatography (Phenomenex C18 column, 10 micron/60×21.2 mm) using 100:0 solvent A:B to 100% B over 27 minutes (A=99% H₂O/1% CH₃CN/0.1% formic acid, B=95% CH₃CN/5% H₂O/0.1% formic acid) provided the desired product 21-1 (678 mg, 62%). ¹H NMR is consistent with assigned structure. LC-MS (ES+): 445, ret. time=1.71.

21-2: To a solution of the crude amine (0.13 mmol) in methanol was added 3-phenoxybenzaldehyde (36 uL, 0.20 mmol) and sodium cyanoborohydride (84 mg, 1.3 mmol). The reaction solution was adjusted to pH 6 by adding acetic acid and stirred at overnight. During the course of the reaction, an additional portion of phenoxybenaldehyde (1.6 g, 7.2 mmol) was added to the reaction solution after 6 h. The reaction mixture was quenched with saturated NaHCO₃ solution and extracted with diethyl ether. The organic extract was dried over MgSO₄, filtered and concentrated to provide the crude product. HPLC chromatography (Phenomenex C18 column, 10 micron/60×21.2 mm) using 85:15 solvent A:B to 100% B over 25 minutes (A=99% H₂O/1% CH₃CN/0.1% formic acid, B=95% CH₃CN/5% H₂O/0.1% formic acid) provided the desired product 21-2 (9 mg, 16%). ¹H NMR is consistent with assigned structure. LC-MS (ES+): 475, ret. time=1.70.

### 22-1, 22-2: N-{1-[3-(2-Methoxy-phenoxy)-benzyl]-piperidin-4-yl}-2-phenyl-4-pyrrolidin-1-yl-butyramide/4-Diethylamino-N-{1-[3-(2-methoxy-phenoxy)-benzyl]-piperidin-4-yl}-2-phenyl-butyramide

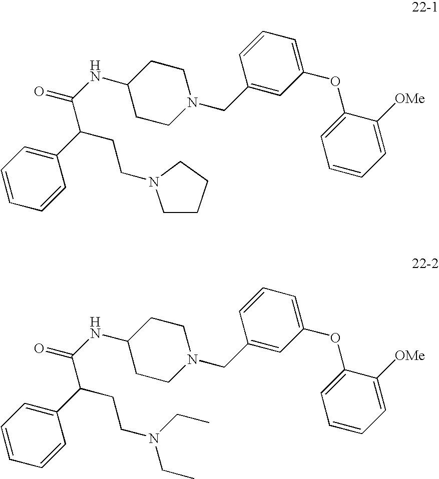

To a solution of 19 (154 mg, 0.32 mmol) in dichloromethane (2 mL) was added methanesulfonyl chloride (28 uL, 0.32 mmol), and triethylamine (58 uL, 0.42 mmol). The resultant solution was stirred at rt for 4 h. The reaction solution was quenched with 1 N HCl solution and extracted with methylene chloride. The organic extracts were dried over MgSO₄, filtered and concentrated to provide the crude chloride compound as a brown residue. This product was used in subsequent reactions without further purification. 22-1: To a solution of the crude chloride (˜0.16 mmol) in dioxane (1 mL) was added pyrrolidine (0.540 mL, 6.2 mmol). The resultant solution was heated for 12 h at 60° C. Upon cooling to rt, the reaction solution was concentrated and the crude product was further purified by flash chromatography (10% methanol in dichloromethane) (18 mg, 21%). ¹H NMR is consistent with assigned structure. LC-MS (ES+): 528, ret. time=1.91.

22-2: To a solution of the crude chloride (˜0.16 mmol) in dioxane (1 mL) was added diethyl amine (0.66 mL, 6.2 mmol). The resultant solution was heated for 48 h at 60° C. After 12 h, an additional diethylamine was added (0.66 mL, 6.2 mmol). Upon cooling to rt, the reaction solution was concentrated and the crude product was further purified by flash chromatography (10% methanol in dichloromethane) to provide the desired product 22-2 (27 mg, 32%). ¹H NMR is consistent with assigned structure. LC-MS (ES+): 530, ret. time=1.14.

### 23: Piperidine-1,4-dicarboxylic Acid 1-benzyl Ester 4-ethyl Ester

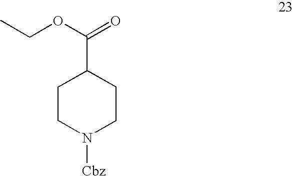

To an iced cooled solution of ethyl isonipecoate (15.4 mL, 0.1 mol) and triethylamine (27.8 mL, 0.2 mol) in dichloromethane (300 mL) was added Cbz-Cl (15.7 mL, 0.11 mol) dropwise using a slow addition funnel. The reaction solution was stirred at rt for 2 days. To this solution was added 1 N HCl and product was extracted with dichloromethane. The organic extracts were dried over MgSO₄, filtered and concentrated to provide the desired product 23 as a pink semi-solid (16 g, 55%). This product was used without further purification. ¹HNMR is consistent with assigned structure. LC-MS (ES+): 292, ret time=2.49.

### 24: 4-Hydroxymethyl-piperidine-1-carboxylic Acid Benzyl Ester

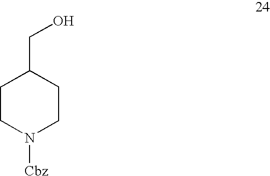

To a solution of 23 (5.82 g, 20 mmol) in toluene (60 mL) cooled to −60° C., was added diisobutylaluminium hydride (40 mL, 40 mmol). The resultant solution was warmed to rt overnight. The reaction solution was quenched with 1 N HCl and extracted with ethyl ether. The organic extracts were dried over MgSO₄, filtered and concentrated to provide the desired product as an oil. Flash chromatography (20% ethyl acetate in hexanes) provided the desired product 24 as an oil (2.0 g, 40%). ¹H NMR is consistent with assigned structure.

### 25: 4-Formyl-piperidine-1-carboxylic Acid Benzyl Ester

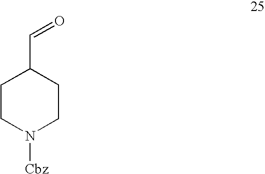

To a solution of DMSO (1.6 mL, 23 mmol) in methylene chloride (50 mL) cooled to −60° C., was added oxalyl chloride (1.25 mL, 14.3 mmol) dropwise. The resultant solution was stirred at 60° C. for 15 minutes followed by the addition of 24 (2.8 g, 9.5 mmol) as a solution in methylene chloride. The solution was stirred for an additional 15 minutes at −60° C. After that time, triethylamine (6.65 mL, 47.8 mmol) was added in one portion and the reaction solution was warmed to rt over 1.5 hour. The reaction solution was quenched with 1 N HCl, extracted with dichloromethane, dried over MgSO₄, and concentrated to provide the crude product as an yellow oil. Flash chromatography (20-30% ethyl acetate in hexanes) provided the desired product 25 as a clear oil. ¹H NMR is consistent with assigned structure.

### 26: 4-(2,3-dihydro-1H-indol-3-yl)-piperidine-1-carboxylic Acid Benzyl Ester

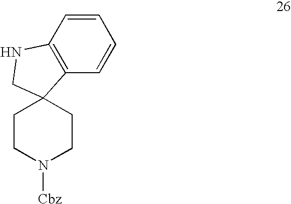

To a degassed solution of toluene and acetonitrile (49:1 v/v, 15 mL total) was added phenyl hydrazine (0.45 mL, 4.5 mmol) and trifluoromethyl acetic acid (1.1 mL, 13.6 mmol). The reaction solution was heated to 35° C. To the reaction mixture was added dropwise aldehyde 25 (1.2 g, 4.1 mmol), as a solution in the toluene/acetonitrile solution (3 mL total). The resultant solution was heated at 35° C. for 20 h. The solution was then cooled to −10° C. and to it was added NaBH₄ (233 mg, 62 mmol). The reaction mixture was quenched with 1 N HCl and extracted with ethyl ether. The organic extracts were dried over MgSO₄, filtered and concentrated to provide the desired product. Flash chromatography (20-30% ethyl acetate in hexanes) provided 26 (585 mg, 44%). ¹H NMR is consistent with assigned structure. LC-MS (ES+): 323, ret. time=1.89.

### 27: 4-(2,3-dihydro-1-methanesulfonyl-indol-3-yl)-piperidine-1-carboxylic Acid Benzyl Ester

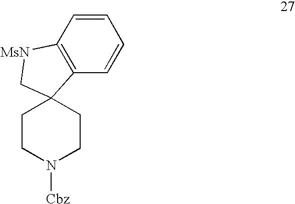

To a solution of 26 (585 mg, 1.8 mmol) in dichloromethane (5 mL) and triethylamine (0.33 mL, 2.4 mmol) cooled to 0° C. was added was added methanesulfonylchloride (0.158 mL, 2.0 mmol). The reaction solution was warmed to rt over 5 h. The reaction mixture was quenched with 1 N HCl and extracted with ethyl acetate. The organic extracts were dried over MgSO₄, filtered and concentrated to provide the crude product as an oil. Flash chromatography using 20-30% ethyl acetate in hexanes provided the desired product 27 (550 mg, 76%) as a white foam. ¹H NMR is consistent with assigned structure. LC-MS (ES+): 401, ret time=2.71.

### 28-1, 28-2: N-[3-(2-methoxy-phenoxy)-benzyl]-4-(2,3-dihydro-1-(methanesulfonyl)-indol-3-yl)-piperidine/N-[3-(2-chloro-phenoxy)-benzyl]-4-(2,3-dihydro-1-(methanesulfonyl)-indol-3-yl)-piperidine

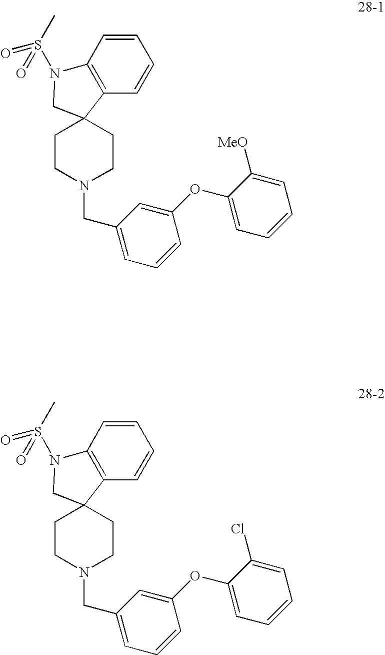

Amine 27 (510 mg, 1.3 mmol), 10% Pd—C (107 mg) and ethanol (20 mL) were combined in a pressure vessel. The vessel was pressurized to 45 psi with hydrogen gas then heated at 65° C. for 24 h. The reaction vessel was cooled to rt and Pd—C removed by filtration through a plug of celite. The celite pad was washed with extra amounts of ethanol. The organic solution was concentrated to provide the desired product (258 mg, 68%). The product was used directly in the next reaction.

28-1: To the crude hydrogenation product (124 mg, 0.47 mmol) and o-methoxy-aldehyde 1-1 (138 mg, 0.61 mmol) in methanol, was added sodium cyanoborohydride (29 mg, 0.47 mmol). The reaction solution was acidified to pH 6 with acetic acid and stirred at rt overnight. The reaction solution was quenched with 1N HCl solution and extracted with methylene chloride. The organic extracts were dried over MgSO4, filtered and concentrated to provide the crude product. Flash chromotography (20% ethyl acetate in hexanes) provided the desired 28-1 as a white foam. ¹H NMR is consistent with assigned structure. Anal. Calcd: C, 67.74; H, 6.32, N, 5.85. Found; C, 67.64, H, 6.34, N, 5.74. LC-MS (ES+): 479, ret time=1.71.

28-2: To the crude hydrogenation product (124 mg, 0.47 mmol) and o-chloro-aldehyde 1-6 (142 mg, 0.61 mmol) in methanol, was added sodium cyanoborohydride (29 mg, 0.47 mmol). The reaction solution was acidified to pH 6 with acetic acid and stirred at rt overnight. The reaction solution was quenched with 1N HCl solution and extracted with methylene chloride. The organic extracts were dried over MgSO₄, filtered and concentrated to provide the crude product. Flash chromotography (20% ethyl acetate in hexanes) provided the desired product 28-2 as a white foam. ¹H NMR is consistent with assigned structure. Anal. calcd: C, 64.65; H, 5.63, N, 5.80. Found; C, 64.40, H, 5.74, N, 5.74. LC-MS (ES+): 482, ret time=1.80.

### Preparation of N-(3-{Ethyl-[3-phenoxy-benzyl]-amino}-propyl)-2-acetamide

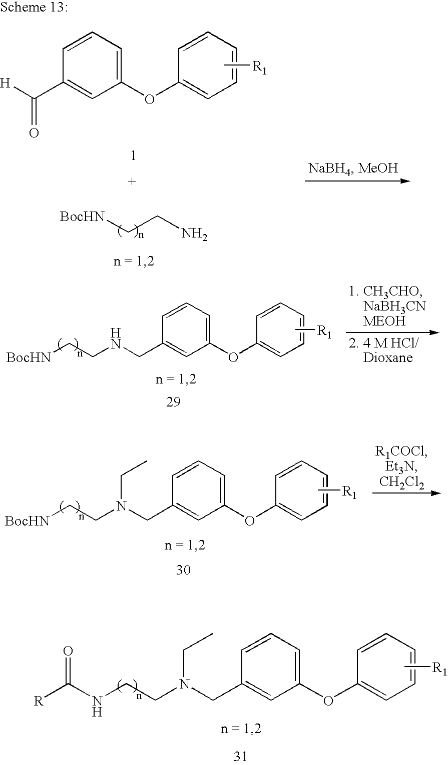

### 29: (3-{Ethyl-[3-(2-methoxy-phenoxy)-benzyl]-amino}-propyl)-carbamic Acid Tert-Butyl Ester

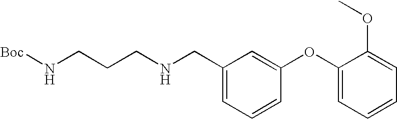

A solution of N-(3-aminopropyl)-carbamic acid t-butyl ester (2.61 g, 15 mmol) and o-methoxy-aldehyde 1-1 (3.42 g, 15 mmol) in methanol (60 mL) was heated to reflux for 48 h. The resultant solution was cooled to rt and to it was added sodium borohydride (0.561 mg, 15 mmol). The reaction solution was stirred for 1 day at rt then quenched with 1N HCl and extracted with methylene chloride. The organic extract was dried over magnesium sulfate, filtered and concentrated to provide the crude product as an oil. This material was used directly in the next reaction without further purification. ¹H NMR is consistent with assigned structure. LC-MS (ES+): 387, ret time=1.50.

### 30: N−1-Ethyl-N1-[3-(2-methoxy-phenoxy)-benzyl]-propane-1,3-diamine Dihydrochloride

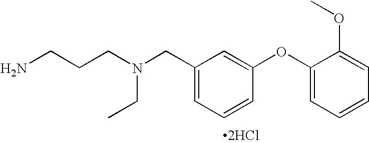

To the crude product (˜15 mmol) 29 dissolved in methanol (40 mL) was added acetaldehyde (2.5 mL, 45 mmol) and NaBH₃CN (1 g, 15 mmol). The resultant solution was stirred overnight at rt. The reaction solution was quenched with saturated ammonium chloride solution, extracted with methylene chloride. The organic extracts were dried over MgSO₄, filtered and concentrated to provide the desired product as an oil. This material was dissolved with 4N HCl in dioxane (80 mL), stirred at rt for 3 h, and concentrated to provide 30 (5 g, 92%) as an off-white solid. This material was used directly in the next reaction without further purification. ¹H NMR is consistent with assigned structure. LC-MS (ES+): 315, ret. time.

### 31: N-(3-{Ethyl-[3-(2-methoxy-phenoxy)-benzyl]-amino}-propyl)-2-phenyl-acetamide

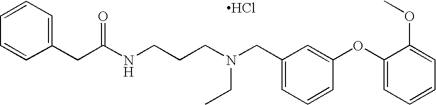

To a solution of diamine 30 (0.359 g, 1 mmol) in methylene chloride (5 mL) was added triethylamine (0.4 mL, 3 mmol) and phenylacetyl chloride (132 uL, 1.1 mmol). The resultant solution was agitated on the orbital shaker overnight. The crude reaction solution was concentrated in vacuo to provide the product as an oil. Flash chromatography on SiO₂ provided the desired product 31 as on oil, which was converted to its HCl salt following conventional methods. ¹H NMR is consistent with assigned structure. LC-MS (ES+): 463, ret. time=0.79 min. 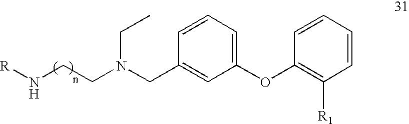
31-1: 2-(4-Benzyloxy-phenyl)-N-{2-[ethyl-(3-phenoxy-benzyl)-amino]-ethyl}-acetamide
31-2: N-{2-[Ethyl-(3-phenoxy-benzyl)-amino]-ethyl}-2-(3-methoxy-phenyl)-acetamide
31-3: N-{2-[Ethyl-(3-phenoxy-benzyl)-amino]-ethyl}-2-thiophen-2-yl-acetamide
31-4: N-{2-[Ethyl-(3-phenoxy-benzyl)-amino]-ethyl}-2-(4-fluoro-phenyl)-acetamide
31-5: N-{2-[Ethyl-(3-phenoxy-benzyl)-amino]-ethyl}-4-fluoro-benzamide
31-6: 2-(3,4-Dimethoxy-phenyl)-N-{2-[ethyl-(3-phenoxy-benzyl)-amino]-ethyl}-acetamide
31-7: N-{2-[Ethyl-(3-phenoxy-benzyl)-amino]-ethyl}-2-phenoxy-acetamide
31-8: 3-{2-[Ethyl-(3-phenoxy-benzyl)-amino]-ethyl}-1-methyl-1-phenyl-urea
31-9: N-{2-[Ethyl-(3-phenoxy-benzyl)-amino]-ethyl}-3-phenyl-propionamide
31-10: N-{3-[Ethyl-(3-phenoxy-benzyl)-amino]-propyl}-2,2-diphenyl-acetamide
31-11: 2-(4-Benzyloxy-phenyl)-N-{3-[ethyl-(3-phenoxy-benzyl)-amino]-propyl}-acetamide
31-12: N-{3-[Ethyl-(3-phenoxy-benzyl)-amino]-propyl}-2-(3-methoxy-phenyl)-acetamide
31-13: 2-(2-Bromo-phenyl)-N-{3-[ethyl-(3-phenoxy-benzyl)-amino]-propyl}-acetamide0
31-14: N-{3-[Ethyl-(3-phenoxy-benzyl)-amino]-propyl}-2-(4-fluoro-phenyl)-acetamide
31-15: N-{3-[Ethyl-(3-phenoxy-benzyl)-amino]-propyl}-4-fluoro-benzamide
31-16: N-{3-[Ethyl-(3-phenoxy-benzyl)-amino]-propyl}-2-phenoxy-acetamide
31-17: N-{3-[Ethyl-(3-phenoxy-benzyl)-amino]-propyl}-3-phenyl-acrylamide
31-18: 3-Cyclopentyl-N-{3-[ethyl-(3-phenoxy-benzyl)-amino]-propyl}-propionamide
31-19: N-{3-[Ethyl-(3-phenoxy-benzyl)-amino]-propyl}-3-phenyl-propionamide
31-20: N-{3-[Ethyl-(3-phenoxy-benzyl)-amino]-propyl}-2-thiophen-2-yl-acetamide
31-21: 3,5,5-Trimethyl-hexanoic acid {3-[ethyl-(3-phenoxy-benzyl)-amino]-propyl}-amide
31-22: N-(3-{Ethyl-[3-(2-methoxy-phenoxy)-benzyl]-amino}-propyl)-2-(3-methoxy-phenyl)-acetamide
31-23: N-(3-{Ethyl-[3-(2-methoxy-phenoxy)-benzyl]-amino}-propyl)-2-thiophen-2-yl-acetamide
31-24: N-(3-{Ethyl-[3-(2-methoxy-phenoxy)-benzyl]-amino}-propyl)-4-fluoro-benzamide
31-25: N-(3-{Ethyl-[3-(2-methoxy-phenoxy)-benzyl]-amino}-propyl)-2-(4-fluoro-phenyl)-acetamide
31-26: 2-(3,4-Dimethoxy-phenyl)-N-(3-{ethyl-[3-(2-methoxy-phenoxy)-benzyl]-amino}-propyl)-acetamide
31-27: N-(3-{Ethyl-[3-(2-methoxy-phenoxy)-benzyl]-amino}-propyl)-C-phenyl-methanesulfonamide
31-28: 3-(3-{Ethyl-[3-(2-methoxy-phenoxy)-benzyl]-amino}-propyl)-1-methyl-1-phenyl-urea
31-29: (3-{Ethyl-[3-(2-methoxy-phenoxy)-benzyl]-amino}-propyl)-carbamic acid 4-methoxy-phenyl ester
31-30: 2-(2-Bromo-phenyl)-N-(3-{ethyl-[3-(2-methoxy-phenoxy)-benzyl]-amino}-propyl)-acetamide
31-31: N-(3-{Ethyl-[3-(2-methoxy-phenoxy)-benzyl]-amino}-propyl)-3-phenyl-propionamide

31-32: N-(3-{Ethyl-[3-(2-methoxy-phenoxy)-benzyl]-amino}-propyl)-2-phenyl-acetamide

TABLE 9

| | | | | Retention | |
|---|---|---|---|---|---|
| No. | R | n | R₁ | time (min) | M + 1 |
| 31-1 | 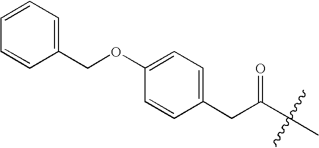 | 1 | H | 2 | 495 |
| 31-2 | 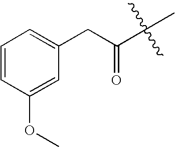 | 1 | H | 1.64 | 419 |
| 31-3 | 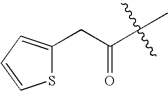 | 1 | H | 1.64 | 395 |
| 31-4 | 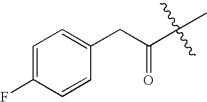 | 1 | H | 1.67 | 407 |
| 31-5 | 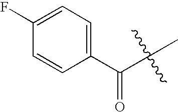 | 1 | H | 1.68 | 393 |
| 31-6 | 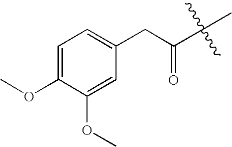 | 1 | H | 1.58 | 449 |
| 31-7 | 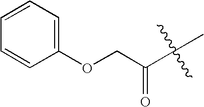 | 1 | H | 1.7 | 405 |
| 31-8 | 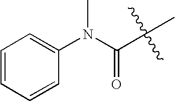 | 1 | H | 1.67 | 404 |
| 31-9 | 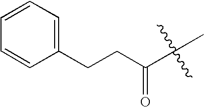 | 1 | H | 1.61 | 403 |
| | | | | Retention | |
| No. | R | R₁ | n | time (min) | M + 1 |
| 31-10 | 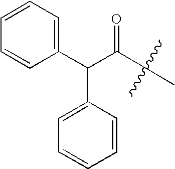 | H | 2 | 2 | 479 |
| 31-11 | 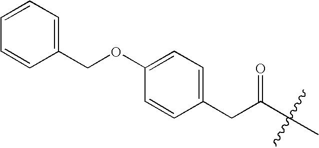 | H | 2 | 2 | 509 |
| 31-12 | 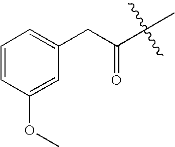 | H | 2 | 1.68 | 433 |
| 31-13 | 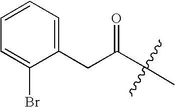 | H | 2 | 1.79 | 482 |
| 31-14 | 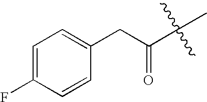 | H | 2 | 1.7 | 421 |
| 31-15 | 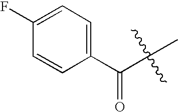 | H | 2 | 1.69 | 407 |
| 31-16 | 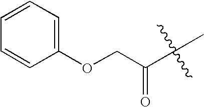 | H | 2 | 1.75 | 419 |
| 31-17 | 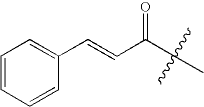 | H | 2 | 1.78 | 415 |
| 31-18 | 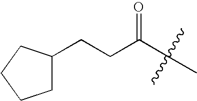 | H | 2 | 1.85 | 409 |
| 31-19 | 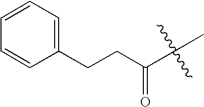 | H | 2 | 1.78 | 417 |
| 31-20 | 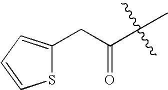 | H | 2 | 1.62 | 409 |
| 31-21 | 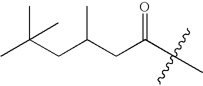 | H | 2 | 2.01 | 425 |
| 31-22 | 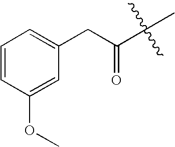 | OMe | 2 | 1.58 | 463 |
| 31-23 | 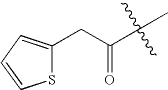 | OMe | 2 | 1.61 | 439 |
| 31-24 | 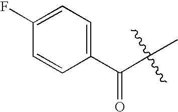 | OMe | 2 | 1.58 | 437 |
| 31-25 | 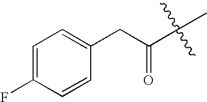 | OMe | 2 | 1.63 | 451 |
| 31-26 | 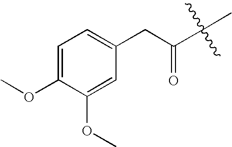 | OMe | 2 | 1.53 | 493 |
| 31-27 | 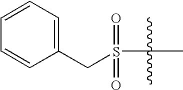 | OMe | 2 | 1.62 | 469 |
| 31-28 | 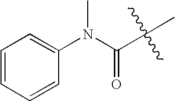 | OMe | 2 | 1.64 | 448 |
| 31-29 | 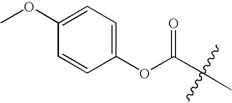 | OMe | 2 | 1.62 | 465 |
| 31-30 | 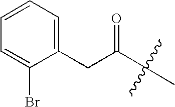 | OMe | 2 | 1.72 | 512 |
| 31-31 | 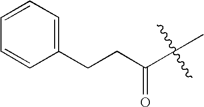 | OMe | 2 | 1.62 | 447 |
| 31-32 | 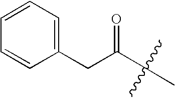 | OMe | 2 | 1.6 | 433 |

### Preparation of 2-{1-[3-phenoxy-benzyl]-piperidin-4-ylamino}-3-phenyl-alkyl Acid Methyl Ester

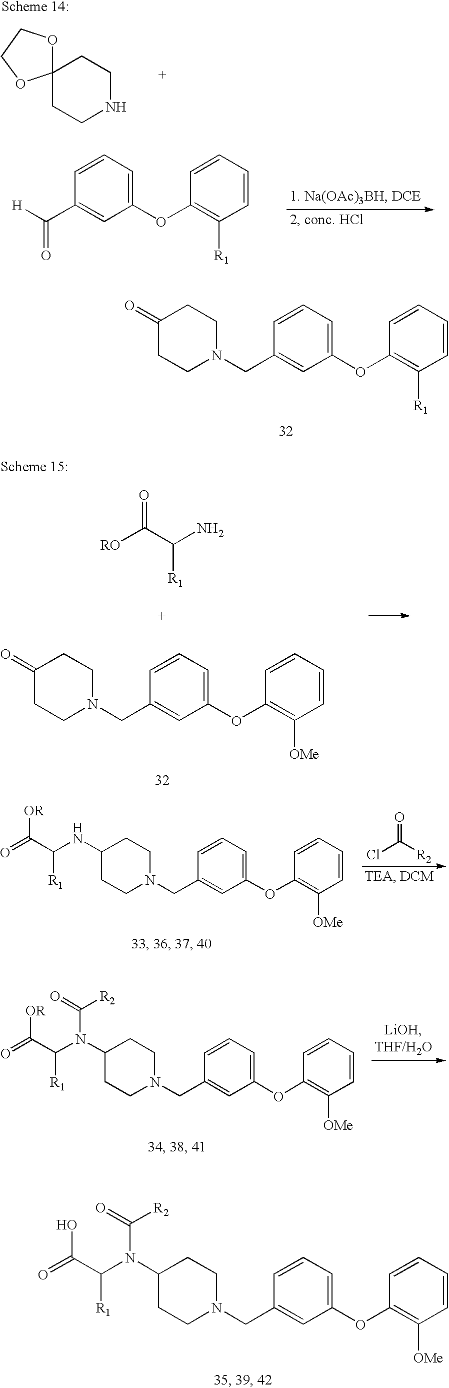

### 33: {1-[3-(2-Methoxy-phenoxy)-benzyl]-piperidin-4-ylamino}-acetic Acid Methyl Ester

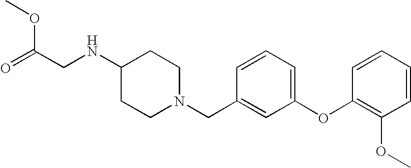

To a solution of ketone 32-2 (500 mg, 1.6 mmol) and glycine methyl ester HCl (301 mg, 2.4 mmol) in methanol (10 mL) was added sodium cyanoborohydride (100 mg, 1.6 mmol). The resultant solution was stirred at rt overnight. The reaction mixture was quenched with saturated NaHCO₃ solution and extracted with ethyl ether. The organic layer was dried over MgSO₄, filtered and concentrated to provide the desired product 33 which was clean enough to use without further purification. ¹H NMR is consistent with assigned structure. LC-MS (ES+): 385, ret. time=0.86 min.

### 34: [{1-[3-(2-Methoxy-phenoxy)-benzyl]-piperidin-4-yl}-(2-phenyl-butyryl)-amino]-acetic Acid Methyl Ester

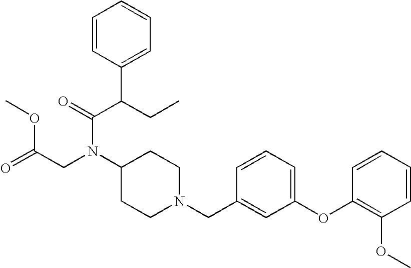

To a solution of amine 33 (175 mg, 0.46 mmol) in dichloromethane (5 mL) were added 2-Phenyl-butyryl chloride (86 uL, 0.51 mmol) and triethylamine (0.32 mL, 2.3 mmol). The resultant solution was stirred at rt for 2 h then quenched with saturated sodium bicarbonate solution. The organic layer was dried over MgSO₄, filtered and concentrated to provide the crude product 34. Flash chromatography using (95:5 EtOAc: Hexanes w/0.5% TEA) provided the desired product 34. ¹H NMR is consistent with assigned structure. LC-MS (ES+): 531, ret. time=1.72 min.

### 35: [{1-[3-(2-Methoxy-phenoxy)-benzyl]-piperidin-4-yl}-(2-phenyl-butyryl)-amino]-acetic Acid

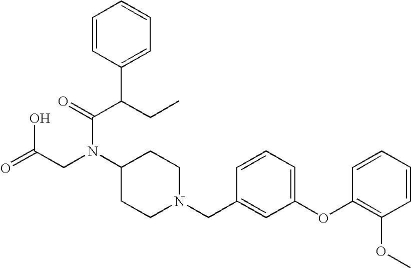

To a solution of amine 34 (94 mg, 0.17 mmol) in THF/water (4:1, 10 mL) was added lithium hydroxide (8.2 mg, 0.19 mmol). The resultant solution was stirred at rt for 7 h. The reaction solution was quenched with ammonium acetate solution and extracted with ethyl ether. The organic layer was dried over MgSO₄, filtered and concentrated to provide the crude product 35. HPLC chromatography (phenomenex C18 column, 10 micron/60×21.2 mm) using 85:15 solvent A:B to 100% B over 25 minutes (A=99% H₂O/1% CH₃CN/0.1% formic acid, B=95% CH₃CN/5% H₂O/0.1% formic acid) provided the desired product 35. ¹H NMR is consistent with the assigned structure. LC-MS (S+): 517, ret. time=1.67 min.

### 36: {1-[3-(2-Methoxy-phenoxy)-benzyl]-piperidin-4-ylamino}-acetic Acid Benzyl Ester

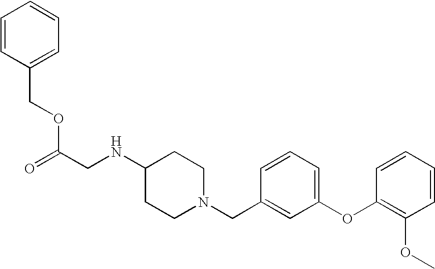

To a solution of ketone 32-2 (500 mg, 1.6 mmol) and glycine benzyl ester HCl (324 mg, 1.6 mmol) in methanol (10 mL) was added sodium cyanoborohydride (101 mg, 1.6 mmol). The resultant solution was stirred at rt overnight. The reaction mixture was quenched with saturated NaHCO₃ solution and extracted with ethyl ether. The organic layer was dried over MgSO₄, filtered and concentrated to provide the desired product 36. Flash chromatography using (95:5 EtOAc: hexanes w/0.5% TEA) provided the desired product. ¹H NMR is consistent with assigned structure. LC-MS (ES+): 461, ret. time=1.22 min.

### 37: (S)-2-{1-[3-(2-Methoxy-phenoxy)-benzyl]-piperidin-4-ylamino}-3-phenyl-propionic Acid Methyl Ester

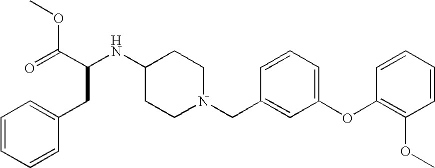

To a solution of ketone 32-2 (160 mg, 0.5 mmol) and (L)-phenylalanine methyl ester HCl (301 mg, 0.5 mmol) in methanol (5 mL) was added sodium cyanoborohydride (64 mg, 1.0 mmol). The resultant solution was stirred at rt overnight. The reaction mixture was quenched with saturated NaHCO₃ solution and extracted with ethyl ether. The organic layer was dried over MgSO₄, filtered and concentrated to provide the desired product 37. Flash chromatography using (70% ethyl acetate in hexanes) provided the desired product 37 (243 mg, 100%). ¹H NMR is consistent with assigned structure. LC-MS (ES+): 475, ret. time=1.44 min.

### 38: (S)-2-(Acetyl-{1-[3-(2-methoxy-phenoxy)-benzyl]-piperidin-4-yl}-amino)-3-phenyl-propionic Acid Methyl Ester

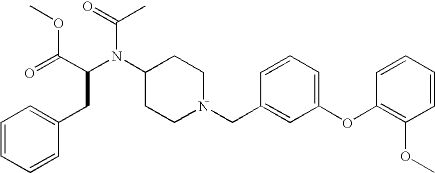

To a solution of amine 37 (175 mg, 0.46 mmol) in dichloromethane (5 mL) were added aetyl chloride (39 uL, 0.55 mmol) and triethylamine (0.146 mL, 2.3 mmol). The resultant solution was stirred at rt overnight then quenched with saturated sodium bicarbonate solution. The organic layer was dried over MgSO₄, filtered and concentrated to provide the crude product 38. Flash chromatography using (95:5 EtOAc: hexanes w/0.5% TEA) provided the desired product 38. ¹H NMR is consistent with assigned structure. LC-MS (ES+): 517, ret. time=1.57 min.

### 39: (S)-2-(Acetyl-{1-[3-(2-methoxy-phenoxy)-benzyl]-piperidin-4-yl}-amino)-3-phenyl-propionic Acid

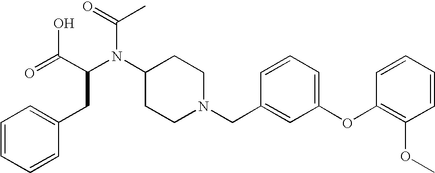

To a solution of amine 38 (74 mg, 0.14 mmol) in THF:water (4:1, 10 mL) was added lithium hydroxide (7.2 mg, 0.19 mmol). The resultant solution was stirred at rt for 7 h. The reaction solution was quenched with ammonium acetate solution and extracted with ethyl ether. The organic layer was dried over MgSO₄, filtered and concentrated to provide the product 39 (57 mg, 81%). ¹H NMR is consistent with the assigned structure. LC-MS (ES+): 503, ret. time=1.59 min.

### 40: 3-(4-Chloro-phenyl)-2-{1-[3-(2-methoxy-phenoxy)-benzyl]-piperidin-4-ylamino}-propionic Acid Methyl Ester

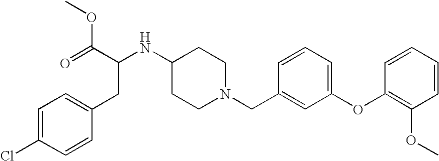

To a solution of ketone 32-2 (240 mg, 0.77 mmol) and p-chloro-phenylalanine methyl ester HCl (202 mg, 0.81 mmol) in methanol (8 mL) was added sodium cyanoborohydride (97 mg, 1.5 mmol). The resultant solution was stirred at rt overnight. The reaction mixture was quenched with saturated NaHCO₃ solution and extracted with ethyl ether. The organic layer was dried over MgSO₄, filtered and concentrated to provide the desired product 40. Flash chromatography using (70% ethyl acetate in hexanes) provided the desired product 40 (193 mg, 49%). ¹H NMR is consistent with assigned structure. LC-MS (ES+): 509, ret. time=1.65 min.

### 41: 2-(Acetyl-{1-[3-(2-methoxy-phenoxy)-benzyl]-piperidin-4-yl}-amino)-3-(4-chloro-phenyl)-propionic Acid Methyl Ester

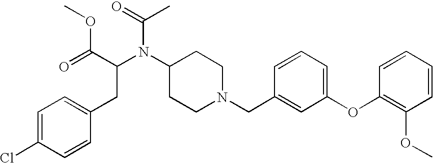

To a solution of amine 40 (193 mg, 0.38 mmol) in dichloromethane (5 mL) were added acetyl chloride (28 uL, 0.39 mmol) and triethylamine (0.106 mL, 0.76 mmol). The resultant solution was stirred at rt overnight then quenched with saturated sodium bicarbonate solution. The organic layer was dried over MgSO₄, filtered and concentrated to provide the crude product 41. Flash chromatography using (95:5 EtOAc: hexanes w/0.5% TEA) provided the desired product 41. ¹H NMR is consistent with assigned structure. LC-MS (ES+): 551, ret. time=1.65 min.

### 42: 2-(Acetyl-{1-[3-(2-methoxy-phenoxy)-benzyl]-piperidin-4-yl}-amino)-3-(4-chloro-phenyl)-propionic Acid

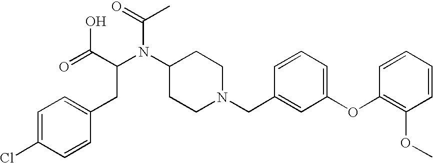

To a solution of amine 41 (77 mg, 0.14 mmol) in THF/water (4:1, 10 mL) was added lithium hydroxide (7.2 mg, 0.17 mmol). The resultant solution was stirred at rt for 7 h. The reaction solution was quenched with ammonium acetate solution and extracted with ethyl ether. The organic layer was dried over MgSO₄, filtered and concentrated to provide the product 42 (38 mg, 46%). ¹H NMR is consistent with the assigned structure. LC-MS (ES+): 537, ret. time=1.71 min.

### 43: 2-Phenyl-pentanedioic acid 5-dimethylamide 1-({1-[3-(2-methoxy-phenoxy)-benzyl]-piperidin-4-yl}-amide)

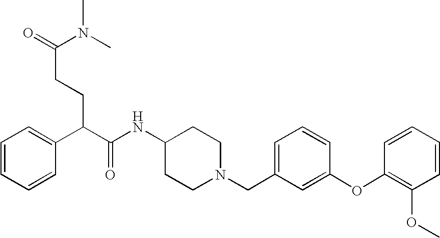

To a solution of amide 3-85 (302 mg, 0.7 mmol) in THF/DMF (1:1, 8 mL), was added sodium hydride (60% dispersion in mineral oil, 62 mg). The resultant solution was stirred at rt for 10 minutes followed by the addition of N,N-dimethyl acrylamide (72 uL, 0.7 mmol). The reaction solution was heated at 40° C. for 2d. The reaction solution was quenched with saturated NH₄Cl solution and extracted with dichloromethane. The organic layer was dried over MgSO₄, filtered and concentrated to provide the crude product 43. Flash chromatography (5-10% methanol in ethyl acetate) provided the desired product 43 as a white foam (121 mg, 33%). ¹H NMR is consistent with the assigned structure. LC-MS (ES+): 530, ret.time=1.51 min.

### 44: 5-Morpholin-4-yl-5-oxo-2-phenyl-pentanoic Acid {1-[3-(2-methoxy-phenoxy)-benzyl]-piperidin-4-yl}-amide

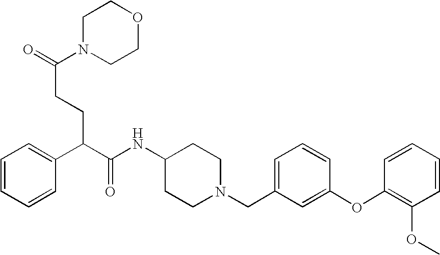

To a solution of amide 3-85 (204 mg, 0.5 mmol) in THF/DMF (1:1, 4 mL), was added sodium hydride (60% dispersion in mineral oil, 42 mg). The resultant solution was stirred at rt for 10 minutes followed by the addition of 1-Morpholin-4-yl-propenone (60 uL, 0.5 mmol). The reaction solution was heated at 40° C. for 2d. The reaction solution was quenched with saturated NH4Cl solution and extracted with dichloromethane. The organic layer was dried over MgSO₄, filtered and concentrated to provide the crude product 44. Flash chromatography (5-10% methanol in ethyl acetate) provided the desired product 44 as a white foam (56 mg, 20%). ¹H NMR is consistent with the assigned structure. LC-MS (ES+): 572, ret.time=1.54 min.

### 45: 4-{1-[3-(2-Methoxy-phenoxy)-benzyl]-piperidin-4-ylcarbamoyl}-4-phenyl-butyric Acid Tert-Butyl Ester

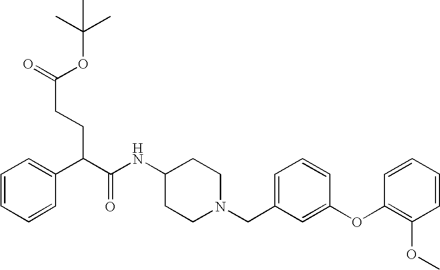

To a solution of amide 3-85 (272 mg, 0.63 mmol) in THF/DMF (1:1, 7 mL), was added sodium hydride (60% dispersion in mineral oil, 56 mg). The resultant solution was stirred at rt for 10 minutes followed by the addition of acrylic acid tert-butyl ester (93 uL, 0.63 mmol). The reaction solution was heated at 40° C. for 2d. The reaction solution was quenched with saturated NH₄Cl solution and extracted with dichloromethane. The organic layer was dried over MgSO₄, filtered and concentrated to provide the crude product 45. HPLC chromatography (phenomenex C18 column, 10 micron/60×21.2 mm) using 75:25 solvent A:B to 100% solvent B over 23 minutes (A=99% H₂O/1% CH₃CN/0.1% formic acid, B=95% CH₃CN/5% H₂O/0.1% formic acid) provided the desired product 45. ¹H NMR is consistent with the assigned structure. LC-MS (ES+): 559, ret. time=2.16 min.

### 46: 2-Oxo-1,2,3,4-tetrahydro-quinoline-4-carboxylic Acid {1-[3-(2-methoxy-phenoxy)-benzyl]-piperidin-4-yl}-amide

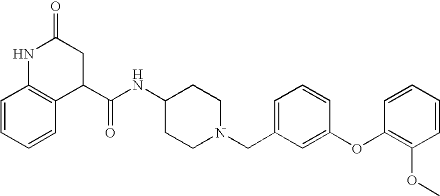

To a solution of 2-Oxo-1,2,3,4-tetrahydro-quinoline-4-carboxylic acid (80 mg, 0.42 mmol) in DCM:THF (1:1, 5 mL) were added hydroxybenzotriazole (57 mg, 0.42 mmol) and 1-[3-(dimethylamino)propyl]-3-ethylcarbodiimide hydrochloride (EDCl) (120 mg, 0.63 mmol). The reaction solution was stirred at rt for 10 minutes followed by the addition of amine 2 (176 mg, 0.5 mmol) and N-methyl morpholine (230 uL, 2.1 mmol). The reaction solution was stirred overnight at rt. The reaction solution was concentrated and the crude residue purified by flash chromatography (2-10% methanol in DCM). ¹H NMR is consistent with the assigned structure. LC-MS (ES+): 486, ret. time=1.35 min.

### 47: Ethyl-{1-[3-(2-methoxy-phenoxy)-benzyl]-piperidin-4-yl}-amine

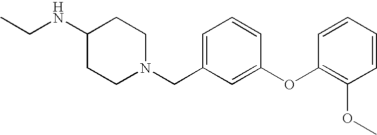

To a solution of ketone 32-2 (200 mg, 0.64 mmol) and ethylamine (2M in THF; 2.5 mL, 2.56 mmol) in dichloroethane (7 mL), was added sodium triacetoxyborohydride (272 mg, 1.28 mmol). The resultant solution was stirred at rt overnight then quenched with aqueous sodium bicarbonate solution and extracted with dichloromethane. The organic layer way dried over MgSO₄, filtered and concentrated to provide the desire product that could be used without further purification. LC-MS (ES+): 341, ret. time=0.83 min.

### 48: N-Ethyl-N-{1-[3-(2-methoxy-phenoxy)-benzyl]-piperidin-4-yl}-2-phenyl-acetamide

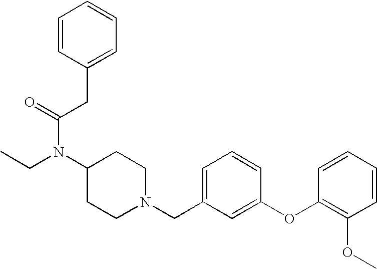

To a solution of amine 47 in dichloromethane was added triethylamine (0.41 mL, 2.9 mmol) and phenylacetyl chloride (86 uL, 0.65 mmol). The resultant solution was stirred overnight. The reaction mixture was quenched with saturated NH₄Cl solution and extracted with dichloromethane. The organic layer was dried over MgSO₄, filtered and concentrated to provide the crude product that was purified by flash chromatography (95:5 ethyl acetate:hexanes w/0.5% TEA). ¹H NMR is consistent with the assigned structure. LC-MS (ES+): 459, ret. time=1.62 min.

### Preparation of [4-(3-Aryloxy-benzyl)-[1,4]diazepan-1-yl]-alkanone

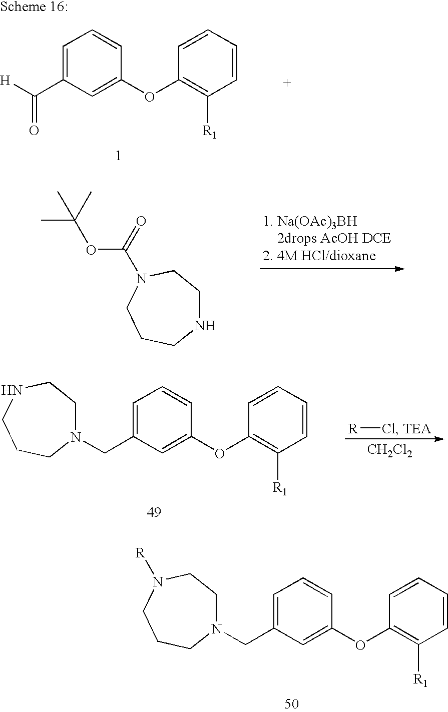

### 49: 1-(3-Phenoxy-benzyl)-[1,4]diazepane

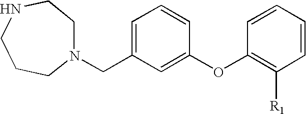

To a flamed 250 mL round bottomed flask charged with N₂ was added dichloroethane (100 mL), 3-phenoxy-benzaldehyde (1-1, 4.13 mL, 24 mmol, 0.96 eq), [1,4]diazepane-1-carboxylic acid tert-butyl ester (5.03 g, 25 mmol, 1.0 eq), sodium triacetoxy-borohydride (15.9 g, 75 mmol, 3 eq), and two drops of acetic acid. The solution was allowed to stir at room temperature overnight. The reaction was quenched by slow addition of methyl alcohol (5 mL) and water (50 mL), extracted with CH₂Cl₂, washed with 1N NaOH, brine, dried over MgSO₄, filtered and concentrated under reduced pressure at 40° C. to provide a yellow oil. The residue was dissolved in 4M HCl in dioxane (35 mL), stirred for two hours and concentrated under reduced pressure at 40° C. to provide a white solid. Trituration with diethylether afforded 8.0 g (90%) of 1-(3-phenoxy-benzyl)-[1,4]diazepane 49 as the bis-hydrochloride salt. ¹H NMR is consistent with the assigned structure. LC-MS (ES+): 283, ret. time: 0.94 min.

### 50: [4-(3-Aryloxy-benzyl)-[1,4]diazepan-1-yl]-alkanone

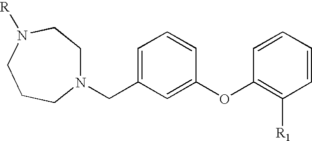

To a scintillation vial was added 1-(3-phenoxy-benzyl)-[1,4]diazepane, 49 (178 mg, 0.5 mmol), triethylamine (0.2 mL, 1.5 mmol), acid chloride (R—Cl/structures are in the data sheets, 0.5 mmol) and CH₂Cl₂ (5 mL). The solution was placed on an orbital shaker overnight, washed with 1N HCl (3×5 mL), NaHCO₃ (3×5 mL), brine, dried over MgSO₄, filtered and concentrated under reduced pressure at 40° C. to provide a yellow oil. Flash chromatography (SiO₂, Biotage 10 g column, eluent: 95:5:0.5 ethyl acetate/hexanes/triethyl amine) afforded a residue which was dissolved in 4M HCl in dioxane (10 mL), stirred for 2 hours and concentrated under reduced pressure at 40° C. to provide a white powder. Trituration with diethylether afforded 50 as the hydrochloride salt. 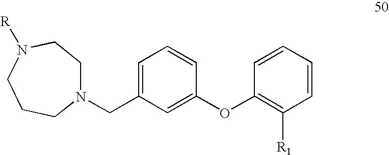
50-1: 2-Phenoxy-1-[4-(3-phenoxy-benzyl)-[1,4]diazepan-1-yl]-ethanone
50-2: (4-Chloro-phenyl)-[4-(3-phenoxy-benzyl)-[1,4]diazepan-1-yl]-methanone
50-3: (4-Fluoro-phenyl)-[4-(3-phenoxy-benzyl)-[1,4]diazepan-1-yl]-methanone
50-4: 3,5,5-Trimethyl-1-[4-(3-phenoxy-benzyl)-[1,4]diazepan-1-yl]-hexan-1-one
50-5: 2-(2-Bromo-phenyl)-1-[4-(3-phenoxy-benzyl)-[1,4]diazepan-1-yl]-ethanone
50-6: 2-(3-Methoxy-phenyl)-1-[4-(3-phenoxy-benzyl)-[1,4]diazepan-1-yl]-ethanone
50-7: 1-[4-(3-Phenoxy-benzyl)-[1,4]diazepan-1-yl]-2-thiophen-2-yl-ethanone
50-8: 2-(3,5-Dimethoxy-phenyl)-1-[4-(3-phenoxy-benzyl)-[1,4]diazepan-1-yl]-ethanone
50-9: 2-(4-Fluoro-phenyl)-1-[4-(3-phenoxy-benzyl)-[1,4]diazepan-1-yl]-ethanone
50-10: 1-[4-(3-Phenoxy-benzyl)-[1,4]diazepan-1-yl]-2,2-diphenyl-ethanone
50-11: 1-[4-(3-Phenoxy-benzyl)-[1,4]diazepan-1-yl]-3-phenyl-propan-1-one
50-12: 1-[4-(3-Phenoxy-benzyl)-[1,4]diazepan-1-yl]-3-phenyl-propenone
50-13: 2-(4-Benzyloxy-phenyl)-1-[4-(3-phenoxy-benzyl)-[1,4]diazepan-1-yl]-ethanone
50-14: 2-(3,4-Dimethoxy-phenyl)-1-{4-[3-(2-methoxy-phenoxy)-benzyl]-[1,4]diazepan-1-yl}-ethanone
50-15: 1-{4-[3-(2-Methoxy-phenoxy)-benzyl]-[1,4]diazepan-1-yl}-2-(3-methoxy phenyl)-ethanone
50-16: 2-(2-Bromo-phenyl)-1-{4-[3-(2-methoxy-phenoxy)-benzyl]-[1,4]diazepan-1-yl} ethanone
50-17: 1-{4-[3-(2-Methoxy-phenoxy)-benzyl]-[1,4]diazepan-1-yl}-2-thiophen-2-yl-ethanone
50-18: 2-(4-Fluoro-phenyl)-1-{4-[3-(2-methoxy-phenoxy)-benzyl]-[1,4]diazepan-1-yl}-ethanone
50-19: (4-Chloro-phenyl)-{4-[3-(2-methoxy-phenoxy)-benzyl]-[1,4]diazepan-1-yl}-methanone
50-20: 1-{4-[3-(2-Methoxy-phenoxy)-benzyl]-[1,4]diazepan-1-yl}-3-phenyl-propan-1-one
50-21: 1-[3-(2-Methoxy-phenoxy)-benzyl]-4-phenylmethanesulfonyl-[1,4]diazepane
50-22: 4-[3-(2-Methoxy-phenoxy)-benzyl]-[1,4]diazepane-1-carboxylic acid 4-methoxy-phenyl ester
50-23: 4-[3-(2-Methoxy-phenoxy)-benzyl]-[1,4]diazepane-1-carboxylic acid methyl-phenyl-amide
50-24: 1-{4-[3-(2-Methoxy-phenoxy)-benzyl]-[1,4]diazepan-1-yl}-2-phenyl-ethanone

50-25: (4-Fluoro-phenyl)-{4-[3-(2-methoxy-phenoxy)-benzyl]-[1,4]diazepan-1-yl}-methanone

TABLE 10

| | | | Retention | |
|---|---|---|---|---|
| | | | Time | |
| No. | R | R₁ | (min) | M + 1 |
| 50-1 | 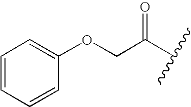 | H | 1.63 | 417 |
| 50-2 | 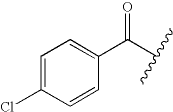 | H | 1.68 | 421 |
| 50-3 | 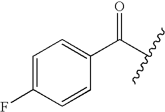 | H | 1.57 | 405 |
| 50-4 | 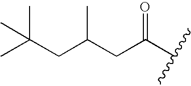 | H | 1.81 | 423 |
| 50-5 | 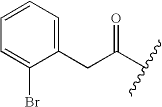 | H | 1.64 | 479, 481 |
| 50-6 | 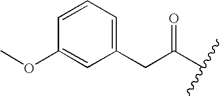 | H | 1.53 | 431 |
| 50-7 | 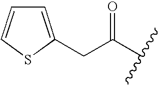 | H | 1.50 | 407 |
| 50-8 | 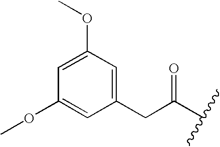 | H | 1.54 | 461 |
| 50-9 | 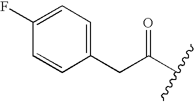 | H | 1.54 | 419 |
| 50-10 | 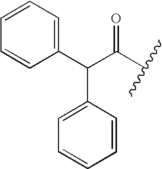 | H | 1.78 | 477 |
| 50-11 | 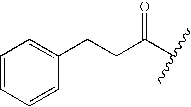 | H | 1.68 | 415 |
| 50-12 | 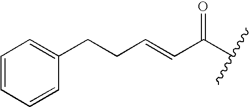 | H | 1.76 | 413 |
| 50-13 | 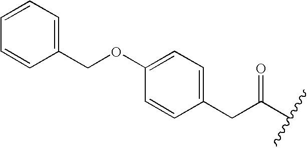 | H | 2.05 | 507 |
| 50-14 | 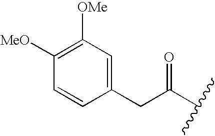 | OMe | 1.48 | 491 |
| 50-15 | 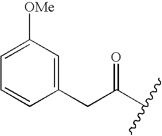 | OMe | 1.58 | 461 |
| 50-16 | 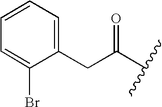 | OMe | 1.62 | 511 |
| 50-17 | 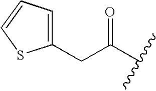 | OMe | 1.46 | 437 |
| 50-18 | 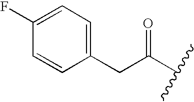 | | | |
| 50-19 | 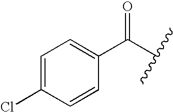 | OMe | 1.61 | 451 |
| 50-20 | 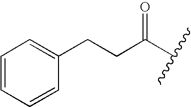 | OMe | 1.58 | 445 |
| 50-21 | 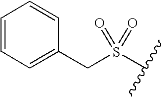 | OMe | 1.62 | 467 |
| 50-22 | 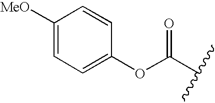 | OMe | 1.60 | 463 |
| 50-23 | 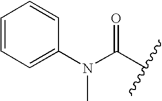 | OMe | 1.52 | 446 |
| 50-24 | 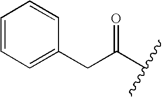 | OMe | 1.51 | 431 |
| 50-25 | 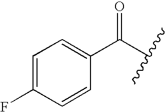 | OMe | 1.47 | 435 |

### Preparation of 1-[3-(2-Methoxy-phenoxy)-benzyl]-pyrrolidin-3-ylamine Derivatives

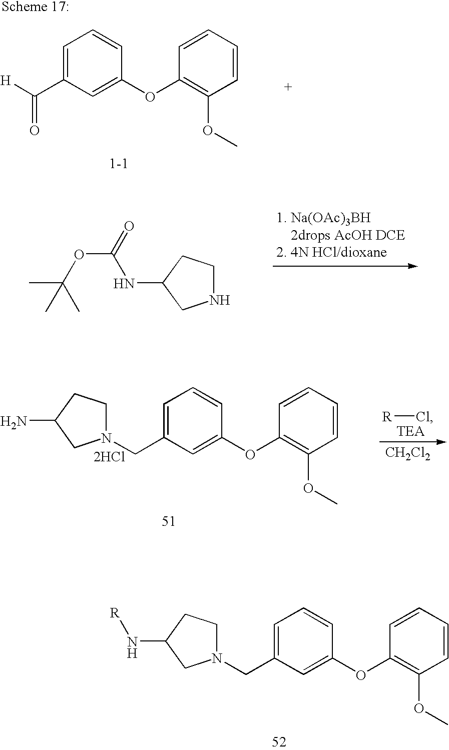

### 51: 1-[3-(2-Methoxy-phenoxy)-benzyl]-pyrrolidin-3-ylamine

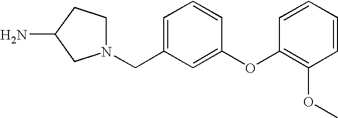

Experimental procedure followed that of 1-(3-phenoxy-benzyl)-[1,4]diazepane 49.

¹H NMR is consistent with the assigned structure. LC-MS (ES+): 299, ret. time=0.85 min.

### 52:1-[3-(2-Methoxy-phenoxy)-benzyl]-pyrrolidin-3-ylamine Derivatives

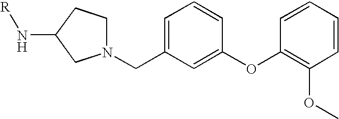

Experimental procedure followed that of 50. 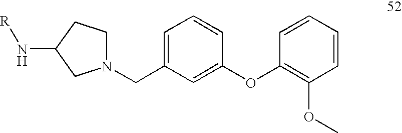
52-1: 2-(3,4-Dimethoxy-phenyl)-N-{1-[3-(2-methoxy-phenoxy)-benzyl]-pyrrolidin-3-yl}-acetamide
52-2: 2-(2-Bromo-phenyl)-N-{1-[3-(2-methoxy-phenoxy)-benzyl]-pyrrolidin-3-yl}-acetamide
52-3: N-{1-[3-(2-Methoxy-phenoxy)-benzyl]-pyrrolidin-3-yl}-2-thiophen-2-yl-acetamide
52-4: 2-(4-Chloro-phenyl)-N-{1-[3-(2-methoxy-phenoxy)-benzyl]-pyrrolidin-3-yl}-acetamide

52-5: N-{1-[3-(2-Methoxy-phenoxy)-benzyl]-pyrrolidin-3-yl}-2-[4-(4-methyl-benzyloxy)-phenyl]-acetamide

TABLE 11

| | | Retention Time | |
|---|---|---|---|
| No. | R | (min) | M + 1 |
| 52-1 | 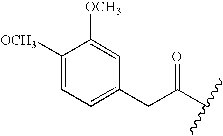 | 1.61 | 477 |
| 52-2 | 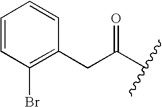 | 1.59 | 497, 495 |
| 52-3 | 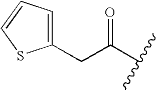 | 1.47 | 423 |
| 52-4 | 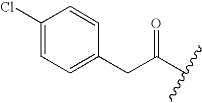 | 1.84 | 451 |
| 52-5 | 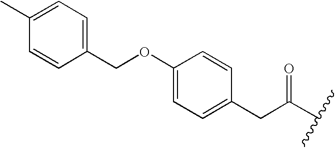 | 2.09 | 523 |

### Preparation of 1-[3-(2-Methoxy-phenoxy)-benzyl]-pyrrolidin-3-(R)-ylamine Derivatives

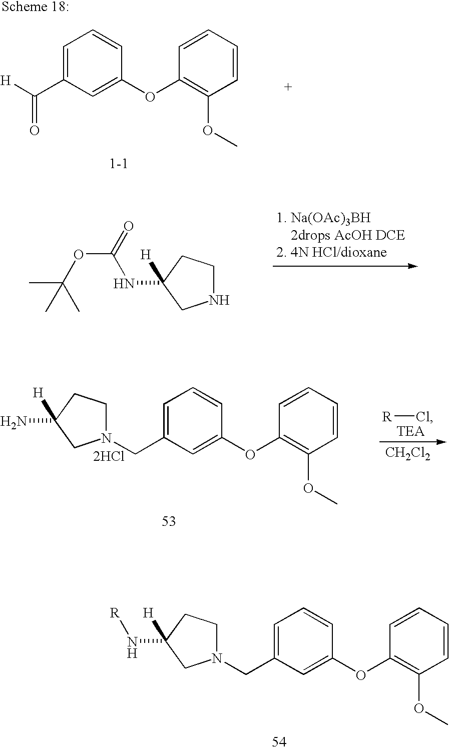

### 53: 1-[3-(2-Methoxy-phenoxy)-benzyl]-pyrrolidin-3-(R)-ylamine

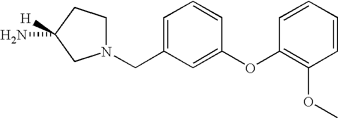

Experimental procedure followed that of 1-(3-phenoxy-benzyl)-[1,4]diazepane 49.

¹H NMR is consistent with the assigned structure. LC-MS (ES+): 299, ret. time 0.85

### 54:1-[3-(2-Methoxy-phenoxy)-benzyl]-pyrrolidin-3-(R)-ylamine Derivatives

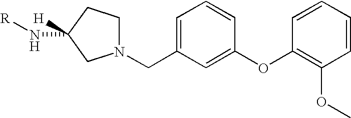

Experimental procedure followed that of 50. 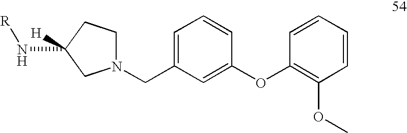
54-1: (R)-2-(3,4-Dimethoxy-phenyl)-N-{1-[3-(2-methoxy-phenoxy)-benzyl]-pyrrolidin-3-yl}-acetamide
54-2: (R)-N-{1-[3-(2-Methoxy-phenoxy)-benzyl]-pyrrolidin-3-yl}-2-(3-methoxy-phenyl)-acetamide
54-3: (R)-2-(2-Bromo-phenyl)-N-{1-[3-(2-methoxy-phenoxy)-benzyl]-pyrrolidin-3-yl}-acetamide
54-4: (R)-N-{1-[3-(2-Methoxy-phenoxy)-benzyl]-pyrrolidin-3-yl}-2-thiophen-2-yl-acetamide
54-5: (R)-2-(4-Fluoro-phenyl)-N-{1-[3-(2-methoxy-phenoxy)-benzyl]-pyrrolidin-3-yl}-acetamide
54-6: (R)-4-Fluoro-N-{1-[3-(2-methoxy-phenoxy)-benzyl]-pyrrolidin-3-yl}-benzamide
54-7: (R)-4-Chloro-N-{1-[3-(2-methoxy-phenoxy)-benzyl]-pyrrolidin-3-yl}-benzamide
54-8: (R)-N-{1-[3-(2-Methoxy-phenoxy)-benzyl]-pyrrolidin-3-yl}-2-phenoxy-acetamide
54-9: (R)-N-{1-[3-(2-Methoxy-phenoxy)-benzyl]-pyrrolidin-3-yl}-3-phenyl-acrylamide
54-10: (R)-N-{1-[3-(2-Methoxy-phenoxy)-benzyl]-pyrrolidin-3-yl}-3-phenyl-propionamide
54-11: (R)-N-{1-[3-(2-Methoxy-phenoxy)-benzyl]-pyrrolidin-3-yl}-C-phenyl-methanesulfonamide
54-12: (R)-N-{1-[3-(2-Methoxy-phenoxy)-benzyl]-pyrrolidin-3-yl}-C-(2-nitro-phenyl)-methanesulfonamide
54-13: (R)-{1-[3-(2-Methoxy-phenoxy)-benzyl]-pyrrolidin-3-yl}-carbamic acid phenyl ester
54-14: (R)-{1-[3-(2-Methoxy-phenoxy)-benzyl]-pyrrolidin-3-yl}-carbamic acid 4-chloro-phenyl ester
54-15: (R)-{1-[3-(2-Methoxy-phenoxy)-benzyl]-pyrrolidin-3-yl}-carbamic acid 4-fluoro-phenyl ester
54-16: (R)-{1-[3-(2-Methoxy-phenoxy)-benzyl]-pyrrolidin-3-yl}-carbamic acid 4-methoxy-phenyl ester
54-17: (R)-3-{1-[3-(2-Methoxy-phenoxy)-benzyl]-pyrrolidin-3-yl}-1-methyl-1-phenyl-urea

54-18: (R)-4-Chloro-N-(4-chloro-benzoyl)-N-{1-[3-(2-methoxy-phenoxy)-benzyl]-pyrrolidin-3-yl}-benzamide

TABLE 12

| | | Retention Time | |
|---|---|---|---|
| No. | R | (min) | M + 1 |
| 54-1 | 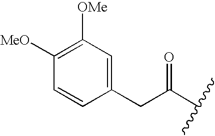 | 1.57 | 477 |
| 54-2 | 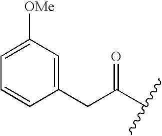 | 1.70 | 447 |
| 54-3 | 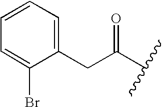 | 1.90 | 496 |
| 54-4 | 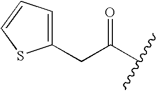 | 1.76 | 423 |
| 54-5 | 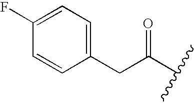 | 1.72 | 435 |
| 54-6 | 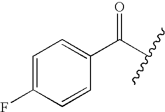 | 1.72 | 421 |
| 54-7 | 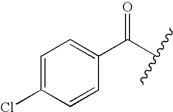 | 1.74 | 433 |
| 54-8 | 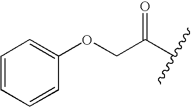 | 1.84 | 437 |
| 54-9 | 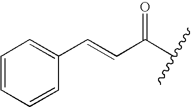 | 1.61 | 429 |
| 54-10 | 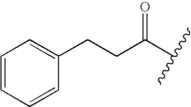 | 1.69 | 431 |
| 54-11 | 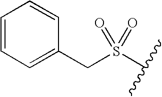 | 1.68 | 453 |
| 54-12 | 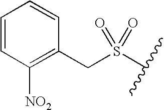 | 1.69 | 498 |
| 54-13 | 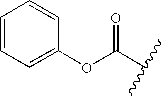 | 1.70 | 419 |
| 54-14 | 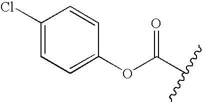 | 1.80 | 453 |
| 54-15 | 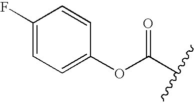 | 1.73 | 437 |
| 54-16 | 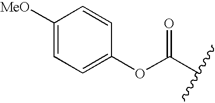 | 1.56 | 449 |
| 54-17 | 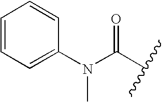 | 1.55 | 432 |
| 54-18 | 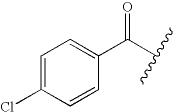 | 2.25 | 575 |

### Preparation of 1-[3-(2-Methoxy-phenoxy)-benzyl]-piperazine Derivatives

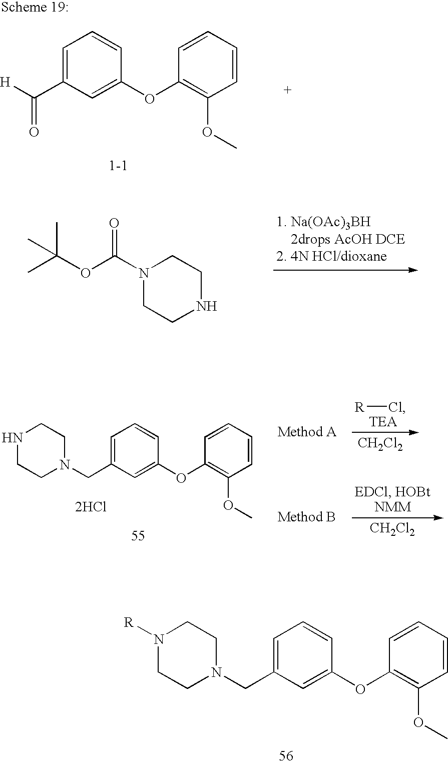

### 55: 1-[3-(2-Methoxy-phenoxy)-benzyl]-piperazine

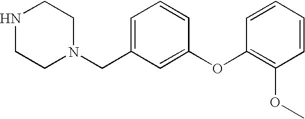

Experimental procedure followed that of 1-(3-phenoxy-benzyl)-[1,4]diazepane 49.

¹H NMR is consistent with the assigned structure. LC-MS (ES+): 299, ret.time=1.01 min.

### 56:1-[3-(2-Methoxy-phenoxy)-benzyl]-piperazine Derivatives

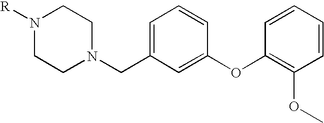 Method A:

Experimental procedure followed that of 50.

Method B:

A solution of 1-(3-dimethylaminopropyl)-3-ethylcarbodiimide hydrochloride (EDCI, 288 mg, 1.5 mmol), 1-hydroxybenzotriazole hydrate (HOBt, 135 mg, 1.0 mmol), and 1 mmol of the corresponding acid in methylene chloride was stirred for 20 minutes at room temperature. To the resulting solution, N-methylmorpholine (NMM, 0.55 mL, 5 mmol) and 1-[1-(2-methoxy-benzyl)-piperidin-4-ylmethyl]-piperidin-4-ylamine (55, 401 mg, 1.2 mmol) were added and the solution was allowed to stir overnight. The resulting solution was washed with 1N NaOH (2×5 mL), water (lx 5 mL), brine (1×5 mL), dried over magnesium sulfate filtered and concentrated under reduced pressure at 40° C. Flash chromatography (5% hexane in ethyl acetate, 0.5% TEA) provided an oil. The residue was dissolved in 4N HCl in dioxane (10 mL), stirred for two hours and concentrated under reduced pressure at 40° C. to provide a solid. Trituration with diethylether afforded 56 as the hydrochloride salt. 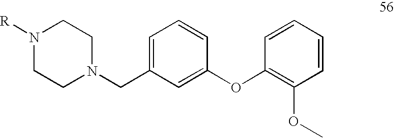
56-1: 2-(3,4-Dimethoxy-phenyl)-1-{4-[3-(2-methoxy-phenoxy)-benzyl]-piperazin-1-yl}-ethanone
56-2: 1-{4-[3-(2-Methoxy-phenoxy)-benzyl]-piperazin-1-yl}-2-(3-methoxy-phenyl)-ethanone
56-3: 1-{4-[3-(2-Methoxy-phenoxy)-benzyl]-piperazin-1-yl}-2-thiophen-2-yl-ethanone
56-4: 2-(4-Fluoro-phenyl)-1-{4-[3-(2-methoxy-phenoxy)-benzyl]-piperazin-1-yl}-ethanone
56-5: (4-Fluoro-phenyl)-{4-[3-(2-methoxy-phenoxy)-benzyl]-piperazin-1-yl}-methanone
56-6: 2-(4-Chloro-phenyl)-1-{4-[3-(2-methoxy-phenoxy)-benzyl]-piperazin-1-yl}-ethanone
56-7: 2-(4-Benzyloxy-phenyl)-1-f{4-[3-(2-methoxy-phenoxy)-benzyl]-piperazin-1-yl}-ethanone
56-8: 1-{4-[3-(2-Methoxy-phenoxy)-benzyl]-piperazin-1-yl}-3-phenyl-propan-1-one
56-9: 4-[3-(2-Methoxy-phenoxy)-benzyl]-piperazine-1-carboxylic acid phenyl ester
56-10: 1-{4-[3-(2-Methoxy-phenoxy)-benzyl]-piperazin-1-yl}-2-phenoxy-ethanone
56-11: 1-[3-(2-Methoxy-phenoxy)-benzyl]-4-(2-nitro-phenylmethanesulfonyl)-piperazine
56-12: 4-[3-(2-Methoxy-phenoxy)-benzyl]-piperazine-1-carboxylic acid 4-fluoro-phenyl ester
56-13: 1-{4-[3-(2-Methoxy-phenoxy)-benzyl]-piperazin-1-yl}-2,2-diphenyl-ethanone
56-14: 4-[3-(2-Methoxy-phenoxy)-benzyl]-piperazine-1-carboxylic acid methyl-phenyl-amide
56-15: 4-[3-(2-Methoxy-phenoxy)-benzyl]-piperazine-1-carboxylic acid 4-methoxy-phenyl ester
56-16: 4-[3-(2-Methoxy-phenoxy)-benzyl]-piperazine-1-carboxylic acid 4-chloro-phenyl ester
56-17: [1-(2,4-Dichloro-phenyl)-cyclopropyl]-{4-[3-(2-methoxy-phenoxy)-benzyl]-piperazin-1-yl}-methanone
56-18: [1-(2-Fluoro-phenyl)-cyclopentyl]-{4-[3-(2-methoxy-phenoxy)-benzyl]-piperazin-1-yl}-methanone
56-19: {4-[3-(2-Methoxy-phenoxy)-benzyl]-piperazin-1-yl}-(1-phenyl-cyclopentyl)-methanone
56-20: {4-[3-(2-Methoxy-phenoxy)-benzyl]-piperazin-1-yl}-(1-p-tolyl-cyclopentyl)-methanone
56-21: 1-{4-[3-(2-Methoxy-phenoxy)-benzyl]-piperazin-1-yl}-2-phenyl-propan-1-one
56-22: 1-{4-[3-(2-Methoxy-phenoxy)-benzyl]-piperazin-1-yl}-2-phenyl-propan-1-one
56-23: 2-(4-Chloro-phenyl)-1-{4-[3-(2-methoxy-phenoxy)-benzyl]-piperazin-1-yl}-2-methyl-propan-1-one
56-24: [1-(4-Chloro-phenyl)-cyclopentyl]-{4-[3-(2-methoxy-phenoxy)-benzyl]-piperazin-1-yl}-methanone
56-25: 2-(4-Chloro-phenyl)-1-{4-[3-(2-methoxy-phenoxy)-benzyl]-piperazin-1-yl}-propan-1-one
56-26: 1-{4-[3-(2-Methoxy-phenoxy)-benzyl]-piperazin-1-yl}-2-phenyl-ethanone
56-27: 1-{4-[3-(2-Methoxy-phenoxy)-benzyl]-piperazin-1-yl}-2-phenyl-butan-1-one
56-28: {4-[3-(2-Methoxy-phenoxy)-benzyl]-piperazin-1-yl}-(1-phenyl-cyclopropyl)-methanone
56-29: [1-(4-Fluoro-phenyl)-cyclopentyl]-{4-[3-(2-methoxy-phenoxy)-benzyl]-piperazin-1-yl}-methanone
56-30: 1-{4-[3-(2-Methoxy-phenoxy)-benzyl]-piperazin-1-yl}-2,2-diphenyl-propan-1-one
56-31: {4-[3-(2-Methoxy-phenoxy)-benzyl]-piperazin-1-yl}-(2-phenyl-cyclopropyl)-methanone
56-32: {4-[3-(2-Methoxy-phenoxy)-benzyl]-piperazin-1-yl}-(1,2,3,4-tetrahydro-naphthalen-2-yl)-methanone
56-33: Bicyclo[4.2.0]octa-1 (6),2,4-trien-7-yl-{4-[3-(2-methoxy-phenoxy)-benzyl]-piperazin-1-yl}-methanone
56-34: 1-{4-[3-(2-Methoxy-phenoxy)-benzyl]-piperazin-1-yl}-3,3-diphenyl-propan-1-one
56-35: 3-(4-Fluoro-phenyl)-1-{4-[3-(2-methoxy-phenoxy)-benzyl]-piperazin-1-yl}-propenone

56-36: [1-(4-Chloro-phenyl)-cyclopropyl]-{4-[3-(2-methoxy-phenoxy)-benzyl]-piperazin-1-yl}-methanone

TABLE 13

| | | Retention Time | |
|---|---|---|---|
| No. | R | (min) | M + 1 |
| 56-1 | 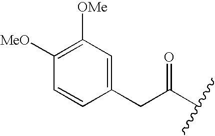 | 1.43 | 477 |
| 56-2 | 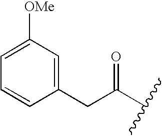 | 1.59 | 447 |
| 56-3 | 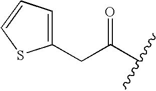 | 1.54 | 423 |
| 56-4 | 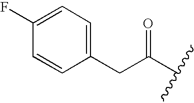 | 1.65 | 435 |
| 56-5 | 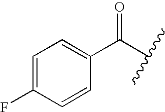 | 1.57 | 421 |
| 56-6 | 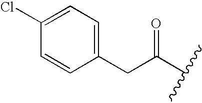 | 1.70 | 451 |
| 56-7 | 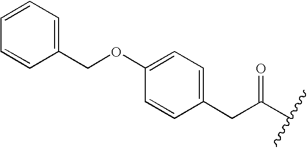 | 1.91 | 523 |
| 56-8 | 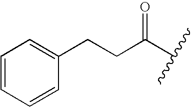 | 1.62 | 431 |
| 56-9 | 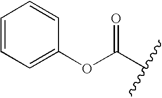 | 1.66 | 419 |
| 56-10 | 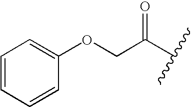 | 1.60 | 433 |
| 56-11 | 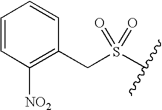 | 1.83 | 498 |
| 56-12 | 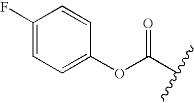 | 1.72 | 437 |
| 56-13 | 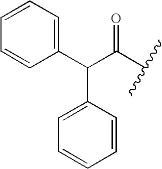 | 1.92 | 493 |
| 56-14 | 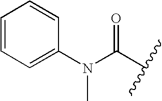 | 1.58 | 432 |
| 56-15 | 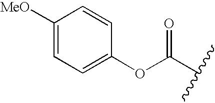 | 1.67 | 449 |
| 56-16 | 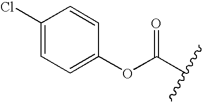 | 1.86 | 453 |
| 56-17 | 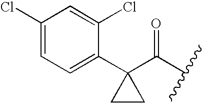 | 1.84 | 513, 511 |
| 56-18 | 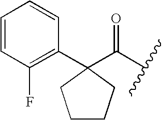 | 1.80 | 490 |
| 56-19 | 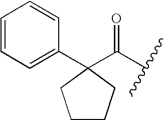 | 1.84 | 472 |
| 56-20 | 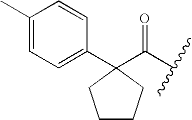 | 1.92 | 486 |
| 56-21 | 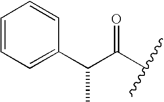 | 1.64 | 432 |
| 56-22 | 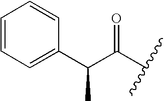 | 1.64 | 432 |
| 56-23 | 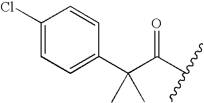 | 1.80 | 479 |
| 56-24 | 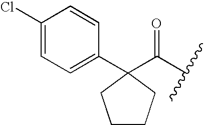 | | |
| 56-25 | 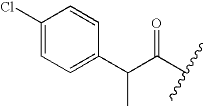 | 1.78 | 466 |
| 56-26 | 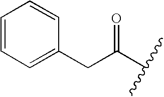 | 1.55 | 418 |
| 56-27 | 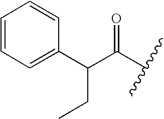 | | |
| 56-28 | 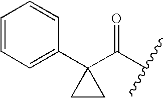 | | |
| 56-29 | 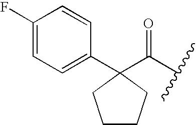 | 1.88 | 490 |
| 56-30 | 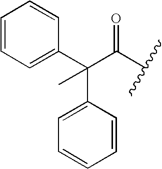 | | |
| 56-31 | 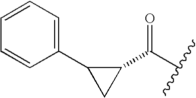 | 1.64 | |
| 56-32 | 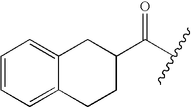 | 1.71 | 457 |
| 56-33 | 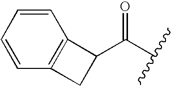 | 1.57 | 429 |
| 56-34 | 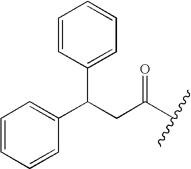 | 1.89 | 508 |
| 56-35 | 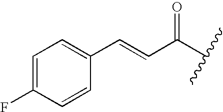 | 1.68 | 448 |
| 56-36 | 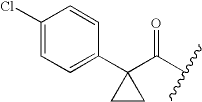 | 1.81 | 478, 476 |

### Preparation of α-{1-[3-(2-Methoxy-phenoxy)-benzyl]-piperidin-4-yl}-methylamine Derivatives

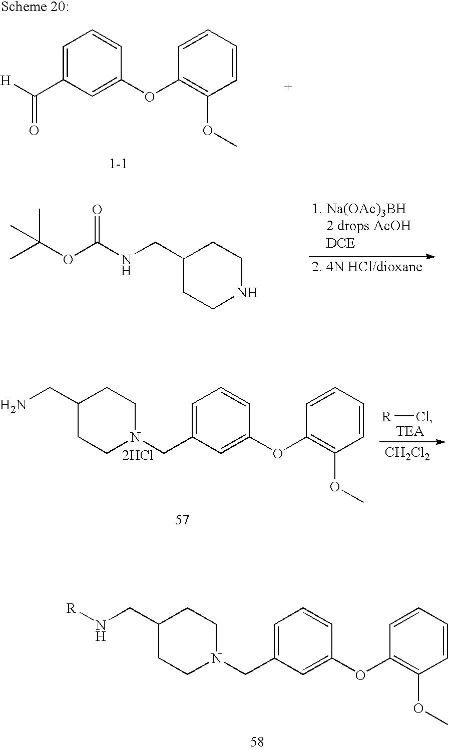

### 57: α-{1-[3-(2-Methoxy-phenoxy)-benzyl]-piperidin-4-yl}-methylamine

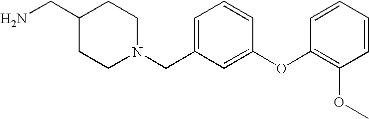

Experimental procedure followed that of 1-(3-phenoxy-benzyl)-[1,4]diazepane 49.

¹H NMR is consistent with the assigned structure. LC-MS (ES+): 327, ret.time=0.95 min.

### 58: α-{1-[3-(2-Methoxy-phenoxy)-benzyl]-piperidin-4-yl}-methylamine Derivatives

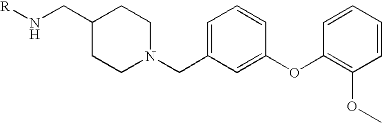

Experimental procedure followed that of 50. 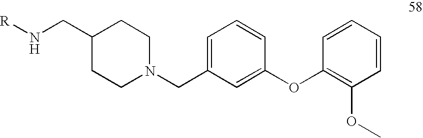
58-1: 2-(3,4-Dimethoxy-phenyl)-N-[2-(3,4-dimethoxy-phenyl)-acetyl]-N-{1-[3-(2-methoxy-phenoxy)-benzyl]-piperidin-4-ylmethyl}-acetamide
58-2: N-{1-[3-(2-Methoxy-phenoxy)-benzyl]-piperidin-4-ylmethyl}-2-(3-methoxy-phenyl)-acetamide
58-3: 2-(2-Bromo-phenyl)-N-{1-[3-(2-methoxy-phenoxy)-benzyl]-piperidin-4-ylmethyl}-acetamide
58-4: N-{1-[3-(2-Methoxy-phenoxy)-benzyl]-piperidin-4-ylmethyl}-2-thiophen-2-yl-acetamide
58-5: 2-(4-Fluoro-phenyl)-N-{1-[3-(2-methoxy-phenoxy)-benzyl]-piperidin-4-ylmethyl}-acetamide
58-6: 2-(4-Chloro-phenyl)-N-{1-[3-(2-methoxy-phenoxy)-benzyl]-piperidin-4-ylmethyl}-acetamide
58-7: N-{1-[3-(2-Methoxy-phenoxy)-benzyl]-piperidin-4-ylmethyl}-3-phenyl-propionamide
58-8: N-{1-[3-(2-Methoxy-phenoxy)-benzyl]-piperidin-4-ylmethyl}-α-phenyl-methanesulfonamide
58-9: {1-[3-(2-Methoxy-phenoxy)-benzyl]-piperidin-4-ylmethyl}-carbamic acid 4-methoxy-phenyl ester
58-10: N-{1-[3-(2-Methoxy-phenoxy)-benzyl]-piperidin-4-ylmethyl}-2-phenyl-acetamide
58-11: 3-{1-[3-(2-Methoxy-phenoxy)-benzyl]-piperidin-4-ylmethyl}-1-methyl-1-phenyl-urea

58-12: 4-Fluoro-N-{1-[3-(2-methoxy-phenoxy)-benzyl]-piperidin-4-ylmethyl}-benzamide

TABLE 14

| | | Retention Time | |
|---|---|---|---|
| No. | R | (min) | M + 1 |
| 58-1 | 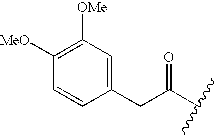 | 1.66 | 683 |
| 58-2 | 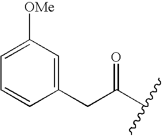 | 1.52 | 476 |
| 58-3 | 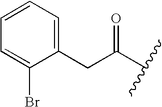 | 1.60 | 523, 521 |
| 58-4 | 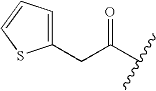 | 1.48 | 451 |
| 58-5 | 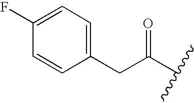 | 1.56 | 463 |
| 58-6 | 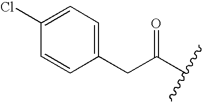 | 1.64 | 479 |
| 58-7 | 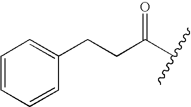 | 1.64 | 459 |
| 58-8 | 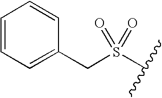 | 1.71 | 481 |
| 58-9 | 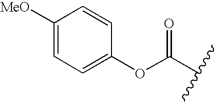 | 1.69 | 477 |
| 58-10 | 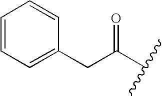 | 1.62 | 445 |
| 58-11 | 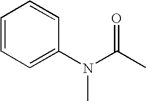 | 1.51 | 460 |
| 58-12 | 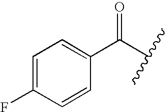 | 1.51 | 449 |

### Preparation of 1-[3-(2-Methoxy-phenoxy)-benzyl]-pyrrolidin-3-ylamine Derivatives

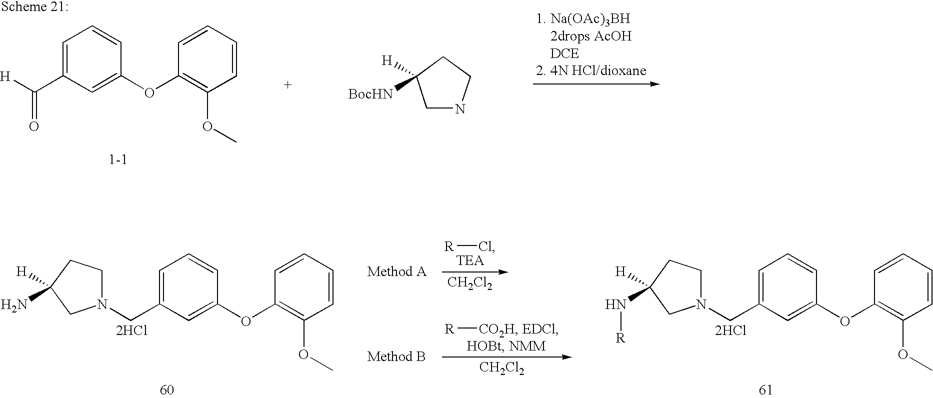

### 59: {1-[3-(2-Methoxy-phenoxy)-benzyl]-pyrrolidin-3-yl}-carbamic Acid Tert-Butyl Ester

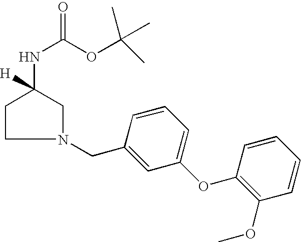

A solution of pyrrolidin-3-yl-carbamic acid tert-butyl ester (1.30 g, 6.98 mmol), 3-phenoxy benzaldehyde (1-1, 1.5 g, 6.57 mmol), Na(OAc)₃BH (4.2 g, 19.82 mmol) and acetic acid (0.5 mL) was stirred in methylene chloride (50 mL) at room temperature under nitrogen for 24 hrs. The resulting solution was washed with 1 N NaOH (3×100 mL), dried over magnesium sulfate, filtered and concentrated under reduced pressure to provide a crude oil, which was used without further purification. ¹H NMR is consistent with the assigned structure. LC-MS (ES+): Formic Acid-Standard, M+1=399, ret. time=1.35 min.

### 60: 1-[3-(2-Methoxy-phenoxy)-benzyl]-pyrrolidin-3-ylamine

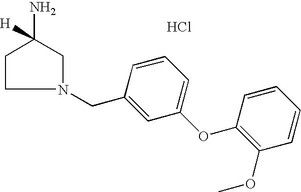

{1-[3-(2-Methoxy-phenoxy)-benzyl]-pyrrolidin-3-yl}-carbamic acid tert-butyl ester was dissolved in 30 mL of 4N HCl in dioxane, stirred at 0° C. for 15 min and allowed to warm to room temperature over 1.5 hrs. The solution was concentrated under reduced pressure and triturated in diethylether to provide 2.34 g (96%) of 1-[3-(2-methoxy-phenoxy)-benzyl]-pyrrolidin-3-ylamine 60 as a white solid. ¹H NMR is consistent with the assigned structure. LC-MS (ES+): Formic Acid-Standard, M+1=299, ret. time=0.76 min.

### 61: 1-[3-(2-Methoxy-phenoxy)-benzyl]-pyrrolidin-3-ylamine Derivatives

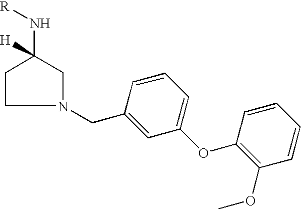 Method A:

To a solution of 1-[3-(2-methoxy-phenoxy)-benzyl]-pyrrolidin-3-ylamine 60 (186 mg, 0.5 mmol) in dichloromethane (5 mL) was added triethylamine (0.2 mL, 1.5 mmol) and acid chloride (0.5 mmol). The solution was stirred overnight at room temperature and concentrated in vacuo to provide an oil. Flash chromatography on SiO₂ (5% hexane in ethyl acetate with 0.5% triethyl amine) provided 61 as an oil which was converted to its HCl salt by treatment with 4N HCl in dioxane. Trituration with diethyl ether afforded 61 as a white powder.

Method B:

A solution of 1-(3-dimethylaminopropyl)-3-ethylcarbodiimide hydrochloride (EDCI, 288 mg, 1.5 mmol), 1-hydroxybenzotriazole hydrate (HOBt, 135 mg, 1.0 mmol), and 1 mmol of the corresponding acid in methylene chloride was stirred for 20 minutes. To the resulting solution, N-methylmorpholine (NMM, 0.55 mL, 5 mmol) and 1-[3-(2-methoxy-phenoxy)-benzyl]-pyrrolidin-3-ylamine (60, 401 mg, 1.2 mmol) were added and the solution was allowed to stir at room temperature overnight. The resulting solution was washed with 1N NaOH (2×5 mL), water (1×5 mL), brine (1×5 mL), dried over magnesium sulfate filtered and concentrated under reduced pressure at 40° C. Flash chromatography (5% hexane in ethyl acetate, 0.5% TEA) provided an oil. The residue was dissolved in 4N HCl in dioxane (10 mL), stirred for two hours and concentrated under reduced pressure at 40° C. to provide a solid. Trituration with diethyl ether afforded 61 as the hydrochloride salt. 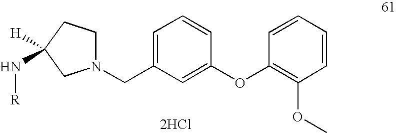
61-1: (S)-2-(2-Bromo-phenyl)-N-{1-[3-(2-methoxy-phenoxy)-benzyl]-pyrrolidin-3-yl}-acetamide.
61-2: (S)-N-{1-[3-(2-Methoxy-phenoxy)-benzyl]-pyrrolidin-3-yl}-2-(3-methoxy-phenyl)-acetamide
61-3: (S)-N-{1-[3-(2-Methoxy-phenoxy)-benzyl]pyrrolidin-3-yl}-2-phenyl-acetamide
61-4: (S)-N-{1-[3-(2-Methoxy-phenoxy)benzyl]-pyrrolidin-3-yl}-2-thiophene-2-yl-acetimide
61-5: (S)-N-{1-[3-(2-Methoxy-phenoxy)-benzyl]-pyrrolidin-3-yl}-3-phenyl-propionamide
61-6: (S)-4-Flouro-N-{1-[3-(2-methoxy-phenoxy)-benzyl]-pyrrolidin-3-yl}-benzamide
61-7: (S)-2-(4-Flouro-phenyl)-N-{1-[3-(2-methoxy-phenoxy)-benzyl]-pyrrolidin-3-yl}-acetamide
61-8: (S)-2-(3,4-Dimethoxy-phenyl)-N-{1-[3-(2-methoxy-phenoxy)-benzyl]-pyrrolidin-3-yl)-acetamide
61-9: (S)-N-{1-[3-(2-Methoxy-phenoxy)-benzyl]-pyrrolidin-3-yl}-benzenesulfonamide
61-10: (S)-N-{1-[3-(2-Methoxy-phenoxy)-benzyl]-pyrrolidin-3-yl}-3-phenyl-acrylamide
61-11: (S)-1-(2,4-Dichloro-phenyl)-cyclopropanecarboxylic acid {1-[3-(2-methoxy-phenoxy)-benzyl]-pyrrolidin-3-yl}-amide
61-12: (S)-1-(2-Fluoro-phenyl)-cyclopentecarboxylic acid {1-[3-(2-methoxy-phenoxy)-benzyl]-pyrrolidin-3-yl}-amide
61-13: (S)-1-Phenyl-cyclopentanecarboxlic acid {1-[3-(20-methoxy-phenoxy)-benzyl]-pyrrolidin-3-yl}-amide
61-14: (S)-1-p-Tolyl-cyclopentanecarboxylic acid {1-[3-(2-methoxy-phenoxy)-benzyl]-pyrrolidin-3-yl}-amide
61-15: (S)-N-{-[3-(2-Methoxy-phenoxy)-benzyl]-pyrrolidin-3-yl}-2-p-tolyl-isobutyramide
61-16: (S)-1-(4-Chloro-phenyl)-cyclopentanecarboxylic acid {1-[3-(2-methoxy-phenoxy)-benzyl]-pyrrolidin-3-yl}-amide
61-17: (S)-2-(4-Chloro-phenyl)-N-{1-[3-(2-methoxy-phenoxy)-benzyl]-pyrrolidin-3-yl}-propionamide
61-18: (S)-1-(4-Chloro-phenyl)-cyclohexanecarboxylic acid {1-[3-(2-methoxy-phenoxy)-benzyl]-pyrrolidin-3-yl}-amide

61-19: (S)-2-(2,6-Dichloro-phenyl)-N-{1-[3-(2-methoxy-phenoxy)-benzyl]-pyrrolidin-3-yl}-acetamide

TABLE 15

| | | Retention time | |
|---|---|---|---|
| No. | R | (min) | M + 1 |
| 61-1 | 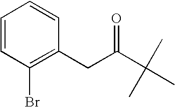 | 1.62 | 495 |
| 61-2 | 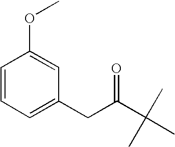 | 1.61 | 447 |
| 61-3 | 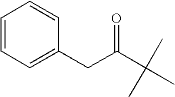 | 1.51 | 417 |
| 61-4 | 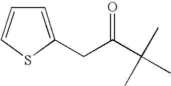 | 1.46 | 424 |
| 61-5 | 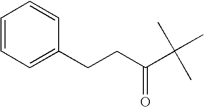 | 1.57 | 432 |
| 61-6 | 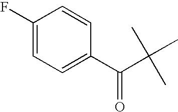 | 1.63 | 421 |
| 61-7 | 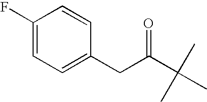 | 1.54 | 435 |
| 61-8 | 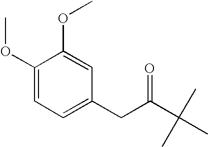 | 1.45 | 478 |
| 61-9 | 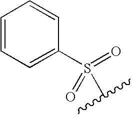 | 1.6 | 453 |
| 61-10 | 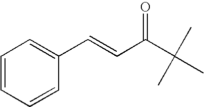 | 1.72 | 429 |
| 61-11 | 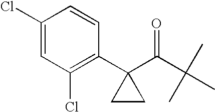 | 1.99 | 511 |
| 61-12 | 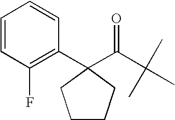 | 1.88 | 489 |
| 61-13 | 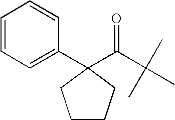 | 1.92 | 471 |
| 61-14 | 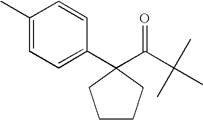 | 2 | 485 |
| 61-15 | 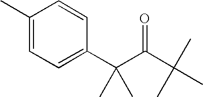 | 1.9 | 479 |
| 61-16 | 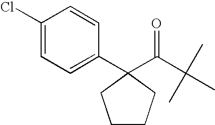 | 2 | 505 |
| 61-17 | 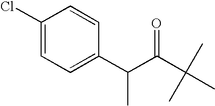 | 1.84 | 465 |
| 61-18 | 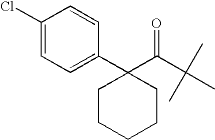 | 2.12 | 519 |
| 61-19 | 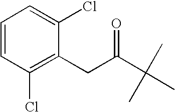 | 1.75 | 485 |

### Preparation of Ethyl-[3-(2-methoxy-phenoxy)-benzyl]-piperidin-3-yl-amine Derivatives

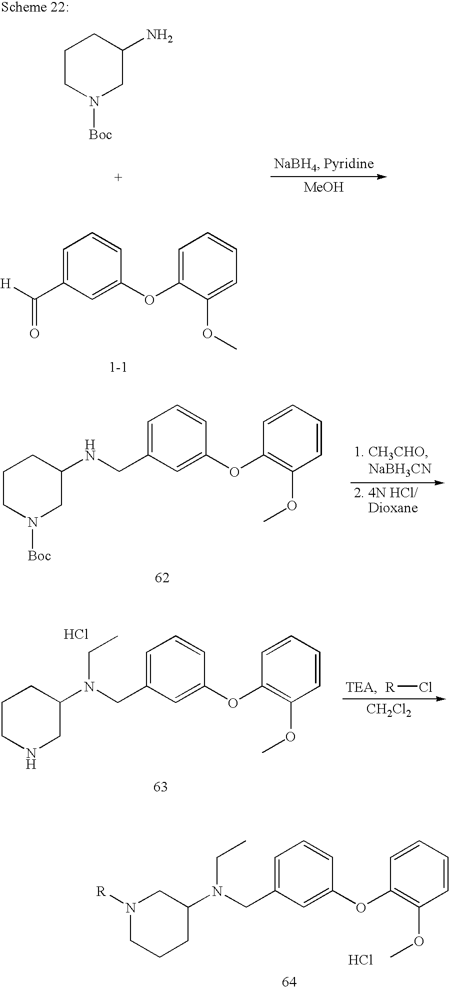

### 62: 3-[3-(2-Methoxy-phenoxy)-benzylamino]-piperidine-1-carboxylic Acid Tert-Butyl Ester

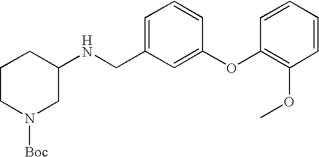

A solution of 3-amino-piperidine-1-carboxylic acid tert-butyl ester (2.35 g, 11.7 mmol), aldehyde 1-1 (2.94 g, 12.9 mmol), pyridine (0.5 ml) in methanol (30 mLs) was heated to reflux at 70° C. for 24 hours. The solution was cooled to 0° C. While stirring over ice, NaBH₄ (443 mg, 11.7 mmol) was added. The reaction was stirred overnight (warming to room temperature) and was quenched with 1 N HCl. The reaction mixture was extracted with ethyl acetate (3×200 mL), washed with NaHCO₃ (2×100 mL), dried over magnesium sulfate and concentrated under reduced pressure to provide 3-[3-(2-methoxy-phenoxy)-benzylamino]-piperidine-1-carboxylic acid tert-butyl ester 62 as an oil, which was used without further purification. ¹H NMR is consistent with the assigned structure.

### 63: Ethyl-[3-(2-methoxy-phenoxy)-benzyl]-piperidin-3-yl-amine

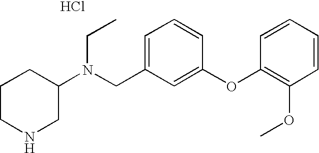

A solution of 3-[3-(2-methoxy-phenoxy)-benzylamino]-piperidine-1-carboxylic acid tert-butyl ester (4.8 g, 11.7 mmol), aldehyde 1-1 (1.54 g, 35 mmol), and NaBH₃CN (735 mg, 11.7 mmol) in methanol (60 mL) was stirred at room temperature overnight. The resulting reaction solution was then taken up in ethyl acetate (3×100 mL), washed with NaHCO₃ (3×100 mL), dried over MgSO₄, filtered and concentrated under reduced pressure. Flash chromatography on SiO₂ (10% ethyl acetate in hexane) provided 3-{ethyl-[3-(2-methoxy-phenoxy)-benzyl]-amino}-piperidine-1-carboxylic acid tert-butyl ester as an oil, which was used without further purification. ¹H NMR is consistent with the assigned structure. LC-MS (ES+): Formic Acid-Standard, M+1=441, ret. time=1.74 min. To the crude product 3-{ethyl-[3-(2-methoxy-phenoxy)-benzyl]-amino}-piperidine-1-carboxylic acid tert-butyl ester 30 mL of 4 M HCl in dioxane was added, stirred for 15 minutes and allowed to warm to room temperature over 1.5 hrs. The solution was concentrated under reduced pressure and triturated with diethyl ether to afford 0.73 g (18%) of ethyl-[3-(2-methoxy-phenoxy)-benzyl]-piperidin-3-yl-amine 63 as a white solid. ¹H NMR is consistent with the assigned structure. LC-MS (ES+):Formic Acid-Standard, M+1=341, ret. time=1.39 min.

### 64: Ethyl-[3-(2-methoxy-phenoxy)-benzyl]-piperidin-3-yl-amine Derivatives

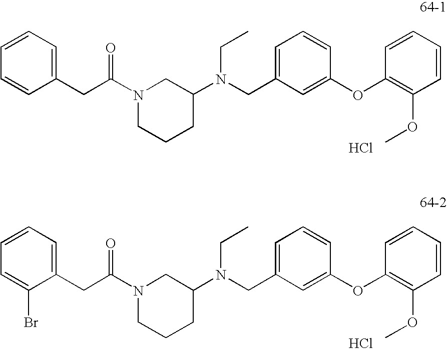

To a solution of ethyl-[3-(2-methoxy-phenoxy)-benzyl]-piperidin-3-yl-amine 63 (170 mg, 0.5 mmol) in dichloromethane (5 mL) was added triethylamine (0.2 mL, 1.5 mmol) and acid chloride (0.5 mmol). The reaction was stirred overnight at room temperature and concentrated in vacuo to provide an oil. Flash chromatography on SiO₂ (40% hexanes in ethyl acetate with 0.5% TEA) provided 64, as an oil which was converted to its HCl salt by treatment with 4.0 M HCl in dioxane. The product was triturated in diethyl ether to afford a powder.
64-1:1-(3-{Ethyl-[3-(2-methoxy-phenoxy)-benzyl]-amino}-piperidin-1-yl)-2-phenyl-ethanone
64-2: 2-(2-Bromo-phenyl)-1-(3-{ethyl-[3-(2-methoxy-phenoxy)-benzyl]-amino}-piperidin-1-yl)-ethanone

### Preparation of N-ethyl-2-{4-hydroxy-1-[3-(2-methoxy-phenoxy)-benzyl]-piperidin-4-yl}-benzenesulfonamide

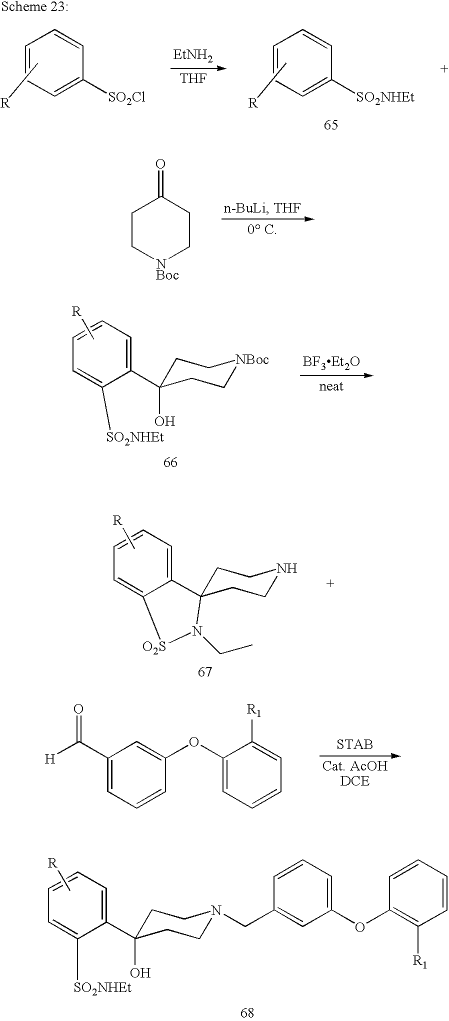

### 65: Benzenesulfonamide

To a solution of benzenesulfonyl chloride (1 eq) in anhydrous THF (1 mL per mmol benzenesulfonyl chloride), amine (2.2 eq) was added at 0° C. The mixture was stirred at RT for 1.5 h. Then sat. NaHCO₃ solution (20 mL) was added to quench the reaction. The phases were separated and the aqueous layer was extracted with EtOAc (3×10 mL). The combined organic phases were dried over MgSO₄ and concentrated to yield a desired product as a white solid or colorless oil. The crude material was pure enough to be used in the next step.

### 65-1: N-Ethyl-4-methyl-benzenesulfonamide

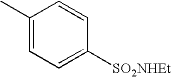

The title compound was prepared according to the general experimental procedure and was obtained as a white solid in 100% yield. ¹H NMR data is consistent with the assigned structure.

### 65-2: 4-Chloro-N-ethyl-benzenesulfonamide

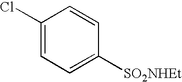

The title compound was prepared according to the general experimental procedure and was obtained as a white solid in 100% yield. ¹H NMR data is consistent with the assigned structure: MS (ESI⁺), M/Z, 221 (M+1), ret. time, 2.29 (HPLC system A).

### 65-3: N-Ethyl-benzenesulfonamide

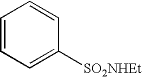

The title compound was prepared according to the general experimental procedure and was obtained as a colorless oil (yield 100%). ¹H NMR data is consistent with the assigned structure: MS (ESI⁺), M/Z, 186 (M+1), ret. time, 1.88 (HPLC system A).

### 65-4: N-Ethyl-α-phenyl-methanesulfonamide

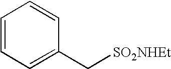

The title compound was prepared according to the general experimental procedure and was obtained as a white solid in a 100% yield. ¹H NMR data is consistent with the assigned structure.

### 65-5: 4-tert-Butyl-N-ethyl-benzenesulfonamide

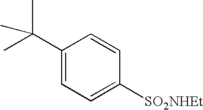

The title compound was prepared according to the general experimental procedure and was obtained as a white solid (yield 100%). ¹H NMR data is consistent with the assigned structure: MS (ESI⁺), M/Z, 242 (M+1), ret. time, 2.77 (HPLC system A).

### 65-6: Biphenyl-4-sulfonic Acid Ethylamide

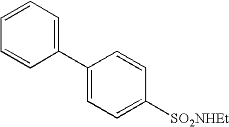

The title compound was prepared according to the general experimental procedure and was obtained as a white solid (yield 100%). ¹H NMR data is consistent with the assigned structure: MS (ESI⁺), M/Z, 262 (M+1), ret. time, 2.63 (HPLC system A).

### 65-7: Naphthalene-2-sulfonic Acid Ethylamide

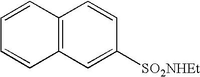

The title compound was prepared according to the general experimental procedure and was obtained as a white solid (yield 100%). ¹H NMR data is consistent with the assigned structure: MS (ESI⁺), M/Z, 236 (M+1), ret. time, 2.39 (HPLC system A).

### 65-8: 4-N-Dimethyl-benzenesulfonamide

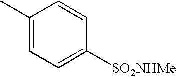

The title compound was prepared according to the general experimental procedure and was obtained as a white solid in 100% yield. ¹H NMR data is consistent with the assigned structure: MS (ESI⁺), M/Z, 186 (M+1), ret. time, 1.91 (HPLC system A).

### 65-9: N-Isopropyl-4-methyl-benzenesulfonamide

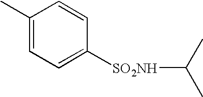

The title compound was prepared according to the general experimental procedure and was obtained as a colorless oil in 100% yield. ¹H NMR data is consistent with the assigned structure: MS (ESI⁺), M/Z, 214 (M+1), ret. time, 2.35 (HPLC system A).

### 65-10: N,N-Diethyl-4-methyl-benzenesulfonamide

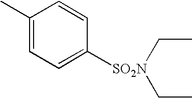

The title compound was prepared according to the general experimental procedure and was obtained as a white solid in a 100% yield. ¹H NMR data is consistent with the assigned structure: MS (ESI⁺), M/Z, 228 (M+1), ret. time, 2.72 (HPLC system A).

### 65-11: N-Ethyl-4-methoxy-benzenesulfonamide

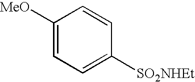

The title compound was prepared according to the general experimental procedure and was obtained as a colorless oil in a 100% yield. ¹H NMR data is consistent with the assigned structure: MS (ESI⁺), M/Z, 216 (M+1), ret. time, 1.99 (HPLC system A).

### 66: Substituted 4-hydroxy-piperidine-1-carboxylic Acid Tert-Butyl Ester

To a solution of benzenesulfonamide 91 (1 eq) in anhydrous THF (5 mL per mmol benzenesulfonamide) under Ar, n-BuLi (2.1 eq, 1.6 M in Hexane) was added dropwise at 0° C. The mixture was stirred at 0° C. for 40 min. Then tert-butyl 4-oxo-piperidinecarboxylate (1.1 eq) in THF (1 mL per mmol tert-butyl 4-oxo-piperidinecarboxylate) was added. The reaction mixture was stirred at RT overnight. H₂O (10 mL) was added to quench the reaction. The phases were separated. The aqueous phase was extracted with EtOAc (3×10 mL). The combined organic phases were dried over MgSO₄. The crude product was purified using silica gel, eluting with EtOAc/hexane (1/4 to 1/2), to give the desired product 66 (yield 35-75%).

### 66-1: 4-(2-Ethylsulfamoyl-4-methyl-phenyl)-4-hydroxy-piperidine-1-carboxylic Acid Tert-Butyl Ester

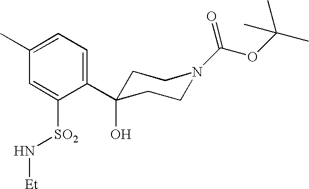

The title compound was prepared according to the general experimental procedure and was obtained as a white solid in a 88% yield. ¹H NMR data is consistent with the assigned structure.

### 66-2: 4-(4-Chloro-2-ethylsulfamoyl-phenyl)-4-hydroxy-piperidine-1-carboxylic Acid Tert-Butyl Ester

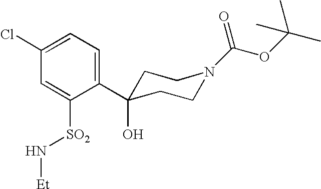

The title compound was prepared according to the general experimental procedure and was obtained as a colorless oil (yield 76%). ¹H NMR data is consistent with the assigned structure: MS (ESI), M/Z, 417 (M−1), ret. time, 2.98 (HPLC system A).

### 66-3: 4-(2-Ethylsulfamoyl-phenyl)-4-hydroxy-piperidine-1-carboxylic Acid Tert-Butyl Ester

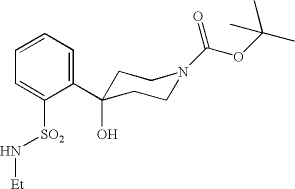

The title compound was prepared according to the general experimental procedure and was obtained as a colorless oil in a 62% yield. ¹H NMR data is consistent with the assigned structure: MS (ESI⁺), M/Z, 383 (M−1), ret. time, 2.78 (HPLC system A).

### 66-4: 4-(2-Ethylsulfamoylmethyl-phenyl)-4-hydroxy-piperidine-1-carboxylic Acid Tert-Butyl Ester

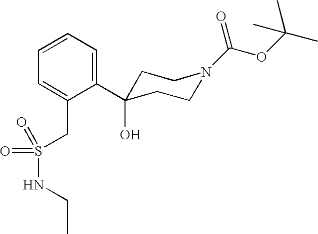

The title compound was prepared according to the general experimental procedure and was obtained as white foam in 87% yield. ¹H NMR data is consistent with the assigned structure.

### 67: Spiro[1,2-benzoisothiazole-3(2H), 4′-piperdines-ethyl-1, 1-dioxide]

A 50 mL round-bottom flask was charged with the starting material 4-hydroxypiperidine 66 (1 eq). BF₃.Et₂O (7.7 eq) was added. The resulting mixture was stirred at RT overnight. 1N NaOH solution was added to basify the mixture. The aqueous phase was extracted with CH₂Cl₂ (3×15 mL). The organic phases were dried over MgSO₄ and concentrated. The desired product 67 was recrystallized from MeOH (yield 60-90%).

### 67-1: Spiro[1,2-benzoisothiazole-3(2H), 4′-piperdines-ethyl-1,1-dioxide-4-methyl]

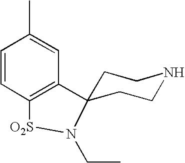

The title compound was prepared according to the general experimental procedure and was obtained as a white solid in a 64% yield. ¹H NMR data is consistent with the assigned structure: MS (ESI⁺), M/Z, 281 (N+1), ret. time, 1.14 (HPLC system A).

### 67-2: Spiro[1,2-benzoisothiazole-3(2H), 4′-piperdines-ethyl-1,1-dioxide-4-chloro]

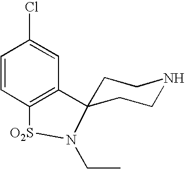

The title compound was prepared according to the general experimental procedure and was obtained as a white solid in a 76% yield. ¹H NMR data is consistent with the assigned structure: MS (ESI⁺), M/Z, 301 (M+1), ret. time, 1.16 (HPLC system A).

### 67-3: Spiro[1,2-benzoisothiazole-3 (2H), 4′-piperdines-ethyl-1,1-dioxide]

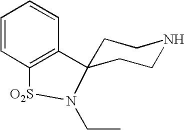

The title compound was prepared according to the general experimental procedure and was obtained as a white solid in a 68% yield ¹H NMR data is consistent with the assigned structure: MS (ESI), M/Z, 267 (M+1), ret. time, 0.98 (HPLC system A).

### 67-4: N-Ethyl-α-[2-(4-hydroxy-piperidin-4-yl)-phenyl]-methanesulfonamide

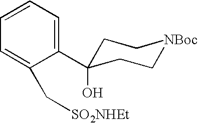

The title compound was prepared according to the general experimental procedure and was obtained as a white solid in a 100% yield. ¹H NMR data is consistent with the assigned structure: MS (ESI⁺), M/Z, 300 (M+1), ret. time, 0.78 (HPLC system A).

### 68: Substituted N-Ethyl-2-[4-hydroxy-1-(3-phenoxy-benzyl)-piperidin-4-yl]-5-methyl-benzenesulfonamide

To a solution of piperidine 67 (1 eq) in anhydrous dichloroethane (10 mL per mmol piperidine 67), aromatic aldehyde 1 (1.1 eq) and sodium triacetoxyborohydride (1.6 eq) were added. Cat. AcOH was added (1 drop). The mixture was stirred at RT overnight. Then sat. NaHCO₃ solution was added to quench the reaction. The phases were separated. The aqueous phase was extracted with EtOAc (3×10 mL). The combined organic phases were dried over MgSO₄. The crude product was purified, using chromatography on silica gel, to give the desired product 68. The formate salt or HCl salt was prepared by treating a solution of free base in Et₂O with 1M formic acid or 1M HCl solution in Et₂O.

### 68-1: N-Ethyl-2-[4-hydroxy-1-(3-phenoxy-benzyl)-piperidin-4-yl]-5-methyl-benzenesulfonamide

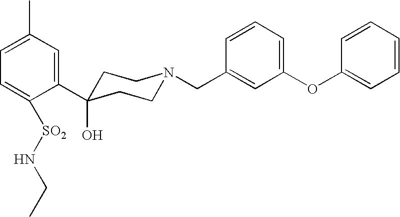

The title compound was prepared according to the general experimental procedure and was obtained as a colorless oil in a 74% yield. ¹H NMR data is consistent with the assigned structure: MS (ESI⁺), M/Z, 481 (M+1), ret. time, 1.86 (HPLC system A); Anal Calcd for C₂₇H₃₂N₂O₄S: C, 67.47; H, 6.71; N, 5.83. Found: C, 67.08; H, 6.76; N, 5.75.

### 68-2: N-Ethyl-2-{4-hydroxy-1-[3-(2-methoxy-phenoxy)-benzyl]-piperidin-4-yl}-5-methyl-benzenesulfonamide

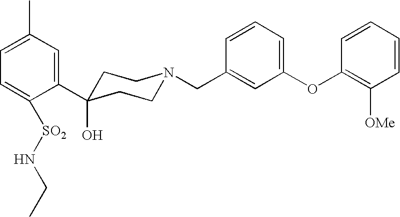

The title compound was prepared according to the general experimental procedure and was obtained as a white solid in a 59% yield. ¹H NMR data is consistent with the assigned structure: MS (ESI⁺), M/Z, 511 (+1), ret. time, 1.76 (HPLC system A); Anal Calcd for C₂₈H₃₄N₂O₅S.CH₂O₂.H₂O: C, 60.61; H, 6.66; N, 4.87. Found: C, 60.52; H, 6.52; N, 4.71.

### 68-3: 2-{1-[3-(2-Chloro-phenoxy)-benzyl]-4-hydroxy-piperidin-4-yl}-N-ethyl-5-methyl-benzenesulfonamide

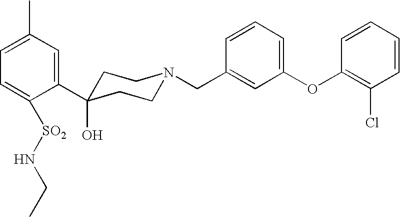

The title compound was prepared according to the general experimental procedure and was obtained as a white solid in a 65% yield. ¹H NMR data is consistent with the assigned structure: MS (ESI⁺), M/Z, 515 (M+1), ret. time, 1.83 (HPLC system A); Anal calcd for C₂₇H₃₁ClN₂O₄S._{0.75}CH₂O₂: C, 60.65; H, 5.96; N, 5.10. Found: C, 60.70; H, 5.96; N, 5.08.

### 68-4: 5-Chloro-N-ethyl-2-[4-hydroxy-1-(3-phenoxy-benzyl)-piperidin-4-yl]-benzenesulfonamide

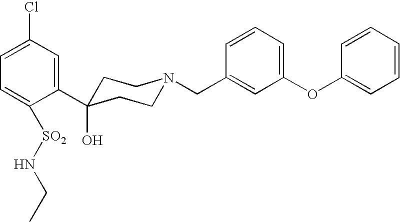

The title compound was prepared according to the general experimental procedure and was obtained as a white solid in a 60% yield. ¹H NMR data is consistent with the assigned structure: MS (ESI⁺), M/Z, 501 (+1), ret. time, 1.79 (HPLC system A); Anal calcd for C₂₆H₂₉ClN₂O₄S.CH₂O₂.4H₂O; C, 58.51; H, 5.78; N, 5.05. Found: C, 58.50; H, 5.55; N, 4.97.

### 68-5: 5-Chloro-N-ethyl-2-{4-hydroxy-1-[3-(2-methoxy-phenoxy)-benzyl]-pip eridin-4-yl}-benzenesulfonamide

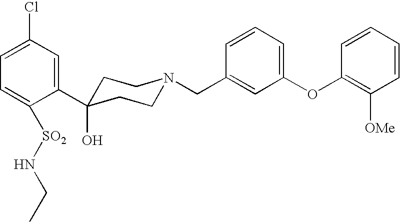

The title compound was prepared according to the general experimental procedure and was obtained as a white solid in a 40% yield. ¹H NMR data is consistent with the assigned structure: MS (ESI⁺), M/Z, 532 (M+1), ret. time, 1.81 (HPLC system A); Anal calcd for C₂₇H₃₁ClN₂O₅S.CH₂O₂1.3H₂O; C, 56.00; H, 5.98; N, 4.66. Found: C, 56.03; H, 5.83; N, 4.36.

### 68-6: N-Ethyl-2-[4-hydroxy-1-(3-phenoxy-benzyl)-piperidin-4-yl]-benzenesulfonamide

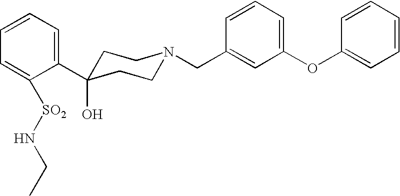

The title compound was prepared according to the general experimental procedure and was obtained as a white solid in a 53% yield. ¹H NMR data is consistent with the assigned structure: MS (ESI⁺), M/Z, 467 (M+1), ret. time, 1.74 TALC system A); Anal calcd for C₂₆H₃₀N₂O₄S.CH₂O₂.0.5H₂O; C, 61.43; H, 6.56; N, 5.21. Found: C, 61.65; H, 6.16; N, 4.83.

### 68-7: N-Ethyl-2-{4-hydroxy-1-[3-(2-methoxy-phenoxy)-benzyl]-piperidin-4-yl}-benzenesulfonamide

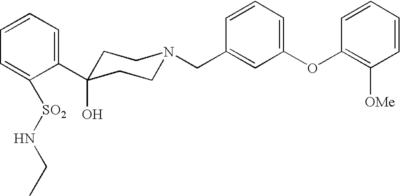

The title compound was prepared according to the general experimental procedure and was obtained as a white solid (yield 53%). ¹H NMR data is consistent with the assigned structure: MS (ESI⁺), M/Z, 497 (M+1), ret. time, 1.63 (HPLC system A); Anal Calcd for C₂₇H₃₂N₂O₅S.CH₂O₂; C, 61.97; H, 6.32; N, 5.16. Found: C, 61.68; H, 6.39; N, 5.03.

### 68-8: N-Ethyl-α-{2-[4-hydroxy-1-(3-phenoxy-benzyl)-piperidin-4-yl]-phenyl}-methanesulfonamide

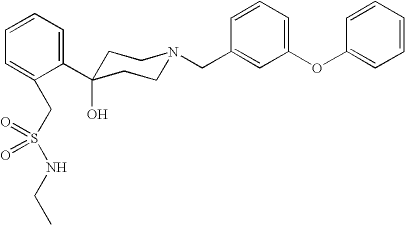

The title compound was prepared according to the general experimental procedure and was obtained as a white solid in 33% yield. ¹H NMR data is consistent with the assigned structure: MS (ESI⁺), M/Z, 481 (M+1), ret. time, 1.61 (HPLC system A); Anal calcd for C₂₇H₃₂N₂O₄S.CH₂O₂.0.75H₂O; C, 62.26; H, 6.62; N, 5.19. Found: C, 62.20; H, 6.64; N, 5.05.

### 68-9: N-Ethyl-C-(2-{4-hydroxy-1-[3-(2-methoxy-phenoxy)-benzyl]-piperidin-4-yl}-phenyl)-methanesulfonamide

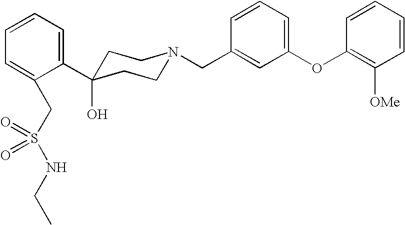

The title compound was prepared according to the general experimental procedure and was obtained as a white solid in 52% yield. ¹H NMR data is consistent with the assigned structure: MS (ESI⁺), M/Z, 511 (M+1), ret. time, 1.67 (HPLC system A); Anal calcd for C₂₈H₃₄N₂O₅S.CH₂O₂.2H₂O; C, 58.77; H, 6.80; N, 4.73. Found: C, 58.89; H, 6.50; N, 4.45.

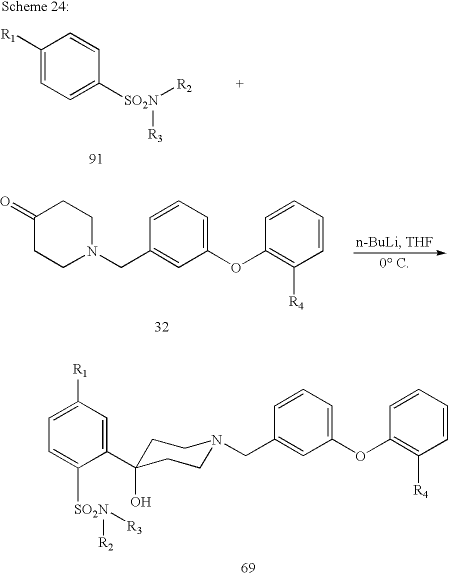

### 69: 4-aryl-4-hydroxy-1-(3-phenoxy-benzyl)-piperidine

To a solution of benzenesulfonamide 91 (1 eq) in anhydrous THF (5 mL per mmol sulfonamide) under Ar, n-BuLi (2 eq, 2.5M solution in hexane) was added dropwise at 0° C. The mixture was stirred at 0° C. for 15 min, then RT for 15 min. Piperidone 32 (1 eq) in anhydrous THF (1 mL per mmol piperidone) was added. The final reaction mixture was stirred at RT overnight. H₂O (20 mL) was added to quench the reaction. The phases were separated. The aqueous phase was extracted with EtOAc (3×15 mL). The combined organic phases were dried over MgSO₄. The crude product was purified using chromatography over silica gel, to give the desired product 69. The formate salt was prepared by treating a solution of free base in Et₂O with 1M formic acid solution in Et₂O.

### 69-1: 3-[4-Hydroxy-1-(3-phenoxy-benzyl)-piperidin-4-yl]-biphenyl-4-sulfonic Acid Ethylamide

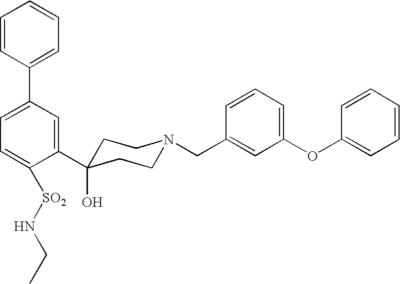

The title compound was prepared according to the general experimental procedure and was obtained as a white solid in a 22% yield. ¹H NMR data is consistent with the assigned structure: MS (ESI⁺), M/Z, 543 (M+1), ret. time, 2.04 (HPLC system A); Anal calcd for C₃₂H₃₄N₂O₄S.CH₂O₂.0.5H₂O; C, 66.31; H, 6.24; N, 4.69. Found: C, 66.02; H, 6.21; N, 4.58.

### 69-2: 2-[4-Hydroxy-1-(3-phenoxy-benzyl)-piperidin-4-yl]-4,N-dimethyl-benzenesulfonamide

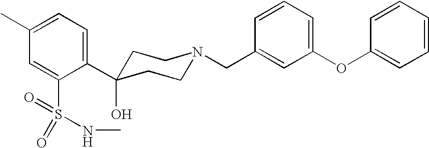

The title compound was prepared according to the general experimental procedure and was obtained as a white solid in a 31% yield. ¹H NMR data is consistent with the assigned structure: MS (ESI⁺), M/Z, 467 (M+1), ret. time, 1.74 (HPLC system A); Anal calcd for C₂₆H₃₀N₂O₄S.0.5CH₂O₂; C, 65.01; H, 6.38; N, 5.72. Found: C, 65.08; H, 6.43; N, 5.68.

### 69-3: 2-{4-Hydroxy-1-[3-(2-methoxy-phenoxy)-benzyl]-piperidin-4-yl}-4,N-dimethyl-benzenesulfonamide

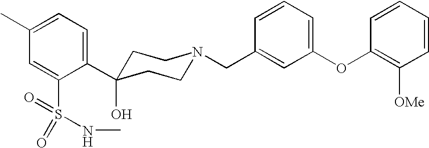

The title compound was prepared according to the general experimental procedure and was obtained as a white solid in a 15% yield. ¹H NMR data is consistent with the assigned structure: MS (ESI⁺), M/Z, 497 (M+1), ret. time, 1.74 (HPLC system A); Anal calcd for C₂₇H₃₂N₂O₅S-0.8CH₂O₂; C, 60.37; H, 6.44; N, 5.03. Found: C, 60.35; H, 6.27; N, 4.86.

### 69-4: 2-[4-Hydroxy-1-(3-phenoxy-benzyl)-piperidin-4-yl]-N-isopropyl-4-methyl-benzenesulfonamide

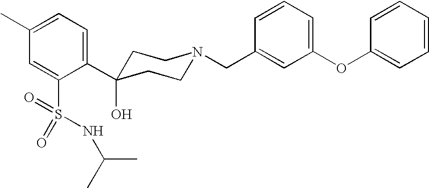

The title compound was prepared according to the general experimental procedure and was obtained as a white solid in a 34% yield. ¹H NMR data is consistent with the assigned structure: MS (ESI⁺), M/Z, 495 (M+1), ret. time, 1.90 (HPLC system A); Anal calcd for C₂₈H₃₄N₂O₄S.0.25CH₂O₂; C, 67.04; H, 6.87; N, 5.53. Found: C, 67.06; H, 6.78; N, 5.40.

### 69-5: 2-{4-Hydroxy-1-[3-(2-methoxy-phenoxy)-benzyl]-piperidin-4-yl}-N-isopropyl-4-methyl-benzenesulfonamide

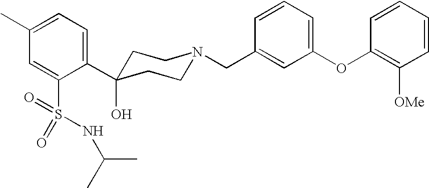

The title compound was prepared according to the general experimental procedure and was obtained as a white solid in a 21% yield. ¹H NMR data is consistent with the assigned structure: MS (ESI⁺), M/Z, 525 (M+1), ret. time, 1.97 (HPLC system A); Anal calcd for C₂₉H₃₆N₂O₅S.0.5CH₂O₂; C, 64.69; H, 6.81; N, 5.11. Found: C, 64.90; H, 6.86; N, 5.04.

### 69-6: N,N-Diethyl-2-[4-hydroxy-1-(3-phenoxy-benzyl)-piperidin-4-yl]-4-methyl-benzenesulfonamide

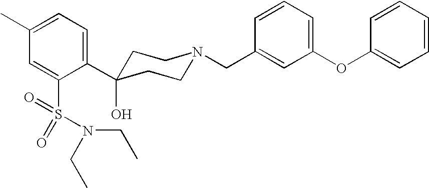

The title compound was prepared according to the general experimental procedure (using 1 eq n-BuLi) and was obtained as a white solid in a 34% yield. This compound was converted into acetic acid salt. ¹H NMR data is consistent with the assigned structure: MS (ESI⁺), M/Z, 509 (M+1), ret. time, 2.01 (HPLC system A); Anal calcd for C₂₉H₃₆N₂O₄S.0.2AcOH; C, 67.82; H, 7.12; N, 5.38. Found: C, 67.75; H, 7.19; N, 5.02.

### 69-7: N,N-Diethyl-2-{4-hydroxy-1-[3-(2-methoxy-phenoxy)-benzyl]-piperidin-4-yl}-4-methyl-benzenesulfonamide

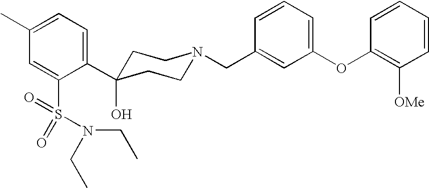

The title compound was prepared according to the general experimental procedure (using 1 eq n-BuLi) and was obtained as a white solid in a 23% yield. ¹H NMR data is consistent with the assigned structure: MS (ESI⁺), M/Z, 539 (M+1), ret. time, 1.95 (HPLC system A); Anal calcd for C₃₀H₃₈N₂O₅S.CH₂O₂.0.75H₂O; C, 62.24; H, 6.99; N, 4.68. Found: C, 62.16; H, 6.89; N, 4.47.

### 69-8: 3-{4-Hydroxy-1-[3-(2-methoxy-phenoxy)-benzyl]-piperidin-4-yl}-naphthalene-2-sulfonic Acid Ethylamide

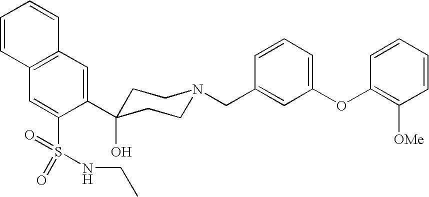

The title compound was prepared according to the general experimental procedure and was obtained as a white solid in 7% yield. ¹H NMR data is consistent with the assigned structure: MS (ESI⁺), M/Z, 547 (+1), ret. time, 2.05 (HPLC system A); Anal calcd for C₃₁H₃₄N₂O₅S.CH₂O₂; C, 64.85; H, 6.12; N, 4.73. Found: C, 65.08; H, 6.37; N, 4.35.

### 69-9: 3-[4-Hydroxy-1-(3-phenoxy-benzyl)-piperidin-4-yl]-naphthalene-2-sulfonic Acid Ethylamide

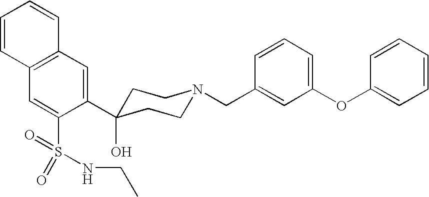

The title compound was prepared according to the general experimental procedure and was obtained as a white solid in a 18% yield. ¹H NMR data is consistent with the assigned structure: MS (ESI⁺), M/Z, 517 (+1), ret. time, 2.02 (HPLC system A); Anal calcd for C₃₀H₃₂N₂O₄S.CH₂O₂.0.5H₂O; C, 65.13; H, 6.70; N, 4.90. Found: C, 65.17; H, 6.40; N, 4.60.

### 69-10: 4-tert-Butyl-N-ethyl-2-{4-hydroxy-1-[3-(2-methoxy-phenoxy)-benzyl]-piperidin-4′-yl}-benzenesulfonamide

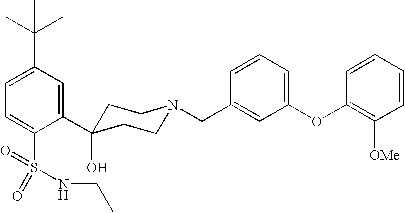

The title compound was prepared according to the general experimental procedure and was obtained as a white solid in a 42% yield. ¹H NMR data is consistent with the assigned structure: MS (ESI⁺), M/Z, 553 (M+1), ret. time, 2.15 (HPLC system A); Anal calcd for C₃₁H₄₀N₂O₅S.CH₂O₂-1H₂O; C, 62.32; H, 7.19; N, 4.54. Found: C, 62.52; H, 6.92; N, 4.51.

### 69-11: N-Ethyl-2-{4-hydroxy-1-[3-(2-methoxy-phenoxy)-benzyl]-piperidin-4-yl}-4-methoxy-benzenesulfonamide

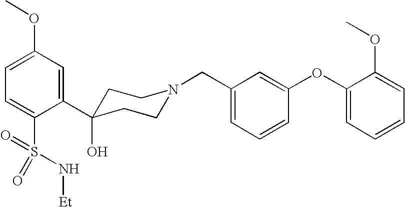

The title compound was prepared according to the general experimental procedure and was obtained as a white solid in a 14% yield. ¹H NMR data is consistent with the assigned structure: MS (ESI⁺), M/Z, 527 (M+1), ret. time, 1.90 (HPLC system A); Anal calcd for C₂₈H₃₄N₂O₆S.CH₂O₂.1H₂O; C, 58.97; H, 6.48; N, 4.74. Found: C, 58.70; H, 6.39; N, 4.50.

### 69-12: N-Ethyl-2-[4-hydroxy-1-(3-phenoxy-benzyl)-piperidin-4-yl]-4-methoxy-benzenesulfonamide

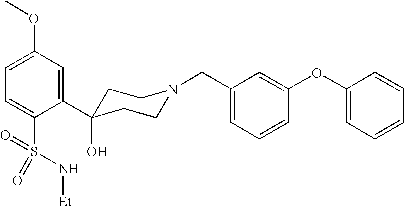

The title compound was prepared according to the general experimental procedure and was obtained as a white solid in 50% yield. ¹H NMR data is consistent with the assigned structure: MS (ESI⁺), M/Z, 497 (M+1), ret. time, 1.90 (HPLC system A); Anal calcd for C₂₇H₃₂N₂O₅S.CH₂O₂.1.25H₂O; C, 59.51; H, 6.51; N, 4.96. Found: C, 59.47; H, 6.45; N, 4.69.

### 69-13: 4-[2-(2,2-Dimethyl-2,5-dihydro-oxazol-4-yl)-phenyl]-1-(3-phenoxy-benzyl)-piperidin-4-ol

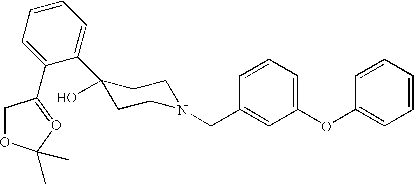

The title compound was prepared according to the general experimental procedure and was obtained as a white in a 36% yield. ¹H NMR data is consistent with the assigned structure: MS (ESI⁺), M/Z, 457 (M+1), ret. time, 1.29 (HPLC system A); Anal calcd for C₃₀H₃₄N₂O₃S.CH₂O₂.0.25H₂O; C, 71.45; H, 7.06; N, 5.38. Found: C, 71.50; H, 7.10; N, 5.24.

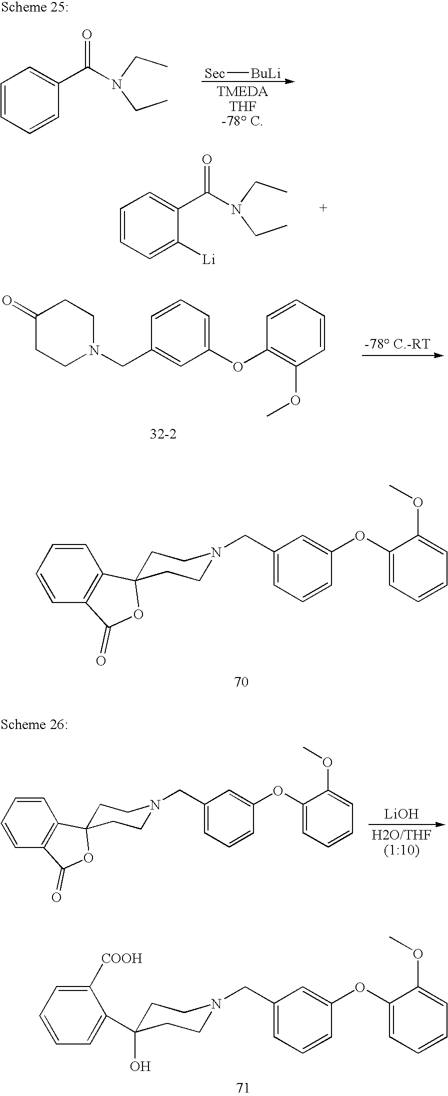

### 70: Spiro[isobenzofuran-1 (3H),4′-piperdin]-3-one,1′-[[3-(2-methoxyphenoxy)phenyl]methyl]

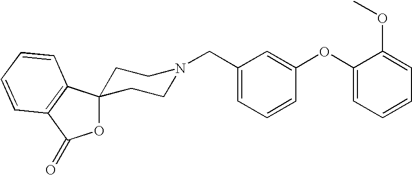

To a solution of N,N-diethylbenzamide (300.00 mg, 1.69 mmol) in anhydrous THF (5 mL) under Ar, TMEDA (0.25 mL, 1.69 mmol) was added. After cooling to −78° C. Sec-BuLi (1.30 mL, 1.69 mmol, 1.3M solution in cyclohexane) was added dropwise. The resulting mixture was stirred for 30 min at −78° C. Then piperidone 32-2 (525.00 mg, 1.69 mmol) in anhydrous THF (1 mL) was added. The final reaction mixture was stirred and allowed to warm from −78° C. to RT overnight. H₂O (10 mL) was added to quench the reaction. The phases were separated. The aqueous phase was extracted with EtOAc (3×15 mL). The combined organic phases were dried over MgSO₄. The crude product was purified using chromatography on silica gel, to give the desired product (258.00 mg, yield 37%). The HCl salt was prepared by treating a solution of free base in Et₂O with 1M HCl solution in Et₂O. ¹H NMR data is consistent with the assigned structure: MS (ESI⁺), M/Z, 415 (M+1), ret. time, 1.74 (HPLC system A); Anal calcd for C₂₆H₂₅NO₄.HCl.1H₂O; C, 66.45; H, 6.01; N, 2.98. Found: C, 66.72; H, 6.01; N, 2.97.

### 71: 2-{4-Hydroxy-1-[3-(2-methoxy-phenoxy)-benzyl]-piperidin-4-yl}-benzoic Acid

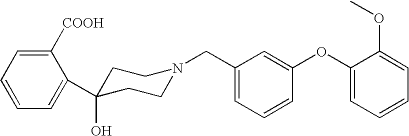

To a solution of 70 (190 mg, 0.45 mmol) in THF/H₂O (10 mL, 10:1), LiOH (109.00 mg, 4.50 mmol) was added. The mixture was heated to reflux overnight, then brought to pH 7 using aqueous 1M HCl. Sat. NH₄Cl solution (10 mL) was added, and the phases were separated. The aqueous phase was extracted with EtOAc (3×5 mL). The combined organic phases were dried over MgSO₄ and concentrated. The desired product was obtained by recrystallization in MeOH (50 mg, yield 25%). ¹H NMR data is consistent with the assigned structure: MS (ESI⁺), M/Z, 432 (M−1), ret. time, 1.73 (HPLC system A); Anal calcd for C₂₆H₂₇NO₅.1.1H₂O; C, 68.89; H, 6.47; N, 3.09. Found: C, 68.70; H, 6.77; N, 3.47.

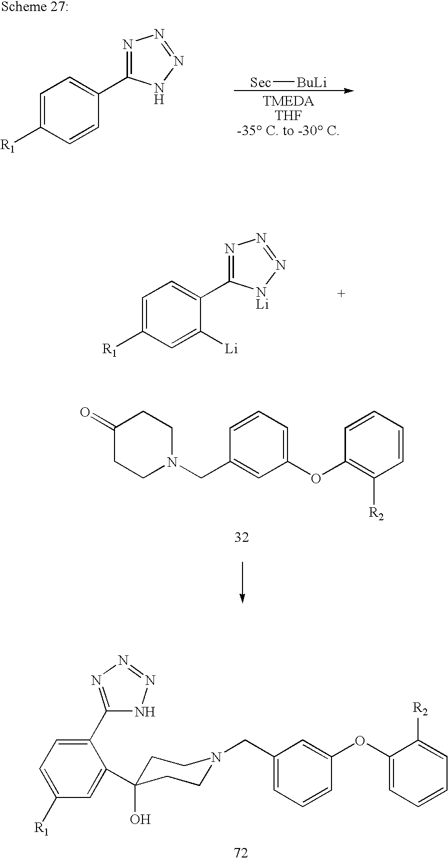

### 72: 1-[3-(2-Methoxy-phenoxy)-benzyl]-4-[2-(1H-tetrazol-5-yl)-aryl]-piperidin-4-ol

To a solution of 5-phenyl-tetrazole (1 eq) in anhydrous THF (5 mL per mmol tetrazole) under Ar, tetramethyl-ethylenediamine (1 eq) was added. After cooling to −35° C.−30° C. Sec-BuLi (3 eq, 1.3M solution in cyclohexane) was added dropwise. The resulting mixture was stirred for 45 min at −35° C.˜−30° C. Then piperidone (1 eq) in anhydrous THF (1 mL) was added. The final reaction mixture was stirred at −35° C.-30° C. for 5 h. Sat. NH₄Cl solution (10 mL) was added to quench the reaction. The pH was adjusted to ˜7 with aqueous 1 M HCl. The phases were separated. The aqueous phase was extracted with THF (3×15 mL). The combined organic phases were dried over MgSO₄. The crude product was purified using silica gel, eluting with EtOAc/MeOH (9:1), to give the desired product as a white solid. The product was recrystallized from MeOH.

### 72-1:1-[3-(2-Methoxy-phenoxy)-benzyl]-4-[2-(1H-tetrazol-5-yl)-phenyl]-piperidin-4-ol

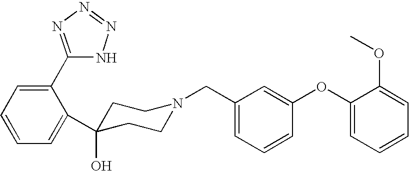

The title compound was prepared according to the general experimental procedure and was obtained as a white solid in 7% yield. ¹H NMR data is consistent with the assigned structure: MS (ESI⁺), M/Z, 458 (M+1), ret. time, 1.72 (HPLC system A); Anal calcd for C₂₆H₂₇N₅O₃.1.6H₂O; C, 64.21; H, 6.24; N, 14.40. Found: C, 64.01; H, 6.10; N, 14.19.

### 72-2: 4-[5-Chloro-2-(1H-tetrazol-5-yl)-phenyl]-1-[3-(2-methoxy-phenoxy)-benzyl]-piperidin-4-ol

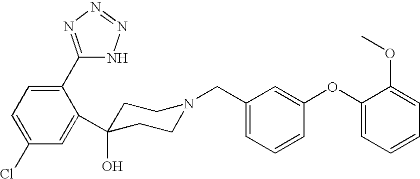

The title compound was prepared according to the general experimental procedure and was obtained as a white solid in 14% yield. ¹H NMR data is consistent with the assigned structure: MS (ESI⁺), M/Z, 492 (M+1), ret. time, 1.92 (HPLC system A); Anal calcd for C₂₆H₂₆ClN₅O_{3.1.6}H₂O; C, 59.96; H, 5.69; N, 13.45. Found: C, 59.96; H, 5.45; N, 13.39.

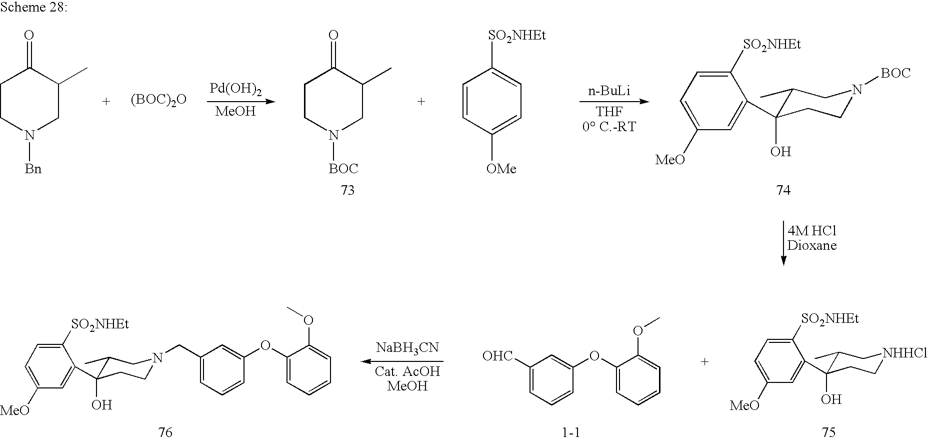

### 73: 3-Methyl-4-oxo-piperidine-1-carboxylic Acid Tert-Butyl Ester

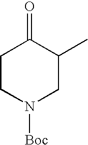

To a solution of 1-benzyl-3-methyl-4-piperidone (6.50 g, 31.97 mmol) in MeOH (50 mL), di-tert-butyl dicarbonate (10.46 g, 47.96 mmol) and Pd(OH)₂ (0.70 g, 10 wt %) were added. The reaction mixture was placed under H₂ on a Parr shaker apparatus at 40 psi overnight at RT. The mixture was filtered through celite and concentrated. The resulting crude product was purified using silica gel, eluting with EtOAc/hexane (1/4), to give the desired product as a white solid (5.73 g, yield 84%). ¹H NMR data is consistent with the assigned structure: MS (ESI⁺), M/Z, 158 (M-56), ret. time, 2.03 (HPLC system A).

### 74: 4-(2-Ethylsulfamoyl-5-methoxy-phenyl)-4-hydroxy-3-methyl-piperidine-1-carboxylic Acid Tert-Butyl Ester

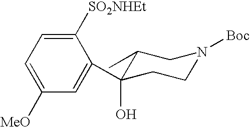

The title compound was prepared according to the general experimental procedure and was obtained as a white foam in a 38% yield. ¹H NMR data is consistent with the assigned structure. This compound was used directly in the next step.

### 75: N-Ethyl-2-(4-hydroxy-3-methyl-piperidin-4-yl)-4-methoxy-benzenesulfonamide Hydrochloride Salt

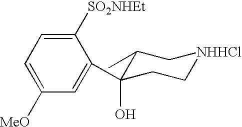

4-(2-Ethylsulfamoyl-5-methoxy-phenyl)-4-hydroxy-3-methyl-piperidine-1-carboxylic acid tert-butyl ester (0.38 g, 0.89 mmol) was dissolved in 4M HCl solution in dioxane (2 mL, 8.00 mmol). The mixture was stirred at RT for 1 h. Then the mixture was concentrated to give a white solid. The white solid was washed with Et₂O (10 mL) to generate 0.32 g of the desired product. ¹H NMR data is consistent with the assigned structure. MS (ESI⁺), M/Z, 329 (M+1), ret. time, 1.25 (HPLC system A).

### 76: N-Ethyl-2-{4-hydroxy-1-[3-(2-methoxy-phenoxy)-benzyl]-3-methyl-piperidin-4-yl}-4-methoxy-benzenesulfonamide

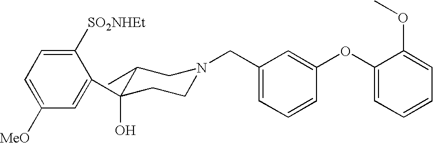

To a solution of N-ethyl-2-(4-hydroxy-3-methyl-piperidin-4-yl)-4-methoxy-benzenesulfonamide hydrochloride salt (1 eq) in anhydrous MeOH (10 mL per mmol piperidine), aromatic aldehyde 1-1 (1.1 eq) and sodium cyanoborohydride (1.6 eq) were added. Cat. AcOH was used to adjust pH to 6. The mixture was stirred at RT overnight. Then sat. NaHCO₃ solution was added to quench the reaction. The phases were separated. The aqueous phase was extracted with EtOAc (3×10 mL). The combined organic phases were dried over MgSO₄. The crude product was purified using chromatography on silica gel to give the desired product. The formate salt or HCl salt was prepared by treating a solution of free base in Et₂O with 1M formic acid or HCl solution in Et₂O. ¹H NMR data is consistent with the assigned structure: MS (ESI⁺), M/Z, 541 (M+1), ret. time, 1.95 (HPLC system A); Anal calcd for C₂₉H₃₆N₂O₆S.CH₂O₂.0.9H₂O; C, 59.76; H, 6.65; N, 4.65. Found: C, 60.06; H, 6.53; N, 4.27.

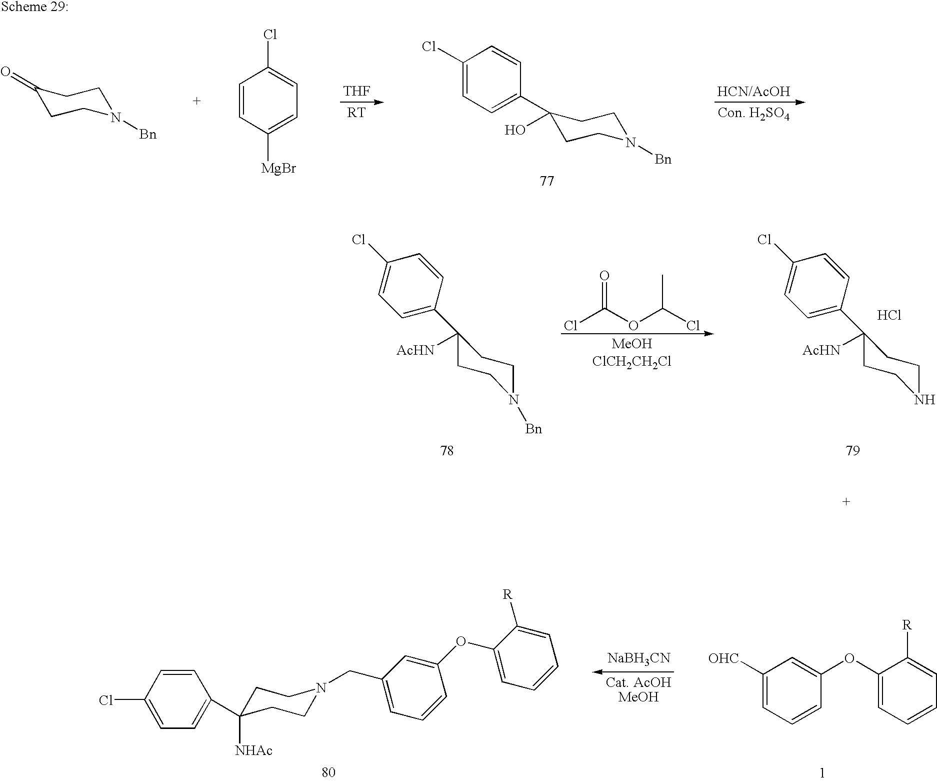

### 77: 1-Benzyl-4-(4-chloro-phenyl)-piperidin-4-ol

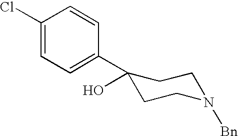

To a solution of N-benzylpiperidone (3.0 mL, 16.2 mmol) in anhydrous THF (100 mL), 4-chlorophenyl magnesium bromide (25 mL, 25 mmol, 1M solution in Et₂O) was added at RT. The mixture was stirred at rt for 5 h. Then sat. NaCl solution (20 mL) was added to quench the reaction. The phased were separated. The aqueous phase was extracted with EtOAc (3×15 mL). The combined organic phases were dried over MgSO₄ and concentrated. The crude product was purified on silica gel, eluting with CH₂Cl₂/MeOH (95:5), to give the desired product as a yellow oil (4.98 g, yield 100%). ¹H NMR data is consistent with the assigned structure: MS (ESI⁺), M/Z, 302 (M+1), ret. time, 1.30 (HPLC system A).

### 78: N-[1-Benzyl-4-(4-chloro-phenyl)-piperidin-4-yl]-acetamide

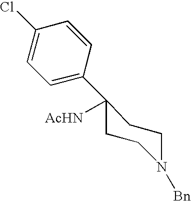

To a solution of acetonitrile (9 mL) in acetic acid (6 mL) was treated with concentrated H₂SO₄ (3 mL), and N-benzyl-[4-(4-chlorophenyl)]-4-hydroxy piperidine 77 (2.78 g, 9.20 mmol) was added. The mixture was stirred at rt for 2 h. Then the reaction mixture was cooled to 0° C. and quenched with 1N NaOH solution. The aqueous phase was extracted with EtOAc (3×20 mL). The combined organic phases were dried over MgSO₄ and concentrated to give a white solid. The solid was triturated with CH₂Cl₂ and Hexane to afford the desired product XH (2.11 g, yield 67%). ¹H NMR data is consistent with the assigned structure: MS (ESI⁺), M/Z, 343 (M+1), ret. time, 1.25 (HPLC system A).

### N-[4-(4-Chloro-phenyl)-piperidin-4-yl]-acetamide Hydrochloride Salt (79)

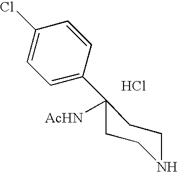

To a solution of N-[1-Benzyl-4-(4-chloro-phenyl)-piperidin-4-yl]-acetamide 78 (2.63 g, 7.68 mmol) in anhydrous dichloroethane (30 mL), 1-chloroethyl chloroformate (1.07 mL, 10.00 mmol) was added. The mixture was heated to reflux for 4 h. After cooling to RT, the solvent was removed. MeOH (10 mL) was added. The mixture was heated to reflux for 4 h. After removing the solvent, the residue was triturated with EtOAc to give a white solid XI (1.64 g, yield 74%). ¹H NMR data is consistent with the assigned structure: MS (ESI⁺), M/Z, 253 (M+1), ret. time, 2.31 (HPLC system A).

### 80-1: N-[4-(4-Chloro-phenyl)-1-(3-phenoxy-benzyl)-piperidin-4-yl]-acetamide

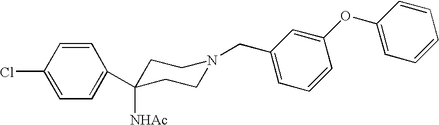

The title compound was prepared according to the experimental procedure for 76 and was obtained as a white solid (free base, yield 53%). ¹H NMR data is consistent with the assigned structure: MS (ESI⁺), M/Z, 435 (M+1), ret. time, 1.87 (HPLC system A); Anal calcd for C₂₆H₂₇ClN₂O₂; C, 71.80; H, 6.26; N, 6.44. Found: C, 71.49; H, 6.00; N, 6.41.

### 80-2: N-{4-(4-Chloro-phenyl)-1-[3-(2-methoxy-phenoxy)-benzyl]-piperidin-4-yl}-acetamide

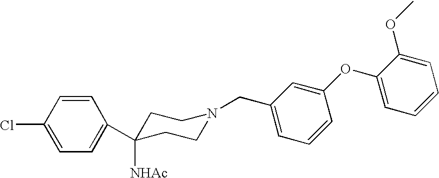

The title compound was prepared according to the experimental procedure for 76 and was obtained as a white solid (free base, yield 46%). ¹H NMR data is consistent with the assigned structure: MS (ESI⁺), M/Z, 465 (M+1), ret. time, 1.84 (HPLC system A); Anal calcd for C₂₇H₂₉ClN₂O₃.0.2H₂O; C, 69.21; H, 6.32; N, 5.98. Found: C, 69.24; H, 6.22; N, 5.84.

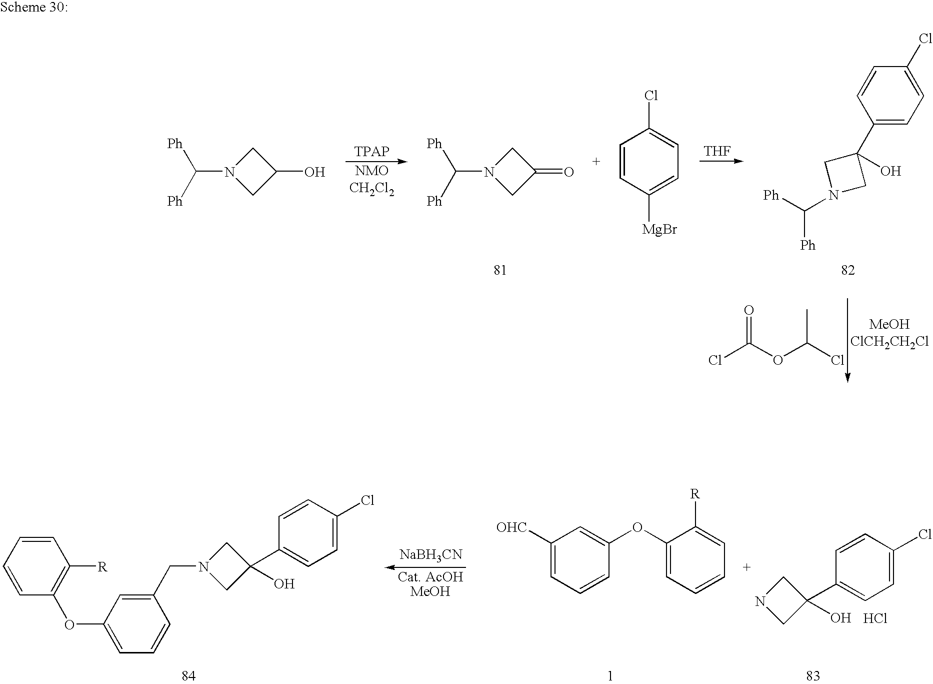

### 81: 1-Benzhydryl-azetidin-3-one

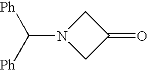

To a solution of 1-benzhydrylazetan-3-ol (1.08 g, 4.51 mmol) in anhydrous dichloromethane (10 mL), tetrapropylammonium perruthenate (0.070 g, 0.2 mmol), N-methymorpholine N-oxide (1.17 g, 10 mmol) and 4 A molecular sieves were added. The mixture was stirred at RT for 2 h. The mixture was filtered and concentrated. The crude product was chromatographed on silica gel, eluting with hexane/EtOAc (3/2), to give 1.06 g of the desired product as a white solid in 99% yield. ¹H NMR data is consistent with the assigned structure.

### 82: 1-Benzhydryl-3-(4-chloro-phenyl)-azetidin-3-ol

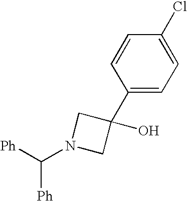

To a solution of 4-chlorophenyl magnesium bromide (4 mL, 4.00 mmol, 1M in THF) at 0° C., ketone 81 (0.59 g, 2.50 mmol) was added. The mixture was warmed to RT and stirred for 2.5 h. Then Sat. NaCl solution was added to quench the reaction. The phases were separated. The aqueous phase was extracted with EtOAc (3×10 mL). The combined organic phases were dried over MgSO₄. The crude product was purified by chromatography on silica gel, to give the desired product 82 (0.75 g, yield 86%). ¹H NMR data is consistent with the assigned structure.

### 83: 3-(4-Chloro-phenyl)-azetidin-3-ol hydrochloride Salt

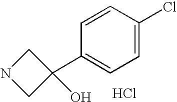

The title compound was prepared according to the experimental procedure for compound 79 and was obtained as a white solid (free base, yield 69%). ¹H NMR data is consistent with the assigned structure: MS (ESI⁺), M/Z, 184 (M+1), ret. time, 0.51 (HPLC system A).

### 84-1: 3-(4-Chloro-phenyl)-1-(3-phenoxy-benzyl)-azetidin-3-ol

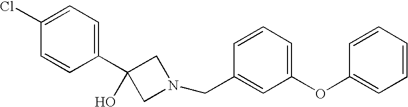

The title compound was prepared according to the general experimental procedure for 76 and was obtained as a white solid (free base, yield 94%). ¹H NMR data is consistent with the assigned structure: MS (ESI⁺), M/Z, 367 (M+1), ret. time, 1.81 (HPLC system A); Anal Calcd for C₂₂H₂₀ClNO₂; C, 72.23; H, 5.51; N, 3.83. Found: C, 71.98; H, 5.65; N, 3.74.

### 84-2: 3-(4-Chloro-phenyl)-1-[3-(2-methoxy-phenoxy)-benzyl]-azetidin-3-ol

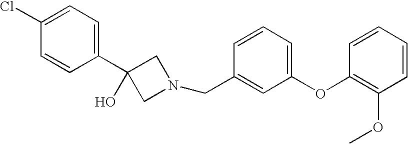

The title compound was prepared according to the general experimental procedure for 76 and was obtained as a white solid (free base, yield 65%). ¹H NMR data is consistent with the assigned structure: MS (ESI⁺), M/Z, 396 (M+1), ret. time, 1.78 (HPLC system A); Anal calcd for C₂₃H₂₂ClNO₃; C, 68.23; H, 5.73; N, 3.46. Found: C, 68.27; H, 5.52; N, 3.43.

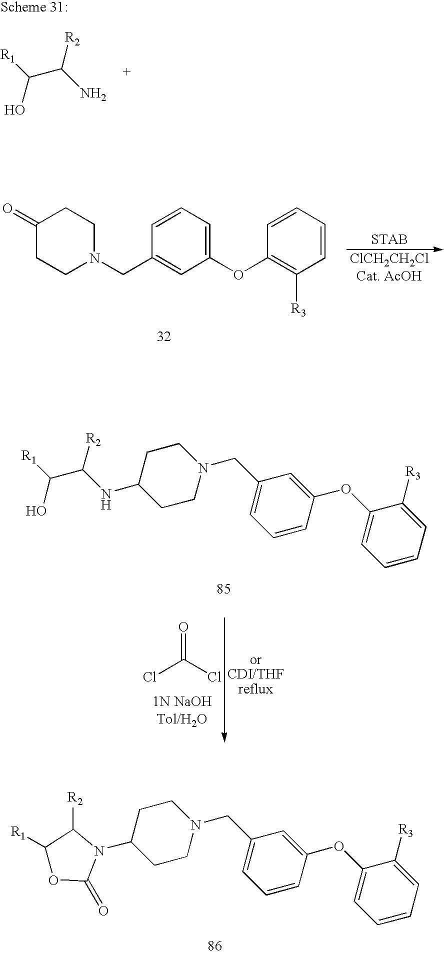

### 85-1: (1R,2S)-2-[1-(3-Phenoxy-benzyl)-piperidin-4-ylamino]-1-phenyl-propan-1-ol

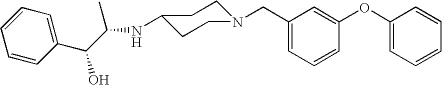

The title compound was prepared according to the general experimental procedure for 68 and was obtained as a white solid (yield 80%). ¹H NMR data is consistent with the assigned structure: MS (ESI⁺), M/Z, 417 (M+1), ret. time, 1.17 (HPLC system A); Anal Calcd for C₂₇H₃₂N₂O₂.2HCl.0.9H₂O; C, 64.13; H, 7.14; N, 5.54. Found: C, 64.21; H, 7.03; N, 5.53.

### 85-2: (1R,2S)-2-{1-[3-(2-Methoxy-phenoxy)-benzyl]-piperidin-4-ylamino}-1-phenyl-propan-1-ol

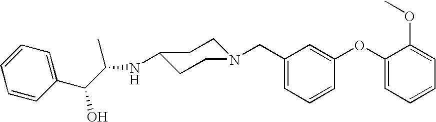

The title compound was prepared according to the general experimental procedure for 68 and was obtained as a white solid (yield 83%). ¹H NMR data is consistent with the assigned structure: MS (ESI⁺), M/Z, 447 (M+1), ret. time, 1.19 (HPLC system A); Anal Calcd for C₂₈H₃₄N₂O₃.2HCl.1.5H₂O; C, 61.53; H, 7.19; N, 5.13. Found: C, 61.66; H, 7.05; N, 5.13.

### 85-3: N-{1-[3-(2-Methoxy-phenoxy)-benzyl]-piperidin-4-yl}-N′-phenyl-ethane-1,2-diamine

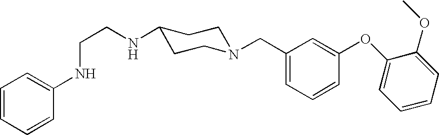

The title compound was prepared according to the general experimental procedure for 68 and was obtained as a white solid (yield 86%). ¹H NMR data is consistent with the assigned structure: MS (ESI⁺), M/Z, 432 (M+1), ret. time, 1.26 (HPLC system A); Anal calcd for C₂₇H₃₃N₃O₂.3HCl.1H₂O; C, 58.02; H, 6.85; N, 7.52. Found: C, 57.95; H, 6.70; N, 7.40.

### 85-4: (2S)-2-{1-[3-(2-Methoxy-phenoxy)-benzyl]-piperidin-4-ylamino}-3-phenyl-propan-1-ol (85-4)

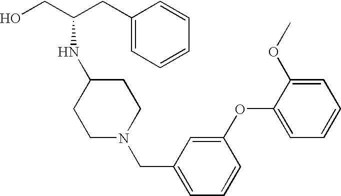

The title compound was prepared according to the general experimental procedure for 68 and was obtained as a white solid in a 77% yield. ¹H NMR data is consistent with the assigned structure: MS (ESI⁺), M/Z, 447 (M+1), ret. time, 1.30 (HPLC system A); Anal calcd for C₂₈H₃₄N₂O₃.2HCl.1.5H₂O; C, 61.53; H, 7.19; N, 5.13. Found: C, 61.49; H, 6.91; N, 5.09.

### 85-5: (2S)-2-[1-(3-Phenoxy-benzyl)-piperidin-4-ylamino]-3-phenyl-propan-1-ol

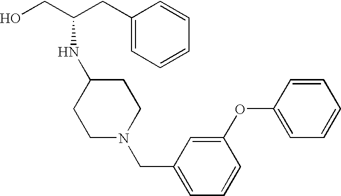

The title compound was prepared according to the general experimental procedure for 68 and was obtained as a white solid in a 84% yield. ¹H NMR data is consistent with the assigned structure: MS (ESI⁺), M/Z, 417 (M+1), ret. time, 1.33 (HPLC system A); Anal Calcd for C₂₇H₃₂N₂O₂.2HCl.0.6H₂O; C, 64.82; H, 7.09; N, 5.60. Found: C, 64.79; H, 7.07; N, 5.62.

### 86: 1-(3-phenoxy-benzyl)-4-substituted Piperidines

Procedure A:

To a solution of amino alcohol or diamine 85 in toluene (1 mL per 0.1 mmol amino alcohol), 1N NaOH solution (0.5 mL per 0.1 mmol amino alcohol) was added. The mixture was cooled to 0° C. Phosgene (1 mL per 0.2 mmol amino alcohol, 20 wt % in toluene) was added. The final mixture was stirred at 0° C. for 1.5 h. Then sat. NaHCO₃ solution (5 mL) was added to quench the reaction. The phases were separated. The aqueous phase was extracted with EtOAc (3×10 mL). The combined organic phases were dried over MgSO₄ and concentrated. The crude product was purified on silica gel, eluting with EtOAc/hexane (1/4 to 1/2), to give the desired product 86 (yield 35-75%).

Procedure B:

To a solution of amino alcohol 85 (1 eq) in anhydrous THF (10 mL per mmol HQ), 1,1′-carbonyldimidazole (3 eq) was added. The reaction was stirred at RT or reflux for overnight. Then sat NaHCO₃ solution (10 mL) was added to quench the reaction. The phases were separated. The aqueous phase was extracted with EtOAc (3×10 mL). The combined organic phases were dried over MgSO₄ and concentrated. The crude product was purified by chromatography on silica gel to give the desired product 86. The HCl salt was prepared by treating a solution of free base in Et₂O with 1M HCl solution in Et₂O.

### 86-1: (1R,2S)-4-Methyl-3-[1-(3-phenoxy-benzyl)-piperidin-4-yl]-5-phenyl-oxazolidin-2-one

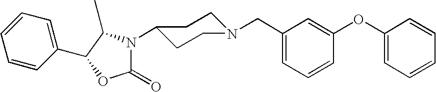

The title compound was prepared according to the general experimental procedure A and was obtained as a white solid in a 37% yield. ¹H NMR data is consistent with the assigned structure: MS (ESI⁺), M/Z, 443 (+1), ret. time, 1.90 (HPLC system A); Anal Calcd for C₂₈H₃₀N₂O₃.HCl; C, 70.21; H, 6.52; N, 5.85. Found: C, 70.01; H, 6.72; N, 5.90.

### 86-2: (1R,2S)-3-{1-[3-(2-Methoxy-phenoxy)-benzyl]-piperidin-4-yl}-4-methyl-5-phenyl-oxazolidin-2-one

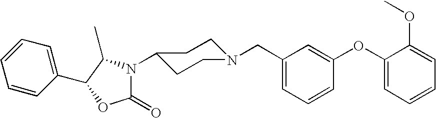

The title compound was prepared according to the general experimental procedure A and was obtained as a white solid in a 25% yield. ¹H NMR data is consistent with the assigned structure: MS (ESI⁺), M/Z, 473 (M+1), ret. time, 1.92 (HPLC system A); Anal Calcd for C₂₉H₃₂N₂O₄.HCl.0.4H₂O; C, 67.47; H, 6.60; N, 5.43. Found: C, 67.63; H, 6.49; N, 5.32.

### 86-3: 1-{1-[3-(2-Methoxy-phenoxy)-benzyl]-piperidin-4-yl}-3-phenyl-imidazolidin-2-one

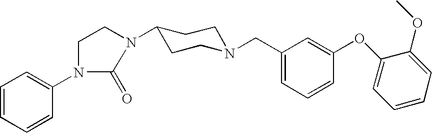

The title compound was prepared according to the general experimental procedure A and was obtained as a white solid in a 43% yield. ¹H NMR data is consistent with the assigned structure: MS (ESI⁺), M/Z, 458 (M+1), ret. time, 1.90 (HPLC system A); Anal Calcd for C₂₈H₃₁N₃O₃.HCl.1.75H₂O; C, 66.26; H, 6.65; N, 8.28. Found: C, 66.44; H, 6.51; N, 8.13.

### 86-4: (4S)-4-Benzyl-3-[1-(3-phenoxy-benzyl)-piperidin-4-yl]-oxazolidin-2-one

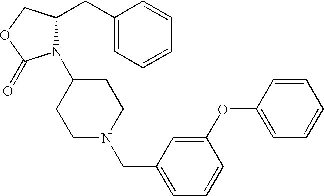

The title compound was prepared according to the general experimental procedure B and was obtained as a white solid in a 93% yield. ¹H NMR data is consistent with the assigned structure: MS (ESI⁺), M/Z, 443 (M+1), ret. time, 1.90 (HPLC system A); Anal Calcd for C₂₈H₃₀N₂O₃.HCl.H₂O; C, 67.06; H, 6.73; N, 5.59. Found: C, 67.18; H, 6.41; N, 5.52.

### 86-5: (4S)-4-Benzyl-3-{1-[3-(2-methoxy-phenoxy)-benzyl]-piperidin-4-yl}-oxazolidin-2-one

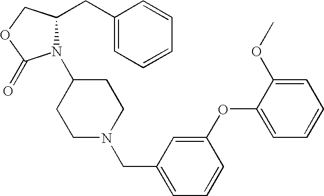

The title compound was prepared according to the general experimental procedure B and was obtained as a white solid in a 67% yield. ¹H NMR data is consistent with the assigned structure: MS (ESI⁺), M/Z, 474 (M+1), ret. time, 1.87 (HPLC system A); Anal Calcd for C₂₉H₃₂N₂O₄.HCl.H₂O; C, 66.09; H, 6.69; N, 5.32. Found: C, 66.14; H, 6.72; N, 5.13.

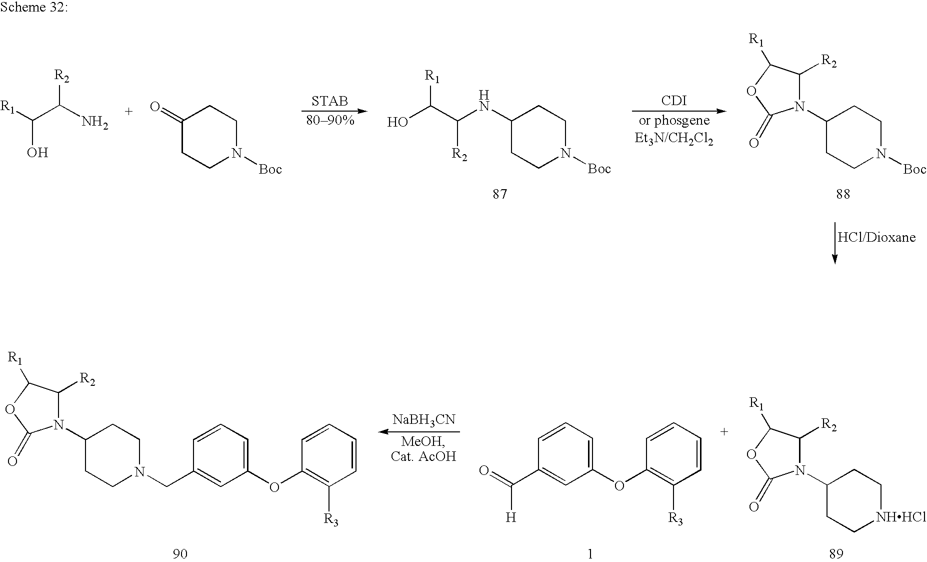

### 87-1: 4-[(1R)-2-Hydroxy-1-phenyl-ethylamino)-piperidine-1-carboxylic Acid Tert-Butyl Ester

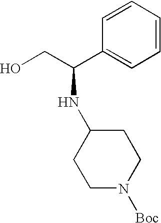

The title compound was prepared according to the general experimental procedure for 68 and was obtained as a white solid in a 88% yield. ¹H NMR data is consistent with the assigned structure: MS (ESI⁺), M/Z, 321 (M+1), ret. time, 1.38 (HPLC system A); Anal Calcd for C₁₈H₂₈N₂O₃; C, 67.47; H, 8.81; N, 8.74. Found: C, 67.42; H, 8.82; N, 8.61.

### 87-2: 4-[(1S)-2-Hydroxy-1-phenyl-ethylamino]-piperidine-1-carboxylic Acid Tert-Butyl Ester

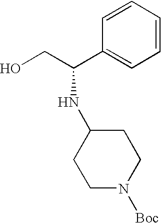

The title compound was prepared according to the general experimental procedure for 68 and was obtained as a white solid in a 94% yield. ¹H NMR data is consistent with the assigned structure: MS (ESI⁺), M/Z, 321 (M+1), ret. time, 1.40 (HPLC system A); Anal calcd for C₁₈H₂₈N₂O₃.0.1H₂O; C, 67.09; H, 8.82; N, 8.69. Found: C, 66.99; H, 8.82; N, 8.60.

### 87-3: 4-(2-Hydroxy-2-phenyl-ethylamino)-piperidine-1-carboxylic Acid Tert-Butyl Ester

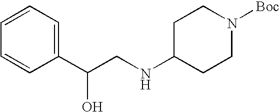

The title compound was prepared according to the general experimental procedure for 68 and was obtained as a white solid in a 80% yield. ¹H NMR data is consistent with the assigned structure: MS (ESI⁺), M/Z, 321 (M+1), ret. time, 1.41 (HPLC system A); Anal Calcd for C₁₈H₂₈N₂O₃.3H₂O; C, 66.35; H, 8.85; N, 8.60. Found: C, 66.37; H, 8.80; N, 8.51.

### 87-4: 4-(2-Hydroxy-1-methyl-ethylamino)-piperidine-1-carboxylic Acid Tert-Butyl Ester

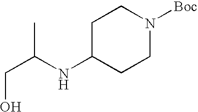

The title compound was prepared according to the general experimental procedure for 68 and was obtained as a white solid in a 88% yield. ¹H NMR data is consistent with the assigned structure: MS (ESI⁺), M/Z, 259 (M+1), ret. time, 1.03 (HPLC system A); Anal calcd for C₁₃H₂₆N₂O₃; C, 60.44; H, 10.14; N, 10.84. Found: C, 60.36; H, 10.15; N, 10.72.

### 88: 2-Oxo-oxazolidin-3-yl-piperidine-1-carboxylic Acid Tert-Butyl Esters

To a solution of amino alcohol 87 (1 eq) in anhydrous CH₂Cl₂ (2 mL per mmol 88), triethylamine (2 eq) and phosgene (2 eq 20 wt % in toluene) were added at 0° C. The reaction mixture was stirred at 0° C. for 2 h. Then sat. NaHCO₃ solution (10 mL) was added to quench the reaction. The phases were separated. The aqueous phase was extracted with EtOAc (3×10 mL). The combined organic phases were dried over MgSO₄. The crude product was purified by chromatography on silica gel, to give the desired product 88.

### 88-1: 4-[(4R)-2-Oxo-4-phenyl-oxazolidin-3-yl]-piperidine-1-carboxylic Acid Tert-Butyl Ester

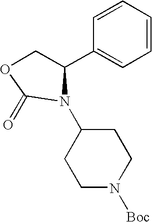

The title compound was prepared according to the general experimental procedure and was obtained as a colorless oil in a 65% yield. ¹H NMR data is consistent with the assigned structure: MS (ESI⁺), M/Z, 347 (M+1), ret. time, 2.53 (HPLC system A); Anal Calcd for C₁₉H₂₆N₂O₄; C, 65.88; H, 7.56; N, 8.09. Found: C, 65.90; H, 7.67; N, 8.02.

### 88-2: 4-[(4S)-2-Oxo-4-phenyl-oxazolidin-3-yl]-piperidine-1-carboxylic Acid Tert-Butyl Ester

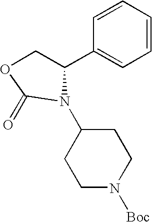

The title compound was prepared according to the general experimental procedure and was obtained as a white solid in a 51% yield. ¹H NMR data is consistent with the assigned structure: MS (ESI⁺), M/Z, 347 (M+1), ret. time, 2.55 (TLC system A); Anal calcd for C₁₉H₂₆N₂O₄; C, 65.88; H, 7.56; N, 8.09. Found: C, 65.97; H, 7.68; N, 8.08.

### 88-3: 4-(2-Oxo-5-phenyl-oxazolidin-3-yl)-piperidine-1-carboxylic Acid Tert-Butyl Ester

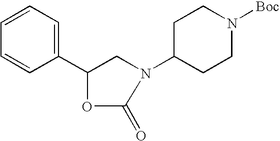

The title compound was prepared according to the general experimental procedure and was obtained as a colorless oil in a 81% yield. ¹H NMR data is consistent with the assigned structure: Anal calcd for C₁₉H₂₆N₂O₄; C, 65.88; H, 7.56; N, 8.09. Found: C, 65.90; H, 7.67; N, 8.02.

### 88-4: 4-(4-Methyl-2-oxo-oxazolidin-3-yl)-piperidine-1-carboxylic Acid Tert-Butyl Ester

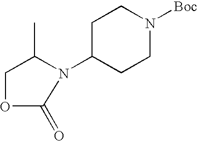

The title compound was prepared according to the general experimental procedure and was obtained as a colorless oil in a 81% yield. ¹H NMR data is consistent with the assigned structure.

### 89: 3-piperidin-4-yl-oxazolidin-2-one

To a solution of 4N HCl solution in dioxane (1 mL per mmol compound 88), the Boc-piperidone 88 was added. The mixture was stirred at RT for 1 h. After concentration, the white solid was washed with Et₂O (3×5 mL) to afford the desired hydrochloride salt 89.

### 89-1: (4R)-4-Phenyl-3-piperidin-4-yl-oxazolidin-2-one

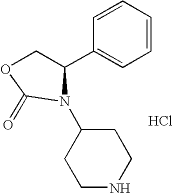

The title compound was prepared according to the general experimental procedure and was obtained as a white solid in a 100% yield. ¹H NMR data is consistent with the assigned structure: MS (ESI⁺), M/Z, 247 (M+1), ret. time, 0.92 (HPLC system A); Anal Calcd for C₁₄H₁₈N₂O₂.HCl.H₂O; C, 55.91; H, 7.04; N, 9.31. Found: C, 56.04; H, 6.97; N, 9.15.

### 89-2: (4S)-4-Phenyl-3-piperidin-4-yl-oxazolidin-2-one

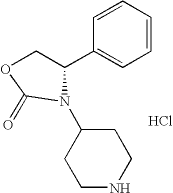

The title compound was prepared according to the general experimental procedure and was obtained as a white solid in a 90% yield. ¹H NMR data is consistent with the assigned structure: MS (ESI⁺), M/Z, 247 (M+1), ret. time, 0.87 (HPLC system A); Anal Calcd for C₁₄H₁₈N₂O₂.HCl.H₂O; C, 55.91; H, 7.04; N, 9.31. Found: C, 56.07; H, 6.84; N, 9.37.

### 89-3: 5-Phenyl-3-piperidin-4-yl-oxazolidin-2-one

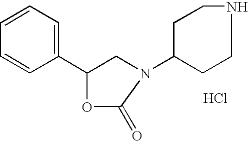

The title compound was prepared according to the general experimental procedure and was obtained as a white solid in a 95% yield. ¹H NMR data is consistent with the assigned structure: MS (ESI⁺), M/Z, 247 (M+1), ret. time, 0.92 (HPLC system A); Anal calcd for C₁₄H₁₈N₂O₂.HCl; C, 59.47; H, 6.77; N, 9.91. Found: C, 59.75; H, 6.88; N, 9.92.

### 89-4: 4-Methyl-3-piperidin-4-yl-oxazolidin-2-one

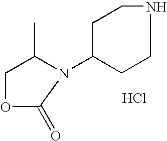

The title compound was prepared according to the general experimental procedure and was obtained as a white solid in a 98% yield. ¹H NMR data is consistent with the assigned structure: MS (ESI⁺), M/Z, 185 (M+1), ret. time, 0.21 (ion trace) (HPLC system A); Anal calcd for C₉H₁₆N₂O₂.HCl; C, 48.98; H, 7.76; N, 12.69. Found: C, 48.72; H, 7.75; N, 12.44.

### 90-1: (4R)-3-{1-[3-(2-Methoxy-phenoxy)-benzyl]-piperidin-4-yl}-4-phenyl-oxazolidin-2-one

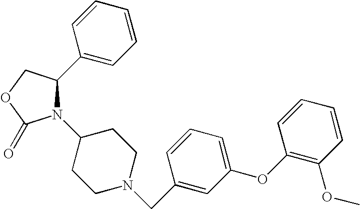

The title compound was prepared according to the general experimental procedure for 68 and was obtained as a white solid in a 33% yield. ¹H NMR data is consistent with the assigned structure: MS (ESI⁺), M/Z, 459 (M+1), ret. time, 1.64 (HPLC system A); Anal calcd for C₂₈H₃₀N₂O₄.HCl.CH₂Cl₂.4H₂O; C, 61.02; H, 5.93; N, 4.95. Found: C, 61.02; H, 5.95; N, 4.93.

### 90-2: (4R)-3-[1-(3-Phenoxy-benzyl)-piperidin-4-yl]-4-phenyl-oxazolidin-2-one

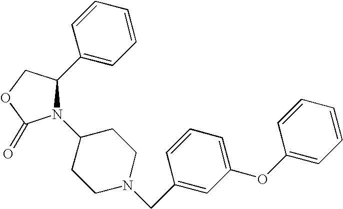

The title compound was prepared according to the general experimental procedure for 68 and was obtained as a white solid in a 47% yield. ¹H NMR data is consistent with the assigned structure: MS (ESI⁺), M/Z, 429 (M+1), ret. time, 1.72 (HPLC system A); Anal Calcd for C₂₇H₂₈N₂O₃.HCl.0.15CH₂Cl₂.0.75H₂O; C, 61.47; H, 5.95; N, 5.17. Found: C, 61.25; H, 5.92; N, 5.43.

### 90-3: (4R)-3-{1-[3-(2-Chloro-phenoxy)-benzyl]-piperidin-4-yl}-4-phenyl-oxazolidin-2-one

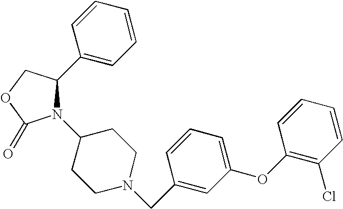

The title compound was prepared according to the general experimental procedure for 68 and was obtained as a white solid in a 44% yield. ¹H NMR data is consistent with the assigned structure: MS (ESI⁺), M/Z, 463 (M+1), ret. time, 1.78 (HPLC system A); Anal calcd for C₂₇H₂₇ClN₂O₃.HCl.0.75CH₂Cl₂.H₂O; C, 57.35; H, 5.46; N, 4.82. Found: C, 57.07; H, 5.44; N, 4.61.

### 90-4: (4S)-3-{1-[3-(2-Methoxy-phenoxy)-benzyl]-piperidin-4-yl}-4-phenyl-oxazolidin-2-one

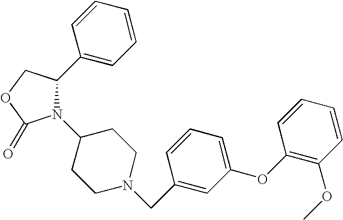

The title compound was prepared according to the general experimental procedure for 68 and was obtained as a white solid in a 72% yield. ¹H NMR data is consistent with the assigned structure: MS (ESI⁺), M/Z, 459 (M+1), ret. time, 1.79 (HPLC system A); Anal calcd for C₂₈H₃₀N₂O₄.HCl.1.5H₂O; C, 64.42; H, 6.56; N, 5.37. Found: C, 64.29; H, 6.42; N, 5.22.

### 90-5: (4S)-3-{1-[3-(2-Chloro-phenoxy)-benzyl]-piperidin-4-yl}-4-phenyl-oxazolidin-2-one

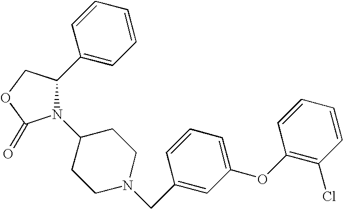

The title compound was prepared according to the general experimental procedure for 68 and was obtained as a white solid in a 46% yield. ¹H NMR data is consistent with the assigned structure: MS (ESI⁺), M/Z, 463 (M+1), ret. time, 1.84 (HPLC system A); Anal Calcd for C₂₇H₂₇ClN₂O₃.HCl.1.5H₂O; C, 61.60; H, 5.94; N, 5.32. Found: C, 61.84; H, 5.85; N, 5.21.

### 90-6: 3-{1-[3-(2-Methoxy-phenoxy)-benzyl]-piperidin-4-yl}-5-phenyl-oxazolidin-2-one

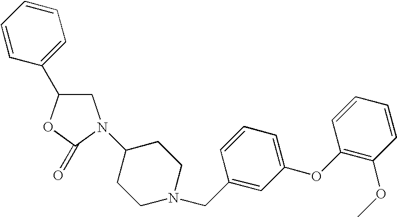

The title compound was prepared according to the general experimental procedure for 68 and was obtained as a white solid in a 50% yield. ¹H NMR data is consistent with the assigned structure: MS (ESI⁺), M/Z, 459 (M+1), ret. time, 1.74 (HPLC system A); Anal calcd for C₂₈H₃₀N₂O₄.HCl.0.25H₂O; C, 67.33; H, 6.36; N, 5.61. Found: C, 67.22; H, 6.43; N, 5.44.

### 90-7: 3-{1-[3-(2-Chloro-phenoxy)-benzyl]-piperidin-4-yl}-5-phenyl-oxazolidin-2-one

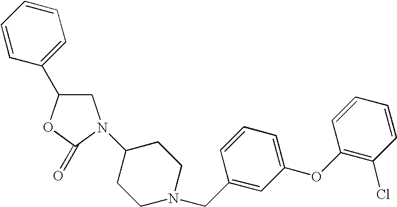

The title compound was prepared according to the general experimental procedure for 68 and was obtained as a white solid in a 37% yield. ¹H NMR data is consistent with the assigned structure: MS (ESI⁺), M/Z, 463 (+1), ret. time, 1.90 (HPLC system A); Anal Calcd for C₂₇H₂₇ClN₂O₃.HCl.H₂O; C, 62.67; H, 5.84; N, 5.41. Found: C, 62.95; H, 5.80; N, 5.30.

### 90-8: 3-{1-[3-(2-Methoxy-phenoxy)-benzyl]-piperidin-4-yl}-4-methyl-oxazolidin-2-one

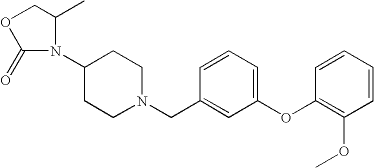

The title compound was prepared according to the general experimental procedure for 68 and was obtained as a white solid in a 59% yield. ¹H NMR data is consistent with the assigned structure: MS (ESI⁺), M/Z, 397 (M+1), ret. time, 1.48 (HPLC system A); Anal calcd for C₂₃H₂₈N₂O₄.HCl.1.5H₂O; C, 60.06; H, 7.01; N, 6.09. Found: C, 59.78; H, 6.70; N, 6.46.

### 92: 4-(4-Chloro-phenyl)-1-(3-phenoxy-benzyl)-piperidin-4-ol

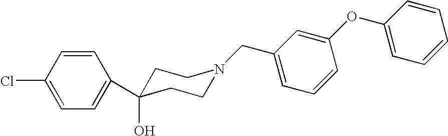

The title compound was prepared according to the general experimental procedure for 68 and was obtained as a white solid (free base, yield 86%). ¹H NMR data is consistent with the assigned structure: MS (ESI⁺), M/Z, 394 (M+1), ret. time, 1.94 (HPLC system A); Anal calcd for C₂₄H₂₄ClNO₂.2H₂O; C, 72.52; H, 6.19; N, 3.52. Found: C, 72.56; H, 6.37; N, 3.36.

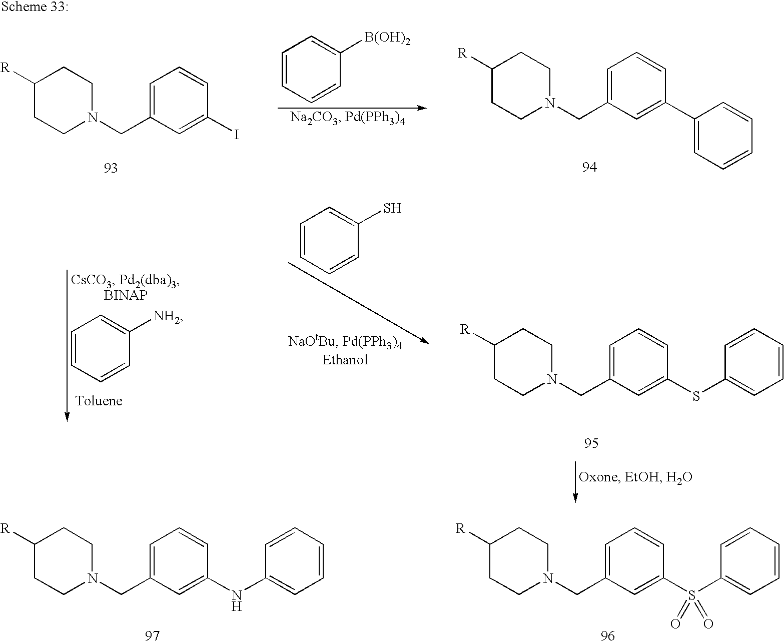

### 93: 1-(3-Iodo-benzyl)-piperdine-4-carboxylic Acid Ethyl Ester

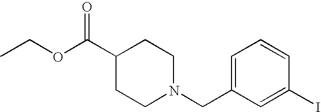

3-Iodobenzylbromide (1.3 equ) and ethylisonipecotate (1.0 equ.) were added together in acetonitrile, followed by 3.0 equ. of diisoproylethylamine. The reaction was allowed to stir at room temperature for 3 h. The reaction mixture was concentrated down and partitioned between 1N HCl and CH₂Cl₂. The aqueous layer was extracted 3× and washed with brine and dried over MgSO₄. The organics were filtered and concentrated down. The product was purified by flash chromatography with 2% MeOH/98% CH₂Cl₂ to give a 94% yield of the corresponding iodide 93-1.

### 95: 1-(3-Phenylsulfanyl-benzyl)-piperdine-4-carboxylic Acid Ethyl Ester

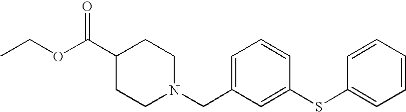

The aryl iodide (1.0 equ) 93-1 was added to ethanol, followed by tetrakistriphenylphospine palladium (0) (0.1 equ), 1.0 equ of benzene thiol and 1.0 equ of sodium tert-butoxide. The reaction was allowed to heat to reflux for 16 h and cooled to room temperature. The mixture was diluted with ether and water. The aqueous layer was extracted 3× with ether and washed with brine and dried over MgSO₄. The organics were filtered and concentrated down. The product was purified by flash chromatography with 4% MeOH/96% CH₂Cl₂ to give a 74% yield of 95-1. Retention time 3.46, LCMS 356.27, ¹H NMR data is consistent with the assigned structure.

### 97: 1-(3-Phenylamino-benzyl)-piperdine-4-carboxylic Acid Ethyl Ester

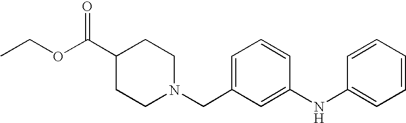

The aryl iodide (1.0 equ) 93-1 was added to aniline (1.2 equ), Pd₂(dba)₃ (0.05 equ), 0.03 equ of BINAP, 1.4 equ. of cesium carbonate in toluene. The reaction mixture was heated to 100° C. for 34 h and cooled to room temperature. The mixture was diluted with ether and filtered. The organics were concentrated down and chromatographed directly. The product was purified by flash chromatography with 2% MeOH/98% CH₂Cl₂ to give 97-1. Retention time 2.32, LCMS 341.23, ¹H NMR data is consistent with the assigned structure.

### 94-1:1-Biphenyl-3-ylmethyl-piperdine-4-carboxylic Acid Ethyl Ester

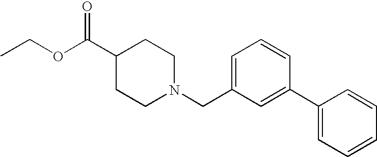

The aryl iodide (1.0 equ) 93-1 was dissolved in toluene (0.4M) and 0.05 equ of tetrakis triphenylphosphine palladium (0) was added and stirred for 10 min. To the above mixture was added 1.1 equ of phenyl boronic acid in ethanol (0.9 M) and a 2 M solution of sodium carbonate. The reaction mixture was refluxed for 14 h and cooled to room temperature. The reaction mixture was diluted with ethyl ether and water. The aqueous layer was seperated and extracted 2× with ether and washed with brine and dried over MgSO₄. The organics were filtered and concentrated down. The product was purified by flash chromatography with 2% MeOH/98% CH₂Cl₂ to give 96% of 94-1. Retention time 2.49, LCMS 324.30, ¹H NMR data is consistent with the assigned structure.

### 96-1:1-(3-Benzenesulfonyl-benzyl)-piperdine-4-carboxylic Acid Ethyl Ester

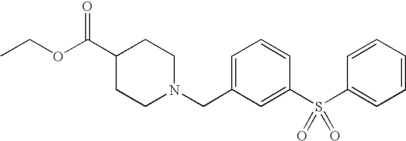

95-1 (1.0 equ.) was added to a solution of oxone in ethanol (2.0 mL) and 0.5 mL of water. This solution was stirred for 3 h at room temperature. The reaction was filtered and CH₂Cl₂ was added. The organic phase was collected and washed with water, brine and dried over MgSO₄, filtered and concentrated down. The product was purified by flash chromatography with 8% MeOH/92% CH₂Cl₂ to give 76% of 96-1. Retention time 2.70, LCMS 388.23, ¹H NMR data is consistent with the assigned structure.

### 93-2: 1-(3-Iodo-benzyl)-4-methyl Piperdine

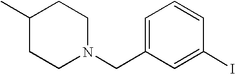

3-Iodobenzylbromide (1.3 equ) and 4-methylpiperdine (1.0 equ.) were added together in acetonitrile, followed by 3.0 equ. of diisoproylethylamine. The reaction was allowed to stir at room temperature for 3 h. The reaction mixture was concentrated down and partitioned between 1N HCl and CH₂Cl₂. The aqueous layer was extracted 3× and washed with brine and dried over MgSO₄. The organics were filtered and concentrated down. The product was purified with 2% MeOH/98% CH₂Cl₂ to give a 76% yield of the corresponding iodide 93-2. Retention time 2.77, LCMS 316.16, ¹H NMR data is consistent with the assigned structure.

### 95-2: 4-Methyl-1-(3-phenylsulfanyl-benzyl)-piperdine

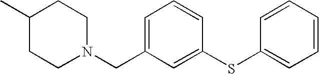

The aryl iodide (1.0 equ) 93-2 was added to ethanol, followed by tetrakistriphenylphospine palladium (0) (0.1 equ), 1.0 equ of benzene thiol and 1.0 equ of sodium tert-butoxide. The reaction was allowed to heat to reflux for 16 h and cooled to room temperature. The mixture was diluted with ether and water. The aqueous layer was extracted 3× with ether and washed with brine and dried over MgSO₄. The organics were filtered and concentrated down. The product was purified with 3% MeOH/97% CH₂Cl₂ to give a 72% yield of 95-2. Retention time 3.19, LCMS 298.23, ¹H NMR data is consistent with the assigned structure.

### 97-2: [3-(4-Methyl-piperidin-1-ylmethyl)-phenyl]-phenylamine

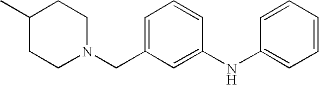

The aryl iodide (1.0 equ) 93-2 was added to aniline (1.2 equ), Pd₂(dba)₃ (0.05 equ), 0.03 equ of BINAP, 1.4 equ. of cesium carbonate in toluene. The reaction mixture was heated to 100° C. for 34 h and cooled to room temperature. The mixture was diluted with ether and filtered. The organics were concentrated down and chromatographed directly. The product was purified by flash chromatography with 2% MeOH/98% CH₂Cl₂ to give 97-2. Retention time 2.31, LCMS 281.2, ¹H NMR data is consistent with the assigned structure.

### 94-2: 1-Biphenyl-3-ylmethyl-4-methyl-piperdine

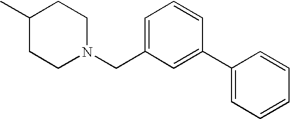

The aryl iodide (1.0 equ) 93-2 was added to 0.05 equ of tetrakis triphenylphosphine palladium (0) in toluene (0.4 M) and stirred for 10 min. To the above mixture was added 1.1 equ of phenyl boronic acid in ethanol (0.9 M) and a 2 M solution of sodium carbonate (4.7 equ.). The reaction mixture was refluxed for 14 h and cooled to room temperature. The reaction mixture was diluted with ethyl ether and water. The aqueous layer was seperated and extracted 2× with ether and washed with brine and dried over MgSO₄. The organics were filtered and concentrated down. The product was purified by flash chromatography with 2% MeOH/98% CH₂Cl₂ to give 85% yield of 94-2. Retention time 3.48, LCMS 266.21, ¹H NMR data is consistent with the assigned structure.

### 96-2: 1-(3-Benzenesulfonyl-benzyl)-4-methyl-piperdine

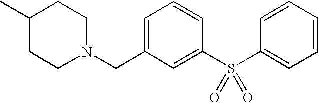

95-2 (1.0 equ.) was added to a solution of oxone in ethanol (2.0 mL) and 0.5 mL of water. This solution was stirred for 3 h at room temperature. The reaction was filtered and CH₂Cl₂ was added. The organic phase was collected and washed with water, brine and dried over MgSO₄, filtered and concentrated down. The product was purified by flash chromatography with 5% MeOH/95% CH₂Cl₂ to give 40% of 96-2. Retention time 2.31, LCMS 281.22, ¹H NMR data is consistent with the assigned structure.

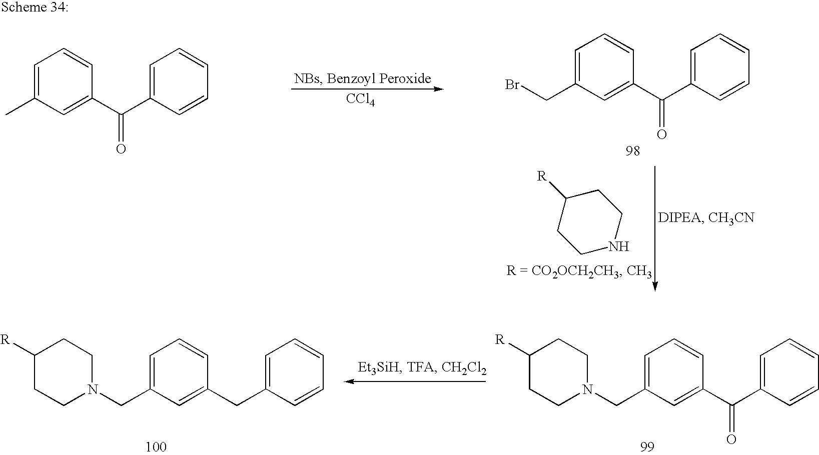

### 99-1:1-(3-Benzoyl-benzyl)-piperdine-4-carboxylic Acid Ethyl Ester

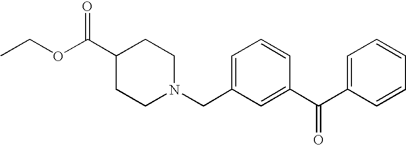

To a solution of 3-methylbenzophenone (1.0 equ.) in benzene was added 1.1 equ of NBS and 0.007 equ. of benzoyl peroxide. The reaction was heated to reflux for 5 h, during which time the mixture went from colorless to orange. The reaction mixture was cooled to room temperature and filtered. The filtrate was concentrated down to yield the benzylic bromide 98, which was used directly in the next reaction.

The bromide 98 was added to 4-isonipecotate piperdine (1.3 equ.) in acetonitrile (0.1M) along with 3.51 mL of DIPEA. The reaction mixture was allowed to stir at room temperature for 3 h and concentrated down. The product was purified by flash chromatography with 2% MeOH/98% CH₂Cl₂ to give 25% (2 steps) of 99-1. Retention time 2.94, LCMS 352.28, ¹H NMR data is consistent with the assigned structure.

### 100-1: 1-(3-Benzyl-benzyl)-piperdine-4-carboxylic Acid Ethyl Ester

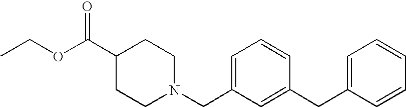

99-1 was added along with TFA at 0° C., 2.25 equ of triethylsilane was added dropwise, once the addition was complete the reaction was allowed to stir at room temperature for 18 h. Water was added to quench the reaction and stirred for 1 h, additionally methylene chloride was added. The organics were removed and washed with brine and dried over MgSO₄, filtered and concentrated down. The product was purified by flash chromatography with 4% MeOH/96% CH₂Cl₂ to give 86% of 100-1. Retention time 2.89, LCMS 338.26, ¹H NMR data is consistent with the assigned structure.

### 99-2: [3-(4-Methyl-piperdin-1-ylmethyl)-phenyl]-phenyl-methanone

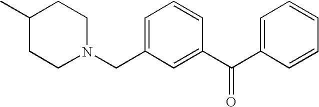

To a solution of 3-methylbenzophenone (1.0 equ.) in benzene was added 1.1 equ of NBS and 0.007 equ. of benzoyl peroxide. The reaction was heated to reflux for 5 h, during which time the mixture went from colorless to orange. The reaction mixture was cooled to room temperature and filtered. The filtrate was concentrated down to yield the benzylic bromide 98, which was used directly in the next reaction.

The bromide 98 was added with 4-methyl piperdine (1.3 equ.) in acetonitrile (0.1M) along with 3.51 mL of DIPEA. The reaction mixture was allowed to stir at room temperature for 3 h and concentrated down. The product was purified by flash chromatography with 2% MeOH/98% CH₂Cl₂ to give 50% (2 steps) of 99-2. Retention time 2.37, LCMS 294.21, ¹H NMR data is consistent with the assigned structure.

### 100-2: 1-(3-Benzyl-benzyl)-4-methyl-piperdine

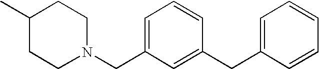

99-2 was added along with TFA (1.3M) at 0° C. To the above was added 2.25 equ of triethylsilane dropwise, once the addition was complete the reaction was allowed to stir at room temperature for 18 h. Water was added to quench the reaction and stirred for 1 h, additionally methylene chloride was added. The organics were removed and washed with brine and dried over MgSO₄, filtered and concentrated down. The product was purified by flash chromatography with 5% MeOH/95,% CH₂Cl₂ to give 68% of 100-2. Retention time 2.46, LCMS 280.23, ¹H NMR data is consistent with the assigned structure.

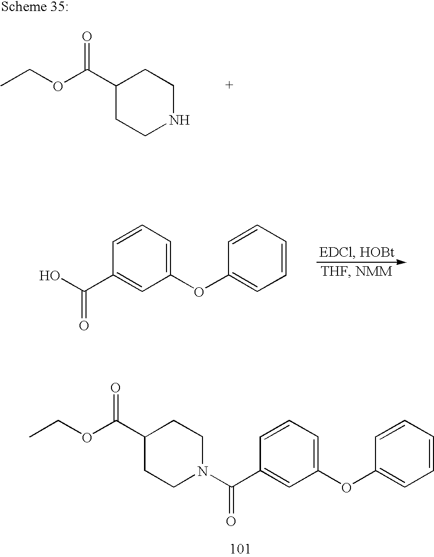

### 101: 1-[3-Phenoxy-benzoyl)-piperdin-4-carboxylic Acid Ethyl Ester

3-phenoxybenzoic acid (1.0 equ) and ethylisonipecotate (1.05 equ) were mixed with HOBt (1.5 equ.), EDCI (1.3 equ.) in THF with N-methyl morpholine (2.0 equ.). The reaction was allowed to stir at room temperature for 10 h. The reaction was diluted with ethyl acetate and washed with 1N HCl, 1N NaOH and brine. The organics were dried over Mg₂SO₄, filtered and concentrated down. The product was purified by flash chromatography with 1.5% MeOH/98.5% CH₂Cl₂ to give in 98% yield of 101 1-[3-phenoxy-benzoyl)-piperdin-4-carboxylic acid ethyl ester. Retention time 2.87, LCMS354.27, ¹H NMR data is consistent with the assigned structure.

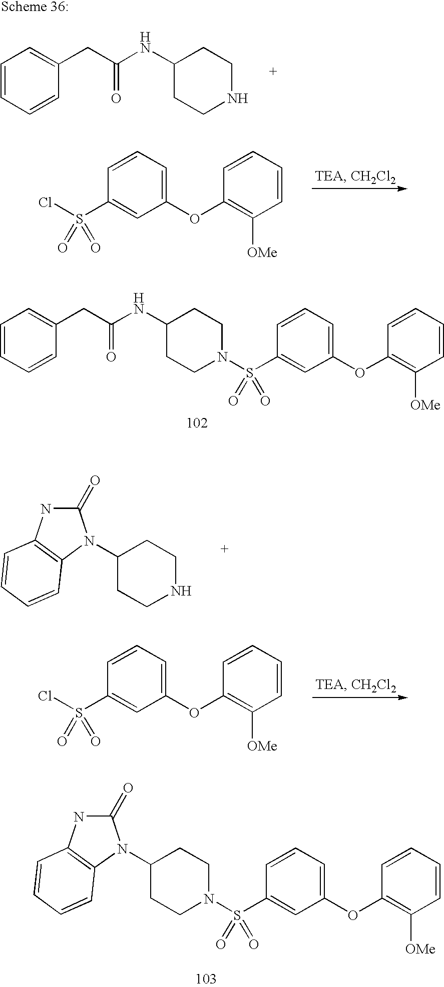

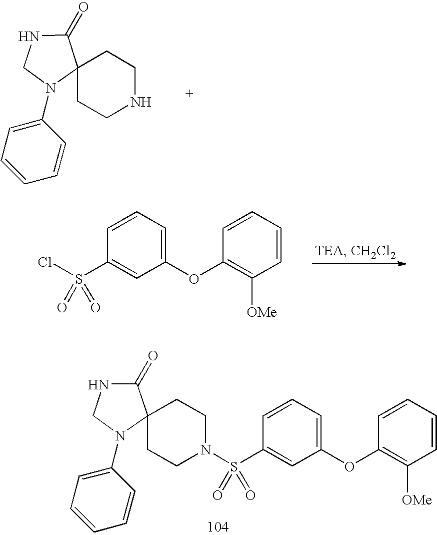

### 102: N-{1-[3(2-Methoxy-phenoxy)-benzenesulfonyl]-piperdin-4-yl}-2-phenyl-acetamide

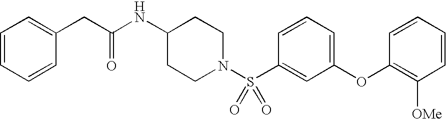

4-(phenylacetamide)piperdine (1.0 equ.), 3-(2-methoxyphenoxy)phenylsulfonyl chloride, and triethylamine (1.5 equ) were added together in CH₂Cl₂ and allowed to stir at room temperature for 3 h. The reaction was concentrated down and purified by flash chromatography with 100% CH₂Cl₂ to 4% MeOH/96% CH₂Cl₂ to give 45% of 102. Retention time 2.59, LCMS 480.96, ¹H NMR data is consistent with the assigned structure.

### 103: 1-{1-[3(2-Methoxy-phenoxy)-benzenesulfonyl]-piperdin-4-yl}-1,3-dihydro-benzoimidazol-2-one

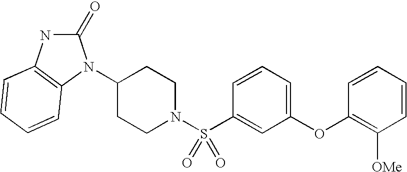

4-(2-keto-1-benzimidazole)piperdine (1.0 equ.), 3-(2-methoxyphenoxy)phenyl sulfonyl chloride, and triethylamine (1.5 equ) were added together in CH₂Cl₂ and allowed to stir at room temperature for 3 h. The reaction was concentrated down and purified by flash chromatography with 100% CH₂Cl₂ to 4% MeOH/96% CH₂Cl₂ to give 31% of 103. Retention time 2.51, LCMS 479.94, ¹H NMR data is consistent with the assigned structure.

### 104: 8-[3(2-Methoxy-phenoxy)-benzenesulfonyl]-1-phenyl-1,3,8-triaza-spiro[4.5]decan-4-one

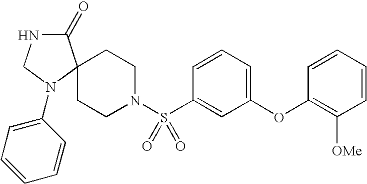

1-phenyl-1,3,8-triazaspiro[4,5]-decan-4-one (1.0 equ.), 3-(2-methoxyphenoxy)phenylsulfonyl chloride, and triethylamine (1.5 equ) were added together in CH₂Cl₂ and allowed to stir at room temperature for 3 h. The reaction was concentrated down and purified by flash chromatography with 100% CH₂Cl₂ to 4% MeOH/96% CH₂Cl₂ to give 45% of 104. Retention time 2.72, LCMS 493.96, ¹H NMR data is consistent with the assigned structure.

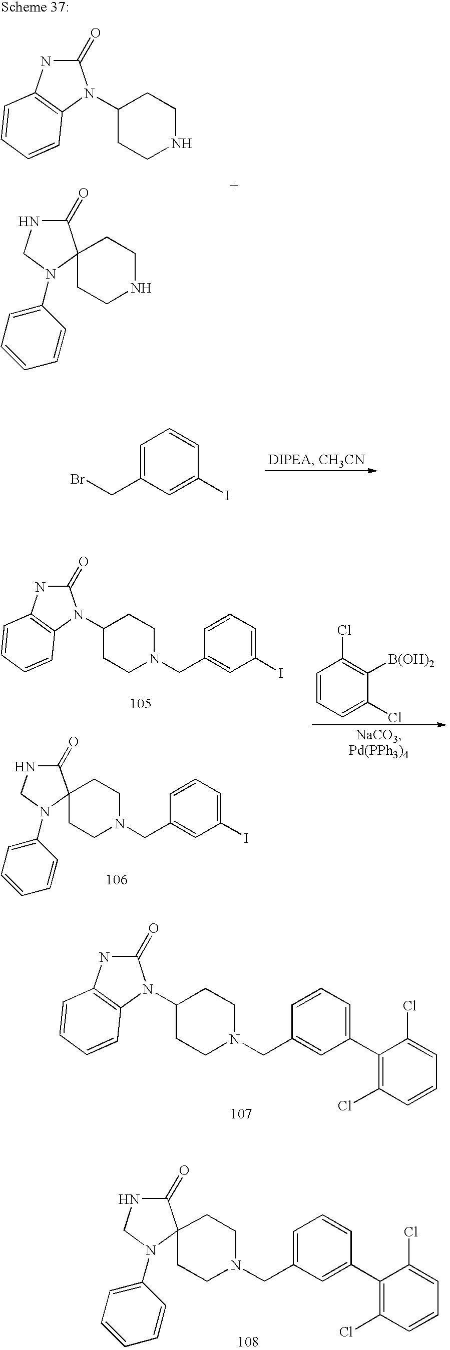

### 105: 1-[1-(3-Iodo-benzyl)-piperdin-4-yl]-1,3-dihydro-benzimidazol-2-one

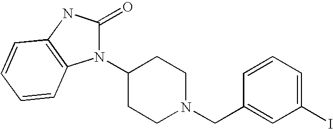

3-Iodobenzylbromide (1.0 equ) and 4-(2-keto-1-benzimidazole)piperdine (1.3 equ.) were added together in acetonitrile, followed by 3.0 equ. of diisoproylethylamine. The reaction was allowed to stir at room temperature for 3 h. The reaction mixture was concentrated down and partitioned between 1N HCl and CH₂Cl₂. The aqueous layer was extracted 3× and washed with brine and dried over MgSO₄. The organics were filtered and concentrated down. The product was purified by flash chromatography with 2% MeOH/98% CH₂Cl₂ to give the corresponding iodide 105.

### 106: 8-(3-Iodo-benzyl)-1-phenyl-1,3,8-triaza-spiro[4.5]-decan-4-one

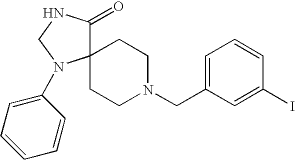

3-Iodobenzylbromide (1.0 equ) and 1-phenyl-1,3,8-triazaspiro[4,5]-decan-4-one (1.3 equ.) were added together in acetonitrile, followed by 3.0 equ. of diisoproylethylamine. The reaction was allowed to stir at room temperature for 3 h. The reaction mixture was concentrated down and partitioned between 1N HCl and CH₂Cl₂. The aqueous layer was extracted 3× and washed with brine and dried over MgSO₄. The organics were filtered and concentrated down. The product was purified by flash chromatography with 2% MeOH/98% CH₂Cl₂ to give the corresponding iodide 106.

### 107: 1-[1-(2′,6′-Dichloro-biphenyl-3-ylmethyl)-piperdin-4-yl]-1,3-dihydro-benzoimidazol-2-one

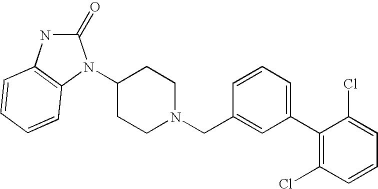

The aryl iodide (1.0 equ) 105 was added to 0.05 equ of tetrakis triphenylphosphine palladium (0) in toluene (0.4 M) and stirred for 10 min. To the above mixture was added 1.1 equ of 2,6-dichlorophenyl boronic acid in ethanol (0.9 M) and a 2 M solution of sodium carbonate (4.7 equ.). The reaction mixture was refluxed for 14 h and cooled to room temperature. The reaction mixture was diluted with ethyl ether and water. The aqueous layer was seperated and extracted 2× with ether and washed with brine and dried over MgSO₄. The organics were filtered and concentrated down. The product was purified by flash chromatography with 100% EtOAc to give 23% yield of 107. Retention time 1.68, LCMS 452.01, ¹H NMR data is consistent with the assigned structure.

### 108: 8-(2′,6′-Dichloro-biphenyl-3-ylmethyl)-1-phenyl-1,3,8-triaza-spiro[4.5]decan-4-one

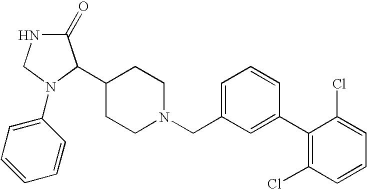

The aryl iodide (1.0 equ) 106 was added to 0.05 equ of tetrakistriphenylphosphine palladium (0) in toluene (0.4 M) and stirred for 10 min. To the above mixture was added 1.1 equ of 2,6-dichlorophenyl boronic acid in ethanol (0.9 M) and a 2 M solution of sodium carbonate (4.7 equ.). The reaction mixture was refluxed for 14 h and cooled to room temperature. The reaction mixture was diluted with ethyl ether and water. The aqueous layer was seperated and extracted 2× with ether and washed with brine and dried over MgSO₄. The organics were filtered and concentrated down. The product was purified by flash chromatography with 100% EtOAc to give 23% yield of 108. Retention time 1.76, LCMS 465.97, ¹H NMR data is consistent with the assigned structure.

Experimental II: CCR8 Binding Protocol

L1.2-CCR8 cells are stable recombinant L1.2 cells overexpressing the CCR8 receptor. The cells were routinely cultured and passaged in RPMI based medium. The incubators were set at 37° C., 6% CO₂ and 90% relative humidity. The density of the cell suspension was maintained around 0.7 to 1.0 million cells per ml. Cells were removed from the culture after about 2 months and replaced with freshly thawed cells of lower passage number. On Day 1, the cells were split to be approximately 0.5 millions/ml for next day assay by dilution into fresh RPMI medium in the morning. N-butyric acid (500 mM) to a final concentration of 5 mM was added into the cell suspension (1:100 dilution) in late afternoon. On Day 2, the cells were harvested by spinning down the cells for 5 minutes (1350 rpm) in a table top centrifuge, and the cells were washed with 35 ml of assay binding buffer once, and then re-suspend the cells into the binding buffer at 2 millions cells per ml of the buffer.

A 10-point dose-response curve (final concentrations are 100 μM, 33.3 μM, 11.1 μM, 3.70 μM, 0.411 μM, 0.137 μM, 0.0457 μM, 0.0152 μM, 0.00508 μM) was prepared by diluting a 20 mM solution of the compounds 1:2 (6 μL into 6 μL DMSO) and then serially diluting the sample 1:3 (4 μL into 8 μL DMSO). To prepare a screen for the compounds (at 10 μM and 1 μM), a 20 mM solution of the compounds was diluted 1:20 (1 μL into 19 μL DMSO). The sample was then subsequently diluted 1:10 dilution (2 μL into 18 μL of DMSO).

To prepare the compound plate, 1 μL from each of the above DMSO solutions was transferred into each well of a polypropylene 96-well plate for the following binding experiment. 1 μL of DMSO was stamped into each well of the blank control. 50 μL of the L1.2-CCR8 cell suspension (2 million cells/mL) was added into each well of the compound plate (100,000 cells/well), and pipette up and down three times to mix. Then 1 mL of the 10 μM cold I-309 solution was added into control wells, A11, B11, C11 and D11 as non-specific control. The cells were incubated with the compounds for 40 min. at room temperature. Then 50 μL of 0.2 nM¹²⁵ I-I-309 solution was added into each well of the above plate. The radioligand was added to the mixture of cells and compounds and incubated at room temperature for one hour. 100 μL of 0.33% PEI solution was added into each well of the filter plate (GF/B), and incubated for about half an hour at room temperature. The samples were harvested using Packard cell harvester, the plates were washed with 4 wells of cold assay wash buffer, the harvester was opened, and the plate was dried under vacuum for about 30 seconds. The filter plate was then air-dried overnight, the plates were bottom-sealed, 500 μL MicroScint-20 fluid was added to each well, and the top of the plate sealed using Topseal. The plate was read on the Topcount.

All patents, literature citations and publications disclosed within the application are herein incorporated by reference.

While this invention has been particularly shown and described with references to preferred embodiments thereof, it will be understood by those skilled in the art that various changes in form and details may be made therein without departing from the scope of the invention encompassed by the appended claims.

1. A compound having the formula: 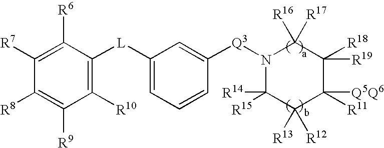
or physiologically acceptable salt thereof; wherein
L is selected from the group consisting of a O, S, NR^{a}, a bond, SO₂, —C(═O), and (CR′R″)ₘ;
R^{a} is selected from the group consisting of hydrogen, optionally substituted alkyl, optionally substituted alkylaryl, and optionally substituted cycloalkyl;
a is 0 to 3;
b is 0 to 3;
m is 1 to 8;
R′ and R″ are independently selected from the group consisting of hydrogen, optionally substituted alkyl, cyano and optionally substituted alkenyl;
R⁶, R⁷, R⁸, R⁹ and R¹⁰ are independently selected from the group consisting of hydrogen, hydroxyl, halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂NR¹R², trifluoromethyl, aryl, aralkyl, heteroaryl and heteroaralkyl;
R¹ and R² are independently selected from the group consisting of hydrogen and optionally substituted alkyl;
Q³ is optionally substituted alkyl;
R¹¹, R¹², R¹³, R¹⁴, R¹⁵, R¹⁶, R¹⁷, R¹⁸ and R¹⁹ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂NR⁴¹R⁴², trifluoromethyl, aryl, aralkyl, heteroaryl and heteroaralkyl;
R⁴¹ and R⁴² are each independently selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, optionally substituted amino, trifluoromethyl, aryl, aralkyl, heteroaryl and heteroaralkyl; or R⁴¹ and R⁴² may be linked via a C₂-C₈ optionally substituted alkyl or alkenyl bridge where one or more carbons may be replaced by O, S or NR⁴⁶;
Q⁵ is selected from the group consisting of 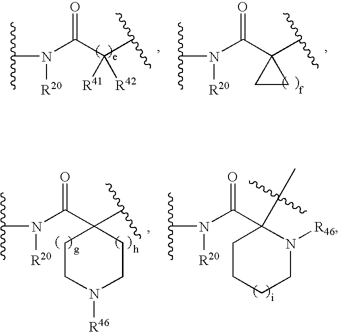
—CH₂—, and a bond;
e is 1 to 3;
f is 1 to 7;
g is 0 to 3;
h is 0 to 3;
i is 0 or 1;
R²⁰ and R⁴⁶ are independently hydrogen, hydroxyl, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido, —C(═O)O(R⁴¹), —C(═O)(R⁴¹), —SO₂NR⁴¹R⁴², trifluoromethyl, aryl, aralkyl, heteroaryl or heteroaralkyl; and
Q⁶ is selected from the group consisting of optionally substituted aromatic ring, optionally substituted non-aromatic heterocycle, and optionally substituted heteroaromatic ring; or
R¹⁸ or R¹⁹ together with Q⁵Q⁶ and the atoms to which they are bonded form an optionally substituted non-aromatic carbocyclic group, optionally substituted non-aromatic heterocyclic group, optionally substituted aryl ring or optionally substituted heteroaryl ring;
with the proviso that the compound is not

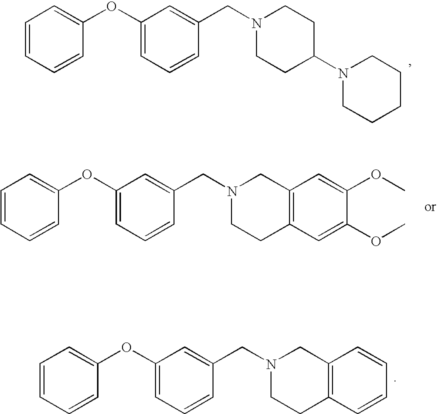

2. The compound according to claim 1 wherein
L is O;
R¹⁸ and R¹⁹ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂NR⁴¹R⁴², trifluoromethyl, aryl, aralkyl, heteroaryl and heteroaralkyl;
Q⁵ is selected from the group consisting of 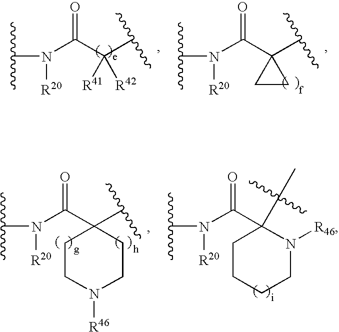
—CH₂, and a bond;
Q⁶ is selected from the group consisting of optionally substituted aromatic ring, optionally substituted non-aromatic heterocycle, and optionally substituted heteroaromatic ring.

3. The compound according to claim 2 wherein R⁶ is selected from the group consisting of halogen, hydrogen and C₁-C₁₀ alkoxy; and R⁷, R⁸, R⁹ and R¹⁰ are hydrogen.

4. The compound according to claim 3 wherein R⁶ is selected from the group consisting of halogen and C₁-C₁₀ alkoxy, wherein said halogen is chloro and said C₁-C₁₀ alkoxy is methoxy.

5. The compound according to claim 3 wherein Q³ is —CH₂—.

6. The compound according to claim 2 wherein R¹¹ is selected from the group consisting of hydrogen, —COOH and —C(O)OR⁴¹; wherein
R⁴¹ is hydrogen, optionally substituted alkyl, optionally substituted alkenyl or optionally substituted alkynyl.

7. The compound according to claim 6 wherein
a is 1;
bis 1; and
R¹², R¹³, R¹⁴, R⁵, R¹⁶, R¹⁷, R¹⁸ and R¹⁹ are hydrogen.

8. The compound according to claim 2 wherein Q⁵ is selected from the group consisting of

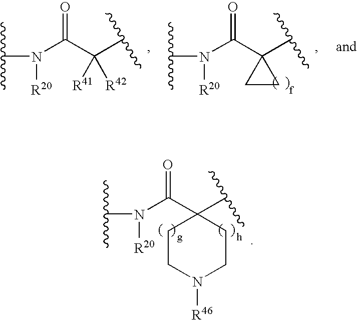

9. The compound according to claim 8 wherein Q5 is 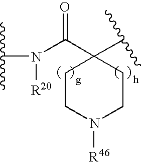
wherein g is 1, and h is 1.

10. The compound according to claim 8 wherein Q⁵ is 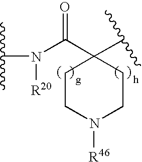
g is 1 and h is 0; such that Q⁵ has a formula selected from the group consisting of

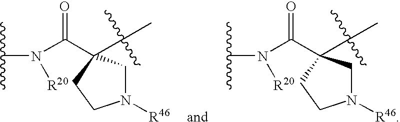

11. The compound according to claim 9 wherein R⁴⁶ is selected from the group consisting of hydrogen, optionally substituted alkyl, —C(═O)OR⁴¹, —SO2NR⁴¹R⁴², —C(═O)R⁴¹ and 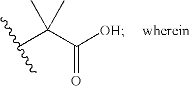
R⁴¹ is optionally substited alkyl; and
R⁴² is selected from the group consisiting of hydrogen and optionally substited alkyl.

12. The compound according to claim 8 wherein
R⁶ is selected from the group consisting of hydrogen, halogen and C₁-C₁₀ alkoxy;
R⁷, R⁸R⁹, R¹, R¹², R¹³, R¹⁴, R¹⁵, R¹⁶, R¹⁷, R¹⁸ and R¹⁹ are hydrogen;
R¹¹ is selected from the group consisting of hydrogen and —C(═O)OR⁴¹;
Q³ is —CH₂—;
a is 0 or 1;
b is 1; and
R⁴¹ is selected from the group consisiting of hydrogen and optionally substited alkyl.

13. The compound according to claim 12 wherein Q⁶ is optionally substituted aromatic ring.

14. The compound according to claim 2 wherein Q⁶ is selected from the group consisting of 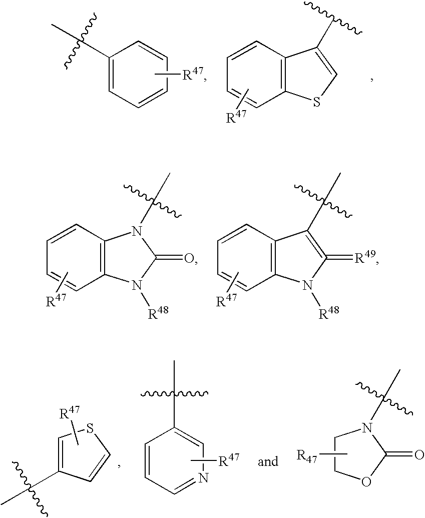
R⁴⁷ is independently selected for each position capable of substitution from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(41), —SO₂NR⁴¹R⁴², trifluoromethyl, aryl, aralkyl, heteroaryl and heteroaralkyl;
R⁴⁸ is selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, —C(═O)O(R⁴¹), —C(═O)(R⁴¹), trifluoromethyl, aryl, aralkyl, heteroaryl and heteroaralkyl; and
R⁴⁹ is selected from the group consisting of hydrogen, hydroxyl, optionally substituted alkyl, optionally substituted alkenyl, optionally substituted alkynyl, optionally substituted cycloalkyl, optionally cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), SO₂NR⁴¹R⁴², trifluoromethyl, aryl, aralkyl, heteroaryl and heteroaralkyl.

15. The compound according to claim 10 wherein Q⁶ is

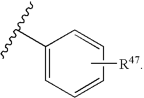

16. The compound according to claim 15 wherein R⁴⁷ is independently chosen for each position capable of substitution from the group consisting of halogen and hydrogen.

17. The compound according to claim 3 wherein a is one, b is one, and Q⁵ is selected from the group consisting of 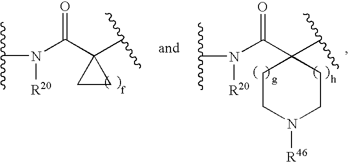
wherein R²⁰ is hydrogen and the compound has a formula selected from the group consisting of

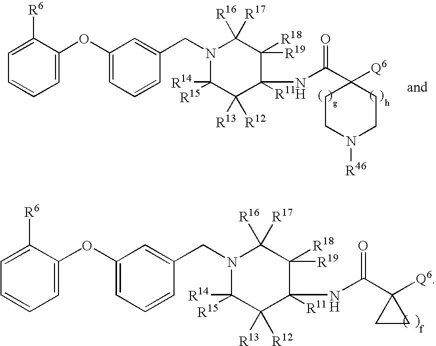

18. The compound according to claim 17 wherein the compound has the formula 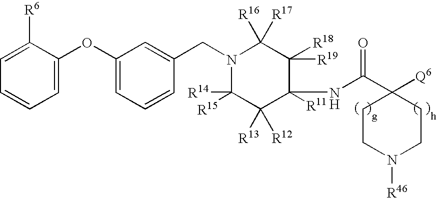
wherein g is one, and h is zero; such that the compound has a formula selected from the group consisting of

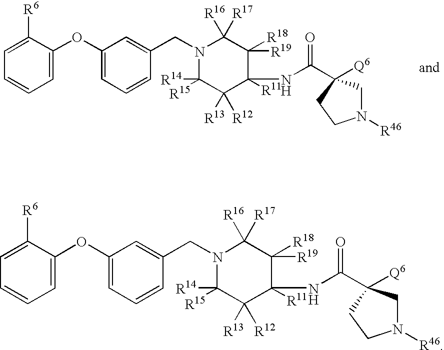

19. The compound according to claim 18 wherein
R⁵ is chloro or methoxy;
R¹¹, R¹², R¹³, R¹⁴, R¹⁵, R¹⁶, R¹⁷, R¹⁸ and R¹⁹ are hydrogen;
Q⁶ phenyl; and
R⁴⁶ is hydrogen, optionally substituted alkyl, —C(═O)OR⁴¹, —SO₂NR⁴¹R⁴², —C(═O)R⁴¹ and 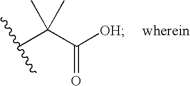
R⁴¹ is optionally substited alkyl; and
R⁴² is selected from the group consisiting of hydrogen and optionally substited alkyl.

20. The compound according to claim 2 wherein
R¹¹ is —OH;
Q⁵ is a bond; and
Q⁶ is an optionally substituted aromatic ring.

21. The compound according to claim 20 wherein
R¹², R¹³, R¹⁴, R¹⁵, R¹⁶, R¹⁷, R¹⁸ and R¹⁹ are hydrogen; and
Q⁶ is 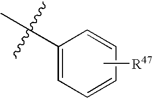
R⁴⁷ is independently selected for each position capable of substitution from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂NR⁴¹R⁴², trifluoromethyl, aryl, aralkyl, heteroaryl and heteroaralkyl.

22. The compound according to claim 20 wherein
R¹² and R¹³ are methyl;
R¹⁸ and R¹⁹ are hydrogen; and
R⁴⁷ is halogen or heteroaryl; wherein said heteroaryl is tetrazolyl.

23. A method of treating an inflammatory disorder or viral disorder comprising administering to a subject in need thereof an effective amount of a compound having the formula: 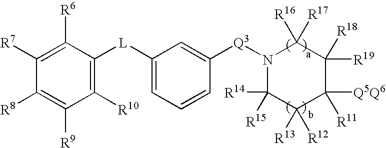
or physiologically acceptable salt thereof; wherein
L is selected from the group consisting of a O, S, NR^{a}, a bond, SO₂, —C(═O), and (CR′R″)ₘ;
R^{a} is selected from the group consisting of hydrogen, optionally substituted alkyl, optionally substituted alkylaryl, and optionally substituted cycloalkyl;
a is 0 to 3;
b is 0 to 3;
m is 1 to 8;
R′ and R″ are independently selected from the group consisting of hydrogen, optionally substituted alkyl, cyano and optionally substituted alkenyl;
R⁶, R⁷, R⁸, R⁹ and R¹⁰ are independently selected from the group consisting of hydrogen, hydroxyl, halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂NR¹R², trifluoromethyl, aryl, aralkyl, heteroaryl and heteroaralkyl;
R¹ and R² are independently selected from the group consisting of hydrogen and optionally substituted alkyl;
Q³ is optionally substituted alkyl;
R¹¹, R¹², R¹³, R¹⁴, R¹⁵, R¹⁶, R¹⁷, R¹⁸ and R¹⁹ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂NR⁴¹R⁴², trifluoromethyl, aryl, aralkyl, heteroaryl and heteroaralkyl;
R⁴¹ and R⁴² are each independently selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, optionally substituted amino, trifluoromethyl, aryl, aralkyl, heteroaryl and heteroaralkyl; or R⁴¹ and R⁴² may be linked via a C₂-C₈ optionally substituted alkyl or alkenyl bridge where one or more carbons may be replaced by O, S or NR⁴⁶;
Q⁵ is selected from the group consisting of 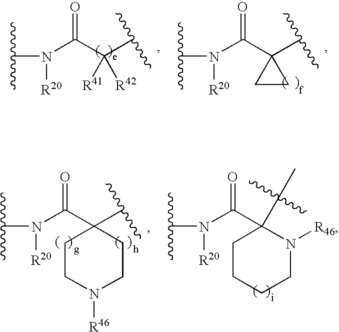
—CH₂—, and a bond;
e is 1 to 3;
f is 1 to 7;
g is 0 to 3;
h is 0 to 3;
R⁴⁶ is hydrogen, hydroxyl, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido, —C(═O)O(R⁴¹), —C(═O)(R⁴¹), —SO₂NR⁴¹R⁴², trifluoromethyl, aryl, aralkyl, heteroaryl or heteroaralkyl; and
Q⁶ is selected from the group consisting of optionally substituted aromatic ring, optionally substituted non-aromatic heterocycle, and optionally substituted heteroaromatic ring; or
R¹⁸ or R¹⁹ together with Q⁵Q⁶ and the atoms to which they are bonded form an optionally substituted non-aromatic carbocyclic group, optionally substituted non-aromatic heterocyclic group, optionally substituted aryl ring or optionally substituted heteroaryl ring.

24. A compound having the formula: 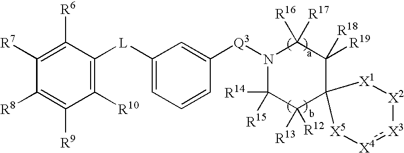
or physiologically acceptable salt thereof; wherein
L is selected from the group consisting of a O, S, NR^{a}, a bond, SO₂, —C(═O)—, and (CR′R″)ₘ;
R^{a} is selected from the group consisting of hydrogen, optionally substituted alkyl, optionally substituted alkylaryl, and optionally substituted cycloalkyl;
a is 0 to 3;
b is 0 to 3;
m is 1 to 8;
R′ and R″ are independently selected from the group consisting of hydrogen, optionally substituted alkyl, cyano and optionally substituted alkenyl;
R⁶, R⁷, R⁸, R⁹ and R¹⁰ are independently selected from the group consisting of hydrogen, hydroxyl, halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂NR¹R², trifluoromethyl, aryl, aralkyl, heteroaryl and heteroaralkyl;
R¹ and R² are independently selected from the group consisting of hydrogen and optionally substituted alkyl;
X¹ is selected from the group consisting of CR²⁶R²⁷, NR²⁸, —C(—O)—, O and a bond;
X² is selected from the group consisting of CR²⁹R³⁰, NR³¹, —C(═O)— and O;
X³ is selected from the group consisting of CR³²R³³, —C(R³²)═, NR³⁴, —N═, —C(═O)— and O;
X⁴ is selected from the group consisting of CR³⁵R³⁶, NR³⁷, ═N—, —C(═O)— and O;
X⁵ is selected from the group consisting of CR³⁸R³⁹, NR⁴⁰, —C(═O)— and O;
R¹², R¹³, R¹⁴, R¹⁵, R¹⁶, R¹⁷, R¹⁸, R¹⁹, R²⁶, R²⁷, R²⁹, R³⁰, R³², R³³, R³⁵, R³⁶, R³⁸ and R³⁹ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)(R⁴¹), —SO₂NR⁴¹R⁴², trifluoromethyl, aryl, aralkyl, heteroaryl and heteroaralkyl;
R²⁸, R³¹, R³⁴, R³⁷ and R⁴⁰ are independently selected from the group consisting of hydrogen, hydroxyl, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido, —C(═O)O(R⁴¹), —C(═O)(R⁴¹), —SO₂NR⁴¹R⁴², trifluoromethyl, aryl, aralkyl, heteroaryl or heteroaralkyl;
with the proviso that when X⁴ is CR³⁵R³⁶ and X³ is CR³²R³³ or X⁵ is CR³⁸R³⁹, R³⁵ and R³⁸ or R³² and R³⁵ optionally form a non-aromatic carbocyclic group, a non-aromatic heterocyclic group, aryl ring or heteroaryl ring;
R⁴¹ and R⁴² are each independently selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, optionally substituted amino, trifluoromethyl, aryl, aralkyl, heteroaryl and heteroaralkyl; or R⁴¹ and R⁴² may be linked via a C₂-C₈ optionally substituted alkyl or alkenyl bridge where one or more carbons may be replaced by O, S or NR⁴⁶;
R⁴⁶ is hydrogen, hydroxyl, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido, —C(O)O(R⁴¹), —C(═O)(R⁴¹), —SO₂NR⁴¹R⁴², trifluoromethyl, aryl, aralkyl, heteroaryl or heteroaralkyl; and
Q³ is optionally substituted alkyl.

25. The compound according to claim 24 wherein L is O.

26. The compound according to claim 25 wherein R⁶ is selected from the group consisting of halogen, hydrogen and C₁-C₁₀ alkoxy; and R⁷, R⁸, R⁹ and R¹⁰ are hydrogen.

27. The compound according to claim 26 wherein R⁶ is selected from the group consisting of halogen and C₁-C₁₀ alkoxy, wherein said halogen is chloro and said C₁-C₁₀ alkoxy is methoxy.

28. The compound according to claim 27 wherein Q³ is —CH₂—.

29. The compound according to claim 28 wherein
a is 1;
b is 1; and
R¹², R¹³, R¹⁴, R¹⁵, R¹⁶, R¹⁷, R¹⁸ and R¹⁹ are hydrogen.

30. The compound according to claim 29 wherein
X¹ is a bond;
X² is NR³¹;
X³ is —C(R³²)═;
X⁴ is ═N—; and
X⁵ is —C(═O)—.

31. A method of treating an inflammatory disorder or viral disorder comprising administering to a subject in need thereof an effective amount of a compound according to claim 24.

32. A compound having the formula: 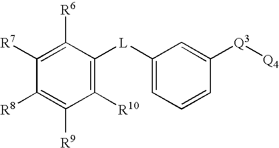
or physiologically acceptable salt thereof; wherein
L is selected from the group consisting of a O, S, NR^{a}, a bond, SO₂, —C(═O)— and (CR′R″)ₘ;
R^{a} is selected from the group consisting of hydrogen, optionally substituted alkyl, optionally substituted alkylaryl, and optionally substituted cycloalkyl;
m is 1 to 8;
R′ and R″ are independently selected from the group consisting of hydrogen, optionally substituted alkyl, cyano and optionally substituted alkenyl;
R⁶ is selected from the group consisting of halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂NR², trifluoromethyl, aryl, aralkyl, heteroaryl and heteroaralk
R⁷, R⁸, R⁹ and R¹⁰ are independently selected from the group consisting of hydrogen, hydroxyl, halogen, optionally substituted C₁-C₁₀ alkyl, optionally substituted C₂-C₁₀ alkenyl, optionally substituted C₂-C₁₀ alkynyl, optionally substituted C₃-C₁₀ cycloalkyl, optionally substituted C₃-C₁₀ cycloalkenyl, optionally substituted C₃-C₁₀ cycloalkynyl, optionally substituted C₃-C₁₀ cycloalkoxy, cyano, C₁-C₁₀ alkoxy, C₂-C₁₀ alkenyloxy, C₂-C₁₀ alkynyloxy, benzyloxy, optionally substituted amino, optionally substituted amido, —O(CF₃), —C(═O)O(R¹), —C(═O)(R¹), —SO₂NR¹R², trifluoromethyl, aryl, aralkyl, heteroaryl and heteroaralkyl;
R¹ and R² are independently selected from the group consisting of hydrogen and optionally substituted alkyl;
Q³ is optionally substituted alkyl;
Q⁴ is selected from the group consisting of 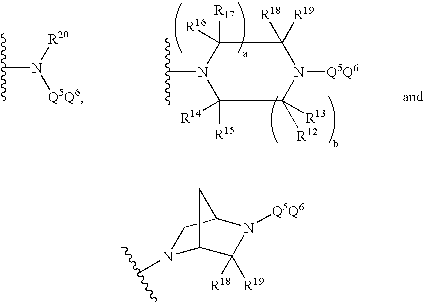
a is 0 to 3;
b is 0 to 3;
R¹², R¹³, R¹⁴, R¹⁵, R¹⁶, R¹⁷, R¹⁸, R¹⁹ and R²⁰ are each independently selected from the group consisting of hydrogen, hydroxyl, halogen, optionally substituted alkyl, optionally substituted alkenyl, optionally substituted alkynyl, optionally substituted cycloalkyl, optionally substituted cycloalkenyl, optionally substituted cycloalkynyl, cyano, alkoxy, alkenyloxy, alkynyloxy, benzyloxy, optionally substituted amino, optionally substituted amido, —O(CF₃), —C(═O)O(R⁴¹), —C(═O)R⁴¹, —SO₂C(═O)R⁴¹, SO₂, SO₂NR⁴¹R⁴², trifluoromethyl, aryl, aralkyl, heteroaryl and heteroaralkyl;
Q⁵ is selected from the group consisting of a bond, —C(R⁴¹R⁴²)_{d}—C(═O)—NR⁴³—, —C(═O)—C(R⁴¹R⁴²)_{d}—C(═O)—C(R⁴¹R⁴²)_{d}—, —(CR⁴¹R⁴²)_{d}—C(═O)—, —C(═O)—NR⁴⁴, —C(═S)—NR⁴⁴—, —C(═O)—CH═CH—, —C(R⁴¹R⁴²)_{d}—, —C(═O)—CH═CH—CH₂—, —C(═O)—CH═CH—, —C(R⁴¹R⁴²)_{d}—NR⁴³—, —SO₂(CR⁴¹R⁴²)_{d}—, —C(═O)—(CR⁴¹R⁴²)_{d}—, —C(═O)—(CR⁴¹R⁴²)_{d}—, —C(═O)—(CR⁴¹R⁴²)_{d}—O—, or is absent;
Q⁶ is selected from the group consisting of optionally substituted aromatic ring, optionally substituted non-aromatic heterocycle, and optionally substituted heteroaromatic ring; or
R¹⁸ or R¹⁹ together with Q⁵Q⁶ and the atoms to which they are bonded form an optionally substituted non-aromatic carbocyclic group, optionally substituted non-aromatic heterocyclic group, optionally substituted aryl ring or optionally substituted heteroaryl ring;
d is 0, 1, 2, 3, 4, 5, 6, 7 or 8;
R⁴¹ and R⁴² are each independently selected from the group consisting of hydrogen, optionally substituted alkyl, optionally substituted alkenyl, optionally substituted alkynyl, optionally substituted cycloalkyl, optionally substituted cycloalkenyl, optionally substituted cycloalkynyl, optionally substituted amino, trifluoromethyl, aryl, aralkyl, heteroaryl and heteroaralkyl; or R⁴¹ and R⁴² may be linked via a C₂-C₈ optionally substituted alkyl or alkenyl bridge where one or more carbons may be replaced by O, S or NR⁴⁶;
R⁴³, R⁴⁴ and R⁴⁶ are independently selected from the group consisting of hydrogen, hydroxyl, optionally substituted alkyl, optionally substituted alkenyl, optionally substituted alkynyl, optionally substituted cycloalkyl, optionally cycloalkenyl, optionally substituted cycloalkynyl, optionally substituted amino, optionally substituted amido, —C(═O)O(R⁴¹), —C(═O)(R⁴¹), —SO₂NR⁴¹R⁴², trifluoromethyl, aryl, aralkyl, heteroaryl or heteroaralky.

33. A method of treating an inflammatory disorder or viral disorder comprising administering to a subject in need thereof an effective amount of a compound according to claim 32.
